(19) United States
(12) Patent Application Publication
(10) Pub. No.: US 2023/0279056 A1
(43) Pub. Date: Sep. 7, 2023

(54) MODULAR AND GENERALIZABLE BIOSENSOR PLATFORM BASED ON DE NOVO DESIGNED PROTEIN SWITCHES

(71) Applicant: University of Washington, Seattle, WA (US)

(72) Inventors: Alfredo QUIJANO RUBIO, Seattle, WA (US); Jooyoung PARK, Seattle, WA (US); Hsien-Wei YEH, Seattle, WA (US); David BAKER, Seattle, WA (US); Longxing CAO, Seattle, WA (US); Brian COVENTRY, Seattle, WA (US); Inna GORESHNIK, Seattle, WA (US); Lisa KOZODOY, Seattle, WA (US); Lance Joseph STEWART, Seattle, WA (US)

(21) Appl. No.: 17/999,524

(22) PCT Filed: May 25, 2021
(86) PCT No.: PCT/US2021/034104

§ 371 Date: Nov. 21, 2022

Related U.S. Application Data
(60) Provisional application No. 63/067,643, filed on Aug. 19, 2020.
(60) Provisional application No. 63/051,549, filed on Jul. 14, 2020.
(60) Provisional application No. 63/030,836, filed on May 27, 2020.

(51) Int. Cl. C07K 14/00; G01N 33/542

(52) U.S. Cl. CPC C07K 14/001; G01N 33/542

(57) ABSTRACT

The disclosure provides cage proteins comprising a helical bundle, wherein the cage protein comprises a structural region and a latch region, wherein the latch region comprises one or more target binding polypeptide, wherein the cage protein further comprises a first reporter protein domain, wherein the first reporter protein domain undergoes a detectable change in reporting activity when bound to a second split reporter protein domain, and wherein the structural region interacts with the latch region to prevent solution access to the one or more target binding polypeptide.

### CROSS REFERENCE

This application claims priority to U.S. Provisional Pat. Application Serial Nos. 63/030,836 filed May 27, 2020; 63/051,549 filed Jul. 14, 2020 and 63/067,643 filed Aug. 19, 2020, each incorporated by reference herein in its entirety.

### FEDERAL FUNDING STATEMENT

This invention was made with government support under Grant no. FA8750-17-C-0219 awarded by the Defense Advanced Research Project Agency (DARPA). The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on May 25, 2021 having the file name “20-1075-WO_Sequence-Listing_ST25.txt” and is 32,910 kb in size.

### BACKGROUND

Sensor proteins have emerged as an active area of research. Traditional ELISA methods require multiple liquid-handling steps, preventing its use at the bedside. Lateral flow immunochromatographic assays are fast and cheap, but they have limited sensitivity, reproducibility, and poor quantitative performance. ELISA and lateral flow also require two binding modules for the target being sensed, one for capture and the other for readout. One main hurdle of protein sensor construction is finding analyte binding domains that undergo sufficient conformational changes. The most commonly used binding domains (e.g., antibodies) undergo only minor structural changes of the loops upon ligand binding. Coupling an appropriate reporter with optimal geometry to amplify the conformational change is also key to a successful biosensor. However, computationally designing small molecule binding sites into protein interfaces and generating semisynthetic protein sensors are both quite challenging problems currently. Therefore, generalized approaches for designing biosensors with a simple and robust computational protocol empirical optimization are needed.

### SUMMARY

In one aspect, the disclosure provides cage proteins comprising a helical bundle, wherein the cage protein comprises a structural region and a latch region, wherein the latch region comprises one or more target binding polypeptide, wherein the cage protein further comprises a first reporter protein domain, wherein the first reporter protein domain undergoes a detectable change in reporting activity when bound to a second split reporter protein domain, and wherein the structural region interacts with the latch region to prevent solution access to the one or more target binding polypeptide. In one embodiment, the cage protein further comprises the second reporter protein domain, wherein one of the first reporter protein domain and the second reporter domain is present in the latch region and the other is present in the structural region, wherein an interaction of the first reporter protein domain and the second reporter protein domain is diminished in the presence of target to which the one or more target binding polypeptide binds. In another embodiment, the second reporter protein domain is not present in the cage protein. In another embodiment, the first reporter protein domain, and the second reporter domain when present, comprise a reporter protein domain selected from the group consisting of luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease). In one embodiment, the one or more target binding polypeptide is capable of binding to a target including but not limited to an antibody, a toxin, a diagnostic biomarker, a viral particle, a disease biomarker, a metabolite or a biochemical analyte.

In another aspect, the disclosure provides key proteins capable of binding to the structural region of a cage protein of any embodiment of the disclosure that does not include the second reporter protein domain, wherein binding of the key protein to the cage protein only occurs in the presence of a target to which the cage protein one or more target binding polypeptide can bind, wherein the key protein comprises a second repc wherein interaction of the key protein second reporter protein domain and the cage protein first reporter protein domain causes a detectable change in reporting activity from the first reporter protein domain . In various embodiments, the second reporter protein domain comprises a reporter protein domain selected from the group consisting of luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease).

In another aspect, the disclosure provides biosensors, comprising
(a) the cage protein of embodiment of the disclosure wherein the cage does not include the second reporter protein domain; and
(b) the key protein of any embodiment of the disclosure;
wherein the key protein can only bind to the cage protein in the presence of a target to which the cage protein one or more target binding polypeptide can bind; and wherein binding of the first reporter protein domain of the cage protein to the second reporter protein domain of the key protein causes a detectable change in reporting activity from the first reporter protein domain.

In a further aspect, the disclosure provides methods for detecting a target, comprising
(a) contacting the cage protein of any embodiment of the disclosure where the cage protein comprises the second reporter protein domain, or the biosensor of any embodiment of the disclosure with a biological sample under conditions to promote binding of the cage protein one or more target binding polypeptide to a target present in the biological sample, causing a detectable change in reporting activity from the first reporter protein domain; and
(b) detecting the change in reporting activity from the first reporter protein domain, wherein the change in reporting activity identifies the sample as containing the target.

In further aspects, the disclosure provides methods for designing a biosensor, cage protein, or key protein comprising the steps of any method described herein, nucleic acids encoding the cage protein or key protein of any embodiment of the disclosure vectors comprising the nucleic acid of embodiment of the disclosure operatively linked to a suitable control element, such as a promoter, cells (such as recombinant cells) comprising the cage protein, key protein, composition, nucleic acid, or expression vector of any embodiment of the disclosure, pharmaceutical compositions comprising the cage protein, key protein, composition, nucleic acid, expression vector, or cell of any embodiment of the disclosure, and a pharmaceutically acceptable carrier, an epitope comprising or consisting of the amino acid sequence of SEQ ID NO: 27384, and methods detecting Troponin I in a sample, comprising contacting a biological sample with the epitope under conditions suitable to promote binding of Troponin I in the sample to the epitope to form a binding complex, and detecting binding complexes that demonstrate presence of Troponin I in the sample.

### FIGURE LEGENDS

FIGS. 1(a-f). De novo design of multi state allosteric biosensors. a, Sensor schematic. The biosensor consists of two protein components: lucCage and lucKey, which exist in a closed (Off) and open state (On). The closed form of lucCage (left) cannot bind to lucKey, thus, preventing the split luciferase SmBit fragment from interacting with LgBit. The open form (right) can bind both target and key, and allows SmBit to combine with LgBit on lucKey to reconstitute luciferase activity. b, Thermodynamics of biosensor activation. The free energy cost ΔGₒₚₑₙ of the transition from closed cage (species 1) to open cage (species 2) disfavors association of key (species 5) and reconstitution of luciferase activity (species 6) in the absence of target. In the presence of the target, the combined free energies of target binding (2→3; ΔG_{LT}), key binding (3→4; ΔG_{CK}), and SmBit-LgBit association (4→7; ΔG_{R}) overcome the unfavorable ΔGₒₚₑₙ, driving opening of the lucCage and reconstitution of luciferase activity. c, Biosensor design strategy based on thermodynamics. For each biosensor, the designable parameters are ΔGₒₚₑₙ and ΔG_{CK}; ΔG_{R} is the same for all targets, and ΔG_{LT} is pre-specified for each target. For sensitive but low background analyte detection, ΔGₒₚₑₙ and ΔG_{CK} must be designed such that the closed state (species 1) is substantially lower in free energy than the open state (species 6) in the absence of target, but higher in free energy than the open state in the presence of target (species 7). d-f, Numerical simulations of the coupled equilibria shown in b for different values of (d) Kₒₚₑₙ, (e) K_{LT}, and (f) [lucKey]ₜₒₜ and [lucCage]ₜₒₜ. Kₒₚₑₙ, K_{LT}, K_{CK} were set to 1 × 10⁻³, 1 nM, and 10 nM respectively, and the concentration of the sensor components to 10: 100 nM (lucCage:lucKey) except where explicitly indicated. d, Increasing ΔGₒₚₑₙ shifts response to higher anal The sensor limit of detection is approximately 0.1 × K_{LT}; the driving force for opening the switch becomes too weak below this concentration. f, The effective target detection range can be tuned by changing the sensor component concentrations.

FIGS. 2(a-d). Design and characterization of de novo biosensors incorporating small proteins as sensing domains. a, General strategy and structural validation for caging small protein domains into LOCKR switches. Left: design model of the de novo binder HB 1.9549.2 bound to the stem region of influenza hemagglutinin (HA, ribbon representation) ¹⁵. Right: crystal structure of sCageHA_267_1S, comprising HB 1.9549.2 grafted into a shortened and stabilized version of the LOCKR switch (sCage, ribbon representation). Middle: All residues of HB1.9549.2 involved in binding to HA (top) except for F273 are buried in the closed state of the switch (bottom) to block its interaction. The labels indicate the same set of amino acids in the two panels (F2 in the top panel corresponds to F273 in the lower panel). b-d, Functional characterization of 3 allosteric biosensors: lucCageBot (detection of botulinum neurotoxin B (BoNT/B)), lucCageProA (detection of Fc domain), and lucCageHer2 (detection of Her2 receptor). Left: structural models of the indicated biosensors (ribbon representation) incorporating a de novo designed binder for BoNT/B (Bot.671.2), the C domain of the generic antibody binding protein Protein A (SpaC) and a Her2-binding affibody respectively, grafted into lucCage comprising a caged SmBiT fragment. Middle: kinetic measurement of luminescence intensity upon addition of 50 nM of analyte (BoNT/B, IgG Fc, or Her2) to a mixture of 10 nM of each lucCage and 10 nM of lucKey. Right: detection over a wide range of analyte concentrations by changing the biosensor concentration (50, 5 and 1 nM lucCage and lucKey; cyan, magenta and black lines respectively).

FIGS. 3(a-h). Design and characterization of biosensors for cardiac troponin I and for an anti-HBV antibody. a, Design of lucCageTrop, a sensor for cardiac Troponin I. Left: Structure of cardiac troponin (PDB ID: 4Y99); Right: Design model of lucCageTrop, the cTnI sensor in the closed state containing segments of cTnT and cTnC. b, Left: Kinetics of luminescence increase upon addition of 1 nM cTnI to 0.1 nM lucCageTrop sensor + 0.1 nM of lucKey. Right: A wide analyte (cTnI) detection range can be achieved by changing the concentration of the sensor components (lines). The grey area indicates the cTnI concentration range relevant to the diagnosis of acute myocardial infarction (AMI); the dotted line indicates clinical AMI cut-off defined by W.H.O. (0.6 ng/mL, 25 pM). c, Design models of lucCageHBV and lucCageHBVα, containing SmBit, and one or two tandem antigenic epitopes from the Hepatitis B Virus (HBV) PreS1 protein, respectively (two epitope copies) has higher affinity for the anti-HBV antibody HzKR127-3.2 (Kd= 0.68 nM) than lucCageHBV (one epitope copy) (Kd= 20 nM) as demonstrated by biolayer interferometry. e, Left: Kinetics of bioluminescence signal increase upon addition of 10n anti-HBV antibody to 1 nM lucCageHBVα + 1 nM lucKey. Right: By varying the concentrations of the sensor components, sensitive anti-HBV antibody detection can be achieved over a wide concentration range. f, Schematic of the detection mechanism for HBV protein PreS1 using lucCageHBV. g, Kinetics of bioluminescence following addition of the anti-HBV antibody (step 1) and subsequently PreS1 (step 2). The bioluminescence decreases upon PreS1 addition as PreS1 competes with the sensor for the antibody. h, Sensitive detection of PreS1 can be achieved over the relevant post-HBV infection concentration levels (grey area). The sensor is pre-mixed with the anti-HBV antibody; the PreS1 detection range can be tuned by varying the concentration of antibody (indicated by colored labels).

FIGS. 4(a-d). Design of biosensors for detection of anti-SARS-CoV-2 antibodies and SARS-CoV-2 RBD. a, SARS-CoV-2 viral structure representation showing the major structural proteins: Envelope protein (E), membrane protein (M), nucleocapsid protein (N), and the Spike protein (S) containing the receptor-binding domain (RBD). Linear epitopes for the M and N proteins were selected based on published immunogenicity data. b, Left panel: structural model of lucCageSARS2-M. Two copies of the SARS-CoV-2 Membrane protein a.a. 1-17 epitope are grafted into lucCage connected with a flexible spacer. Middle panel: kinetics of luminescent activation of lucCageSARS2-M (50 nM) + lucKey (50 nM) upon addition of anti-SARS-CoV-1 Membrane protein rabbit polyclonal antibodies at 100 nM (ProSci, 3527). These antibodies, originally raised against a peptide corresponding to 13 amino acids near the amino-terminus of SARS-CoV Matrix protein, cross-react with residues 1-17 of the SARS-CoV-2 Membrane protein. Right panel: response of lucCageSARS2-M (5 nM) + lucKey (5 nM) to varying concentrations of target anti-M pAb. c, Left panel: structural model of lucCageSARS2-N. Two copies of the SARS-CoV-2 Nucleocapsid protein 369-382 epitope are grafted into lucCage connected with a flexible spacer. Middle panel: kinetics of luminescent activation of lucCageSARS2-N (50 nM) + lucKey (50 nM) upon addition of 100 nM anti-SARS-CoV-1-N mouse monoclonal antibody (clone 18F629.1). This antibody originally raised against residues 354-385 of the SARS-CoV-1 Nucleocapsid protein cross-reacts with residues 369-382 of the SARS-CoV-2 Nucleocapsid protein. Right panel: response of lucCageSARS2-N (50 nM) + lucKey (50 nM) to varying concentration of target (anti-N mAb). d, Functional characterization of lucCageRBD, a SARS-CoV-2 RBD sensor. Left panel: structural model of lucCageRBD showing the LCB1 binder comprising a caged SmBiT fragment. Second panel: kinetic measurement of luminescence intensity upon addition of 16.7 nM of RBD to a mixture of 1 nM of lucCageRBD and 1 nM of lucKey. Third panel: detection over a wide range of analyte concentrations by changing the biosensor concentration (10 and 1 nM lucCage and lucKey). Right panel: Limit of detection (LOD) determination of lucCageRBD and lucKey at 1 nM each for detection of RBD in solution. LOD was determined to be 15 pM.

FIG. 5. Biosensor specificity. Each sensor at 1 nM was incubated with 50 nM of its cognate target (black lines) and the targets for the other biosensors (grey lines). Targets are Bcl-2, BoNT/B, human IgG Fc, Her2, cardiac Troponin I, anti-HBV antibody (HzKR127-3.2), anti-SARS-CoV-1-M polyclonal antibody and SARS-CoV-2 RBD. All experiments were performed in triplicate, representative data are shown, and data are presented as mean values +/- s.d.

FIGS. 6(a-g). Determination of the optimal SmBit position in lucCage and characterization of lucCageBim, a Bcl-2 biosensor. a, Protein models showing the different threading positions of SmBiT and the Bim peptide on the latch helix of the de novo LOCKR switch. b, Experimental screening of 11 de novo Bcl-2 sensors. Eleven variants were generated by combining the SmBit and Bim positions in (a) and characterized by activation of their luminescence upon addition of Bcl-2. Luminescence measurements were performed with each design (20 nM) and lucKey (20 nM) in the presence or absence of Bcl-2 (200 nM). SmBiT312-Bim339 (hence referred to as lucCageBim) was selected for posterior characterization due to its higher brightness, dynamic range and stability. c-g, Characterization of lucCageBim. c, Structural design model in ribbon representation. d, Blow-up showing the predicted interface of SmBiT and Cage. e, Blow-up showing the predicted interface of Bim and Cage. f, Kinetic luminescence measurements upon addition of Bcl-2 (200 nM) to a mix of lucCageBim (20 nM) and lucKey (20 nM). g, Tunable sensitivity of lucCageBim to Bcl-2 by changing the concentrations of sensor (lucCageBim and lucKey) components (curves).

FIGS. 7(a-d). Functional screening of sCageHA designs and crystal structure of sCageHA_267-1S. a, Structural models of sCageHA designs with the embedded de novo binder HB 1.9549.2. The HB1.9549.2 protein was grafted into a parental six-helix bundle (sCage) at different positions along the latch helix including three consecutive glycine residues. The black arrows indicate the additionally introduced single V255S (1S) or double V255S/I270S (2S) mutation(s) on the latch. b, Experimental validation of five sCageHA designs binding to HA in the presence or absence of the key by biolayer concentration of the sCages and the key were 1 µM and 2 µM, respectively. sCageHA_267-1S exhibited the highest fold of activation. c, Structural comparison showing the flexible nature of sCage to enable caging of HB1.9549.2. The structural model of sCage and the crystal structure of sCageHA_267-1S are superposed, and a narrow section (black box) is shown in an orthogonal view for detail. The N-terminal helix of HB1.9549.2 is displaced from the latch helix (α6) by 3.2 Å (middle panel) with a concomitant displacement of α5 and partial disruption of a hydrogen-bond network involving Q16 and N214 of sCage (right panels). d, A blow-up view of the intramolecular interactions of sCageHA_267-1S. The HA-binding residues are highlighted . Both the N-terminal helix (α1) and the following helix (α2) of HB1.9549.2 interact with the cage. The intramolecular interactions are all hydrophobic. The bulky hydrophobic side chain of F285 tightly abuts against the backbone atoms of α5 of sCage, which is unlikely to happen without a bending of α5. Unfavorable interactions are also found: F273 is solvent-exposed, and the Y287 hydroxyl group is buried in the apolar environment. The rightmost panel shows the quality of the electron density map.

FIGS. 8(a-d). Design and characterization of a Botulinum neurotoxin B sensor.a, Structural models of the botulinum neurotoxin B (BoNT/B) sensor designs showing the different threading positions of Bot.0671.2 (PDB ID: 5VID) on the latch of lucCage. The SmBit peptide is shown in ribbon representation. I328S and L345S indicate mutations introduced to tune the latch-cage interface (1S=I328S, 2S=I328S/L345S)², and “GGG” indicates the presence of three consecutive glycine residues between the latch and the grafted protein. The black box shows a close-up view of the interface of Cage and Bot.0671.2 n the 349_2S design. b, Experimental screening of 9 de novo BoNT/B sensors. Luminescence measurements were performed for each design (20 nM) and lucKey (20 nM) in the presence or absence of the BoNT/B protein (200 nM). The luminescence values for each design were normalized to 100 in the absence of BoNT/B. Design 349_2S was selected as the best candidate due to high sensitivity and stability, and was named lucCageBot. c, Determination of lucCagerBot sensitivity. Bioluminescence was measured over 6000 s in the presence of serially diluted BoNT/B protein. From top to bottom - lucCageBot:lucKey concentration (nM) = 50:5, 5:5, 1:10, 0.5:0.5. d, Limit of detection (LOD) calculations for the sensor at different concentrations. From top to bottom - lucCageBot:lucKey concentration (nM) = 50:5, 5:5, 1:10, 0.5:0.5. Error bars represent SD.

FIGS. 9 (a-d). Design and characterization of an Fc domain sensor. a, Structural models of the Fc sensor designs showing the different threading positions of the S. aureus Protein A domain C (PDB ID: 4WWI) on the latch of lucCage. The Sn in ribbon representation. I328S and L345S indicate mutations introduced to tune the latch-cage interface, (1S=I328S, 2S=I328S/L345S)², and “GGG” indicates the presence of three consecutive glycine residues between the latch and the grafted protein. b, Experimental screening of 6 de novo Fc domain sensors. Luminescence measurements were performed for each design (20 nM) and lucKey (20 nM) in the presence or absence of recombinant human IgG1 Fc (200 nM). The luminescence values were normalized to 100 in the absence of Fc. Design 351_2S was selected as the best candidate due to high sensitivity and stability, and was named lucCageProA. c, Determination of lucCageProA’s sensitivity. Bioluminescence was measured over 6000 s in the presence of serially diluted Fc protein. From top to bottom -lucCageBot:lucKey concentration (nM) = 50:5, 5:5, 1:10, 0.5:0.5. d, Limit of detection (LOD) calculations for the sensor at different concentrations. From top to bottom -lucCageBot:lucKey concentration (nM) = 50:5, 5:5, 1:10, 0.5:0.5. Error bars represent SD.

FIGS. 10(a-d). Design and characterization of a Her2 sensor. a, Structural models of the Her2 sensor designs showing the different threading positions of the Her2 affibody protein (PDB ID: 3MZW) on the latch of lucCage. The SmBit peptide is shown in ribbon representation. I328S and L345S indicate mutations introduced to tune the latch-cage interface, (1S=I328S, 2S=I328S/L345S)², and “GGG” indicates the presence of three consecutive glycine residues between the latch and the grafted protein. The black boxes show a blow-up view of the interface of Cage and the Her2 affibody in the 354_2S design. b, Experimental screening of 7 de novo Her2 sensors. Luminescence measurements were taken for each design (20 nM) and lucKey (20 nM) in the presence or absence of the ectodomain of Her2 (200 nM). The luminescence values were normalized to 100 in the absence of Her2 ectodomain. Design 354_2S was selected as the best candidate due to high sensitivity and stability, and was named lucCageHer2. c, Determination of lucCagerHer2′s sensitivity. Bioluminescence was measured over 6000 s in the presence of serially diluted Her2 ectodomain protein. From top to bottom - lucCageBot:lucKey concentration (nM) = 50:5, 5:5, 1:10, 0.5:0.5. d, Limit of detection (LOD) calculations for the sensor at different concentrations. From top to bottom - lucCageBot:lucKey concentration (nM) = 50:5, 5:5, 1:10, 0.5:0.5. Error bars represent SD.

FIGS. 11(a-f). Design, selection, and engineering of lucCageTrop for cardiac Troponin I detection. a, Experimental screening of designed sensors for cardiac Troponin I (cTnI). Fragments of cardiac Troponin T, namely cTnTf1-f6, were computationally grafted into lucCage at different positions of the latch. All designs were produced in E. coli and experimentally screened at 20 nM and 20 nM lucKey for an increase in presence of cTnI (100 nM). The luminescence values were normalized to 100 in the absence of cTnI. Design 336-cTnTf6-K342A was selected as the best candidate (named lucCageTrop627) based on its sensitivity, activation fold-change, and stability.

cTnTf1:226-EDQLREKAKELWQTI-240 (SEQ ID NO:27385)

cTnTf2:226-EDQLREKAKELWQTIYN-242 (SEQ ID NO:27386)

cTnTf3:226-EDQLREKAKELWQTIYNLEAE-246 (SEQ ID NO:27
387)

cTnTf4:226-EDQLREKAKELWQTIYNLEAEKFD-249 (SEQ ID NO
:27388)

cTnTf5:226-EDQLREKAKELWQTIYNLEAEKFDLQE-252 (SEQ ID
NO:27389)

cTnTf6:226-EDQLREKAKELWQTIYNLEAEKFDLQEKFKQQKYEINVL
RNRINDNQ-272 (SEQ ID NO:27390)

b, Models of lucCageTrop627 and lucCageTrop, an improved version by fusion of cardiac Troponin C (cTnC) at the C-terminus of lucCageTrop627. The models are shown in ribbon representation comprising SmBit a fragment of cTnT (PDB ID: 4Y99), and cTnC (PDB ID: 4Y99). The black box shows a close-up view of the interface of Cage and cTnT in the lucCageTrop design. c, The binding affinity of lucCageTrop627 and lucCageTrop to cTnI was measured by biolayer interferometry. lucCageTrop showed 7-fold higher affinity to cTnI than lucCageTrop627. d, Comparison of bioluminescence kinetics between lucCageTrop627 (top) and lucCageTrop (bottom) in the presence of serially diluted cTnI. Higher binding affinity leads to improved dynamic range and sensitivity of the sensor. e, Determination of lucCageTrop’s sensitivity. Bioluminescence was measured over 6000 s in the presence of serially diluted cTnI. From top to bottom - lucCageTrop:lucKey concentration (nM) = 1:10, 1:1, 0.5:0.5, 0.1:0.1. f, Limit of detection (LOD) calculations for the sensor at different concentrations. From top to bottom - lucCageTrop:lucKey concentration (nM) = 1:10, 1:1, 0.5:0.5, 0.1:0.1. Error bars represent SD.

FIGS. 12(a-f). Design and characterization of an anti-HBV antibody sensor. a, The energy-minimized models of lucCage designs are shown with the threaded segments of SmBit and the antigenic motif of PreS, respectively. The black box shows a blown-up view of the cage-motif interface of the HBV344 design. b, Experimental screening of all designs performed by monitoring the luminescence of each lucCage (20 nM) and lucKey (20 nM) in the presence or absence of the anti-HBV antibody HzKR127-3.2 (100 nM). The luminescence values were normalized to 100 in the absence of anti-HBV. The design HBV344 was selected due to its better performance and was named lucCageHBV. c,d, Determination of lucCageHBV sensitivity. Bioluminescence was measured over 6000 s in the presence of serially diluted HzKR127-3.2. From top to bottom - lucCageHBV:lucKey concentration (nM) = 50:5, 5:5, 1:1. The maximum values of the curves in c, are used to obtain the curves in d. e, Limit of detection (LOD) calculations for the concentrations. From top to bottom - lucCageHBV:lucKey concentration (nM) = 50:5, 5:5, 1:1. f, Luminescence kinetics after the addition of the antibody (anti-HBV, first arrow). From top to bottom - anti-HBV antibody concentrations = 100, 50, 12.5 nM. At 6000 s, different concentrations of the PreS1 domain were injected into the wells, and the decreased luminescence signals were used to detect PreS1. Error bars represent SD.

FIGS. 13(a-d). Experimental characterization of lucCageHBVα for improved detection of an anti-HBV antibody. a, Structural model of lucCageHBVα with a blow-up detail of the predicted interface between the PreS1 epitope and lucCage. The design comprises two copies of the epitope PreS1 (a.a. 35-46) GANSNNPDWDFNGGSGGGSSGFGANSNNPDWDFNPN (SEQ ID NO: 27630), spaced by a flexible linker to enable bivalent interaction with the antibody. The SmBit peptide is shown in ribbon representation. b, Determination of lucCageHBVα detection sensitivity to the presence of the antibody HzKR127-3.2 (anti-HBV). Bioluminescence was measured over 6000 s in the presence of serially diluted HzKR127-3.2. From top to bottom - lucCageHBVα:lucKey concentration (nM) = 50:5, 5:5, 1:10, 0.5:0.5. c, The linear region of a calibration curve was used to determine the limit of detection (LOD) and the dynamic range of antibody detection. d, Bioluminescence images acquired with a BioRad ChemiDoc imaging system. From top to bottom - lucCageHBVα:lucKey concentration (nM) = 50:5, 5:5, 1:10. Changes in bioluminescence intensity levels were detected as a function of the concentration of HzKR127-3.2.

FIGS. 14(a-d). Design and characterization of sensors for anti-SARS-CoV-2 antibodies. a-b, Experimental screening of de novo sensors for antibodies against the SARS-CoV-2 membrane protein (a), and the nucleocapsid protein (b). Selected epitopes of the membrane protein (M1, M3 and M4;

M1_1-31:MADSNGTITVEELKKLLEQWNLVIGFLFLTWI (SEQ ID N
O:27659);

M3_1-17:MADSNGTITVEELKKLLE (SEQ ID NO:27660);

M4_8-24:ITVEELKKLLEQWNLVI (SEQ ID NO:27661))

and the nucleocapsid protein (N6 single (PKKDKKKKADETQALPQRQKK; SEQ ID NO:27662) and N62 single (KKDKKKKADETQAL; SEQ ID NO: 27663) were computationally grafted into lucCage at different positions of the latch. Each design comprised two tandem copies of each epitope, separated by a flexible linker, to take advantage of the bivalent binding of antibodies. All designs were experimentally screened for increase in luminescence at 20 nM of each lucCage design and 20 nM of lucKey in the presence of anti-M rabbit polyclonal antibodies (ProSci, 3527) (a) or anti-N mouse monoclonal antibody at 100 nM (clone 18F6 luminescence values were normalized to 100 in the absence of antibodies. Designs M3_1-17_334 and N62_369-382_340 were selected as the best candidates due to high sensitivity and stability, and were named lucCageSARS2-M and ucCageSARS2-N respectively. c, Left panel: structural model of lucCageSARS2-M, showing a blow-up of the predicted interface between the M3 epitope and lucCage. Middle panel: determination of lucCageSARS2-M (MADSNGTITVEELKKLLEGGSGGMADSNGTITVEELKKLLE (SEQ ID NO: 27392)) sensitivity to anti-M pAb. Bioluminescence was measured over 4000 s in the presence of serially diluted anti-M pAb. From top to bottom - lucCageSARS2-M:lucKey concentration (nM) = 50:50, 5:5. Right panel: limit of detection (LOD) calculations for the sensor at different concentrations. d, Left panel: structural model of lucCageSARS2-N, showing a blow-up of the predicted interface between the N62 epitope and lucCage. Middle panel: determination of lucCageSARS2-N (KKDKKKKADETQALGGSGGKKDKKKKADETQAL; SEQ ID NO:27548) sensitivity to anti-N mAb. Bioluminescence was measured over 4000 s for lucCageSARS2-N + lucKey at 50 nM in the presence of serially diluted anti-N antibody. Right panel: LOD calculations for the sensor. Error bars represent SD.

FIGS. 15(a-e). a, Experimental screening of de novo sensors for the receptor-binding domain (RBD) of the SARS-CoV-2 Spike protein. All designs were experimentally screened for increase in luminescence at 20 nM of each lucCage design and 20 nM of lucKey in the presence of 200 nM RBD. The luminescence values were normalized to 100 in the absence of RBD. Design lucCageRBDdelta4_348 was selected as the best candidate due to high sensitivity and stability, and was named lucCageRBD. b, Structural model of lucCageRBD composed of the LCB1 binder grafted into lucCage comprising a caged SmBiT fragment. The black boxes show a blow-up view of the interface of Cage and LCB1 binder in the lucCageRBD design. c, Determination of lucCagerRBD’s sensitivity. Bioluminescence was measured over 10000 s in the presence of serially diluted RBD protein. From top to bottom -lucCageRBD:lucKey concentration (nM) = 1:1, 1:10, 10:10. d, Limit of detection (LOD) calculations for the sensor at different concentrations. From top to bottom -lucCageRBD:lucKey concentration (nM) = 1:1, 1:10, 10:10. e, Bioluminescence images acquired with a BioRad ChemiDoc imaging system. Changes in bioluminescence intensity levels were detected as a function of the concentration of RBD with lucCageRBD at 1 nM and lucKey at 10 nM.

FIG. 16. General principle of LOCKR-based biosensor and expanding readouts by various split protein assembly.

FIGS. 17 (a-c). (a) Schematic diagram, emission spectrum, an changes of BRET ratios of intermolecular HBV antibody BRET sensor (S0512). (b) Schematic diagram, emission spectrum, and standard curve of intramolecular HBV antibody BRET sensor (B0622). The linker optimization was performed for optimal BRET efficiency. (c) Emission spectrum and dose-dependent changes of BRET ratios of B0622_6 to the presence of HBV antibody (DFISREVSKGEELIKENMRSK is SEQ ID NO:27655; DFISREEELIKENMRSK is SEQ ID NO: 27656; DFISRELIKENMRSK is SEQ ID NO: 27657; and DFISREKENMRSK is SEQ ID NO: 27658). 2 nM of sensor concentration and 20, 5, 0 nM (left to right) of MBP_Key were used.

FIG. 18. Schematic diagram, the hydrolysis mechanism of Nitrocefin (colorimetric substrate), and the dose-dependent changes of β-lactamase activities to human cardiac Troponin I (cTnI) for colorimetric Troponin I sensor (LacATrop). β-lactamase activities were monitored at OD490. The initial rate of β-lactamase in each cTnI was calculated as β-lactamase activities. Photo below showed the dose-dependent color changed in solution from yellow to reddish in the presence of cTnI.

FIGS. 19(a-d). CoV LOCKR Diagnostic. A. The strategy for both negative and positive controls is illustrated. The negative control will receive an added excess of synthetic linear peptide epitope to occupy all epitope binding sites on available antibodies. The positive control sample will contain lucCage-ProA / lucKey components to measure the presence of IgG or IgM antibodies wherein the Latch component of the lucCage contains the Fc domain antibody binding Protein A. B. Functional positive control lucCage-ProA component (have already been identified (and are capable of detecting polyclonal rabbit IgG antibodies (middle panel) together with a lucKey within minutes after addition vs. buffer containing only LucKey (black line) in the presence of Nano-Glo® reagents (Promega). The right panel demonstrates the sensitivity of the system for as little as 10 nM of IgG, with normalized luminescence at different concentrations of sensor (lucCage + lucKey) at 1, 10, and 5 nM, incubated with different concentrations of IgG. C. Evaluation of LOCKR Biosensor Specificity. Sensors at 10 nM (LucCageSARS2-N at 50 nM) were incubated with 50 nM of cognate target, the targets for the other biosensors or buffer. Strong responses were observed only for the cognate targets. D. POCD CoV LOCKR Device. The device—pre-filled in a sterile package (left)—includes in one channel the (+) positive control lucCage-ProA / lucKey reagents which are designed to activate upon binding IgG, (s) the test sample lucCage-Coronavirus-Epitope / lucKey reagents, and (-) the negative control reagents which are lucCage-Coronavirus-Epitope / lucKey plus excess peptide epitope [~1 mM].

FIGS. 20(a-c). CoV LOCKR Diagnostic. Designed LOCKR provide a kinetic “all in solution” assay to detect the presence of epitope-specific antibodies. A. At the start, lucCage-Epitope and lucKey proteins are present in solution that is dark in the “OFF” state. B. Upon addition of a fluid containing antibodies capable of binding to the epitope of interest the Latch binding interface of the lucCage is exposed allowing the lucKey domain to bind, positioning the fused large bit of split luciferase to bind to the small bit of split luciferase. This results in reconstitution of luciferase luminescence (“ON”). C. Addition of recombinant antigen containing the Epitope of interest will shift the equilibrium of antibody binding from the Latch to the antigen, causing less reconstitution of split luciferase activity, resulting in a dim light emittance (“DIM”).

FIG. 21. Indirect Detection. The sensor platforms of the disclosure can be repurposed to accommodate an “indirect detection” approach, in which the split reporter protein (intermolecular or intramolecular embodiments; an intermolecular embodiment is shown in FIG. 21) is reconstituted by pre-incubation of the biosensor with the target (exemplified by an anti-HBV antibody) for the target binding polypeptide, resulting in fluorescence activation in this example. The activated biosensor is then incubated with a sample to detect the presence of an antigen to which the antibody binds (in this example Hepatitis B virus antigen (PreS 1)), resulting in binding of the antibody to the antigen, loss of interaction between the split reporter protein components, and reduction/elimination of reporting activity (in this case, loss of fluorescence activity).

FIG. 22. Control Samples for CoV LOCKR Diagnostic. A. The strategy for both negative and positive controls is illustrated. The negative control will receive an added excess of synthetic linear peptide epitope to occupy all epitope binding sites on available antibodies in the sample. While the positive control sample will contain lucCage-ProA / lucKey components to measure the presence of IgG or IgM antibodies wherein the Latch component of the lucCage contains the Fc domain antibody binding protein Protein A. B. Functional positive control lucCage-ProA component have already been identified (middle panel) and are capable of detecting polyclonal rabbit IgG antibodies together with a lucKey within minutes after addition vs. buffer containing only LucKey (black line) in the presence of Nano-Glo® reagents (Promega). The right panel demonstrates the sensitivity of the system for as little as 10 nM of IgG, with normalized luminescence at different concentrations of sensor (lucCage + lucKey) at 1, 10, and 5 nM, incubated with different concentrations of IgG.

### DETAILED DESCRIPTION

All references cited are herein incorporated by reference in thei application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R.I. Freshney. 1987. Liss, Inc. New York, NY), Gene Transfer and Expression Protocols, pp. 109-128, ed. E.J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: may be present or may be absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides cage proteins comprising a helical bundle, wherein the cage protein comprises a structural region and a latch region, wherein the latch region comprises one or more target binding polypeptide, wherein the cage protein further comprises a first reporter protein domain, wherein the first reporter pr [symbol] a detectable change in reporting activity when bound to a second reporter protein domain, and wherein the structural region interacts with the latch region to prevent solution access to the one or more target binding polypeptide.

Cage proteins and their use in protein switches are generally described in US patent application publication number US20200239524, incorporated by reference herein in its entirety. The present disclosure provides a significant improvement to such cage proteins and proteins switches by incorporating reporters and one or more target binding polypeptide, permitting use as a modular and generalizable biosensor platform that can enable a wide range of readouts for different sensing purposes as disclosed herein.

The cage polypeptide comprises a latch region and a structural region (i.e.: the remainder of the cage polypeptide that is not the latch region). The latch region may be present near either terminus of the cage polypeptide. In one embodiment, the latch region is placed at the C-terminal helix. In various embodiments, the latch region may comprise a part or all of a single alpha helix in the cage polypeptide at the N-terminal or C-terminal portions. In various other embodiments, the latch region may comprise a part or all of a first, second, third, fourth, fifth, sixth, or seventh alpha helix in the cage polypeptide. In other embodiments, the latch region may comprise all or part of two or more different alpha helices in the cage polypeptide; for example, a C-terminal part of one alpha helix and an N-terminal portion of the next alpha helix, all of two consecutive alpha helices, etc.

The examples provide extensive details on exemplary cage proteins and reporting activities. Any suitable reporting protein domains may be used that involves two separate protein components (for example, BRET and FRET formats, as described herein), or reporting proteins that can be split into two (or more) protein domains and its activity can be reconstituted when the when the two (or more) split protein domains are joined.

The detectable change may be any increase or a decrease in the relevant reporting activity, as deemed suitable for an intended purpose. Various non-limiting embodiments of detectable changes in reporting activity that can be utilized are described below when discussing the biosensors of the disclosure, and in the examples.

In one embodiment, the cage protein further comprises the second reporter protein domain, wherein one of the first reporter protein domain and the second reporter domain is present in the latch region and the other is present in the structural region, wherein an interaction of the first reporter protein domain and the second reporter protein domain is diminished in the presence of target to which the one or more target [symbol] binds.

In another embodiment, the second reporter protein domain is not present in the cage protein and is present in another component (i.e.: the “key”, described below), or may be present elsewhere.

In one embodiment, cage protein the helical bundle comprises between 2-9, 2-8, 2-7, 3-9, 3-8, 3-7, 4-9, 4-8, 4-7, 5-9, 5-8, 5-7, 6-9, 6-8, 6-7, 2-6, 3-6, 4-6, 5-6, 2-5, 3-5, 4-5, 2-4, 3-4, 2-3, 2, 3, 4, 5, 6, 7, 8, or 9 alpha helices.

In another embodiment, each helix in the structural region of the cage protein may independently be between 18-60, 18-55, 18-50, 18-45, 22-60, 22-55, 22-50, 22-45, 25-60, 25-55, 25-50, 25-45, 28-60, 28-55, 28-50, 28-45, 32-60, 32-55, 32-50, 32-45, 35-60, 35-55, 35-50, 35-45, 38-60, 38-55, 38-50, 38-45, 40-60, 40-58, 40-55, 40-50, or 40-45 amino acids in length.

In another embodiment, the latch region may be extended in the designs of the present disclosure due to presence of the one or more target binding polypeptide within the latch region, and thus an alpha helix/alpha helices in the latch region may be significantly longer than in the structural region, limited only by the length of the target binding polypeptide present in the latch.

In any of these embodiments, adjacent alpha helices in the cage protein may optionally be linked by amino acid linkers. Amino acid linkers connecting each alpha helix can be of any suitable length or amino acid composition as appropriate for an intended use. In one non-limiting embodiment, each amino acid linker is independently between 2 and 10 amino acids in length, not including any further functional sequences that may be fused to the linker. In various non-limiting embodiments, each amino acid linker is independently 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-7, 3-7, 4-7, 5-7, 6-7, 2-6, 3-6, 4-6, 5-6, 2-5, 3-5, 4-5, 2-4, 3-4, 2-3, 2, 3, 4, 5, 6, 7, 8, 9, or 10 amino acids in length. In all embodiments, the linkers may be structured or flexible (e.g. poly-GS). These linkers may encode further functional sequences, as deemed appropriate for an intended use.

The latch region may be present at any suitable location on the cage protein as deemed appropriate for an intended purpose. In one embodiment, the latch region is at the C-terminus of the cage protein. In another embodiment, the latch region may be at the N-terminus of the cage protein.

Similarly, the first reporter protein domain may be present at [symbol] a the cage protein as deemed appropriate for an intended purpose. In one embodiment, the first reporter protein domain is present in the latch region. In one embodiment, the first reporter protein domain is at the C-terminus of the latch region or within 20, 19, 18, 17, 16, 15, 14, 13, 12, 11, 10, 9, 8, 7, 6, 5, 4, 3, 2, or 1 amino acid of the C-terminus of the latch region. In another embodiment, the first reporter protein domain is at or within 20, 19, 18, 17, 16, 15, 14, 13, 12, 11, 10, 9, 8, 7, 6, 5, 4, 3, 2, or 1 amino acid of the N-terminus of the latch region.

In another embodiment, the second reporter protein may be present in the cage protein; in this embodiment, the second reporter protein domain may be present in the structural region. In one such embodiment, the second reporter protein may be present at the N-terminus of the structural region, or may be within 10, 9, 8, 7, 6, 5, 4, 3, 2, or 1 amino acid of the N-terminus of the structural region.

The cage protein comprises one or more (i.e., 1, 2, 3, etc.) target binding polypeptides. In one embodiment, the cage protein comprises one target binding polypeptide. In another embodiment, the cage protein comprises two target binding polypeptides. In one embodiment, the one or more target binding polypeptide and the first reporter protein domain are separated by at least 10 amino acids in the latch region of the cage protein. In another embodiment, the one or more target binding polypeptide is at or within 10, 9, 8, 7, 6, 5, 4, 3, 2, or 1 amino acid of the C-terminus of the latch region.

Any suitable reporting protein domains may be used that involves two separate protein components (for example, BRET and FRET formats, as described herein), or reporting proteins that can be split into two (or more) protein domains and its activity can be reconstituted when the when the two (or more) split protein domains are joined. In one embodiment, the first reporter protein domain, and the second reporter domain when present in the cage protein, comprise reporter protein domains selected from the group consisting of luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease).

In one embodiment, the cage protein does not include the secor one such embodiment, the first reporter protein domain comprises:

(a) an amino acid sequence at least 70%, 75%, 80%, 85%, 90%, 91%, 92%, 93%, 94%, 95%, 96%, 97%, 98%, or 100% identical to the amino acid sequence of SEQ ID NO: 27359 and 27664-27672: VTGYRLFEEIL (SmBit) (SEQ ID NO:27359), VTGYRLFEKIL (SEQ ID NO:27664), VTGYRLFEKIS (SEQ ID NO:27665), VSGWRLFKKIS (SEQ ID NO:27666), VEGYRLFEKIS (SEQ ID NO:27667), VTGYRLFEKES (SEQ ID NO:27668), VTGWRLFEKIL (SEQ ID NO:27669), VTGWRLFKEIL (SEQ ID NO:27670), VTGYRLFKEIL (SEQ ID NO:27671), LAGWRLFKKIS (SEQ ID NO:27672);

(b) an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS: 27360-27361:

VFAHPETL VKVKDAEDQLGA RVGYIELDLN SGKILESFRP EERFPMMSTF KVLLCGAVLS
RVDAGQEQLG RRIHYSQNDL VEYSPVTEKH LTDGMTVREL CSAAITMSDN TAANLLLTTI
GGPKELTAFL HNMGDHVTRL DRWEPELNEA IPNDERDTTT PAAMATTLRK LLTGENGR
(split β-lactamase A; SEQ ID NO:27360)

and

LLTLASRQQLIDWME ADKVAGPLLR SALPAGWFIA DKSGAGERGS RGIIAALGPD
GKPSRIVVIY TTGSQATMDE RNRQIAEIGA SLIKHW (Split beta lactamase B; SEQ ID
NO: 27361);

(c) an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27362-27378, wherein underlined residues are amino acid linkers or other optional residues that may be present or absent, and when present may be any amino acid sequence, and wherein any N-terminal methionine residues may be present or absent:

VFTLEDFVGDWRQTAGYNLSQVLEQGGVSSLFQNLGVSVTPIQRIVLSGE
NGLKIDIHVIIPYEGLSGDQMGQIEKIFKVVYPVDNHHFKVILHYGTLVI
DGVTPNMIDYFGRPYEGIAVFDGKKITVTGTLWNGNKIIDERLINPDGSL
LFRVTINGVTGWRLHERILA (TeLuc; SEQ ID NO: 27362)

(full luminescent or fluorescent protein that can be used to create FRET and/or BRET sensors);

LIK ENMRSKLYLE GSVNGHQFKC THEGEGKPYE GKQTNRIKW EGGPLPFAFD ILATHFMYGS
KVFIKYPADL PDYFKQSFPE GFTWERVMVF EDGGVLTATQ DTSLQDGELI YNVKVRGVNF
PANGPVMQKK TLGWEPSTET MYPADGGLEG RCDKALKLVG GGHLHVNFKT TYKSKKPVKM
PGVHYVDRRL ERIKEADNET YVEQYEHAVA RYSNLGGMD ELYK (CyOFP variant; SEQ ID
NO: 27363)

(full luminescent or fluorescent protein that can be used to create FRET and/or BRET sensors);

VSKGEELIK ENMRSKLYLE GSVNGHQFKC THEGEGKPYE GKQTNRIKW EGGPLPFAFD ILATHFMYGS
KVFIKYPADL PDYFKQSFPE GFTWERVMVF EDGGVLTATQ DTSLQDGELI YNVKVRGVNF
PANGPVMQKK TLGWEPSTET MYPADGGLEG RCDKALKLVG GGHLHVNFKT TYKSKKPVKM
PGVHYVDRRL ERIKEADNET YVEQYEHAVA RYSNLGGMD ELYK (CyOFP v;
NO: 27364)

(full luminescent or fluorescent protein that can be used BRET sensors);

[symbol]

EELIK ENMRSKLYLE GSVNGHQFKC THEGEGKPYE GKQTNRIKW EGGPLPFAFD ILATHFMYGS
KVFIKYPADL PDYFKQSFPE GFTWERVMVF EDGGVLTATQ DTSLQDGELI YNVKVRGVNF
PANGPVMQKK TLGWEPSTET MYPADGGLEG RCDKALKLVG GGHLHVNFKT TYKSKKPVKM
PGVHYVDRRL ERIKEADNET YVEQYEHAVA RYSNLGGMD ELYK (CuOFP variant; SEQ ID
NO: 27365)

(full luminescent or fluorescent protein that can be used to create FRET and/or BRET sensors);

KVFTLGDFVGDWRQTAGYNQAQVLEQGGLTSLFQNLGVSVTPIQRIVLSG
ENGLKIDIHVIIPYEGLSCDQMAQIEKIFKVVYPVDDHHFKAILHYGTLV
IDGVTPNMIDYFGQPYEGIAKFDGKKITVTGTLWNGNTIIDERLINPDGS
LLFRVTINGVTGWRLHERILA (LumiLuc; SEQ IDNO: 27366)

(full luminescent or fluorescent protein that can be used to create FRETand/or BRET sensors);

MVSKGEEDNM ASLPATHELH IFGSINGVDF DMVGQGTGNP NDGYEELNLK STKGDLQFSP W
ILVPHIGYG FHQYLPYPDG MSPFQAAMVD GSGYQVHRTM QFEDGASLTV NYRYTYEGSH IKG
EAQVKGT GFPADGPVMT NSLTAADWCR SKKTYPNDKT IISTFKWSYT TGNGKRYRST ARTTY
TFAKP MAANYLKNQP MYVFRKTELK HSKTELNFKE WQKAFTDVMG MDELYK
(mNeonGreen; SEQ ID NO: 27367)

(full luminescent or fluorescent protein that can be used to create FRET and/or BRET sensors);

MVSKGEAVIK EFMRFKVHME GSMNGHEFEI EGEGEGRPYE GTQTAKLKVT KGGPLPFSWD
ILSPQFMYGS RAFIKHPADI PDYYKQSFPE GFKWERVMNF EDGGAVTVTQ DTSLEDGTL
I YKVKLRGTNF PPDGPVMQKK TMGWEASTER LYPEDGVLKG DIKMALRLKD GGRYLADF
KT TYKAKKPVQM PGAYNVDRKL DITSHNEDYT WEQYERSEG RHSTGGMDEL YK
(mScarlet-i; SEQ ID NO: 27368)

(full luminescent or fluorescent protein that can be used to create FRET and/or BRET sensors);

SGKSYPTVSADYQKAVEKAKKRLGGFIAEKRCAPLMLRLAWHSAGTFDKR
TKTGGPFGTIRYPAELAHSANSGLDIAVRLLEPLKAEFPILSYADFYQLA
GVVAVEVTGGPEVPFHPGREDKPELPPEGRLPDATKGSDHLRDVFGKAMG
LTDQDIVALSGGHTLGAAHKERSGFEGPWTSNPLVFDNSYFTELLSGEKE GGGGSGGGGS (APEX2-1-200; SEQ ID NO:27369);

GGGGSGGGGS GLLQLPSDKALLSDPVFRPLVDKYAADEDAFFADYAEAHQKLSELGFADA
(APEX2-201-250; SEQ ID NO:27370);

MGSHHHHHHGSGSENLYFQGSGGS
VRPLNCIVA VSQNMGIGKN GDLPWPPLRN ESKYFQRMTT TSSVEGKQNL
VIMGRKTWFS IPEKNRPLKD RINIVLSREL KEPPRGAHFL AKSLDDALRL
IEQPELGGGGSGGGGS (DHFR A (1-105); SEQ ID NO:27371);

SGSGDPDEARKAIARVKRESKRIVEDAERLIREAAAASEKISREAERLIREAAAASEKISRE
GGGGSGGGGS ASKV DMVWIVGGSS VYQEAMNQPG HLRLFVTRIM QEFESDTFFP
EIDLGKYKLL PEYPGVLSEV QEEKGIKYKF EVYEKKD (DHFR_B (106-186); SEQ ID
NO:27372) ;

QLTPTFYDNSCPNVSNIVRDIIVNELRSDPRIAASILRLHFHDCFVNGCD
ASILLDNTTSFRTEKDAFGNANSARGFSVIDRMKAAVESACPGTVSCADL
LTIAAQQSVTLAGGPSWRVPLGRRDSLQAFLDLANANLPAPFFTLPQLKD
SFRNVGLNRSSDLVALSGGHTFGKSQCRFIMDRLYNFSNTGLPDPTLNTT
Y](sHRPa is the large split HRP fragment. It consi
sts1-213 of horseradish peroxidase (HRP) with the
following 4mutations: T21I, P78S, R93G, N175S) (SE
Q ID NO:27373);

NLSALVDFDLRTPTIFDNKYYVNLEEQKGLIQSDQELFSSPDATDTIPLV
RSFANSTQTFFNAFVEAMDRMGNITPLTGTQGQIRRNCRVVNSNGGSGS
(sHRPb is the small split HRPfragment. It consists
of amino acids 214-308 of horseradishperoxidase (
HRP) with the following 2 mutations: N255D, L299R)
(SEQID NO:27374);

GESLFKGPRDYNPISSTICHLTNESDGHTTSLYGIGFGPFIITNKHLFRR
NNGTLLVQSLHGVFKVKNTTTLQQHLIDGRDMIIIRMPKDFPPFPQKLKF
REPQREERICLVTTNFQTGGGGSGGGGS (N_Tev(1-118) (SEQ ID
NO:27375);

GGGGSGGGGSKSMSSMVSDTSCTFPSSDGIFWKHWIQTKDGQCGSPLVST
RDGFIVGIHSASNFTNTNNYFTSVPKNFMELLTNQEAQQWVSGWRLNADS
VLWGGHKVFMDKP C_Tev (119-221) (SEQID NO:27376);

MASYPCHQHA SAFDQAARSR GHSNRRTALR PRRQQEATEV RLEQKMPTLL
RVYIDGPHGM GKTTTTQLLV ALGSRDDIVY VPEPMTYWQV LGASETIANI
YTTQHRLDQG EISAGDAAVV MTSAQITMGM PYAVTDAVLA PHIGGEAGSS
HAPPPALTLI FDRHPIAALL CYPAARYLMG SMTPQAVLAF VALIPPTLPG
TNIVLGALPE DRHIDRLAKR QRPGERLDLA MLAAIRRVYG LLANTVRYLQ
GGGSWREDWG QLSGT GGGGSGGGGS (thymidine kinase_TK_A (1-265) (SEQ ID
NO: 27377); and/or

GGGGSGGGGS AVPPQ GAEPQSNAGP RPHIGDTLFT LFRAPELLAP
NGDLYNVFAW ALDVLAKRLR PMHVFILDYD QSPAGCRDAL LQLTSGMVQT
HVTTPGSIPT ICDLARTFAR EMGEAN (thymidine kinase_TK_B (266-376) (SEQ
ID NO: 27378)

This embodiment of the cage protein comprising a reporter protein domain will interact with the second biosensor component “key” protein (discussed below) comprising a second reporter domain in presence of a target analyte.

In another embodiment, the cage comprises the second reporter protein domain, wherein

(a) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NOS: 27359, and 27664-27672;

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27379, wherein the N-terminal methionine residue may be present or absent:

MVFTLEDFVGDWEQTAAYNLDQVLEQGGVSSLLQNLAVSVTPIQRIVRSG
ENALKIIEVFKVVYPVDDHHFKVILPYGTLVIDGVTPNMLNYFGRPYEGI
AVFDGKKITVTGTILFRVTINS (LgBiT) (SEQ ID NO: 27379)
;

(b) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27360

VFAHPETL VKVKDAEDQLGA RVGYIELDLN SGKILESFRP EERFPMMSTF KVLLCGAVLS
RVDAGQEQLG RRIHYSQNDL VEYSPVTEKH LTDGMTVREL CSAAITMSDN TAANLLLTTI
GGPKELTAFL HNMGDHVTRL DRWEPELNEA IPNDERDTTT PAAMATTLRK LLTGENGR (split β-
lactamase A) (SEQ ID NO: 27360),

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27361:

LLTLASRQQLIDWME ADKVAGPLLR SALPAGWFIA DKSGAGERGS RGIIAALGPD GKPSRIVVIY
TTGSQATMDE RNRQIAEIGA SLIKHW (Split beta lactamase B) (SEQ ID NO:
27361) ;

(c) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27362:

VFTLEDFVGDWRQTAGYNLSQVLEQGGVSSLFQNLGVSVTPIQRIVLSGE
NGLKIDIHVIIPYEGLSGDQMGQIEKIFKVVYPVDNHHFKVILHYGTLVI
DGVTPNMIDYFGRPYEGIAVFDGKKITVTGTLWNGNKIIDERLINPDGSL
LFRVTINGVTGWRLHERILA (TeLuc) (SEQ ID NO:27362),

(full luminescent or fluorescent protein that can be used to create FRET and/or BRET sensors)

and the other comprises an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27363-27365:

LIK ENMRSKLYLE GSVNGHQFKC THEGEGKPYE GKQTNRIKW EGGPLPFAFD ILATHFMYGS
KVFIKYPADL PDYFKQSFPE GFTWERVMVF EDGGVLTATQ DTSLQDGELI YNVKVRGVNF
PANGPVMQKK TLGWEPSTET MYPADGGLEG RCDKALKLVG GGHLHVNFKT TYKSKKPVKM
PGVHYVDRRL ERIKEADNET YVEQYEHAVA RYSNLGGMD ELYK (CyOFP variant) (SEQ ID
NO: 27363)

(full luminescent or fluorescent protein that can be used to create FRET and/or BRET sensors);

[symbol]

VSKGEELIK ENMRSKLYLE GSVNGHQFKC THEGEGKPYE GKQTNRIKW EGC
KVFIKYPADL PDYFKQSFPE GFTWERVMVF EDGGVLTATQ DTSLQDGELI YNVKVRGVNF
PANGPVMQKK TLGWEPSTET MYPADGGLEG RCDKALKLVG GGHLHVNFKT TYKSKKPVKM
PGVHYVDRRL ERIKEADNET YVEQYEHAVA RYSNLGGMD ELYK(CyOFP variant) (SEQ ID
NO: 27364)

(full luminescent or fluorescent protein that can be used to create FRET and/or BRET sensors); and

EELIK ENMRSKLYLE GSVNGHQFKC THEGEGKPYE GKQTNRIKW EGGPLPFAFD ILATHFMYGS
KVFIKYPADL PDYFKQSFPE GFTWERVMVF EDGGVLTATQ DTSLQDGELI YNVKVRGVNF
PANGPVMQKK TLGWEPSTET MYPADGGLEG RCDKALKLVG GGHLHVNFKT TYKSKKPVKM
PGVHYVDRRL ERIKEADNET YVEQYEHAVA RYSNLGGMD ELYK(CyOFP variant) (SEQ ID NO:27365)

(full luminescent or fluorescent protein that can be used to create FRET and/or BRET sensors);

(d) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27366:

KVFTLGDFVGDWRQTAGYNQAQVLEQGGLTSLFQNLGVSVTPIQRIVLSG
ENGLKIDIHVIIPYEGLSCDQMAQIEKIFKVVYPVDDHHFKAILHYGTLV
IDGVTPNMIDYFGQPYEGIAKFDGKKITVTGTLWNGNTIIDERLINPDGS
LLFRVTINGVTGWRLHERILA (LemiLuc) (SEQ ID NO:27366)

(full luminescent or fluorescent protein that can be used to create FRET and/or BRET sensors),

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27368, wherein the N-terminal methionine residue may be present or absent:

MVSKGEAVIK EFMRFKVHME GSMNGHEFEI EGEGEGRPYE GTQTAKLKVT KGGPLPFSWD ILSPQFMYG
S RAFIKHPADI PDYYKQSFPE GFKWERVMNF EDGGAVTVTQ DTSLEDGTLI YKVKLRGTNF PPDGPVM
QKK TMGWEASTER LYPEDGVLKG DIKMALRLKD GGRYLADFKT TYKAKKPVQM PGAYNVDRKL DITSH
NEDYT WEQYERSEG RHSTGGMDEL YK (mScarlet-i) (SEQ ID NO:27368)

(full luminescent or fluorescent protein that can be used to create FRET and/or BRET sensors);

(e) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27367, wherein the N-terminal methionine residue may be present or absent:

[symbol]

MVSKGEEDNM ASLPATHELH IFGSINGVDF DMVGQGTGNP NDGYEELNLK S
G FHQYLPYPDG MSPFQAAMVD GSGYQVHRTM QFEDGASLTV NYRYTYEGSH
VMT NSLTAADWCR SKKTYPNDKT IISTFKWSYT TGNGKRYRST ARTTYTFAKP MAANYLKNQP MYVFR
KTELK HSKTELNFKE WQKAFTDVMG MDELYK (mNeonGreen) (SEQ ID NO:27367),

(full luminescent or fluorescent protein that can be used to create FRET and/or BRET sensors),

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27368, wherein the N-terminal methionine residue may be present or absent:

MVSKGEAVIK EFMRFKVHME GSMNGHEFEI EGEGEGRPYE GTQTAKLKVT KGGPLPFSWD ILSPQFMYG
S RAFIKHPADI PDYYKQSFPE GFKWERVMNF EDGGAVTVTQ DTSLEDGTLI YKVKLRGTNF PPDGPVM
QKK TMGWEASTER LYPEDGVLKG DIKMALRLKD GGRYLADFKT TYKAKKPVQM PGAYNVDRKL DITSH
NEDYT WEQYERSEG RHSTGGMDEL YK (mScarlet-i) (SEQ ID NO: 27368)

(full luminescent or fluorescent protein that can be used to create FRET and/or BRET sensors);

(f) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence SEQ ID NO: 27369, wherein underlined residues are optional residues that may be present or absent, and when present may be any amino acid sequence

SGKSYPTVSADYQKAVEKAKKRLGGFIAEKRCAPLMLRLAWHSAGTFDKR
TKTGGPFGTIRYPAELAHSANSGLDIAVRLLEPLKAEFPILSYADFYQLA
GVVAVEVTGGPEVPFHPGREDKPELPPEGRLPDATKGSDHLRDVFGKAMG
LTDQDIVALSGGHTLGAAHKERSGFEGPWTSNPLVFDNSYFTELLSGEKE GGGGSGGGGS (APEX2-1-200) (SEQ ID NO:27369)

(split engineered variant of soybean ascorbate peroxidase protein for chemiluminescent and colorimetric detection system);

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27370, wherein underlined residues are optional residues that may be present or absent, and when present may be any amino acid sequence

GGGGSGGGGS GLLQLPSDKALLSDPVFRPLVDKYAADEDAFFADYAEAHQKLSELGFADA (APEX2-
201-250) (SEQ ID NO:27370)

(split engineered variant of soybean ascorbate peroxidase protein for chemiluminescent and colorimetric detection system);

(g) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27371, wherein underlined residues are optional residues that may be present or absent, and when present may be any amino acid sequence

MGSHHHHHHGSGSENLYFQGSGGS
VRPLNCIVA VSQNMGIGKN GDLPWPPLRN ESKYFQRMTT TSSVEGKQNL
VIMGRKTWFS IPEKNRPLKD RINIVLSREL KEPPRGAHFL AKSLDDALRL
IEQPELGGGGSGGGGS (DHFR A (1-105)); (SEQ ID NO: 27371)

(split dihydrofolate reductase protein reporter for cell survival or fluorescence)

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27372, wherein underlined residues are optional residues that may be present or absent, and when present may be any amino acid sequence

SGSG DPDEARKAIARVKRESKRIVEDAERLIREAAAASEKISREAERLIREAAAASEKISRE
GGGGSGGGGS ASKV DMVWIVGGSS VYQEAMNQPG HLRLFVTRIM QEFESDTFFP
EIDLGKYKLL PEYPGVLSEV QEEKGIKYKF EVYEKKD (DHFR_B (106-186));(SEQ ID NO:27372)

(split dihydrofolate reductase protein reporter for cell survival or fluorescence);

(h) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27373, wherein underlined residues are optional residues that may be present or absent, and when present may be any amino acid sequence

QLTPTFYDNSCPNVSNIVRDIIVNELRSDPRIAASILRLHFHDCFVNGCD
ASILLDNTTSFRTEKDAFGNANSARGFSVIDRMKAAVESACPGTVSCADL
LTIAAQQSVTLAGGPSWRVPLGRRDSLQAFLDLANANLPAPFFTLPQLKD
SFRNVGLNRSSDLVALSGGHTFGKSQCRFIMDRLYNFSNTGLPDPTLNTT
YLQTLRGLCPLNGGSGS (sHRPais the large split HRP fra
gment. It consists of amino acids 1-213 ofhorserad
ish peroxidase (HRP) with the following 4 mutation
s: T21I, P78S,R93G, N175S: plasmid 73147 (SEQ ID N
O: 27373) ;

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27374, wherein underlined residues are optional residues that may be present or absent, and when present may be any amino acid sequence

NLSALVDFDLRTPTIFDNKYYVNLEEQKGLIQSDQELFSSPDATDTIPLVRSFANSTQTFFNAFVEAMDRMGNIT
PLTGTQGQIRRNCRVVNSNGGSGS (sHRPb is the small split HRP fragment. It
consists of amino acids 214-308 of horseradish peroxidase (HRP) with the
following 2 mutations: N255D, L299R: plasmid 73148) (SEQ ID NO: 27374) ;

(i) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27375, wherein underlined residues are optional residues that may be p [symbol] when present may be any amino acid sequence

GESLFKGPRDYNPISSTICHLTNESDGHTTSLYGIGFGPFIITNKHLFRR
NNGTLLVQSLHGVFKVKNTTTLQQHLIDGRDMIIIRMPKDFPPFPQKLKF
REPQREERICLVTTNFQTGGGGSGGGGSN_Tev (1-118) (SEQID N
O: 27375)

(Split TEV protease);

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27376, wherein underlined residues are optional residues that may be present or absent, and when present may be any amino acid sequence

GGGGSGGGGSKSMSSMVSDTSCTFPSSDGIFWKHWIQTKDGQCGSPLVSTRDGFIVGIHSASNFTNTNNYFTSV
PKNFMELLTNQEAQQWVSGWRLNADSVLWGGHKVFMDKP (C_Tev (119-221)); (SEQ ID NO:
27376)

(Split TEV protease);

(j) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27377, wherein underlined residues are optional residues that may be present or absent, and when present may be any amino acid sequence, and wherein the N-terminal methionine residue may be present or absent:

MASYPCHQHA SAFDQAARSR GHSNRRTALR PRRQQEATEV RLEQKMPTLL
RVYIDGPHGM GKTTTTQLLV ALGSRDDIVY VPEPMTYWQV LGASETIANI
YTTQHRLDQG EISAGDAAVV MTSAQITMGM PYAVTDAVLA PHIGGEAGSS
HAPPPALTLI FDRHPIAALL CYPAARYLMG SMTPQAVLAF VALIPPTLPG
TNIVLGALPE DRHIDRLAKR QRPGERLDLA MLAAIRRVYG LLANTVRYLQ
GGGSWREDWG QLSGT GGGGSGGGGS (thymidine kinase_TK_A (1-265)) (SEQ ID NO:
27377) ;

and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27378, wherein underlined residues are optional residues that may be present or absent, and when present may be any amino acid sequence

GGGGSGGGGS AVPPQ GAEPQSNAGP RPHIGDTLFT LFRAPELLAP
NGDLYNVFAW ALDVLAKRLR PMHVFILDYD QSPAGCRDAL LQLTSGMVQT
HVTTPGSIPT ICDLARTFAR EMGEAN (thymidine kinase_TK_B (266-376) (SEQ ID NO:
27378)

These embodiments of the cage protein comprising two reporter protein domains interact with the second biosensor component “key” in presence of a target analyte. The conformational change induced by this interaction enables the approxi [symbol] for the two reporter proteins in the cage protein, allowing analyte quantification by measuring increase (or decrease) in reporter signal.

Any suitable target binding polypeptide that binds a target of interest may be used in the cage proteins of the disclosure as deemed appropriate for an intended use. As noted above, the cage protein may comprise 1, 2, 3, 4 or more target binding polypeptides, as exemplified herein. In one embodiment, the cage protein comprises 1 target binding polypeptide. In another embodiment, the cage protein comprises 2, 3, or 4 target binding polypeptides. In embodiments comprising 2 or more target binding polypeptides, each target binding polypeptide may be the same or may be different.

Similarly, the target of the one or more target binding polypeptides may be any target as suitable for an intended purpose for which one or more target binding polypeptides are available. In one non-limiting embodiment, the one or more target binding polypeptide is capable of binding to a target including but not limited to an antibody, a toxin, a diagnostic biomarker, a viral particle, a disease biomarker, a metabolite or a biochemical analyte of interest. In embodiments where there are 2 or more target binding polypeptides, each target binding polypeptide may bind the same target, or may independently bind to different targets. In embodiments where the 2 or more target binding polypeptides bind to the same target, they may bind to the same region of the target (for example, to add avidity to the interaction), or may bind to different regions of the target.

As will be understood by those of skill in the art, the one or more target binding polypeptides may comprise any type of polypeptide, including but not limited to dennovo designed proteins, affibodies, affimers, ankyrin repeat proteins (naturally occurring or designed), nanobodies, etc.

In one embodiment, the one or more target binding polypeptide is capable of binding to an antibody target. In another embodiment, the one or more target binding polypeptide comprises one or more epitope recognized by antibodies against a viral target. In a further embodiment, the one or more target binding polypeptide comprises one or more epitope recognized by antibodies against SARS-Cov-2. In various other embodiments described herein, the one or more target binding polypeptide is capable of binding to a disease marker or toxin, Bcl-2, Her2 receptor, Botulinum neurotoxin B, cardiac Troponin I, albumin, epithelial growth factor receptor, prostate-specific membrane antigen (PSMA), citrullinated peptides, brain natriuretic peptides, or any other suitable target.

In various non-limiting embodiments, the one or more target bi comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:27380-27430.

TABLE 1

Exemplary target binding polypeptides

| | | |
|---|---|---|
| Biosensors | Sensing domain | Sensing domain sequence |
| lucCageBim | Bim | EIWIAQELRRIGDEFNAYYAAA (SEQ ID NO:27380) |
| lucCageBoT | Bot.0671.2 | MFAELKAKFFLEIGDRDAARNALRKAGYSDEEAER IIRKYELE (SEQ ID NO:27381) |
| lucCageProA | Protein A domain C (SpaC) | EQQNAFYEILHLPNLTEEQRNGFIQSLKDDPSVSK EILAEAKKLNDAQAPK (SEQ ID NO: 27382) |
| lucCagHer2 | Her2 affibody | EMRNAYWEIALLPNLNNQQKRAFIRSLYDDPSQSA NLLAEAKKLNDAQAPK (SEQ ID NO:27383) |
| lucCageTrop | cTnI + cTnC | EDQLREKAKELWQTIYNLEAEKFDLQEKFKQQKYE INVLRNRINDNQKVSKTKDDSKGKSEEELSDLFRM FDKNADGYIDLEELKIMLQATGETITEDDIEELMK DGDKNNDGRIDYDEFLEFMKGVE (SEQ IDNO:27384) - cTnTf1:226-EDQLREKAKELWQTI-240 (SEQ ID NO:27385) - cTnTf2:226-EDQLREKAKELWQTIYN-242 (SEQ ID NO:27386) - cTnTf3:226-EDQLREKAKELWQTIYNLEAE-246 (SEQ ID NO:27387) - cTnTf4:226-EDQLREKAKELWQTIYNLEAEKFD-249 (SEQ ID NO:27388) - cTnTf5:226-EDQLREKAKELWQTIYNLEAEKFDLQ E-252 (SEQ ID NO:27389) - cTnTf6:226-EDQLREKAKELWQTIYNLEAEKFDLQ EKFKQQKYEINVLRNRINDNQ-272 (SEQ ID NO:27390) - EDQLREAAKELWQTIYNLEAEKFDLQEKFKQQ KYEINVLRNRINDNQKVSKTKDDSKGKSEEEL SDLFRMFDKNADGYIDLEELKIMLQATGETIT EDDIEELMKDGDKNNDGRIDYDEFLEFMKGVE (SEQ ID NO:27391) |
| lucCageSARS2-M | SARS-CoV-2 nucleocapsid protein (a.a. 369-382) 2x | MADSNGTITVEELKKLLEGGSGGMADSNGTITVEELKKLLE (SEQ ID NO:27392) - MADSNGTITVEELKKLLEQWNLV IGFLFLTWIGGSGGMADSNGTIT VEELKKLLEQWNLVIGFLFLTWI (SEQ ID NO:27393) - ITVEELKKLLEQWNLVIGGSGGI TVEELKKLLEQWNLVI (SEQ ID NO:27394) |
| lucCageSARS2-N | SARS-CoV-2 membrane protein (a.a. 1-17) 2x | N62:KKDKKKKADETQALGGSGGKKDKKKKADETQ AL (SEQ ID NO:27548 N6:PKKDKKKKADETQALPQRQKKGGSGGPKKDKK KKADETQALPQRQKK (SEQ ID NO:27547) |
| sCageHA | HB1.9549.2 | TFACRIAAKIAAEFGYSEEQIKELLKNAGCSEDEA RDAVEYLR (SEQ ID NO:27396) |

>LCB1-1

DKEWILQKIYEIMRLLDELGHAEASMRVSDLIYEFMKKGDERLLEEAERL
LEEVER (SEQ ID NO:27397)

>LCB1-2

DKEEILNKIYEIMRLLDELGNAEASMRVSDLILEFMKKGDERLLEEAERL
LEEVER (SEQ ID NO: 27398)

>LCB1-3

DKEWILQKIYEIMRLLDELGHAEASMRVSDLIYEFMKQGDERLLEEAERL
LEEVER (SEQ ID NO: 27399)

>LCB1-4

DKENILQKIYEIMKTLDQLGHAEASMQVSDLIYEFMKQGDERLLEEAERL
LEEVER (SEQ ID NO: 27400)

>LCB1-5

DKENILQKIYEIMKTLDQLGHAEASMNVSDLIYEFMKQGDERLLEEAERL
LEEVER (SEQ ID NO: 27401)

LCB1_v1.1_Cys

DKENILQKIYEIMKTLDQLGHAEASMQVSDLIYEFMKQGDERLLEEAERL
LEEVERC(SEQ ID NO: 27402)

>LCB1_v1.2

DKENILQKIYEIMKTLDQLGHAEASMYVSDLIYEFMKQGDERLLEEAERL
LEEVER (SEQ ID NO: 27403)

>LCB1_v1.3

DKENILQKIYEIMKTLEQLGHAEASMQVSDLIYEFMKQGDERLLEEAERL
LEEVER (SEQ ID NO: 27404)

>LCB1_v1.4

DKENILQKIYEIMKTLEQLGHAEASMQVSDLIYEFMKQGDENLLEEAEQL
LQEVER (SEQ ID NO: 27405)

>LCB1_v1.5 (LCB1_v1.3 with N-link Glycosylation)

DKENILQKIYEIMKTLEQLGHAEASMNVSDLIYEFMKQGDERLLEEAERL
LEEVER (SEQ ID NO: 27406)

>LCB2-1

SDDEDSVRYLLYMAELRYEQGNPEKAKKILEMAEFIAKRNNNEELERLVR
EVKKRL (SEQ ID NO: 27407)

>LCB2-2

SDDEDAVRYLLYMAELLYKQGNPEEAKKLLELAEFIAKRNNNEELERLVR
EVKKRL (SEQ ID NO: 27408)

>LCB3-1

NDDELHMLMTDLVYEALHFAKDEEIKKRVFQLFELADKAYKNNDRQKLEK
VVEELKELLERLLS (SEQ IDNO: 27409)

>LCB3-2

NDDELLMLVTDLVAEALLFAKDEEIKKRVFTLFELADKAYKNNDRDTLSK
VVSELKELLERLQ (SEQ ID NO:27410)

>LCB3_v1.2

NDDELHMQMTDLVYEALHFAKDEEIQKHVFQLFEKATKAYKNKDRQKLEK
VVEELKPNO: 27411)

>LCB3-4

NDDELHMQMTDLVYEALHFAKDEEIQKHVFQLFENATKAYKNKDRQKLEK
VVEELKELLERLLS (SEQ IDNO: 27412)

>LCB3_v1.1

NDDELHMQMTDLVYEALHFAKDEEFQKHVFQLFEKATKAYKNNDRQKLEK
VVEELKELLERLLS (SEQ IDNO: 27413)

>LCB3_v1.3

NDDELHMQMTDLVYEALHFAKDEEFQKHVFQLFEKATKAYKNKDRQKLEK
VVEELKELLERLLS (SEQ IDNO: 27414)

>LCB3_v1.4

NDDELHMQMTDLVYEALHKAKDEEFQKHVFQLFEKATKARKNKDRQKLEK
VVEELKELLERLLS (SEQ IDNO: 27415)

>LCB3_v1.5

NDDELHMQMTDLVYEALHKAKDEEMQKRVFQLFEQADKAYKTKDRQKLEK
VVEELKELLERLLS (SEQ IDNO: 27416)

>LCB4-1

QREKRLKQLEMLLEYAIERNDPYLMFDVAVEMLRLAEENNDERIIERAKR
ILEEYE (SEQ ID NO: 27417)

>LCB4-2

DREERLKYLEMLLELAVERNDPYLIFDVAIELLRLAEENNDERIYERAKR
ILEEVE (SEQ ID NO: 27418)

>LCB5-1

SLEELKEQVKELKKELSPEMRRLIEEALRFLEEGNPAMAMMVLSDLVYQL
GDPRVIDLYMLVTKT (SEQ IDNO: 27419)

>LCB5-2

SLEEVKEILKELKKELSPEDRRLIEEALRLLEEGNPAMASMVLSDLVFLL
GDPRVIELLMLVTKT (SEQ IDNO: 27420)

>LCB6-1

DREQRLVRFLVRLASKFNLSPEQILQLFEVLEELLERGVSEEEIRKQLEE
VAKELG (SEQ ID NO: 27421)

>LCB6-2

DREQRLVRFLVRLASKFNLSMEQILILFDVLEELLERGVSEEEIRKILEE
VAKEL (SEQ ID NO: 27422)

>LCB7-1

DDDIRYLIYMAKLRLEQGNPEEAEKVLEMARFLAERLGMEELLKEVRELL
RKIEELR (SEQ ID NO:27423)

>LCB7-2

DDDVRYLIYMAKLLLEQGNPEEAEKVLESARFAAELLGNEELLKEVRELL
RKIEELR (SEQ ID NO:27424)

>LCB8-1

PIIELLREAKEKNDEFAISDALYLVNELLQRTGDPRLEEVLYLIWRALKE
KDPRLLDRAIELFER (SEQ IDNO: 27425)

>LCB8-2

PVTELLREAKEKNDPMAISDALFLVFELAQRTGDPRLEEVLFLIWRALKE
KDPRLLINO: 27426)

>AHB1-1

DEDLEELERLYRKAEEVAKEAKDASRRGDDERAKEQMERAMRLFDQVFEL
AQELQEKQTDGNRQKATHLDKAVKEAADELYQRVR(SEQ ID NO: 274
27)

>AHB1-2

DEDLEELERLYRKAEEVAKEAEEASRRGDKERAKELLERALHLFDQVFEL
AQELQEKLTDEKRQKATHLDKAVHEAADELYQRVR(SEQ ID NO: 274
28)

>AHB2-1

ELEERVMHLLDQVSELAHELLHKLTGEELQRATHFDKWANEAILELIKSD
DEREIREIEEEARRILEHLEELARK(SEQ ID NO: 27429)

>AHB2-2_

ELEEQVMHVLDQVSELAHELLHKLTGEELERAAYFNWWATEMMLELIKSD
DEREIREIEEEARRILEHLEELARK(SEQ ID NO: 27430)

The polypeptides of SEQ ID NOS: 27397-27430 bind with high affinity to the SARS-CoV-2 Spike glycoprotein receptor binding domain (RBD). The polypeptides of SEQ ID NOS: 27397-27430have been subjected to extensive mutational analysis, permitting determination of allowable substitutions at each residue within the polypeptide. Allowable substitutions are as shown in Table 3 (The number denotes the residue number, and the letters denote the single letter amino acids that can be present at that residue).

Thus, in one embodiment, the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:27397-27430, or selected from SEQ ID NOS: 27397-27406, 27409-27416, 27427-27430. In another embodiment, amino acid substitutions relative to the reference target binding polypeptide amino acid sequence (i.e.: one of SEQ ID NOS: 27397-27430) are selected from the allowable amino acid substitutions provided in Table 1.

The residue numbers of the interface residues which are within 8A to the RBD target are listed below in Table 2.

TABLE 2

| | |
|---|---|
| ‘LCB1’: | [3, 6, 7, 10, 13, 17, 20, 22, 23, 25, 26, 29, 32, 33, 36], |
| ‘LCB2’: | [1, 2, 5, 6, 9, 12, 13, 16, 20, 32, 35, 39], |
| ‘LCB3’: | [1, 3, 4, 6, 7, 10, 11, 13, 14, 15, 18, 27, 30, 33, 34, 37], |
| ‘LCB4’: | [8, 11, 12, 15, 23, 24, 26, 27, 28, 30, 31, 34, 56], |
| ‘LCB5’: | [35, 37, 38, 40, 41, 44, 47, 48, 53, 56, 60, 63], |
| ‘LCB6’: | [3, 4, 7, 8, 11, 12, 14, 15, 21, 24, 25, 28, 31, 32, 35], |
| ‘LCB7’: | [2, 3, 6, 7, 9, 10, 13, 17, 29, 32, 33, 36], |
| ‘LCB8’: | [14, 15, 16, 19, 22, 23, 26, 29, 30, 38, 41, 42, 45], |
| ‘AHB1’, | [34, 38, 41, 45, 48, 49, 52, 63, 64, 67, 68, 70, 71, 74, 78, 81, 82, 85], |
| ‘AHB2’, | [4, 7, 11, 14, 15, 18, 21, 26, 29, 30, 33, 34, 36, 37, 40, 43, 44, 47, 48]. |

In another embodiment, interface residues are identical to those in the reference target binding polypeptide (i.e.: one of SEQ ID NOS:27397-27430 or are conservatively substituted relative to interface residues in the reference target binding polypeptide as detailed in Table 2).

TABLE 3

LCB1 (SEQ ID NOS: 27397-27406)
1 -- A, C, D, E, F, G, H, I, K, L, M, N, P, Q, R, S, T, V, W, Y
2 -- A, D, E, F, G, H, I, K, L, M, N, P, Q, R, S, T, V, W, Y
3 -- A, D, E, F, G, H, K, L, M, N, P, Q, R, S, T, V, W, Y
4 -- A, C, D, E, F, G, H, I, K, L, M, N, P, Q, R, S, T, V, W, Y
5 -- A, C, D, E, F, G, H, I, K, L, M, N, P, Q, R, S, T, V, W, Y
6 -- A, C, I, L, M, Q, T, V
7-- A, C, D, E, F, G, H, M, N, P, Q, R, S, V, W, Y
8 -- A, C, D, E, F, G, H, I, K, L, M, N, Q, R, S, T, V, W, Y
9 -- C, I, L, M, N, Q, T, V
10 -- C, F, V, W, Y
11 -- A, C, D, E, F, G, H, I, K, L, M, N, Q, R, S, T, V, W, Y
12 -- A, C, D, H, I, L, M, N, S, T, V, Y
13 -- C, I, M, Q
14 -- A, C, D, E, F, G, H, I, K, L, M, N, Q, R, S, T, V, W, Y
15 -- A, C, D, E, F, G, H, I, K, L, M, N, Q, R, S, T, V, W, Y
16 -- C, F, I, L, M, T, V
17 -- A, C, D, E, F, G, H, I, K, L, M, N, Q, R, S, T, V, W, Y
18 -- A, C, D, E, F, G, H, I, K, L, M, N, Q, R, S, T, V, W, Y
19 -- A, C, D, E, F, G, H, I, K, L, M, N, Q, R, S, T, V, W, Y
20 -- A, C, D, E, F, G, H, K, L, M, N, Q, R, S, T, W
21 -- A, C, D, E, F, G, H, I, K, L, M, N, Q, R, S, T, V, W, Y
22 -- A, C, D, F, G, H, I, L, M, N, P, Q, S, T, V, W, Y
23 -- C, E, M, N, P, Q, S, T, V
24 -- A, C, D, E, F, G, H, K, L, M, N, P, Q, R, S, T, V,, Y
25 -- A, C, G, M, N, Q, S, T, V
26 -- M, N, V
27 -- A, C, D, E, F, G, H, I, K, L, M, N, Q, R, S, T, V, W, Y
28 -- A, C, G, I, L, S, T, V
29 -- A, C, S, V, W
30 -- D
31 -- A, C, D, F, G, H, I, K, L, M, N, Q, R, S, T, V, W, Y
32 -- C, F, H, I, L, M, N, P, T, V
33 -- A, C, D, E, F, G, H, I, K, L, M, N, P, Q, R, S, T, V, W, Y
34 -- A, C, D, E, F, G, H, I, K, L, M, N, P, Q, R, S, T, V, W, Y
35 -- A, C, D, F, H, M, Q, V, W, Y
36 -- A, C, D, E, G, H, I, L, M, N, Q, R, S, T, V, W, Y
37 -- A, C, D, E, F, G, H, I, K, L, M, N, P, Q, R, S, T, V, W, Y
38 -- A, C, D, E, F, G, H, I, K, L, M, N, Q, R, S, T, V, W, Y
39 X-- A, C, D, E, F, G, H, K, L, M, N, P, Q, R, S, T, V, W, Y
40 -- D, E, G, H, N, P, Q
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- A, C, D, E, F, G, H, I, K, L, M, Q, R, S, V, W, Y
45 X-- A, C, D, E, F, G, H, I, K, L, M, N, P, Q, R, S, T, V, W, Y
46 -- A, C, D, E, F, G, H, I, K, L, M, N, P, Q, R, S, T, V, W, Y
47 -- A, C, G, P, S, T, V
48-- A, C, D, E, F, G, H, I, K, L, M, N, Q, R, S, T, V, W, Y
49 -- A, C, D, E, F, G, H, I, K, L, M, N, Q, R, S, T, V, W, Y
50 -- A, C, D, E, F, G, H, I, K, L, M, N, Q, R, S, T, V, W, Y
51 -- A, C, E, F, G, H, I, K, L, M, N, Q, R, S, T, V, W, Y
52 -- A, C, D, E, F, G, H, I, K, L, M, N, P, Q, R, S, T, V, W, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- A, C, D, E, F, G, H, I, K, L, M, N, P, Q, R, S, T, V, W, Y

LCB2 (SEQ ID NOS: 27407- 27408)
1-- A, C, D, E, G, N, P, S, T
2 -- D, M, P, Q, Y
3 -- A, D, E, N, Q
4 -- C, D, E, V
5 -- D
6 -- A, C, D, E, G, N, Q, S, T, V
-- A, C, G, I, L, M, P, S, T, V
8 -- A, C, E, F, G, H, I, K, L, M, N, Q, R, S, T, V, W, Y
9 -- D, N, Y
10 -- I, L, T
11 -- C, E, G, I, L, M, W
12 -- F, H, Y
13 -- E, M, Q, R, V
14 -- A, C, E, F, G, H, I, K, L, M, N, Q, R, S, T, V, W, Y
15 -- A, C, D, E, G, H, I, K, L, M, N, Q, R, S, T, V
16 -- C, H, L, T
17 -- A, C, D, E, F, G, H, I, K, L, M, N, P, Q, R, S, T, V, W, Y
18 -- A, C, D, E, F, G, H, I, K, L, M, N, Q, R, S, T, V, W, Y
19 -- A, C, D, E, F, G, H, I, K, L, M, N, P, Q, R, S, T, V, W, Y
20 -- A, C, E, F, G, H, I, K, L, M, N, P, Q, R, S, T, V, Y
21 -- A, C, D, E, F, G, H, I, K, L, M, N, P, Q, R, S, T, V, W, Y
22 -- A, C, D, E, G, I, K, L, N, P, Q, R, S, T, V
23 -- A, C, D, E, F, G, H, I, K, L, M, N, P, Q, R, S, T, V, W, Y
24 -- A, C, D, E, F, G, H, I, K, L, M, N, P, Q, R, S, T,V, W, Y
25 -- A, C, E, G, H, I, K, N, P, Q, R, S, T, Y
26 -- A, C, D, E, F, G, H, I, K, L, M, N, P, Q, R, S, T, V, W, Y
27 -- A, C, D, E, F, G, H, I, K, L, M, N, P, Q, R, S, T, V, W, Y
28 -- H, K, R, T, Y
29 -- C, D, E, H, I, K, L, M, N, P, Q, R, S, T, V, Y
30 -- A, C, D, E, F, G, H, I, K, L, M, N, P, Q, S, T, V,W, Y
31 -- A, C, D, E, F, G, H, I, K, L, M, N, P, Q, R, S, T, V, Y
32 -- F, H, I, K, L, M, P, Q, R, Y
33 -- A, C, G, P, S, T
34 -- A, C, D, E, F, G, H, I, K, L, M, N, Q, R, S, T, V, W, Y
35 -- F, H, Y
36 -- A, C, E, H, I, L, M, S, V
37 -- A, C, E, G, H, L, M, Q, R, S, T, V, W
38 -- A, C, D, E, F, G, H, I, K, L, M, N, P, Q, R, S, T, V, W, Y
39 -- A, C, D, E, G, H, I, K, L, M, N, P, Q, R, S, T, V
40 -- A, C, D, E, G, H, I, K, L, M, N, P, Q, R, S, T, V, W, Y
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- A, C, D, E, F, G, H, I, K, L, M, N, P, Q, R, S, T, V, W, Y
45 -- A, C, E, F, I, L, M, P, S, T, V, W, Y
46 -- A, C, D, E, F, G, H, I, K, L, M, N, P, Q, R, S, T, V, W, Y
47 -- A, C, D, E, F, G, H, I, K, L, M, N, P, Q, R, S, T, V, W, Y
48 -- A, C, D, E, F, G, H, I, K, L, M, N, P, Q, R, S, T, V, W, Y
49 -- A, C, E, F, G, H, I, K, L, M, N, P, Q, R, S, T, V, W, Y
50 -- A, C, D, E, F, G, H, I, K, L, M, N, P, Q, R, S, T, V, W, Y
51 -- A, C, D, E, F, G, H, I, K, L, M, N, P, Q, R, S, T, V, W, Y
52 -- A, C, D, E, F, G, H, I, K, L, M, N, P, Q, R, S, T, V, W, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- A, C, D, E, F, G, H, I, K, L, M, N, P, Q, R, S, T, V, W, Y

LCB3 (SEQ ID NOS: 27409- 27416)
1 -- C, E, F, I, M, N, T, W
2 -- A, C, D, E, F, G, H, I, K, L, M, N, P, Q, R, S, T, V, W, Y
3 -- D, G, L, M, N, S, Y
4 -- A, C, E, F, H, K, Q, T
5 -- A, C, E, F, G, H, I, K, L, M, N, Q, R, S, T, V, W, Y
6 -- A, C, D, E, F, G, H, I, K, L, M, N, P, Q, R, S, T, V, W, Y
7 -- A, C, D, F, I, L, M, P, R, S, V, W
8 -- A, C, D, E, F, G, H, I, K, L, M, N, Q, R, S, T, V, W, Y
9 -- A, C, E, F, G, H, I, L, M, N, Q, R, S, T, V, Y
10 -- A, C, F, G, H, K, M, N, Q, R, S, T, Y
11 -- D, F, H, L, M, N, Q
12 -- A, C, D, E, F, G, H, I, K, L, M, N, P, Q, R, S, T, V, W, Y
13 -- A, F, I, L, M, N, Q, S, T, V
14-- A, C, F, G, H, I, K, L, M, N, P, Q, R, S, T, V, W, Y
15 -- A, C, D, E, F, G, H, I, K, L, M, N, P, Q, R, S, T, V, W, Y
16 -- A, C, D, E, F, G, H, I, L, M, N, P, Q, R, S, T, V, W, Y
17 -- A, C, D, F, G, H, I, K, L, M, N, P, Q, R, S, T, V, W
18 -- A, C, D, E, F, G, H, I, L, M, N, P, Q, R, S, T, V, W, Y
19 -- A, C, D, E, F, G, H, I, K, L, M, N, P, Q, R, S, T, V, W, Y
20 -- A, C, D, E, F, G, H, I, K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- A, C, D, E, F, G, H, I, K, L, M, N, P, Q, R, S, T, V, W, Y
24 -- A, C, D, E, F, G, H, I, K, L, M, N, P, Q, R, S, T, V, W, Y
25 -- A, C, D, E, F, G, H, I, K, L, M, N, P, Q, R, S, T, V, W, Y
26 -- A, C, D, E, F, G, H, I, K, L, M, N, P, Q, R, S, T, V, W, Y
27 -- A, C, D, E, F, G, H, I, K, L, M, N, P, Q, R, S, T, V, W, Y
28 -- A, C, D, E, F, G, H, I, K, L, M, N, P, Q, R, S, T, V, W, Y
29 -- A, C, D, E, F, G, I, L, M, N, P, S, T, V, W, Y
30 -- C, E, F, H, L, N, S, W, Y
31 -- A, C, D, E, F, G, H, I, K, L, M, N, P, Q, R, S, T, V, W, Y
32 -- A, C, D, E, F, G, H, I, K, L, M, N, P, Q, R, S, T, V, W, Y
33 -- A, C, E, F, I, K, P, Q, S, V, W, Y
34 -- A, D, E, F, G, H, M, N, P, Q, R, S, V, W, Y
35 -- A, C, D, E, F, G, H, I, K, L, M, N, P, Q, R, S, T, V, W, Y
36 -- A, C, E, G, H, I, M, N, Q, S, T, V
37 -- A, C, D, E, F, G, H, I, K, L, M, N, P, Q, R, S, T, V, W, Y
38 -- A, C, D, E, F, G, H, I, K, L, M, N, P, Q, R, S, T, V, W, Y
39 -- A, C, D, E, F, G, H, I, K, L, M, N, P, Q, R, S, T, V, W, Y
40 -- A, C, D, E, F, G, H, K, L, M, N, P, Q, R, S, T, V, W, Y
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- A, C, D, E, F, G, H, I, K, L, M, N, P, Q, R, S, T, V, W, Y
45 -- A, C, D, E, F, G, H, I, K, L, M, N, P, Q, R, S, T, V, W, Y
46 -- A, C, D, E, F, G, H, I, K, L, M, N, P, Q, R, S, T, V, W, Y
47 -- A, C, D, E, F, G, H, I, K, L, M, N, P, Q, R, S, T, V, W, Y
48 -- A, C, E, F, G, I, K, L, M, N, P, Q, S, T, V, W
49 -- A, C, D, E, F, G, H, I, K, L, M, N, P, Q, R, S, T, V, W, Y
50 -- A, C, D, E, F, G, H, I, K, L, M, N, P, Q, R, S, T, V, W, Y
51 -- A, C, D, E, F, G, H, I, K, L, M, N, P, Q, R, S, T, V, W, Y
52 -- A, C, D, E, F, G, H, I, K, L, M, N, P, Q, R, S, T, V, W, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, E, F, G, H, I, K, L, M, N, Q, S, T, V, W, Y
56 -- A, C, D, E, F, G, H, I, K, L, M, N, P, Q, R, S, T, V, W, Y
57 -- A, C, D, E, F, G, H, I, K, L, M, N, P, Q, R, S, T, V, W, Y
58 -- A, C, D, E, F, G, H, I, K, L, M, N, P, Q, R, S, T, V, W, Y
59 -- A, C, D, E, F, G, H, I, K, L, M, N, P, Q, R, S, T, V, W, Y
60 -- A, C, D, E, F, G, H, I, K, L, M, N, P, Q, R, S, T, V, W, Y
61 -- A, C, D, E, F, G, H, I, K, L, M, N, P, Q, R, S, T, V, W, Y
62 -- A, C, D, E, F, G, H, I, K, L, M, N, P, Q, R, S, T, V, W, Y
63 -- A, C, D, E, F, G, H, I, K, L, M, N, P, Q, R, S, T, V, W, Y
64 -- A, C, D, E, F, G, H, I, K, L, M, N, P, Q, R, S, T, V, W, Y

LCB4 (SEQ ID NO: 27417- 27418)
1 -- A, C, D, E, F, G, H, I, K, L, M, N, P, Q, R, S, T, V, W, Y
2 -- A, C, D, E, F, G, H, I, K, L, M, N, P, Q, R, S, T, V, W, Y
3 -- A, C, D, E, F, G, H, I, K, L, M, N, P, Q, R, S, T, V, W, Y
4 -- A, C, D, E, F, G, H, I, K, L, M, N, P, Q, R, S, T, V, W, Y
5 --\, D, H, K, N, Q, R, Y
6 -- A, C, F, G, I, K, L, M, P, Q, R, S, T, V, Y
7 -- A, C, D, E, F, G, H, I, K, L, M, N, P, Q, R, S, T, V, W, Y
8 -- A, C, H, I, M, N, Q, R, S, T, V, Y
9 -- A, C, D, G, H, I, K, L, M, N, Q, R, S, T, V, Y
10 -- A, C, D, E, M, N, P, Q, S, T, V
11 -- C, D, G, H, I, K, L, M, N, P, R, S, T, V
12 -- F, G, I, L
13 -- F, I, L, M, S, V, Y
14 -- A, C, D, E, G, L, M, N, Q, R, S, T, V
15 -- C, E, F, G, H, I, L, M, S, V, W, Y
16 -- A, G, T, Y
17 -- A, C, D, E, F, G, H, I, K, L, M, N, Q, R, S, T, V, W, Y
18 -- A, C, D, E, F, G, H, I, K, L, M, N, Q, R, S, T, V, W, Y
19 -- A, C, D, E, F, G, H, I, K, L, M, N, Q, R, S, T, V, W, Y
20 -- A, C, D, E, F, G, H, I, K, L, M, N, P, Q, R, S, T, V, W, Y
21 -- C, D, Q, Y
22 -- A, C, D, E, F, G, H, I, K, L, M, N, P, Q, R, S, T, V, W, Y
23 -- E, F, H, Y
24 -- A, F, G, I, L, M, W
25 -- A, C, E, G, H, I, K, L, M, N, Q, R, S, T, V, Y
26 -- C, F, H, I, L, N, S, T, V, W
27 -- D, Q, W, Y
28 -- A, C, D, I, L, V, Y
29 -- A, C, E, G, K, L, N, Q, R, S, T
30 -- C, I, L, M, P, T, V
31 -- C, D, E
32 -- A, C, E, I, L, M, Q, S, T, V, Y
33 -- A, C, E, F, G, H, I, K, L, M, Q, R, S, T, V, Y
34 -- C, D, F, G, H, L, M, N, P, R, S, T, W, Y
35 -- A, C, E, F, G, H, I, K, L, N, P, R, T, V, W
36 -- A, C, G, S, T, V
37 -- A, C, D, E, G, H, I, K, L, M, N, P, Q, R, S, T, V, Y
38 -- A, C, D, E, F, G, H, I, K, L, M, N, P, Q, R, S, T, V, W, Y
39 -- A, C, D, E, F, G, H, I, K, L, M, N, P, Q, R, S, T, V, W, Y
40 -- A, C, D, E, F, G, H, I, K, L, M, N, Q, R, S, T, V, Y
41 -- A, C, D, E, G, H, K, N, Q, S, W
42-- A, C, D, E, F, G, H, I, K, L, M, N, P, Q, R, S, T, V, W, Y
43 -- A, C, D, E, F, G, H, I, K, L, M, N, P, Q, R, S, T, V, Y
44 -- A, E, F, G, H, I, K, L, M, N, Q, R, S, T, V
45 -- A, C, E, F, G, H, I, K, L, M, N, Q, R, S, T, V, W, Y
46 -- A, C, D, E, F, G, H, I, K, L, M, N, Q, R, S, T, V, W, Y
47 -- A, C, D, E, F, G, H, I, K, L, M, N, Q, R, S, T, V, W, Y
48 -- A, C, M, S, T, V
49 -- A, H, I, K, L, M, N, Q, R, S, T, V, W, Y
50 -- A, C, D, E, F, G, H, I, K, L, M, N, P, Q, R, S, T, V, W, Y
51 -- A, F, I, K, L, M, R, T, V, W, Y
52 -- F, I, K, L, M, V
53 -- A, C, D, E, F, G, H, I, K, L, M, N, Q, R, S, T, V, W, Y
54 -- A, C, D, E, F, G, H, I, K, L, M, N, P, Q, R, S, T, V, W, Y
55 -- A, C, F, G, H, I, K, L, M, N, Q, R, S, T, V, W, Y
56 -- A, C, D, E, F, G, H, I, K, L, M,N, P, Q, R, S, T, V, W, Y

LCB5 (SEQ ID NO: 27419- 27420)
1 -- A, C, D, E, F, G, H, I, K, L, M, N, P, Q, R, S, T, V, W, Y
2 -- A, C, D, E, F, G, H, I, K, L, M, N, P, Q, R, S, T, V, W, Y
3 -- A, C, D, E, F, G, H, I, K, L, M, N, P, Q, R, S, T, V, W, Y
4 -- A, C, D, E, F, G, H, I, K, L, M, N, P, Q, R, S, T, V, W, Y
5 -- A, C, D, E, F, G, H, I, K, L, M, N, P, Q, R, S, T, V, W, Y
6 -- A, C, D, E, F, G, H, I, K, L, M, N, P, Q, R, S, T, V, W, Y
7 -- A, C, D, E, F, G, H, I, K, L, M, N, P, Q, R, S, T, V, W, Y
8 -- A, C, D, E, F, G, H, I, K, L, M, N, P, Q, R, S, T, V, W, Y
9 -- A, C, E, F, G, H, I, L, M, N, Q, S, T, V, W, Y
10 -- A, C, D, E, F, G, H, I, K, L, M, N, P, Q, R, S, T, V, W, Y
11 -- A, C, D, E, F, G, H, I, K, L, M, N, P, Q, R, S, T, V, W, Y
12 -- A, C, D, E, F, G, H, I, L, M, N, P, Q, R, S, T, V, W, Y
13 -- A, C, D, E, F, G, H, I, K, L, M, N, P, Q, R, S, T, V, W, Y
14 -- A, C, D, E, F, G, H, I, K, L, M, N, P, Q, R, S, T, V, W, Y
15 -- A, C, D, E, F, G, H, I, K, L, M, N, P, Q, R, S, T, V, W, Y
16 -- A, C, D, E, F, G, H, I, K, L, M, N, P, Q, R, S, T, V, W, Y
17 -- A, C, D, E, F, G, H, I, K, L, M, N, P, Q, R, S, T, V, W, Y
18 -- A, C, D, E, F, G, H, I, K, L, M, N, P, Q, R, S, T, V, W, Y
19 -- A, C, D, E, F, G, H, I, K, L, M, N, P, Q, R, S, T, V, W, Y
20 -- A, C, D, E, F, G, H, I, K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- A, C, D, E, F, G, H, I, L, M, N, P, Q, R, S, T, W, Y
24 -- A, C, D, E, F, G, H, I, L, M, N, P, Q, S,T, V, W, Y
25 -- A, C, D, E, F, G, H, I, K, L, M, N, P, Q, R, S, T, V, W, Y
26 -- A, C, D, E, F, G, H, I, K, L, M, N, P, Q, R, S, T, V, W, Y
27 -- A, C, G, H, I, S, T, V
28 -- A, C, D, E, F, G, H, I, K, L, M, N, Q, R, S, T, V, W, Y
29 -- A, C, D, E, F, G, H, I, K, L, M, N, P, Q, R, S, T, V, W, Y
30 -- A, C, D, E, F, G, H, I, K, L, M, N, P, Q, R, S, T, V, W, Y
31 -- A, C, E, F, H, I, K, L, M, N, Q, S, T, V, W, Y
32 -- A, C, D, E, F, G,H, I, K, L, M,N, P, Q, R, S, T, V, W, Y
33 -- A, C, D, E, F, G, H, I, K, L, M, N, P, Q, R, S, T, V, W, Y
34 -- A, C, D, E, F, G, H, I, K, L, M, N, P, Q, R, S, T, V, W, Y
35 -- A, C, D, E, F, G, H, I, K, L, M, N, Q, R, S, T, V, W, Y
36 -- A, C, D, E, F, G, H, I, K, L, M, N, P, Q, R, S, T, V, W, Y
37 -- A, C, D, E, F, G, H, I, L, M, N, P, Q, R, S, T, V, W, Y
38 -- A, C, D, E, G, I, L, M, N, P, Q, S, T, V, W
39 -- A, C, F, G, L, M, N, S, T, V, W
40 -- A, C, E, F, G, H, I, K, L, M, N, Q, R, S, T, V, Y
41 -- C, H, I, L, M, P, R
42 -- A, C, E, G, H, I, L, M, P, T, V, Y
43 -- C, I, L, M, Q, T, V
44 -- A, C, D, F, G, H, I, M, S, T
45 -- D, Y
46 -- A, C, D, F, I, L, R,V
47 -- C, E, G, I, V
48 -- F, I, V, W, Y
49 -- A, C, D, E, F, G, H, I, K, L, M, N, Q, R, S, T, V, W, Y
50 -- A, C, E, F, G, H, I, K, L, M, N, Q, R, S, T, V, W, Y
51 -- A, C, D, E, F, G, H, I, K, L, M, N, P, Q, R, S, T, V, W, Y
52 -- C, D, E, H, I, K, N, P, Q, R, S, T, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- F, I, L, M, T, V, W
57 -- A, C, D, E, F, G, H, N, P, Q, R, S, T, W, Y
58 -- A, C, D, E, F, G, H, I, K, L, M, N, P, Q, R, S, T, V, W, Y
59 -- A, C, F, I, L, M, T, V, Y
60 -- C, F, M, N, V, Y
61 -- A, C, D, E, F, G, H, I, K, L, M, N, Q, R, S, T, V, W, Y
62 -- A, C, F, G, I, L, M, S, T, V, W
63 -- A, C, E, F, G, H, I, K, L, M, N, Q, R, S, T, V, W, Y
64 -- A, C, E, F, G, H, K, L, N, P, R, S, T, W, Y
65 -- A, C, D, E, F, G, H, I, K, L, M, N, P, Q, R, S, T, V, W, Y

LCB6 (SEQ ID NO: 27421- 27422)
1 -- A, C, D, E, F, G, H, I, K, L, M, N, P, Q, R, S, T, V, W, Y
2 -- A, C, D, E, F, G, H, I, K, L, M, N, P, Q, R, S, T, V, W, Y
3 -- E, W
4 -- A, C, D, E, F, G, H, I, K, L, M, N, P, Q, R, S, T, V, W, Y
5 -- A, C, D, E, F, G, H, I, K, L, M, N, P, Q, R, S, T, V, W, Y
6 -- F, L, M, R, S
7 -- H, T, V
8 -- A, C, D, E, F, G, H, I, K, L, M, N, P, Q, R, S, T, V, W, Y
9 -- F, M
10 -- A, K, L, W
11 -- D, E, G, V, Y
12 -- A, C, D, E, F, G, H, I, K, L, M, N, P, Q, R, S, T, V, W, Y
13 -- E, L
14 -- A, C, D, E, F, G, H, I, K, L, M, N, P, Q, R, S, T, V, W, Y
15 -- A, C, D, E, F, G, H, I, K, L, M, N, P, Q, R, S, T, V, W, Y
16 -- A, C, D, E, F, G, H, I, K,L, M, N, P, Q, R, S, T, V, W, Y
17 -- F, N, P, S
18 -- A, C, D, E, F, G, H, I, K, L, M, N, P, Q, R, S, T, V, W, Y
19 -- L, N, Q, V
20 -- A, C, D, E, F, G, H, I, K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- C, D, P, Q, R, W
24 -- A, C, D, E, F, G, H, I, K, L, M, N, P, Q, R, S, T, V, W, Y
25 -- A, C, D, E, F, G, H, I, K, L, M, N, P, Q, R, S, T, V, W, Y
26 -- A, C, D, E, F, G, H, I, K, L, M, N, P, Q, R, S, T, V, W, Y
27 -- D, H, L, S, W
28 -- A, C, D, E, F, G, H, I, K, L, M, N, P, Q, R, S, T, V, W, Y
29 -- A, C, D, E, F, G, H, I, K, L, M, N, P, Q, R, S, T, V, W, Y
30 -- L, Q, V, W
31 -- I, K, L, S
32 -- A, C, D, E, F, G, H, I, K, L, M, N, P, Q, R, S, T, V, W, Y
33 -- A, C, D, E, F, G, H, I, K, L, M, N, P, Q, R, S, T, V, W, Y
34 -- A, F, L, T, V
35 -- C, D, G, H, K, L, N, T
36 -- A, C, D, E, F, G, H, I, K, L, M, N, P, Q, R, S, T, V, W, Y
37 -- A, C, D, E, F, G, H, I, K, L, M, N, P, Q, R, S, T, V, W, Y
38 -- A, C, D, E, F, G, H, I, K, L, M, N, P, Q, R, S, T, V, W, Y
39 -- A, C, D, E, F, G, H, I, K, L, M, N, P, Q, R, S, T, V, W, Y
40 -- A, C, D, E, F, G, H, I, K, L, M, N, P, Q, R, S, T, V, W, Y
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- F, I
45 -- A, C, D, E, F, G, H, I, K, L, M, N, P, Q, R, S, T, V, W, Y
46 -- A, C, D, E, F, G, H, I, K, L, M, N, P, Q, R, S, T, V, W, Y
47 -- A, C, D, E, F, G, H, I, K, L, M, N, P, Q, R, S, T, V, W, Y
48 -- L, Q, R, T
49 -- A, C, D, E, F, G, H, I, K, L, M, N, P, Q, R, S, T, V, W, Y
50 -- A, C, D, E, F, G, H, I, K, L, M, N, P, Q, R, S, T, V, W, Y
51 -- C, V, Y
52 -- A, E, H, K
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- C, F, H, L, P, W, Y
56 -- A, C, D, E, F, G, H, I, K, L, M, N, P, Q, R, S, T, V, W, Y

LCB7 (SEQ ID NO: 27423- 27424)
1 -- A, C, D, E, F, G, H, I, K, L, M, N, P, Q, R, S, T, V, W, Y
2 -- A, C, D, E, F, G, H, I, K, L, M, N, P, Q, R, S, T, V, W, Y
3 -- A, C, D, E, F, G, H, I, K, L, M, N, P, Q, R, S, T, V, W, Y
4 -- I, T, V
5-- A, C, D, E, F, G, H, I, K, L, M, N, P, Q, R, S, T, V, W, Y
6 -- A, C, D, E, F, G, H, I, K, L, M, N, P, Q, R, S, T, V, W, Y
7 -- L, P, Y
8 -- A, C, D, E, F, G, H, I, K, L, M, N, P, Q, R, S, T, V, W, Y
9 -- A, C, D, E, F, G, H, I, K, L, M, N, P, Q, R, S, T, V, W, Y
10 -- A, C, D, E, F, G, H, I, K, L, M, N, P, Q, R, S, T, V, W,Y
11 -- A
12 -- A, C, D, E, F, G, H, I, K, L, M, N, P, Q, R, S, T, V, W, Y
13 -- A, L, P
14 -- H, L, R, T, Y
15 -- A, C, D, E, F, G, H, I, K, L, M, N, P, Q, R, S, T, V, W, Y
16 -- A, C, D, E, F, G, H, I, K, L, M, N, P, Q, R, S, T, V, W, Y
17 -- A, C, D, E, F, G, H, I, K, L, M, N, P, Q, R, S, T, V, W, Y
18 -- A, C, D, E, F, G, H, I, K, L, M, N, P, Q, R, S, T, V, W, Y
19 -- A, C, D, E, F, G, H, I, K, L, M, N, P, Q, R, S, T, V, W, Y
20 -- A, C, D, E, F, G, H, I, K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- A, S
24 -- A, C, D, E, F, G, H, I, K, L, M, N, P, Q, R, S, T, V, W, Y
25 -- A, C, D, E, F, G, H, I, K, L, M, N, P, Q, R, S, T, V, W, Y
26 -- C, G, S, V, Y
27 -- K, L, M, W
28 -- A, C, D, E, F, G, H, I, K, L, M, N, P, Q, R, S, T, V, W, Y
29 -- A, C, D, E, F, G, H, I, K, L, M, N, P, Q, R, S, T, V, W, Y
30 -- A, Y
31 -- A, C, D, E, F, G, H, I, K, L, M, N, P, Q, R, S, T, V, W, Y
32 -- A, C, D, E, F, G, H, I, K, L, M, N, P, Q, R, S, T, V, W, Y
33 -- A, C, F, I, K, L, V, W
34 -- A, H, L
35 -- A, C, D, E, F, G, H, I, K, L, M, N, P, Q, R, S, T, V, W, Y
36 -- A, C, D, E, F, G, H, I, K, L, M, N, P, Q, R, S, T, V, W, Y
37 -- A, C, D, E, F, G, H, I, K, L, M, N, P, Q, R, S, T, V, W, Y
38 -- A, C, D, E, F, G, H, I, K, L, M, N, P, Q, R, S, T, V, W, Y
39 -- A, C, K, L, M, N
40 -- A, C, D, E, F, G, H, I, K, L, M, N, P, Q, R, S, T, V, W, Y
41 -- A, C, D, E, F, G, H, I, K, L, M, N, P, Q, R, S, T, V, W, Y
42 -- A, C, D, L, V
43 -- A, C, D, E, F, G, H, I, K, L, M, N, P, Q, R, S, T, V, W, Y
44 -- A, C, D, E, F, G, H, I, K, L, M, N, P, Q, R, S, T, V, W, Y
45 -- A, C, D, E, F, G, H, I, K, L, M, N, P, Q, R, S, T, V, W, Y
46 -- Q, S, V
47 -- A, C, D, E, F, G, H, I, K, L, M, N, P, Q, R, S, T, V, W, Y
48 -- A, C, D, E, F, G, H, I, K, L, M, N, P, Q, R, S, T, V, W, Y
49 -- E, L
50 -- A, C, D, E, F, G, H, I, K, L, M, N, P, Q, R, S, T, V, W, Y
51 -- A, C, D, E, F, G, H, I, K, L, M, N, P, Q, R, S, T, V, W, Y
52 -- A, C, D, E, F, G, H, I, K, L, M, N, P, Q, R, S, T, V, W, Y
53 -- I
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- L, M, N, R
57 -- A, C, D, E, F, G, H, I, K, L, M, N, P, Q, R, S, T, V, W, Y

LCB8 (SEQ ID NO: 27425- 27426)
1 -- A, C, D, E, F, G, H, I, K, L, M, N, P, Q, R, S, T, V, W, Y
2 -- C, F, I, L, M, S, V, W, Y
3 -- A, C, E, F, G, H, I, K, L, M, N, P, Q, R, S, T, V, W, Y
4 -- A, C, D, E, F, G, H, I, K, L, M, N, Q, R, S, T, V, W, Y
5 -- A, C, F, G, I, K, L, M, Q, S, T, V, W, Y
6 -- H, I, K, L, M
7 -- A, H, I, K, L, M, N, P, Q, R, W, Y
8 -- A, C, D, E, F, G, H, I, K, L, M, N, Q, R, S, T, V, W, Y
9 -- A, C, F, G, I, L, M, S, Y
10 -- A, F, H, K, L, M, Q, R, S
11 -- A, C, D, E, F, G, H, I, K, L, M, N, Q, R, S, T, V, W, Y
12 -- A, C, D, E, G, H, I, K, L, M, N, Q, R, S, T, V, W, Y
13 -- A, C, D, E, F, G, H, M, N, Q, S, W, Y
14 -- C, D, E, H, N, Q, S
15 -- A, D, E, F, H, I, L, M, N, P, Q, S, T, V, W, Y
16 -- C, F, M, N, R, Y
17 -- A, C, I, L, M, Q, R, V
18 -- A, C, F, H, I, L, M, T, V, Y
19 -- I, Q, S
20 -- D, N
21 -- A, C, G, S, V
22 -- A, C, I, L, M, V
23 -- C, F, R, T, W, Y
24 -- A, C, D, E, F, G, H, I, L, M, N, Q, R, S, T, V, W, Y
25 -- C, E, S, T, V, Y
26 -- A, C, D, E, F, G, H, N, Q, S, T
27 -- A, C, D, E, G, H, I, K, L, M, N, Q, R, S, T, V
28 -- C, E, F, G, H, I, K, L, M, Q, R, W, Y
29 -- A, C, F, G, H, I, K, L, M, N, Q, R, S, T, V, Y
30 -- A, C, E, G, H, K, M, N, P, Q, R, T
31 -- A, C, D, E, F, G, H, I, K, L, M, N, Q, R, S, T, V, Y
32 -- A, C, D, E, G, H, I, K, N, Q, R, S, T, W
33 -- A, C, E, G, H, K, M, N, P, Q, R, S, W, Y
34 -- C, D, E, F, H, M, N, W, Y
35 -- A, C, D, E, F, G, H, I, K, L, M, N, P, Q, R, S, T, V, Y
36 -- A, C, D, E, F, G, H, K, L, M, N, Q, R, S, T, V, W, Y
37 -- F, G, H, I, L, M, S, T, Y
38 -- D, E, H, Q, W, Y
39 -- C, D, E, F, G, H, K, L, M, N, P, Q, R, S, T, V, W, Y
40 -- A, C, E, G, H, I, K, M, P, V, Y
41 -- C, F, H, I, K, L, M, R, S, T, V
42 -- E, F, I, T, W, Y
43 -- A, C, D, E, F, H, I, L, M, N, Q, R, S, T, V, W, Y
44 -- C, G, I, K, L, M, T, V, Y
45 -- G, S, W, Y
46 -- C, I, K, L, M, N, Q, R, S, T
47 -- A, C, E, N, Q, S, T, V
48 -- C, D, E, F, H, I, L,M, W
49 -- C, D, F, H, K, L, M, N, Q, R, T
50 -- A, C, D, E, N, Y
51 -- A, C, D, E, F, G, H, I, K, L, M, N, P, Q, R, S, T, V, W, Y
52 -- A, C, D, E, G, H, K, L, M, N, Q, R, S, T
53 -- A, C, D, E, F, G, H, I, L, M, N, P, Q, S, T, V, W, Y
54 -- A, C, D, E, F, G, H, I, K, L, M, N, P, Q, R, S, T, V, W, Y
55 -- A, C, D, E, F, G, H, I, K, L, M, N, P, Q, S, T, V, W, Y
56 -- C, I, L, M
57 -- A, C, D, E, G, I, N, Q, S, T
58 -- A, C, D, E, F, G, H, I, K, L, M, N, P, Q, R, S, T, V, W, Y
59 -- A, C, G, P, S
60 -- A, C, E, F, G, I, L, M, N, Q, S, T, V
61 -- A, C, D, E, F, G, H, I, K, L, M, N, P, Q, R, S, T, V, W, Y
62 -- A, C, D, E, F, G, H, I, K, L, M, N, Q, R, S, T, V, W, Y
63 -- A, C, E, F, G, H, I, L, M, N, Q, S, T, V, W, Y
64 -- A, C, D, E, G, H, I, K, L, M, N, P, Q, S, T, V
65 -- A, C, D, E, G, H, I, K, L, M, N, P, Q, R, S, T, W, Y

AHB1 (SEQ ID NOS: 27427- 27428)
1 -- A, C, D, E, F, G, H, I, K, L, M, N, P, Q, R, S, T, V, W, Y
2 -- A, C, D, E, F, G, H, I, K, L, M, N, P, Q, R, S, T, V, W, Y
3 -- A, C, D, E, F, G, H, I, K, L, M, N, P, Q, R, S, T, V, W, Y
4 -- A, C, D, E, F, G, H, I, K, L, M, N, P, Q, R, S, T, V, W, Y
5 -- A, C, D, E, F, G, H, I, K, L, M, N, P, Q, R, S, T, V, W, Y
6 -- A, C, D, E, F, G, H, I, K, L, M, N, P, Q, R, S, T, V, W, Y
7 -- A, C, D, E, F, G, H, I, K, L, M, N, P, Q, R, S, T, V, W, Y
8 -- A, C, D, E, F, G, H, I, K, L, M, N, P, Q, R, S, T, V, W, Y
9 -- A, C, D, E, F, G, H, I, K, L, M, N, P, Q, R, S, T, V, W, Y
10 -- A, C, F, H, I, K, L, M, N, Q, R, S, T, V, W, Y
11 -- F, N, Y
12 -- A, C, D, E, F, G, H, I, K, L, M, N, P, Q, R, S, T, V, W, Y
13 -- A, C, D, E, F, G, H, I, K, L, M, N, P, Q, R, S, T, V, W, Y
14 -- A, D, G
15 -- A, C, D, E, G, H, I, K, L, M, N, Q, R, S, T, V
16 -- A, C, D, E, F, G, H, I, K, L, M, N, P, Q, R, S, T, V, W, Y
17 -- A, C, D, E, F, G, H, I, K, L, M, N, Q, R, S, T, V, W, Y
18 -- A, C, D, E, F, G, H, I, L, M, N, Q, S, T, V, W, Y
19 -- A, C, D, E, F, G, H, I, K, L, M, N, P, Q, R, S, T, V, W, Y
20 -- A, C, D, E, F, G, H, I, K, L, M, N, P, Q, R, S, T, V, W, Y
21 -- A, C, E, G, S, V
22 -- A, C, D, E, F, G, H, I, K, L, M, N, P, Q, R, S, T, V, W, Y
23 -- A, C, D, E, F, G, H, I, K, L, M, N, P, Q, R, S, T, V, W, Y
24 -- A, C, D, E, F, H, K, L, M, N, Q, R, S, T, V, Y
25 -- A, C, D, F, G, H, L, M, N, Q, R, S, T, V, W, Y
26 -- A, C, D, E, F, G, H, I, K, L, M, N, P, Q, R, S, T, V, W, Y
27 -- A, C, D, E, F, G, H, I, K, L, M, N, P, Q, R, S, T, V, W, Y
28 -- A, C, D, E, F, G, H, K, L, M, N, P, Q, R, S, T, Y
29 -- A, C, D, E, F, G, H, I, K, L, M, N, Q, R, S, T, V, W, Y
30 -- A, D, E, F, G, H, I, K, L, M, N, P, Q, R, S, T, V, W, Y
31 -- A, C, D, E, F, G, H, I, K, L, M, N, P, Q, R, S, T, V, W, Y
32 -- A, C, D, E, F, G, H, I, K, L, M, N, P, Q, R, S, T, V, W, Y
33 -- A, G, S
34 -- A, C, D, E, F, G, H, I, K, L, M, N, P, Q, R, S, T, V, W, Y
35 -- A, C, D, E, F, G, H, I, K, L, M, N, P, Q, R, S, T, V, W, Y
36 -- A, C, D, E, F, G, H, K, L, M, N, P, Q, R, S, T, V, W, Y
37 -- A, C, D, E, F, G, H, I, K, L, M, N, P, Q, R, S, T, V, W, Y
38 -- A, C, E, G, H, M, P, Q
39 -- A, C, D, E, F, G, H, I, K, L, M, N, P, Q, R, S, T, V, W, Y
40 -- A, C, D, E, G, K, N, Q, R, S, T
41 -- A, C, D, E, F, G, H, I, L, M, N, P, Q, S, T, V, W, Y
42 -- A, C, D, E, F, G, H, I, K, L, M, N, P, Q, R, S, T, V, W, Y
43 -- A, C, D, E, F, G, H, I, K, L, M, N, Q, S, T, V, W, Y
44 -- E, F, H, Q, S, W, Y
45 -- D, N
46 -- A, C, D, E, F, G, H, I, K, L, M, N, P, Q, R, S, T, V, W, Y
47 -, T, V
48 -- F, S, W, Y
49 -- A, C, D, E, F, G, H, I, K, L, M, N, Q, R, S, T, V, W, Y
50 -- A, C, F, H, I, K, L, M, N, Q, R, S, T, V, W, Y
51 -- A, D, G, H, N, S
52 -- H, K, Q, R
53 -- A, C, D, E, F, G, H, I, K, L, M, N, P, Q, R, S, T, V, W, Y
54 -- A, C, H, I, K, L, M, N, P, Q, R, S, T, V
55 -- A, C, E, G, H, K, N, Q, R, S, T
56 -- A, C, D, E, F, G, H, I, K, L, M, N, P, Q, R, S, T, V, W, Y
57 -- A, C, D, E, F, G, H, I, K, L, M, N, P, Q, R, S, T, V, W, Y
58 -- A, C, D, E, F, G, H, I, K, L, M, N, P, Q, R, S, T, V, W, Y
59 -- A, C, D, E, F, G, H, I, K, L, M, N, P, Q, R, S, T, V, W, Y
60 -- A, C, D, E, F, G, H, I, K, L, M, N, P, Q, R, S, T, V, W, Y
61 -- A, D, E, F, G, H, I, K, L, M, N, P, Q, R, S, T, V, W, Y
62 -- A, C, D, E, F, G, H, I, K, L, M, N, P, Q, R, S, T, V, W, Y
63 -- A, C, D, E, F, G, H, I, K, L, M, N, P, Q, R, S, T, V, W, Y
64 -- A, C, D, E, F, G, H, I, K, L, M, N, P, Q, R, S, T, V, W, Y
65 -- A, C, D, E, F, G, H, I, K, L, M, N, P, Q, R, S, T, V, W, Y
66 -- A, C, D, E, F, G, H, I, K, L, M, N, P, Q, R, S, T, V, W, Y
67 -- A, C, D, E, F, G, H, I, K, L, M, N, P, Q, R, S, T, V, W, Y
68 -- A, C, D, E, F, G, H, I, K, L, M, N, P, Q, R, S, T, V, W, Y
69 -- A, C, D, E, F, G, H, I, K, L, M, N, P, Q, R, S, T, V, W, Y
70 -- A, C, D, E, F, G, H, I, K, L, M, N, P, Q, R, S, T, V, W, Y
71 -- A, C, D, E, F, G, H, I, K, L, M, N, P, Q, R, S, T, V, W, Y
72 -- A, C, D, E, F, G, H, I, K, L, M, N, P, Q, R, S, T, V, W, Y
73 -- A, C, D, E, F, G, H, I, K, L, M, N, P, Q, R, S, T, V, W, Y
74 -- A, C, D, E, F, G, H, I, K, L, M, N, P, Q, R, S, T, V, W, Y
75 -- A, C, D, E, F, G, H, I, K, L, M, N, P, Q, R, S, T, V, W, Y
76 -- A, C, D, E, F, G, H, I, K, L, M, N, P, Q, R, S, T, V, W, Y
77 -- A, C, D, E, F, G, H, I, K, L, M, N, P, Q, R, S, T, V, W, Y
78 -- A, C, D, E, F, G, H, I, K, L, M, N, P, Q, R, S, T, V, W, Y
79 -- A, C, D, E, F, G, H, I, K, L, M, N, P, Q, R, S, T, V, W, Y
80 -- A, C, D, E, F, G, H, I, K, L, M, N, P, Q, R, S, T, V, W, Y
81 -- A, C, D, E, F, G, H, I, K, L, M, N, P, Q, R, S, T, V, W, Y
82 -- A, C, D, E, F, G, H, I, K, L, M, N, P, Q, R, S, T, V, W, Y
83 -- A, C, D, E, F, G, H, I, K, L, M, N, P, Q, R, S, T, V, W, Y
84 -- A, C, D, E, F, G, H, I, K, L, M, N, P, Q, R, S, T, V, W, Y
85 -- A, C, D, E, F, G, H, I, K, L, M, N, P, Q, R, S, T, V, W, Y

AHB2 (SEQ ID NO: 27429- 27430)
1 -- C, G, A, V, F, Y, W, S, Q, D, E, R, K
2 -- C, P, G, V, I, M, L, F, Y, W, S, N, Q, D, E, R, H
3 -- C, G, A, V, I, F, S, T, D, E, K
4 -- C, P, G, A, V, I, M, L, F, Y, W, S, T, N, Q, D, E, R, K, H
5 -- C, P, G, A, V, M, L, Y, W, S, N, Q, D, E, R, K, H
6 -- G, A, V, I, F, S, T, D, H
7 -- C, P, G, V, I, M, L, F, W, S, T, N, Q, E, R, K, H
8 -- C, P, G, A, V, M, L, Y, W, S, T, N, Q, D, E, R, K, H
9 -- C, P, G, A, V, I, M, L, F, W, S, T, N, Q, D, E, R, K, H
10 -- C, P, G, A, V, I, L, Y, W, S, T, N, E, R, K
11 -- C, P, G, A, V, I, M, L, F, Y, W, S, T, N, Q, D, E, R, H
12 -- C, P, G, A, V, I, L, F, Y, W, S, T, N, Q, D, E, R, K, H
13 -- C, G, A, V, M, L, F, W, S, T, N, E, H
14 -- C, P, G, A, V, I, Y, S, T, N, D, E, R, H
15 -- C, G, A, V, I, M, L, F, Y, W, S, T, N, Q, D, E, R, K
16 -- C, P, G, A, V, I, M, L, F, Y, W, S, T, N, Q, D, E, R, K, H
17 -- C, P, G, A, V, L, Y, W, S, T, Q, D, E, R
18 -- C, P, A, V, I, M, F, Y, N, Q, R, K, H
19 -- C, P, G, A, V, I, M, L, F, Y, W, S, T, N, Q, D, E, R, K, H
20 -- C, P, G, A, V, M, L, Y, W, N, Q, E, R, K, H
21 -- C, P, G, A, V, I, M, L, F, Y, W, S, N, Q, E, R, K, H
22 -- C, P, G, A, V, M, L, F, Y, S, T, N, Q, D, E, R, K, H
23 -- C, P, G, A, V, I, M, L, F, Y, W, S, T, N, Q, E, R, K
24 -- C, P, G, A, V, I, M, L, F, Y, W, S, Q, E, R, H
25 -- C, P, G, A, V, I, M, L, F, Y, W, S, T, N, Q, D, R, H
26 -- C, G, A, V, L, Y, S, N, D, R, K, H
27 -- C, P, G, A, V, I, M, L, F, Y, W, S, T, N, Q, D, E, R, K, H
28 -- C, P, G, A, V, I, M, L, F, Y, W, S, T, N, Q, D, E, R, K, H
29 -- C, P, G, V, I, M, L, F, Y, W, S, T, N, Q, D, R, K, H
30 -- C, P, G, A, V, I, M, L, F, Y, W, S, T, N, Q, D, E, R, K, H
31 -- C, G, A, V, I, M, L, F, Y, W, S, T, Q, D, E, R, K, H
32 -- P, G, A, V, I, L, W, S, T, D, R, H
33 -- C, P, G, A, V, I, M, L, F, Y, W, S, T, N, Q, E, R, K, H
34 -- C, G, A, V, I, M, L, F, Y, W, S, T, N, Q, D, E, R, K, H
35 -- C, P, G, A, V, I, M, L, F, Y, W, S, T, N, Q, D, E, R, K, H
36 -- C, P, G, A, V, I, L, F, Y, S, T, N, Q, D, E, R, H
37 -- C, G, A, V, I, M, L, F, Y, W, S, T, N, Q, D, E, R, K, H
38 -- C, P, G, A, V, I, M, L, F, Y, W, S, T, Q, E, R, K
39 -- C, P, G, A, V, I, W, S, Q, E, R, H
40 -- C, P, G, A, V, I, L, Y, W, S, T, N, D, E, R, K, H
41 -- C, P, G, A, V, I, M, L, Y, W, S, T, N, Q, D, E, R, K, H
42 -- C, P, G, A, V, M, L, Y, W, S, T, N, Q, D, E, R, K, H
43 -- C, G, A, V, I, M, L, F, Y, W, S, T, N, Q, D, E, R, K, H
44 -- C, P, G, A, V, I, M, L, F, W, S, T, Q, D, E, R, H
45 -- C, G, A, V, I, M, L, F, Y, W, S, T, N, Q, D, E, R, K, H
46 -- C, P, G, A, V, I, M, L, F, S, T, Q, E, R, K
47 -- C, G, A, V, I, M, L, F, W, S, T, N, Q, D, E, R, H
48 -- C, P, G, A, V, I, M, L, F, Y, W, S, N, Q, E, R, K
49 -- C, P, G, A, V, M, L, F, Y, W, S, T, N, Q, D, E, R, K, H
50 -- C, P, G, A, V, I, M, L, F, Y, W, S, T, N, Q, D, E, R, K, H
51 -- C, G, A, V, I, M, L, F, Y, W, S, T, N, Q, D, E, R, K, H
52 -- C, P, G, A, V, I, M, L, F, Y, W, S, T, N, Q, D, E, R, K, H
53 -- C, P, G, A, V, I, M, L, F, Y, W, S, T, N, D, E, R, K, H
54 -- C, P, G, A, V, I, M, L, F, Y, W, S, T, N, Q, D, E, R, K, H
55 -- C, P, G, A, V, I, M, L, F, Y, S, T, N, Q, D, E, R, K, H
56 -- C, P, G, A, V, I, M, L, F, Y, W, S, T, N, Q, D, E, R, K, H
57 -- C, P, G, A, V, I, M, L, F, Y, W, S, T, N, Q, D, E, R, K, H
58 -- C, G, A, V, I, M, L, F, Y, W, S, T, N, E, R, K, H
59 -- C, P, G, A, V, I, M, L, F, Y, W, S, T, N, Q, D, E, R, K, H
60 -- C, G, A, V, I, M, L, F, Y, W, S, T, Q, D, E, R, K
61 -- C, P, G, A, V, I, M, L, F, Y, W, S, N, Q, D, E, R, K, H
62 -- C, G, A, V, L, S, T, N, D, E, K, H
63 -- C, P, G, A, V, I, L, F, Y, W, S, T, N, Q, D, E, R, K, H
64 -- C, P, G, A, V, I, M, L, F, Y, W, S, T, N, Q, D, E, R, H
65 -- C, G, A, V, I, M, L, F, Y, S, T, N, R, K, H
66 -- C, P, G, A, V, I, M, L, W, T, Q, E, R
67 --, P, G, A, V, I, M, L, F, Y, W, S, T, N, Q, D, E, R, K, H
68 -- C, P, G, A, V, I, L, F, Y, W, S, T, N, Q, D, E, R, H
69 -- P, G, V, I, M, L, Y, W, S, T, Q, R, K
70 -- C, P, G, A, V, I, M, L, F, Y, W, S, T, N, Q, D, E, R, K, H
71 -- C, G, A, V, L, F, W, S, Q, D, E, R, K
72 -- C, V, I, L, S
73 -- P, G, A, V, S, T, E
74 --, A, L, F, Y, S, T, R, H
75 -- C, P, G, V, I, L, F, W, S, N, D, E, R, K

In one embodiment, the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:27397-27406 and 27431-27466.

TABLE 4

Exemplary LCB1 variants

| | |
|---|---|
| Name | Binder Protein |
| LCB1_4N | DKENILQKIYEIMRLLDELGHAEASMRVSDLIYEFMKKGDERLLEEAERLLEEVER (SEQ ID NO:27431) |
| LCB1_4K | DKEKILQKIYEIMRLLDELGHAEASMRVSDLIYEFMKKGDERLLEEAERLLEEVER (SEQ ID NO:27432) |
| LCB1_14K | DKEWILQKIYEIMKLLDELGHAEASMRVSDLIYEFMKKGDERLLEEAERLLEEVER (SEQ ID NO:27433) |
| LCB1_15T | DKEWILQKIYEIMRTLDELGHAEASMRVSDLIYEFMKKGDERLLEEAERLLEEVER (SEQ ID NO:27435) |
| LCB1_18Q | DKEWILQKIYEIMRLLDQLGHAEASMRVSDLIYEFMKKGDERLLEEAERLLEEVER (SEQ ID NO:27436) |
| LCB1_18K | DKEWILQKIYEIMRLLDKLGHAEASMRVSDLIYEFMKKGDERLLEEAERLLEEVER (SEQ ID NO: 27437) |
| LCB1_27Q | DKEWILQKIYEIMRLLDELGHAEASMQVSDLIYEFMKKGDERLLEEAERLLEEVER (SEQ ID NO: 27438) |
| LCB1_27Y | DKEWILQKIYEIMRLLDELGHAEASMYVSDLIYEFMKKGDERLLEEAERLLEEVER (SEQ ID NO: 27439) |
| LCB1_17E | DKEWILQKIYEIMRLLEELGHAEASMRVSDLIYEFMKKGDERLLEEAERLLEEVER (SEQ ID NO: 27440) |
| LCB1_17R | DKEWILQKIYEIMRLLRELGHAEASMRVSDLIYEFMKKGDERLLEEAERLLEEVER (SEQ ID NO: 27441) |
| LCB1_42N | DKEWILQKIYEIMRLLDELGHAEASMRVSDLIYEFMKKGDE [symbol] (SEQ ID NO: 27442) |
| LCB1_49Q | DKEWILQKIYEIMRLLDELGHAEASMRVSDLIYEFMKKGDERLLEEAEQLLEEVER (SEQ ID NO: 27443) |
| LCB1_52Q | DKEWILQKIYEIMRLLDELGHAEASMRVSDLIYEFMKKGDERLLEEAERLLQEVER (SEQ ID NO: 27444) |
| LCB1_32L | DKEWILQKIYEIMRLLDELGHAEASMRVSDLLYEFMKKGDERLLEEAERLLEEVER (SEQ ID NO: 27445) |
| LCB1_28A | DKEWILQKIYEIMRLLDELGHAEASMRASDLIYEFMKKGDERLLEEAERLLEEVER (SEQ ID NO: 27446) |
| LCB1_v1.3_ACH1 | DKENILQKIYEIMKTLEQLGHAEASMYVSDLIYEFMKQGDERLLEEAERLLEEVER (SEQ ID NO: 27447) |
| LCB1_v1.3_ACH2 | DKENILQKIYEIMKTLEQLGHAEASMQVSDLIYEFMKQGDENLLEEAERLLEEVER (SEQ ID NO: 27448) |
| LCB1_v1.3_ACH3 | DKENILQKIYEIMKTLEQLGHAEASMQVSDLIYEFMKQGDERLLEEAEQLLEEVER (SEQ ID NO: 27449) |
| LCB1_v1.3_ACH4 | DKENILQKIYEIMKTLEQLGHAEASMYVSDLIYEFMKQGDENLLEEAEQLLEEVER (SEQ ID NO: 27450) |
| LCB1_v1.3_ACH5 | DKENILQKIYEIMKTLEQLGHAEASMQVSDLIYEFMKQGDENLLEEAEQLLEEVER (SEQ ID NO: 27451) |
| LCB1_v1.3_1 | DRENILQKIYEIMKELEKLGHAEASMQVSDLIYEFMQDKDERLLEEAERLLEEVKR (SEQ ID NO: 27452) |
| LCB1_vl.3_2 | DRENILQKIYEIMKELRQLGHAEASMQVSDLIYEFMKTKDKRLLEEAERLLEEVKR (SEQ ID NO: 27453) |
| LCB1_v1.3_3 | DRENILQKIYEIMKTLRRLGHAEASMQVSDLIYEFMQDKDKRLLEEAERLLEEVQR (SEQ ID NO: 27454) |
| LCB1_v1.3_4 | DKENVLQKIYEIMKELERLGHAEASMQVSDLIYEFMKTKDERLLEEAERLLEEVKR (SEQ ID NO: 27455) |
| LCBl_v1.3_5 | DRENILQKIYEIMKTLEKLGHAEASMQASDLIYEFMKTKDERLLEEAERLLEEVQR (SEQ ID NO: 27456) |
| LCBl_v1.3_6 | DKENILQKIYEIMKTLRALGHAEASMQVSDLIYEFMQTKDERLLEEAERLLEEVKR (SEQ ID NO: 27457) |
| LCB1_v1.3_7 | DKENVLQKIYEIMKTLEKLGHAEASMQVSDLIYEFMQTKDKRLLEEAERLLEEVQR (SEQ ID NO: 27458) |
| LCB1_v1.3_15 | DRENILQKIYEIMKELEKLGHAEASMQVSDLIYEFMQDKDENLLEEAERLLEEVKR (SEQ ID NO: 27459) |
| LCB1_v1.3_16 | DRENILQKIYEIMKELRQLGHAEASMQVSDLIYEFMKTKDKNLLEEAERLLEEVKR (SEQ ID NO: 27460) |
| LCB1_v1.3_17 | DRENILQKIYEIMKTLRRLGHAEASMQVSDLIYEFMQDKDKNLLEEAERLLEEVQR (SEQ ID NO: 27461) |
| LCB1_v1.3_19 | DRENILQKIYEIMKTLEKLGHAEASMQASDLIYEFMKTKDENLLEEAERLLEEVQR (SEQ ID NO: 27462) |
| LCB1_v1.3_20 | DKENILQKIYEIMKTLRALGHAEASMQVSDLIYEFMQTKDENLLEEAERLLEEVKR (SEQ ID NO: 27463) |
| LCB1_v1.3_21 | DKENVLQKIYEIMKTLEKLGHAEASMQVSDLIYEFMQTKDKNLLEEAERLLEEVQR (SEQ ID NO: 27464) |
| LCB1_v2.2 | DKENVLQKIYEIMKELERLGHAEASMQVSDLIYEFMKTKDENLLEEAERLLEEVKR (SEQ ID NO: 27465) |
| LCB1_v2.2_ompT | DKENVLQKIYEIMKELERLGHAEASMQVSDLIYEFMKTKDENLLEEAERLLEEVTR (SEQ ID NO: 27466) |

[symbol]

indicates text missing or illegible when filed

In another embodiment, the one or more target binding polypeptide comprises an amino acid substitution relative to the amino acid sequence of SEQ ID NO: 27397 at 1, 2, 3, 4, 5, 6, 7, 8, 9, 10, 11, 12, 13, 14, 15, 16, 17, or all 18 residues selected from the group consisting of 2, 4, 5, 14, 15, 17, 18, 27, 28, 32, 37, 38, 39, 41, 42, 49, 52, and 55. In a further embodiment, the substitutions in the one or more target binding polype [symbol] the substitutions listed in Table 5, either individually or in combinations in a given row.

TABLE 5

Exemplary LCB1 mutations

| | | | | | | | | | | | | |
|---|---|---|---|---|---|---|---|---|---|---|---|---|
| Name | Parent | Mutations from WT | | | | | | | | | | |
| LCB1_1 | LCB1 | W4N | R14 K | L15 T | E18 Q | R27 Q | K38 Q | | | | | |
| LCB1_2 | LCB1 | W4N | R14 K | L15 T | E18 Q | R27 Y | K38 Q | | | | | |
| LCB1_3 | LCB1 | W4N | R14 K | L15 T | D17 E | E18 Q | R27 Q | K38 Q | | | | |
| LCB1_4 | LCB1 | W4N | R14 K | L15 T | D17 E | E18 Q | R27 Q | K38 Q | R42 N | R49 Q | E52 Q | |
| LCB1_4N | LCB1 | W4N | | | | | | | | | | |
| LCB1_4K | LCB1 | W4K | | | | | | | | | | |
| LCB1_14K | LCB1 | R14K | | | | | | | | | | |
| LCB1_15T | LCB1 | LIST | | | | | | | | | | |
| LCB1_18Q | LCB1 | E18Q | | | | | | | | | | |
| LCB1_18K | LCB1 | E18K | | | | | | | | | | |
| LCB1_27Q | LCB1 | R27Q | | | | | | | | | | |
| LCB1_27Y | LCB1 | R27Y | | | | | | | | | | |
| LCB1_38Q | LCB1 | K38Q | | | | | | | | | | |
| LCB1_17E | LCB1 | D17E | | | | | | | | | | |
| LCB1_17R | LCB1 | D17R | | | | | | | | | | |
| LCB1_42N | LCB1 | R42N | | | | | | | | | | |
| LCB1_49Q | LCB1 | R49Q | | | | | | | | | | |
| LCB1_52Q | LCB1 | E52Q | | | | | | | | | | |
| LCB1_32L | LCB1 | 132L | | | | | | | | | | |
| LCB1_28A | LCB1 | V28A | | | | | | | | | | |
| LCB1_v1.3 | LCB1 | W4N | R14 K | L15 T | D17 E | E18 Q | R27 Q | K38 Q | | | | |
| LCB1_v1.3_ACH 1 | LCB1_v1.3 | W4N | R14 K | L15 T | D17 E | E18 Q | R27 Y | K38 Q | | | | |
| LCB1_v1.3_ACH 2 | LCB1_v1.3 | W4N | R14 K | L15 T | D17 E | E18 Q | R27 Q | K38 Q | R42 N | | | |
| LCB1_v1.3_ACH 3 | LCB1_v1.3 | W4N | R14 K | L15 T | D17 E | E18 Q | R27 Q | K38 Q | R49 Q | | | |
| LCB1_v1.3_ACH 4 | LCB1_v1.3 | W4N | R14 K | L15 T | D17 E | E18 Q | R27 Y | K38 Q | R42 N | R49 Q | | |
| LCB1_v1.3_ACH 5 | LCB1_v1.3 | W4N | R14 K | L15 T | D17 E | E18 Q | R27 Q | K38 Q | R42 N | R49 Q | | |
| LCB1_v1.3_1 | LCB1_v1.3 | K2R | W4N | R14 K | L15 E | D17 E | E18 K | R27 Q | K37 Q | K38 D | G39 K | E55 K |
| LCB1_v1.3_2 | LCB1_v1.3 | K2R | W4N | R14 K | L15 E | D17 R | E18 Q | R27 Q | K38 T | G39 K | E41 K | E55 K |
| LCB1_v1.3_3 | LCB1_v1.3 | K2R | W4N | R14 K | L15 T | D17 R | E18 R | R27 Q | K37 Q | K38 D | G39 K | E41 K |
| LCB1_v1.3_4 | LCB1_v1.3 | W4N | I5V | R14 K | L15 E | D17 E | E18 R | R27 Q | K38 T | G39 K | E55 K | |
| LCB1_v1.3_5 | LCB1_v1.3 | K2R | W4N | R14 K | L15 T | D17 E | E18 K | R27 Q | V2 8 A | K38 T | G39 K | E55 Q |
| LCB1_v1.3_6 | LCB1_v1.3 | W4N | R14 K | L15 T | D17 R | E18 A | R27 Q | K37 Q | K38 T | G39 K | E55 K | |
| LCB1_v1.3_7 | LCB1_v1.3 | W4N | I5V | R14 K | L15 T | D17 E | E18 K | R27 Q | K37 Q | K38 T | G39 K | E41 K |
| LCB1_vl.3_15 | LCB1_v1.3 | K2R | W4N | R14 K | L15 E | D17 E | E18 K | R27 Q | K37 Q | K38 D | G39 K | R42 N |
| LCB1_v1.3_16 | LCB1_v1.3 | K2R | W4N | R14 K | L15 E | D17 R | E18 Q | R27 Q | [symbol] | [symbol] | [symbol] | [symbol] |
| LCB1_v1.3_17 | LCB1_v1.3 | K2R | W4N | R14 K | L15 T | D17 R | E18 R | R27 Q | [symbol] Q | [symbol] D | [symbol] K | [symbol] K |
| LCB1_v1.3_19 | LCB1_v1.3 | K2R | W4N | R14 K | L15 T | D17 E | E18 K | R27 Q | V2 8 A | K38 T | G39 K | R42 N |
| LCB1_v1.3_20 | LCB1_v1.3 | W4N | R14 K | L15 T | D17 R | E18 A | R27 Q | K37 Q | K38 T | G39 K | R42 N | E55 K |
| LCB1_v1.3_21 | LCB1_v1.3 | W4N | ISV | R14 K | L15 T | D17 E | E18 K | R27 Q | K37 Q | K38 T | G39 K | E41 K |
| LCB1_v2.2 | LCB1_v1.3 | W4N | I5V | R14 K | L15 E | D17 E | E18 R | R27 Q | K38 T | G39 K | R42 N | E55 K |
| LCB1_v2.2_omp T | LCB1_v1,3 | W4N | I5V | R14 K | L15 E | D17 E | E18 R | R27 Q | K38 T | G39 K | R42 N | E55 T |

[symbol]

indicates text missing or illegible when filed

In a further embodiment, the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:27409-27416 and 27467-27493.

TABLE 6

Exemplary LCB3 variants

| | |
|---|---|
| Name | Binder Protein |
| LCB3_8Q | NDDELHMQMTDLVYEALHFAKDEEIKKRVFQLFELADKAYKNNDRQKLEKVVEELKELLE RLLS (SEQ ID NO: 27467) |
| LCB3_8T | NDDELHMTMTDLVYEALHFAKDEEIKKRVFQLFELADKAYKNNDRQKLEKVVEELKELLE RLLS (SEQ ID NO: 27468) |
| LCB3_19K | NDDELHMLMTDLVYEALHKAKDEEIKKRVFQLFELADKAYKNNDRQKLEKVVEELKELLE RLLS (SEQ ID NO: 27469) |
| LCB3_19I | NDDELHMLMTDLVYEALHIAKDEEIKKRVFQLFELADKAYKNNDRQKLEKWEELKELLE RLLS (SEQ ID NO: 27470) |
| LCB3_25F | NDDELHMLMTDLVYEALHFAKDEEFKKRVFQLFELADKAYKNNDRQKLEKVVEELKELLE RLLS (SEQ ID NO: 27471) |
| LCB3_25M | NDDELHMLMTDLVYEALHFAKDEEMKKRVFQLFELADKAYKNNDRQKLEKVVEELKELLE RLLS (SEQ ID NO: 27472) |
| LCB3_26Q | NDDELHMLMTDLVYEALHFAKDEEIQKRVFQLFELADKAYKNNDRQKLEKWEELKELLE RLLS (SEQ ID NO: 27473) |
| LCB3_28H | NDDELHMLMTDLVYEALHFAKDEEIKKHVFQLFELADKAYKNNDRQKLEKWEELKELLE RLLS (SEQ ID NO: 27474) |
| LCB3_35K | NDDELHMLMTDLVYEALHFAKDEEIKKRVFQLFEKADKAYKNNDRQKLEKWEELKELLE RLLS (SEQ ID NO: 27475) |
| LCB3_37T | NDDELHMLMTDLVYEALHFAKDEEIKKRVFQLFELATKAYKNNDRQKLEKWEELKELLE RLLS (SEQ ID NO: 27476) |
| LCB3_40R | NDDELHMLMTDLVYEALHFAKDEEIKKRVFQLFELADKARKNNDRQKLEKWEELKELLE RLLS (SEQ ID NO: 27477) |
| LCB3_43K | NDDELHMLMTDLVYEALHFAKDEEIKKRVFQLFELADKAYKNKDRQKLEKWEELKELLE RLLS (SEQ ID NO: 27478) |
| LCB3_34G | NDDELHMLMTDLVYEALHFAKDEEIKKRVFQLFGLADKAYKNNDRQKLEKWEELKELLE RLLS (SEQ ID NO: 27479) |
| LCB3_34Y | NDDELHMLMTDLVYEALHFAKDEEIKKRVFQLFYLADKAYKNNDRQKLEKWEELKELLE RLLS (SEQ ID NO: 27480) |
| LCB3_34T | NDDELHMLMTDLVYEALHFAKDEEIKKRVFQLFTLADKAYKNNDRQKLEKWEELKELLE RLLS (SEQ ID NO: 27481) |
| LCB3_49K | NDDELHMLMTDLVYEALHFAKDEEIKKRVFQLFELADKAYKNNDRQKLKKWEELKELLE RLLS (SEQ ID NO: 27482) |
| LCB3_v1.2_ ACH1 | NDDELHMQMTDLVYEALHFAKDEEIQKHVFQLFGKATKAYKNKD [symbol] RLLS (SEQ ID NO: 27483) |
| LCB3_v1.2_ ACH2 | NDDELHMQMTDLVYEALHFAKDEEIQKHVFQLFYKATKAYKNKDRQKLEKVVEELKELLE RLLS (SEQ ID NO: 27484) |
| LCB3_v2.2 | NLDELHMQMTDLVYEALHFAKDEEFQKHVFQLFEKATKAYKNKDRQKLEKVVEELKELLE RLLS (SEQ ID NO: 27485) |
| LCB3_v1.3_ 2 | NDDELHMQMTDLVYEALHFAKTEEFQKHVFQLFEKATKAYKNKDRQKLEKVVEELKELLE RLLS (SEQ ID NO: 27486) |
| LCB3_v1.3_ 3 | NDDELHMQMTDLVYEALHFAKDEEFQKHVFQLFEKARKAYKNKDRQKLEKVVEELKELLE RLLS (SEQ ID NO: 27487) |
| LCB3_v1.3_ 4 | NDDELHMQMTDLVWEALHFAKDEEFQKHVFQLFEKARKAYKNKDRQKLEKVVEELKELLE RLLS (SEQ ID NO: 27488) |
| LCB3_v1.3_ 5 | NDDELHMQMTDLVWEALHFAKDEEFQKHVFQLFEKATKAYKNKDRQKLEKVVEELKELLE RLLS (SEQ ID NO: 27489) |
| LCB3_v1.3_ 6 | NEDELHMQMTDLVWEALHFAKDEEFQKHVFQLFEKATKAYKNKDRQKLEKVVEELKELLE RLLS (SEQ ID NO: 27490) |
| LCB3_v1.3_ 7 | NDDELHMQMTDLVWEALHFAKTEEFQKHVFQLFEKATKAYKNKDRQKLEKVVEELKELLE RLLS (SEQ ID NO: 27491) |
| LCB3_v1.3_ 15 | NLDELHMQMTDLVYEALHFAKTEEFQKHVFQLFEKATKAYKNKDRQKLEKVVEELKELLE RLLS (SEQ ID NO: 27492) |
| LCB3_v2.3 | NIDELLMQVTDLIYEALHFAKDEEFQKHAFQLFEKATKAYKNKDKQKLEKVVEELKELLE RILS (SEQ ID NO: 27493) |

In one embodiment, the target binding comprises an amino acid substitution relative to the amino acid sequence of SEQ ID NO:27409 at 1, 2, 3, 4, 5, 6, 7, 8, 9, 10, 11, 12, 13, 14, 15, 16, 17, 18, 19, or all 20 residues selected from the group consisting 2, 6, 8, 9, 13, 14, 19, 22, 25, 26, 28, 29, 34, 35, 37, 40, 43, 45, 49, and 62. In another embodiment, the substitutions are selected from the substitutions listed in Table 7, either individually or in combinations in a given row.

TABLE 7

Exemplary LCB3 mutations

| | | | | | | | | | | |
|---|---|---|---|---|---|---|---|---|---|---|
| Name | Parent | Mutations from WT | | | | | | | | |
| LCB3_1 | LCB3 | L8Q | I25F | K26Q | R28H | L35K | D37T | | | |
| LCB3_2 | LCB3 | L8Q | K26Q | R28H | L35K | D37T | N43K | | | |
| LCB3_3 | LCB3 | L8Q | I25F | K26Q | R28H | L35K | D37T | N43K | | |
| LCB3_4 | LCB3 | L8Q | F19K | I25F | K26Q | R28H | L35K | D37T | Y40R | N43K |
| LCB3_8Q | LCB3 | L8Q | | | | | | | | |
| LCB3_8T | LCB3 | L8T | | | | | | | | |
| LCB3_19K | LCB3 | F19K | | | | | | | | |
| LCB3_19I | LCB3 | F19I | | | | | | | | |
| LCB3_25F | LCB3 | I25F | | | | | | | | |
| LCB3_25M | LCB3 | I25M | | | | | | | | |
| LCB3_26Q | LCB3 | K2 6Q | | | | | | | | |
| LCB3_28H | LCB3 | R28H | | | | | | | | |
| LCB3_35K | LCB3 | L35K | | | | | | | | |
| LCB3_37T | LCB3 | D37T | | | | | | | | |
| LCB3_40R | LCB3 | Y40R | | | | | | | | |
| LCB3_43K | LCB3 | N43K | | | | | | | | |
| LCB3_34G | LCB3 | E34G | | | | | | | | |
| LCB3_34Y | LCB3 | E34Y | | | | | | | | |
| LCB3_34T | LCB3 | E34T | | | | | | | | |
| LCB3_49K | LCB3 | E49K | | | | | | | | |
| LCB3_v1.2 | LCB3 | L8Q | K26Q | R28H | L35K | D37T | N43K | | | |
| LCB3_vl.2_ACH1 | LCB3_v1.2 | L8Q | K26Q | R28H | E34G | L35K | D37T | N43K | | |
| LCB3_vl.2_ACH2 | LCB3_v1.2 | L8Q | K26Q | R28H | E34Y | L35K | D37T | N43K | | |
| LCB3_v2.2 | LCB3_v1.3 | D2L | L8Q | I25F | K26Q | R28H | L35K | D37T | N43K | |
| LCB3_v1.3_2 | LCB3_v1.3 | L8Q | D22T | I25F | K26Q | R28H | L35K | D37T | N43K | |
| LCB3_v1.3_3 | LCB3_v1.3 | L8Q | I25F | K26Q | R28H | L35K | D37R | N43K | | |
| LCB3_v1.3_4 | LCB3_v1.3 | L8Q | Y14W | I25F | K26Q | R28H | L35K | D37R | N43K | |
| LCB3_v1.3_5 | LCB3_v1.3 | L8Q | Y14W | I25F | K26Q | R28H | L35K | D37T | N43K | |
| LCB3_v1.3_6 | LCB3_v1.3 | D2E | L8Q | Y14W | I25F | K26Q | R28H | L35K | D37T | N43K |
| LCB3_v1.3_7 | LCB3_v1.3 | L8Q | Y14W | D22T | I25F | K26Q | R28H | L35K | D37T | N43K |
| LCB3_v1.3 | LCB3_v1.2 | L8Q | I25F | K26Q | R28H | L35K | D37T | N43K | | |
| LCB3_v1.3_15 | LCB3_v1.3 | D2L | L8Q | D22T | I25F | K26Q | R28H | L35K | D37T | N43K |
| LCB3_v2.3 | LCB1_v2.1 | D2I | H6L | L8Q | M9V | V13I | I25F | K2 6Q | R28H | V29A, L35K, D37T, N43K, R45K, L62I |

In one embodiment, the target binding comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27427-27430 and 27494.

AHB2 ELEEQVMHVLDQVSELAHELLHKLTGEELERAAYFNWWATEMMLELIKSDDEREIREIEEEAARILEH
v2 LEELART (SEQ ID NO: 27494)

In one such embodiment, the one or more target binding polypeptide comprises an amino acid substitution relative to the amino acid sequence of SEQ ID NO: 27430 at or both residues selected from the group consisting 63 and 75. In another embodiment, the substitutions comprise R63A and/or K75T.

In a further embodiment, the cage protein comprises the amino 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a cage polypeptide disclosed in US20200239524 (or WO2020/018935), not including optional amino acid residues and not including amino acid residues in the latch region. These cage protein amino acid sequences do not include the one or more target binding polypeptides or the first reporter protein domain (or the second reporter protein domain when present), which can thus be added to the cage proteins of this embodiment.

Exemplary such embodiment are SEQ ID NOS:1-49, 51-52, 54-59, 61, 65, 67-91, 92 -2033, 2034-14317, 27094-27117, 27120-27125, 27,278 to 27,321, and cage polypeptides with an even-numbered SEQ ID NO between SEQ ID NOS: 27126 and 27276), Table 3 (Table 8 in the current application), and/or Table 4 (Table 9 in the current application) of a cage polypeptide disclosed in US20200239524, and reproduced herein and in the sequence listing.

In each embodiment, the N-terminal and/or C-terminal 60 amino acids of each cage protein may be optional, as the terminal 60 amino acid residues may comprise a latch region that can be modified, such as by replacing all or a portion of a latch with the one or more target binding polypeptide and the first reporter protein domain. In one embodiment, the N-terminal 60 amino acid residues are optional; in another embodiment, the C-terminal 60 amino acid residues are optional; in a further embodiment, each of the N-terminal 60 amino acid residues and the C-terminal 60 amino acid residues are optional. In one embodiment, these optional N-terminal and/or C-terminal 60 residues are not included in determining the percent sequence identity. In another embodiment, the optional residues may be included in determining percent sequence identity.

TABLE 8

| Row number | Cage (column 1) | Key (column 2) |
|---|---|---|
| 1 | LOCKR _extend18 (SEQ ID NO:6), BimLOCKR_extend18 (SEQ ID NO:22),, 1fix-long-Bim-t0 (SEQ ID NO: 54), 1fix-long-GFP-t0 (SEQ ID NO: 55), 1fix-short-BIM-t0 (SEQ ID NO: 56), 1fix-short-GFP-t0 (SEQ ID NO: 57), | p18_MBP (SEQ ID NO:27020), p76-long (SEQ ID NO:27027), p76-short (SEQ ID NO:27028), |
| 2 | LOCKRb (SEQ ID NO:7), | key_b (SEQ ID NO:27022) |
| 3 | LOCKRc (SEQ ID NO:8), | key_c (SEQ ID NO:27023) |

TABLE 9

| | | | |
|---|---|---|---|
| Cage Name | Cage Sequence | Key Name | Key Sequence |
| 2plus 1_Cag e_Cte rm_24 06 | SEVDEVVKEVEDLVRRNEELVEEVVRRVEKVVTDDRR LVEEVVREIRKIVKDVEDLARKLDKEELKRVLDEMRE RIERLLEKLRRHSKKLDDELKRLLEELREHSRRVEKR LEDLLKELRERGVDEKVLRKLEKVIREVRERSTRALR KVEEVIRRVREESERALRDLERVVKEVEKRMREAAR (SEQ ID NO:27126) | 2plus1_ Key_Cte rm_2406 | EKVLRKLEKVIREVRE RSTRALRKVEEVIRRV REESERALRDLERVVK EVEKRMREAAR(SEQ ID NO:27127) |
| 2plus 1_Cag e_Cte rm_53 98 | SVEELLRKLEEVLRKIREENERSLKELRDRAREIVKR NRETNRELEEVIKELEKRLSGADKEKVEELVRRIRRI VERVVEEDRRTVEEIEKIAREVVKRDRDSADRVRRTV EDVLRKATGSEDIVRKIERIVETIEREVRESVKKVEE IARDIRRKVDESVKNVEKLLRDVDKKARDRKK (SEQ ID NO:27128) | 2plus1_ Key_Cte rm_5398 | EDIVRKIERIVETIER EVRESVKKVEEIARDI RRKVDESVKNVEKLLR DVDKKARDRKK(SEQ ID NO:27129) |
| 2plus 1_Cag e_Cte rm_54 05 | SESDDVIRKLRELLEELRTHVEKSIRDLRKILEDSTR HAKRSIEELERLLEEVRKKPGDEEVRKTVEEISRRVA ENVKRLEDLYRRMEEEVKKNLDRLRKRVEDIIREVEE ARKKGVDEEKLKDLIRKLRDILRRAAEAHKKLIDDAR ESLERAKREHEKLIDRLKKILEELER 3(SEQ ID NO:27130) | 2plus1_ Key_Cte rm_5405 | EEKLKDLIRKLRDILR RAAEAHKKLIDDARES LERAKREHEKLIDRLK KILEELER(SEQ ID NO:27131) |
| 2plus 1_Cag e_Cte rm_54 06 | DREREVKKRLDEVRERIERLLRRVEEESRRVAEEIRR LIEEVRRRNKKVTEEIRELLKGLKDKEEVRRVLERLR KLNAESDELLERILERLRRLVEATNRLVKAIIEELRR LVEKIVREVPDSEELREELKKLERKIEKVAKEIHDHD KEVTERLEDLLRRITEHARKSDREIEETAR(SEQ ID NO:27132) | 2plus1_ Key_Cte rm_5406 | EELREELKKLERKIEK VAKEIHDHDKEVTERL EDLLRRITEHARKSDR EIEETAR(SEQ ID NO:27133) |
| 2plus 1_Cag e_Cte rm_54 09 | SEAEELLKRLEDRAEEILRRLEEILRTSRKLAEDVLR ELEKLLRESERRIREVLEELRGIKDKKELEDVIREVE KELDESLERSRELLKDVLKKLDDNLKESERLVEDIDR ELAKILEDLKKAGVPKEVVDEIKRIVDEVRERLKRIV DENAKIVEDARRALEKIVKENEEILRRLKKELRELRK (SEQ ID NO:27134) | 2plus1_ Key_Cte rm_5409 | KEVVDEIKRIVDEVRE RLKRIVDENAKIVEDA RRALEKIVKENEEILR RLKKELRELRK(SEQ ID NO:27135) |
| 2plus 1_Cag e_541 4_GFP 11_Ct erm | SEIEKILKEIEDLARRDEEVSKKIVEDIRRLAKEVED TSRDIVRKIEELAKRVLDRLRKDGSKEELEKEVREVV KTLEELVKDNHRLIRRAVEEMKRLVEENHRHSREVVK ELEDLVRELRKGSGSEDSERDHMVLHEYVNAAGITSE KSNEEVKRTVEELVRRMEESNDRVRDLVRRLVEELKR AVD(SEQ ID NO:27140) | 2plus1_ Key_Cte rm_5 414 | EDSERLVREVEDLVRR LVRRSEKSNEEVKRTV EELVRRMEESNDRVRD LVRRLVEELKRAVD(S EQ ID NO:27141) |
| 2plus 1_Cag e_Cte rm_54 21 | SVDEVLKEIEDALRRLKEEVERVLKENEDELRRLEEE VRRVLKEDEELLESLKRGVGESDEVDRVVDEIAKLSA EILEKVKKVVKEIRDSLETVKRRVDDVVRRLKELLDE IKRGSDEKAIRDVAKEIRDRLKELEEEIEEVTRRNLK LLADVEEEIRRVHEKTRRLLETVLRRAT(SEQ ID NO:27146) | 2plus1_ Key_Cte rm_5421 | EKAIRDVAKEIRDRLK ELEEEIEEVTRRNLKL LADVEEEIRRVHEKTR RLLETVLRRAT(SEQ ID NO:27147) |
| 2plus 1_Cag e_Cte rm_54 32 | DEIRKVVKEITDLLKASNDKNRKVVEEIRDLLRKSKK LADELVERLRALVEDLRRRIDKSGDKETAEDIVRRII EELKRILKEIEDLARRINREIERLVEEVERDNRDVNR AIEELLKDIARRGGSEDLKRVEERAREVSRRNEESMR RVKEDADRVSEANKEVLDRVREEVKRLIEEVRETLR ( SEQ ID NO:27148) | 2plus1_ Key_Cte rm_5432 | SEDLKRVEERAREVSR RNEESMRRVKEDADRV SEANKEVLDRVREEVK RLIEEVRETLR(SEQ ID NO:27149) |
| 2plus 1_Cag e_Cte rm_54 35 | STAETVAEEVERVLKHSDDLIKEVEDVNRRVEEEIKR VIRELEEENERLVAEVRKGVKGEILAEIEKRLADNSE KVREVAERAKKLLEENTARVKDILRESRKLVKDLLDE VRGTGSEEAAREIIKRLREVNKRTKEKLDELIKHSEE VLERVKRLIDELRKHSEEVLEDLRRRAK(SEQ ID NO:27150) | 2plus1_ Key_Cte rm_5435 | EEAAREIIKRLREVNK RTKEKLDELIKHSEEV LERVKRLIDELRKHSE EVLEDLRRRAK(SEQ ID NO:27151) |
| 2plus | SRVEEIIEDLRRLLEEIRKENEDSIRRSKELLDRVKE | 2plus1_ | EDKARKVAEVAEKVLR |
| 1_Cag e_Cte rm_54 39 | INDTIIAELERLLKDIEKEVREKGSESEEVKKALRAV LEELEKLLRRVAEINEEVLRRNSKLVEEDERRNREVL KELARLVEELIREIGDEDKARKVAEVAEKVLRDIDKL DRESKEAFRATNEEIAKLDEDTARVAERVKKAIEDLA K(SEQ ID NO:27154) | Key_Cte rm_5439 | D N RVKKAIEDLAK(SEQ ID NO:27155) |
| 2plus 1_Cag e_Cte rm_54 47 | SEADDVLKKLAETVKRIIERLKKLTDDSRRLVEEVHR RNDKLSKESAEAVRKAEERGIDEKDVRKLLEDLKKKS EEVAERNKRILDTLREISKRAEDEVRKVLKELEKTLK ELEDRRPDSEELSAEVKKLLDEVRKALARHKDENDKL LKEIEDSLRRHKEENDRLLEKLKESTR(SEQ ID NO:27156) | 2plus1_ Key_Cte rm_5447 | EELSAEVKKLLDEVRK ALARHKDENDKLLKEI EDSLRRHKEENDRLLE KLKESTR(SEQ ID NO:27157) |
| 2plus 1_Cag e_Cte rm_54 56 | SAEELLREVAELVKRVDEDLRRLLEEVRASNEEVIRR LEEILKRIEEENRKVVEELRRGGVSEDLVRESKRLVD ESRRVIEKLVKESADSVERTRETVDRLREELKRLVEE IAKMVKGGSSEETVKRLLDELRELLERLKRTIEELLK RNRDLLADAEEKARRLLEENRKLLKAARDTAT (SEQ ID NO:27158) | 2plus1_ Key_Cte rm_5465 | EETVKRLLDELRELLE RLKRTIEELLKRNRDL LADAEEKARRLLEENR KLLKAARDTAT(SEQ ID NO:27159) |
| 2plus 1_Cag e_Nte rm_24 06 | SEVDEVVKEVEDLVRRNEELVEEVVRRVEKVVTDDRR LVEEVVREIRKIVKDVEDLARKLDKEELKRVLDEMRE RIERLLEKLRRHSKKLDDELKRLLEELREHSRRVEKR LEDLLKELRERGVDEKVLRKLEKVIREVRERSTRALR KVEEVIRRVREESERALRDLERVVKEVEKRMREAAR ( SEQ ID NO:27162) | 2plus1_ Key_Nte rm_2406 | SEVDEVVKEVEDLVRR NEELVEEVVRRVEKVV TDDRRLVEEVVREIRK IVKDVEDLARK(SEQ ID NO:27163) |
| 2plus 1_Cag e_Nte rm_54 06 | DREREVKKRLDEVRERIERLLRRVEEESRRVAEEIRR LIEEVRRRNKKVTEEIRELLKGLKDKEEVRRVLERLR KLNAESDELLERILERLRRLVEATNRLVKAIIEELRR LVEKIVREVPDSEELREELKKLERKIEKVAKEIHDHD KEVTERLEDLLRRITEHARKSDREIEETAR(SEQ ID NO:27164) | 2plus1_ Key_Nte rm_5406 | DREREVKKRLDEVRER IERLLRRVEEESRRVA EEIRRLIEEVRRRNKK VTEEIRELLKGL(SEQ ID NO:27165) |
| 2plus 1_Cag e_Nte rm_54 09 | SEAEELLKRLEDRAEEILRRLEEILRTSRKLAEDVLR ELEKLLRESERRIREVLEELRGIKDKKELEDVIREVE KELDESLERSRELLKDVLKKLDDNLKESERLVEDIDR ELAKILEDLKKAGVPKEVVDEIKRIVDEVRERLKRIV DENAKIVEDARRALEKIVKENEEILRRLKKELRELRK (SEQ ID NO:27166) | 2plus1_ Key_Nte rm_5409 | SEAEELLKRLEDRAEE ILRRLEEILRTSRKLA EDVLRELEKLLRESER RIREVLEELRGI(SEQ ID NO:27167) |
| 3plus _Cage _529_ GFP11 _{_}Cter m | SEAEDLEELIKELAELLKDVIRKLEKINRRLVKILED IIRRLKEISKEAEEELRKGTVEDKDILRDLERRLREI LEESDRLLEELKRRLEEILRKSKELLRRLEEVLREIL KRAEEVKRSNLPKEELIKEIVKLLEELLRVIEKILED NIRLLEELVEVIKEILEKHLRLLEELVRVIERILREV GKDKDEAERRDHMVLHEYVNAAGITEEVKRRLEEELT RLRETHKKIEKELREALKRVRDRST(SEQ ID NO:27178) | 3plus _K ey_Cter m 529 | KDEAERRRRELKDKLD RLREEHEEVKRRLEEE LTRLRETHKKIEKELR EALKRVRDRST(SEQ ID NO:27179) |
| 3plus 1_Cag e_Cte rm_5 0 0 | SEKEELKRLLDKLLKELKRLSDELKATIDKILKILKE VSEEVKRTADELLDAIRRGGVDEEVLREIKREIEEIE KKLRKVNKEIEDEIREIKKKLDEVDDKITKEVEKIKE ALDKGGVDAKEVIKALKEILKEHADVFEDVLRRLKEI IKRHRDVVKEVLEELRKILEKVAEVLKRQGRSEDELR KVEEDLKRLEDKLKKLLEDYEKKVRELEETLDDLLRK YEETLRRLEKELEEAER(SEQ ID NO:27184) | 3plus1_ Key_Cte rm_500 | EDELRKVEEDLKRLED KLKKLLEDYEKKVREL EETLDDLLRKYEETLR RLEKELEEAER(SEQ ID NO:27185) |
| 3plus 1_Cag e_Cte rm_51 0 | SEKEELLKLIKRVIELLKRVLEEHLRLVEDVIRRLKE LLDSNEKIVREVIEDLKRLLDEVRGDKEELDRIKEKL EEVLERYKRRLEEIKRDLERMLEDYKRELKRIEEDLR RVLEEVERIATRGEGPAEALIDKLRKILERALRELDK LSKKLDELLKKVLEELEKSNREIDKLLKDVLRRVEEG GASEDLLRKAKKVITEVREKLKRNLEDVRRVIEDVKR KSARILEEARRLIEEVERELEKIRK(SEQ ID NO:27190) | 3plus1_ Key_Cte rm_510 | EDLLRKAKKVITEVRE KLKRNLEDVRRVIEDV KRKSARILEEARRLIE EVERELEKIRK(SEQ ID NO:27191) |
| 3plus 1_Cag e 528 | SEAEDLEELIKELAELLKDVIRKLEKINRRLVKILED IIRRLKEISKEAEEELRKGTVEDKDILRDLERRLREI LEESDRLLEELKRRLEEILRKSKELLRRLEEVLREIL | 3plus1_ Key_Cte rm 528 | KDEAERRRRELKDKLD RLREEHEEVKRRLEEE LTRLRETHKKIEKELR |
| _GFP1 1_Cte rm | KRAEEVKRSNLPKEELIKEIVKLLEELLRVIEKILED NIRLLEELVEVIKEILEKHLRLLEELVRVIERILREV GRDHMVLHEYVNAAGITLDRLREEHEEVKRRLEEELT RLRETHKKIEKELREALKRVRDRST(SEQ ID NO:27192) | | E I |
| 3plus 1_Gag e_528 _GFP1 1_Cte rm | SEAEDLEELIKELAELLKDVIRKLEKINRRLVKILED IIRRLKEISKEAEEELRKGTVEDKDILRDLERRLREI LEESDRLLEELKRRLEEILRKSKELLRRLEEVLREIL KRAEEVKRSNLPKEELIKEIVKLLEELLRVIEKILED NIRLLEELVEVIKEILEKHLRLLEELVRVIERILREV GKDKRDHMVLHEYVNAAGITLREEHEEVKRRLEEELT RLRETHKKIEKELREALKRVRDRST(SEQ ID NO:27194) | 3plus1_ Key_Cte rm_528 | KDEAERRRRELKDKLD RLREEHEEVKRRLEEE LTRLRETHKKIEKELR EALKRVRDRST(SEQ ID NO:27195) |
| 3plus 1_Cag e_528 _GFP1 1_Cte rm | SEAEDLEELIKELAELLKDVIRKLEKINRRLVKILED IIRRLKEISKEAEEELRKGTVEDKDILRDLERRLREI LEESDRLLEELKRRLEEILRKSKELLRRLEEVLREIL KRAEEVKRSNLPKEELIKEIVKLLEELLRVIEKILED NIRLLEELVEVIKEILEKHLRLLEELVRVIERILREV GKDKDEAERDHMVLHEYVNAAGITHEEVKRRLEEELT RLRETHKKIEKELREALKRVRDRST(SEQ ID NO:27196) | 3plus1_ Key_Cte rm_528 | KDEAERRRRELKDKLD RLREEHEEVKRRLEEE LTRLRETHKKIEKELR EALKRVRDRST(SEQ ID NO:27197) |
| 3plus 1_Cag e_529 _GFP1 1_Cte rm | SEAEDLEELIKELAELLKDVIRKLEKINRRLVKILED IIRRLKEISKEAEEELRKGTVEDKDILRDLERRLREI LEESDRLLEELKRRLEEILRKSKELLRRLEEVLREIL KRAEEVKRSNLPKEELIKEIVKLLEELLRVIEKILED NIRLLEELVEVIKEILEKHLRLLEELVRVIERILREV GKRDHMVLHEYVNAAGITDRLREEHEEVKRRLEEELT RLRETHKKIEKELREALKRVRDRST(SEQ ID NO:27198) | 3plus1_ Key_Cte rm_529 | KDEAERRRRELKDKLD RLREEHEEVKRRLEEE LTRLRETHKKIEKELR EALKRVRDRST(SEQ ID NO:27199) |
| 3plus 1_Cag e_529 _GFP1 1_Cte rm | SEAEDLEELIKELAELLKDVIRKLEKINRRLVKILED IIRRLKEISKEAEEELRKGTVEDKDILRDLERRLREI LEESDRLLEELKRRLEEILRKSKELLRRLEEVLREIL KRAEEVKRSNLPKEELIKEIVKLLEELLRVIEKILED NIRLLEELVEVIKEILEKHLRLLEELVRVIERILREV GKDRDHMVLHEYVNAAGITRLREEHEEVKRRLEEELT RLRETHKKIEKELREALKRVRDRST(SEQ ID NO:27200) | 3plus1_ Key_Cte rm_529 | KDEAERRRRELKDKLD RLREEHEEVKRRLEEE LTRLRETHKKIEKELR EALKRVRDRST(SEQ ID NO:27201) |
| 3plus 1_Cag e_529 _GFP1 1_Cte rm | SEAEDLEELIKELAELLKDVIRKLEKINRRLVKILED IIRRLKEISKEAEEELRKGTVEDKDILRDLERRLREI LEESDRLLEELKRRLEEILRKSKELLRRLEEVLREIL KRAEEVKRSNLPKEELIKEIVKLLEELLRVIEKILED NIRLLEELVEVIKEILEKHLRLLEELVRVIERILREV GKDKDRDHMVLHEYVNAAGITREEHEEVKRRLEEELT RLRETHKKIEKELREALKRVRDRST(SEQ ID NO:27202) | 3plus1_ Key_Cte rm_529 | KDEAERRRRELKDKLD RLREEHEEVKRRLEEE LTRLRETHKKIEKELR EALKRVRDRST(SEQ ID NO:27203) |
| 3plus 1_Cag e_534 _GFP1 1_Cte rm | DEDRIIEEIARLLEELLRELLELIKKLIETNRRLNEE HERAVRELARLLEELLDRLVKKGISDEKLKRIRERLK RALDDLERLHREINKRLEDLVRELEKLVREILKELKD ALEELRRASARAGGEEVLRRLEEIVKKLLDLVRRILE RLKEIHKDNVRLLRELNERLTRIVEDLVRLIREILRE AGVDERDHMVLHEYVNAAGITIKRLHEDLERKLKESE DELREIEARLEEKIRRLEEKLERKRR(SEQ ID NO:27206) | 3plus1_ Key_Cte rm_534 | EKIAEEIERELEELRR MIKRLHEDLERKLKES EDELREIEARLEEKIR RLEEKLERKRR(SEQ ID NO:27207) |
| 3plus 1_Cag e_534 _GFP1 1_Cte rm | DEDRIIEEIARLLEELLRELLELIKKLIETNRRLNEE HERAVRELARLLEELLDRLVKKGISDEKLKRIRERLK RALDDLERLHREINKRLEDLVRELEKLVREILKELKD ALEELRRASARAGGEEVLRRLEEIVKKLLDLVRRILE RLKEIHKDNVRLLRELNERLTRIVEDLVRLIREILRE AGVDEKIRDHMVLHEYVNAAGITRLHEDLERKLKESE DELREIEARLEEKIRRLEEKLERKRR(SEQ ID NO:27208) | 3plus1_ Key_Cte rm_534 | EKIAEEIERELEELRR MIKRLHEDLERKLKES EDELREIEARLEEKIR RLEEKLERKRR(SEQ ID NO:27209) |
| 3plus 1_Cag e_534 _GFP1 1_Cte rm | DEDRIIEEIARLLEELLRELLELIKKLIETNRRLNEE HERAVRELARLLEELLDRLVKKGISDEKLKRIRERLK RALDDLERLHREINKRLEDLVRELEKLVREILKELKD ALEELRRASARAGGEEVLRRLEEIVKKLLDLVRRILE RLKEIHKDNVRLLRELNERLTRIVEDLVRLIREILRE AGVDEKIAEEIERDHMVLHEYVNAAGITLERKLKESE DELREIEARLEEKIRRLEEKLERKRR (SEQ ID NO:27210) | 3plus1_ Key_Cte rm_534 | E M EDELRE1EARLEEK1R RLEEKLERKRR(SEQ ID NO:27211) |
| 3plus 1_Cag e_Cte rm 53 9 | SEKEKLLKESEEEVRRLRRTLEELLRKYREVLERLRK ELREIEERVRDVVRRLKEVLDRKGLDIDTIIKEVEDL LKTVLDRLRELLDKIRRLTKEAIEVVREIIERIVRHA ERVKDELRKEGGDKEKLDRVDRLIKENTRHLKEILDR IEDLVRRSEKKLRDIIREVRRLIEELRKKAEEIKKGP DERLVKTLIEDVEAVIKRILELITRVAEDNERVLERI IRELTDNLERHLKIVREIVK (SEQ ID NO:27212) | 3plus1_ Key_Cte rm_539 | ERLVKTLIEDVEAVIK RILELITRVAEDNERV LERIIRELTDNLERHL KIVREIVK(SEQ ID NO:27213) |
| 3plus 1_Cag e_Cte rm_54 8 | DKAEVLREALKLLKDLLEELIKIHEESLKRILDLIDT LVKVHEDALRALKELLERSGLDERELRKVERMATESL RTIAKLKEELRDLARRSLEKLREDLKRVDDTLRKVEE KVRRTGPSEELIEELIRTIEKLLKEIVRINEEVLKAV RELLKTLLKLSEDVVRRIEEILRKGGVPEEIDRELKR VVEELRRLHEEIKERLDDVARRSEEELRRIIKKLKEV VKEIRKKLK (SEQ ID NO:27214) | 3plus1_ Key_Cte rm_548 | EEIDRELKRVVEELRR LHEEIKERLDDVARRS EEELRRIIKKLKEVVK EIRKKLK(SEQ ID NO:27215) |
| 3plus 1_Cag e_Cte rm_55 6 | SERELIERWLELHKEILRLIRELVERLLKLHREILDT IKKLIRELLELLEDIARKLGLDKEAKDELREIAKRVE DKLEKLERESRKVEEDLKRKLKELTDESDTVEKRVRD VVRRGTQSREEIAEELLRLDRKLLKAVEELLKEILDL NKKLLDDVRAILEETRRVLEKLLDRVRRGERTDDERR TLTELLKRMEDILEKVERTLKKLLDDSARMAEEVKKT LKELLERSEKVAEDVRK (SEQ ID NO:27216) | 3plus1_ Key_Cte rm_556 | DDERRTLTELLKRMED ILEKVERTLKKLLDDS ARMAEEVKKTLKELLE RSEKVAEDVRK(SEQ ID NO:27217) |
| 3plus 1_Cag e_Cte rm 56 0 | SKKELLEEVVRRAIELLKRHLEKLKRILEEIVRLLEE HLEKVERVLEAILSLLDDLLRRGGDERAIRTLEDVKR RLREILERLADENAKAIKRLADLLDKLEKRNKEAIER LEEILEELKRVRRDEELLRVLETLLKIIEDILRENTK VLEDLLRLVEEILEANLRVVEELLRLAREILTEIVGD EDKLKEIEDELRRLLEELRRLDKAIKDRLRELKKDLD EANRRIKETLKKLLREVEK (SEQ ID NO:27218) | 3plus1_ Key_Cte rm_560 | EDKLKEIEDELRRLLE ELRRLDKAIKDRLREL KKDLDEANRRIKETLK KLLREVEK(SEQ ID NO:27219) |
| 3plus 1_Cag e_568 _GFP1 1_Cte rm | KEIEETLKELEDLNREMVETNRRVLEETRRLNKETVD RVKATLDELAKMLKKLVDDVRKGPTSEELKRLLAELE ELLARVVRRVEELLKKSTDLLERAVKDSADALRRSHE VLKEVASRVKRAKDEGLPREEVLRLLRELLERHAKVL KDIVRVSEKLLREHLKVLREIVEVLEELLERILKVIL DTTRDHMVLHEYVNAAGITKRRLKEVIDRYEDELRKL RKEYKEKIDKYERKLEEIERRERT (SEQ ID NO:27220) | 3plus1_ Key_Cte rm_568 | KAVEELEKALEEIKRR LKEVIDRYEDELRKLR KEYKEKIDKYERKLEE IERRERT(SEQ ID NO:27221) |
| 3plus 1_Cag e_568 _GFP1 1_Cte rm | KEIEETLKELEDLNREMVETNRRVLEETRRLNKETVD RVKATLDELAKMLKKLVDDVRKGPTSEELKRLLAELE ELLARVVRRVEELLKKSTDLLERAVKDSADALRRSHE VLKEVASRVKRAKDEGLPREEVLRLLRELLERHAKVL KDIVRVSEKLLREHLKVLREIVEVLEELLERILKVIL DTTGGDRDHMVLHEYVNAAGITLKEVIDRYEDELRKL RKEYKEKIDKYERKLEEIERRERT (SEQ ID NO:27222) | 3plus1_ Key_Cte rm_568 | KAVEELEKALEEIKRR LKEVIDRYEDELRKLR KEYKEKIDKYERKLEE IERRERT(SEQ ID NO:27223) |
| 3plus 1_Cag e_Cte rm_5 8 1 | SALETVKKLLEDSSEKIERIVEEDERVAKESSDRIRR LVEEDKRVADEILDLIEKIGDTDTLLKLVEEWSRTSK KLLDDVLKLHKDWSDDSRRLLEEILRVHEELIRRVKE ILDREGKPEEVVRELEKVLKESLDTLEEIIRRLDEAN AATVKRVADVIRELEDINRKVLEEIKRGSDDAEAVIK VIEKLIRANKRVWDALLKINEDLVRVNKTVWKELLRV NEKLARDLERVVK (SEQ ID NO:27226) | 3plus1_ Key_Cte rm_581 | AEAVIKVIEKLIRANK RVWDALLKINEDLVRV NKTVWKELLRVNEKLA RDLERVVK(SEQ ID NO:27227) |
| 3plus 1 Cag | SKEEKLKDDVRAVLEDLDRVLKELEKLSEDNLRELKR VLDRITDLHRRILDELRKGIGSEELLRRVEKVLKDNL | 3plus1_ Key Cte | SKAAEDILRVLEKLVK VSREAIKLILELSEHH |
| e_Cte rm_58 5 | DLLRKLVEEHKESSERDLKRVEDLVREIKEVLRKLLE LEDRGTDIRKIEEEIERLLRKIRKAVEESKDLNRRNS ERIEEVARRSEELARRLLKEIRERGDSKAAEDILRVL EKLVKVSREAIKLILELSEHHVRVSTRIARLLLDVAR KLAEVIKEAER(SEQ ID NO:27228) | rm_585 | V K ID NO:27229) |
| 3plus 1_Gag e_Cte rm_58 7 | SEIEDVIRRLRKILEDLERVSEKLLREIKKILDEARR LNEEVIKEIKRVLEDAVRVFRDGSGSKEELAKLVEEL IRELAKLAKEVDEIHKRIVERLKALVEDAERIHRKIV ETLEEIVRGVPSEELKRVVEAIVEVIKEHLKVLADVI RRIIKAIEENAETIKRVLEDIVRVLELVLRGEGSIED LVREVERLIKRIEDSLRELEKTVRELLKRIKEASDKV REDVDRLIKELKEAAD(SEQ ID NO:27230) | 3plus1_ Key_Cte rm_587 | IEDLVREVERLIKRIE DSLRELEKTVRELLKR IKEASDKVREDVDRLI KELKEAAD(SEQ ID NO:27231) |
| 3plus 1_Cag e_Cte rm_60 5 | SREELLDRILEAIAKILEDLKRLIDENLARLEEVVRE LERIIDRNLKLIREILDELKKGSGSEEILEKIKKVDK ELEDLIRRLLKKLEDLIRETERRLREILKRIRDLLKE VKDRDKDLERLLEVLEEVLRVIAELAKELLDSLRKVL KVVEEVLRLLNEVNKEVLDVIRELAKDGGSDEIIRKL DELLKEVEKVHKEVKDRIRKLLEDHKRSLDEVKKKLE RLLERAKEVVEREKK(SEQ ID NO: 27232) | 3plus1_ Key_Cte rm_605 | DEIIRKLDELLKEVEK VHKEVKDRIRKLLEDH KRSLDEVKKKLERLLE AKEVVEREKK (SEQ ID NO:27233) |
| 3plus 1_Cag e_Cte rm_60 7 | SEREELLERIKEILKRVKDKLDEDLKRLKEILEKLKE KADRDLEELRRRIEEVREKLERTGRTDELVKEVLDTV RRNLENLKRLVEDILRKLEENVKNLTDLVREILKLIT ELIKRLEDGGLPKEVLDALRRVLEKLEELLREILERL KRSLEAVKRKIEELLKELERSLDELRRALERIRKEIG DSETAVRAIIRVLEKHLEAVRRVLEELLKVLAEHLET VRELIERLKRVLEEAIEVVERVAR(SEQ ID NO:27234) | 3plus1_ Key_Cte rm_607 | SETAVRAIIRVLEKHL EAVRRVLEELLKVLAE HLETVRELIERLKRVL EEAIEVVERVAR (SEQ ID NO:27235) |
| 3plus 1_Cag e_611 _GFP1 1_Cte rm | SLEEITKRLLELVEENLARHEEILRELLELAKRLAKE DRDILEEVLKLIEELLKLLEDNGSSEEDLKRLLKEVI EELRAVVKRVKDKVVDEVVKRIEDLVKKLKELHDDTLR KLRELVRKIVTDISESGGEAEKVKRVVEKILELVERL AKVVKESVEKLLEILRELAEVSKRVAEALLRLLEELV RVIRIKDERDHMVLHEYVNAAGITLLDELEEVHKRVK KELEDIIEENRRVVKRVRDELREIKRELDE(SEQ ID NO:27238) | 3plus1_ Key_Cte rm_611 | ERTLREVVRKVLEEAK RLLDELEEVHKRVKKE LEDIIEENRRVVKRVR DELREIKRELDE(SEQ ID NO:27239) |
| 3plus 1_Cag e_Cte rm_63 2 | SEKELVDDIRRILEEILRLLRSLLEEVIRLLEENEKL VRRHLKTVIDILRRVAKLLDENGIRTDEADRVLERLE KAHRELLEDYKRALEKIKETLERVLREAEEVVKKIDD ALRKLGGSKEVLKRLLEELLRLVEKIAEEIKRLLSEL VRVTEELVRTNKELLEEAVRVIRKEVGDDSLVREVEE LIKRLEKHIDDLLKTSRDLVKRVLDLVDEVVKRVEDL VERVKEKIDT(SEQ ID NO:27240) | 3plus1_ Key_Cte rm_632 | DSLVREVEELIKRLEK HIDDLLKTSRDLVKRV LDLVDEVVKRVEDLVE RVKEKIDT(SEQ ID NO:27241) |
| 3plus 1_Cag e_646 _GFP1 1_Cte rm | DAEEVVKRLADVLRENDETIRKVVEDLVRIAEENDRL WKKLVEDIAEILRRIVELLRRGGVPEELLDRLAKVVK SIVEKAEKILERLNRVSKAIAEKLKTIVDELNEVSKE IVKRAEDILRKGKDKETVLRALRTLVKEYADLSKEVL ERVERIVREYVKLSDEVVKSLAEIVEELIRIIEDLLR KGNRDHMVLHEYVNAAGITRKLLEDVKKASEDIVREV ERIVRELAKRSDEILKKLEDIVEKLRE(SEQ ID NO:27244) | 3plus1_ Key_Cte rm_646 | EDVKRALEELVSRLRK LLEDVKKASEDIVREV ERIVRELAKRSDEILK KLEDIVEKLRE(SEQ ID NO:27245) |
| 3plus 1_Cag e_646 _GFP1 1_Cte rm | DAEEVVKRLADVLRENDETIRKVVEDLVRIAEENDRL WKKLVEDIAEILRRIVELLRRGGVPEELLDRLAKVVK SIVEKAEKILERLNRVSKAIAEKLKTIVDELNEVSKE IVKRAEDILRKGKDKETVLRALRTLVKEYADLSKEVL ERVERIVREYVKLSDEVVKSLAEIVEELIRIIEDLLR KGNLDEDRDHMVLHEYVNAAGITEDVKKASEDIVREV ERIVRELAKRSDEILKKLEDIVEKLRE(SEQ ID NO:27246) | 3plus1_ Key_Cte rm_646 | EDVKRALEELVSRLRK LLEDVKKASEDIVREV ERIVRELAKRSDEILK KLEDIVEKLRE(SEQ ID NO:27247) |
| 3plus 1_Cag e_646 | DAEEVVKRLADVLRENDETIRKVVEDLVRIAEENDRL WKKLVEDIAEILRRIVELLRRGGVPEELLDRLAKVVK SIVEKAEKILERLNRVSKAIAEKLKTIVDELNEVSKE | 3plus1_ Key_Cte rm_646 | EDVKRALEELVSRLRK LLEDVKKASEDIVREV ERIVRELAKRSDEILK |
| _GFP1 1_Cte rm | IVKRAEDILRKGKDKETVLRALRTLVKEYADLSKEVL ERVERIVREYVKLSDEVVKSLAEIVEELIRIIEDLLR KGNLDEDVRDHMVLHEYVNAAGITDVKKASEDIVREV ERIVRELAKRSDEILKKLEDIVEKLRE (SEQ ID NO:27248) | | K I |
| 3plus 1_Gag e_647 _GFP1 1_Cte rm | DAEEVVKRLADVLRENDETIRKVVEDLVRIAEENDRL WKKLVEDIAEILRRIVELLRRGGVPEELLDRLAKVVK SIVEKAEKILERLNRVSKAIAEKLKTIVDELNEVSKE IVKRAEDILRKGKDKETVLRALRTLVKEYADLSKEVL ERVERIVREYVKLSDEVVKSLAEIVEELIRIIEDLLR KGNLRDHMVLHEYVNAAGITKLLEDVKKASEDIVREV ERIVRELAKRSDEILKKLEDIVEKLRE (SEQ ID NO:27250) | 3plus1_ Key_Cte rm_647 | EDVKRALEELVSRLRK LLEDVKKASEDIVREV ERIVRELAKRSDEILK KLEDIVEKLRE (SEQ ID NO:27251) |
| 3plus 1_Cag e_647 _GFP1 1_Cte rm | DAEEVVKRLADVLRENDETIRKVVEDLVRIAEENDRL WKKLVEDIAEILRRIVELLRRGGVPEELLDRLAKVVK SIVEKAEKILERLNRVSKAIAEKLKTIVDELNEVSKE IVKRAEDILRKGKDKETVLRALRTLVKEYADLSKEVL ERVERIVREYVKLSDEVVKSLAEIVEELIRIIEDLLR KGNLDEDVKRALERDHMVLHEYVNAAGITSEDIVREV ERIVRELAKRSDEILKKLEDIVEKLRE (SEQ ID NO:27252) | 3plus1_ Key_Cte rm_647 | EDVKRALEELVSRLRK LLEDVKKASEDIVREV ERIVRELAKRSDEILK KLEDIVEKLRE (SEQ ID NO:27253) |
| 3plus 1_Cag e_Cte rm_64 7 | DAEEVVKRLADVLRENDETIRKVVEDLVRIAEENDRL WKKLVEDIAEILRRIVELLRRGGVPEELLDRLAKVVK SIVEKAEKILERLNRVSKAIAEKLKTIVDELNEVSKE IVKRAEDILRKGKDKETVLRALRTLVKEYADLSKEVL ERVERIVREYVKLSDEVVKSLAEIVEELIRIIEDLLR KGNLDEDVKRALEELVSRLRKLLEDVKKASEDIVREV ERIVRELAKRSDEILKKLEDIVEKLRE (SEQ ID NO:27254) | 3plus1_ Key_Cte rm_647 | EDVKRALEELVSRLRK LLEDVKKASEDIVREV ERIVRELAKRSDEILK KLEDIVEKLRE (SEQ ID NO:27255) |
| 3plus 1_Cag e_Cte rm_65 3 | DEEETLRRLLERKVELAKEYLDVSKEVIDRTTKLLDE YLKTSKRIVDATVELLERGDLGPDELIKRLAEELERS LRELEEEIKRLKRELEESLKKLKEIIDRLAEEAEKLL AVLKRGEGSEEEALRALASLVRELIEVLRENDERLRD VLRRLIEALRKNNEILERVLRKLVRAAEERGRDESSR EALEEARRRLEELLRELNEITKDLEAKLEKLLRDLNE LTKALEEELKRLLDELKKRTD (SEQ ID NO:27256) | 3plus1_ Key_Cte rm_653 | SREALEEARRRLEELL RELNEITKDLEAKLEK LLRDLNELTKALEEEL KRLLDELKKRTD (SEQ ID NO:27257) |
| 3plus 1_Cag e_Cte rm_65 8 | DEERIIKTLEDINAKLVEDIKRILDKVAELNERLADA IRKILEETKRILEATTRKVRKDGEISEELLRRLEEKL RKLLEDLERVLAEHEDESRRILEEVERLLKRHADASK ELLDRARSVARGVKSDKELVDRLKKLIDDSLESVREL IERLKELLDRLVKSVEDLIRTIKELLDRLVEVLREGV SDKDTLRTVEKLVEDVKRRLDKLLEDYKRLIEEVKKE LDKLLKEYEDALREIKKRIDE (SEQ ID NO:27258) | 3plus1_ Key_Cte rm_658 | KDTLRTVEKLVEDVKR RLDKLLEDYKRLIEEV KKELDKLLKEYEDALR EIKKRIDE (SEQ ID NO:27259) |
| 3plus 1_Cag e_Nte rm_26 3 | SLVDELRKSLERNVRVSEEVARRLKEALKRWVDVVRK VVEDLIRLNEDVVRVVEKVTVDESAIERVRRIIEELN RKLDAVLKKNEDLVRRLTELLDKLLEENRRLVEELDE DLKRRGGTEEVIDTILELIERSIERLKRLLDELLRIV REALKDNKRVADENLKKLKEILDELRKDGVEDEELKR VLEKAADLHRRLKDRHRKLLEDLERIIRELKKKLDEV VEENKRSVDELKR (SEQ ID NO:27262) | 3plus1_ Key_Nte rm_263 | SLVDELRKSLERNVRV SEEVARRLKEALKRWV DVVRKVVEDLIRLNED VVRVVEKV (SEQ ID NO:27263) |
| 3plus 1_Cag e_647 _GFP1 1_Nte rm | DAEEVVKRLADVLRENDETIRKVVEDLVRIAEENDRL WRDHMVLHEYVNAAGITLLRRGGVPEELLDRLAKVVK SIVEKAEKILERLNRVSKAIAEKLKTIVDELNEVSKE IVKRAEDILRKGKDKETVLRALRTLVKEYADLSKEVL ERVERIVREYVKLSDEVVKSLAEIVEELIRIIEDLLR KGNLDEDVKRALEELVSRLRKLLEDVKKASEDIVREV ERIVRELAKRSDEILKKLEDIVEKLRE (SEQ ID NO:27276) | 3plus1_ Key_Nte rm_647 | DAEEVVKRLADVLREN DETIRKVVEDLVRIAE ENDRLWKKLVEDIAEI LRRIVELLRRG (SEQ ID NO:27277) |

In various specific embodiments, the cage proteins comprise an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues, to the amino acid sequence of a cage protein selected from the group consisting of SEQ ID NOS: 27497-27620, wherein the N-terminal protein purification tag (MGSHHHHHHGSGSENLYFQGSGG (SEQ ID NO:27624); or MGSHHHHHHGSENLYFQG (SEQ ID NO:27625); or GSHHHHHHGSGSENLYFQG (SEQ ID NO:27626)) is optional, is not considered in the percent identity comparison, and can be present or absent. In one embodiment the N-terminal protein purification tag is absent.

TABLE 10

Amino acid sequences
(The sequences below contain a 6His-TEV tag for protein purification purposes MGSHHHHHHGSGSENLYFQG (SEQ ID NO: 27495) or variant thereof. The amino acids N-terminal to the structural region are optional and are not considered in the percent identity comparison relevant to the claimed cage protein
(The structural region is in parenthesis) The region C-terminal to the parenthesis constitutes the latch region. The SmBit sequence (VTGYRLFEEIL) (SEQ ID NO:27359) is underlined. The sensing domains are in bold

### lucCageBim Variants (Bc12 Sensors)

- SmBit sequence: VTGYRLFEEIL(SEQ ID NO:27359)

- BIM sequence: EIWIAQELRRIGDEFNAYYA (SEQ ID NO:2
7496)

>n1uc301 _bim331

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)ggsVTGYRLFEEILRVKRESKRIVE
DAERLsREEIWIAQELRRIGDEFNAYYAAASEKISRE (SEQ ID NO:2
7497)

>nluc308_bim331

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARVTGYRLFEEILRIVEDAE
RLsREEIWIAQELRRIGDEFNAYYAAASEKISRE (SEQ ID NO: 274
98)

>nluc312_bim331

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARKAIAVTGYRLFEEILDAE
RLsREEIWIAQELRRIGDEFNAYYAAASEKISRE (SEQ ID NO: 274
99)

>nluc315_bim331

[symbol]

MGSHHHHHHGSGSENLYFQGSGG(SREAARRLQDLNIELARRLLEASTRL
QRLNIRIVYLAVELTDPKRIRDEIKEVRDRSREIIRRAEREIDDAARESR
RILEEARRAIRDAAEESRRILEEGSGSGSDALDELQKLNLELAKLLLKAI
AETQDLNLRAAKAFLEAAAKLQELNIRAVELLVKLTDPATIRRALEHAKR
RSKEIIDEAERAIRAAKRESERIIEEARRLIEKAKEESERIIREGSGSGD
PDIKKLQDLNIELARELLRAHAQLQRLNLELLRELLRALAQLQELNLDLL
RLASEL)TDPDEARKAIARVKVTGYRLFEEILRLsREEIWIAQELRRIGD EFNAYYAAASEKISRE (SEQ ID NO: 27500)

>n1uc301 _bim339

MGSHHHHHHGSGSENLYFQGSGG(SREAARRLQDLNIELARRLLEASTRL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)ggsVTGYRLFEEILRVRRESRRIVE
DAERLsREAAAASERIEIWIAQELRRIGDEFNAYYAE (SEQ ID NO:
27501)

>nluc308_bim339

MGSHHHHHHGSGSENLYFQGSGG(SREAARRLQDLNIELARRLLEASTRL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARVTGYRLFEEILRIVEDAE
RLsREAAAASEKIEIWIAQELRRIGDEFNAYYAE (SEQ ID NO: 275
02)

>nluc312_bim339

MGSHHHHHHGSGSENLYFQGSGG(SREAARRLQDLNIELARRLLEASTRL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARKAIAVTGYRLFEEILDAE
RLsREAAAASEKIEIWIAQELRRIGDEFNAYYAE (SEQ ID NO: 275
03)

>nluc315 _bim339

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL) TDPDEARKAIARVKVTGYRLFEEI LRLsREAAAASEKIEIWIAQELRRIGDEFNAYYAE (SEQ ID NO: 27
504)

>n1uc301 _bim343

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)ggsVTGYRLFEEILRVRRESRRIVE
DAERLsREAAAASERISREAEIWIAQELRRIGDEFNAYYA (SEQ ID N
O: 27505)

>nluc308_bim343

MGSHHHHHHGSGSENLYFQGSGG(SREAARRLQDLNIELARRLLEASTRL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARVTGYRLFEEILRIVEDAE
RLsREAAAASEKISREAEIWIAQELRRIGDEFNAYYA (SEQ ID NO:
27506)

>nluc312_bim343

MGSHHHHHHGSGSENLYFQGSGG(SREAARRLQDLNIELARRLLEASTRL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARKAIAVTGYRLFEEILDAE
RLsREAARRIGDEFNAYYA (SEQ ID NO: 27507)

>nluc315_bim343

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARKAIARVKVTGYRLFEEIL
RLsREAAAASEKISREAEIWIAQELRRIGDEFNAYYA (SEQ ID NO:
27508)

### lucCageTrop Variants (Cardiac Troponin I Sensors)

- SmBit sequence: VTGYRLFEEIL (SEQ ID NO: 27359)

- Variants of cardiac troponin T (cTnT) used sequences:

- cTnTf1:226-EDQLREKAKELWQTI-240 (SEQ ID NO:27385)

- cTnTf2:226-EDQLREKAKELWQTIYN-242 (SEQ ID NO:2738
6)

- cTnTf3:226-EDQLREKAKELWQTIYNLEAE-246 (SEQ ID NO:
27387)

- cTnTf4:226-EDQLREKAKELWQTIYNLEAEKFD-249 (SEQ ID
NO:27388)

- cTnTf5:226-EDQLREKAKELWQTIYNLEAEKFDLQE-252 (SEQ
ID NO:27389)

- cTnTf6:226- EDQLREKAKELWQTIYNLEAEKFDLQEKFKQQKYEI NVLRNRINDNQ-272 (SEQID NO:27390)

-cTnC:

KVSKTKDDSKGKSEEELSDLFRMFDKNADGYIDLEELKIMLQATGETITE DDIEELMKDGDKNNDGRIDYDEFLEFMKGVE (SEQ ID NO:27627)

>336-cTnTf4-K342A (jp625_1fix_nluc312_cTnT336_K342A_359end)

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARKAIAVTGYRLFEEILDAE
RLsREAAAASEDQLREaAKELWQTIYNLEAEKFD (SEQ ID NO: 275
09)

>336-cTnTf6-K342A (jp626_1fix-nluc312_cTnT336_K342A_362end)

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARKAIAVTGYRLFEEILDAE
RLsREAAAASEDQLREaAKELWQTIYNLEAEKFDLQE (SEQ ID NO:
27510)

>336-cTnTf6-K342A (jp627_1fix-nluc312_cTnT336_K342A_0001_382end)

MGSHHHHHHGSGSENLYFQGSGG(SKEAAKKLQDLNIELARKLLEASTKL
QRLNIRLAEALLEAIARLQELNLELVYLAVELTDPKRIRDEIKEVKDKSK
EIIRRAEKEIDDAAKESKKILEEARKAIRDAAEESRKILEEGSGSGSDAL
DELQKLNLELAKLLLKAIAETQDLNLRAAKAFLEAAAKLQELNIRAVELL
VKLTDPATIRRALEHAKRRSKEIIDEAERAIRAAKRESERIIEEARRLIE
KAKEESERIIREGSGSGDPDIKKLQDLNIELARELLRAHAQLQRLNLELL
RELLRALAQLQELNLDLLRLASEL)TDPDEARKAIAVTGYRLFEEILDAE
RLsREAAAASEDQLREaAKELWQTIYNLEAEKFDLQEKFKQQKYEINVLR NRINDNQ (SEQ ID NO: 27511)

>339-cTnTf3 (jp628 lfix-nluc312 cTnT339 359end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL)TDPDEARKAIAVTGYRLFEEILDAERLsRE
AAAASEKIEDQLREKAKELWQTIYNLEAE (SEQ ID NO: 27512)

>339-cTnTf5 (jp629 lfix-nluc312 cTnT339 0001 365end)

[symbol]

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRALAQLQELNLDLLRLASE
L) TDPDEARKAIAVTGYRLFEEILDAERLsREAAAASEKLEAEKFDLQE KFKQQKYEINVLRNRINDNQ (SEQ ID NO: 27513)

>339-cTnTf6 (jp630 lfix-nluc312 cTnT339 0001 385end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL)TDPDEARKAIAVTGYRLFEEILDAERLsRE
AAAASEKIEDQLREKAKELWQTIYNLEAEKFDLQEKFKQQKYEINVLRNR INDNQKFKQQKYEINVLRNRINDNQ (SEQ ID NO: 27514)

>343-cTnTf2 (jp631 lfix-nluc312 cTnT343 359end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL)TDPDEARKAIAVTGYRLFEEILDAERLsRE
AAAASEKISREAEDQLREKAKELWQTIYN (SEQ ID NO: 27515)

>343-cTnTf5 (jp632 lfix-nluc312 cTnT343 0001 369end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL)TDPDEARKAIAVTGYRLFEEILDAERLsRE
AAAASEKISREAEDQLREKAKELWQTIYNLEAEKFDLQEKFKQQKYEINV LRNRINDNQ (SEQ ID NO: 27516)

>343-cTnTf6 (jp633 lfix-nluc312 cTnT343 0001 389end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL) TDPDEARKAIAVTGYRLFEEILDAERLsR
EAAAASEKISREAEDQLREKAKELWQTIYNLEAEKFDLQEKFKQQKYEIN VLRNRINDNQKFKQQKYEINVLRNRINDNQ (SEQ IDNO: 27517)

>345-cTnTf1 (jp634_1fix-nluc312_cTnT345_359end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL)TDPDEARKAIAVTGYRLFEEILDAERLsRE
AAAASEKISREAEREDQLREKAKELWQTI (SEQ ID NO: 27518)

>345-cTnTf5 (jp635 lfix-nluc312 cTnT345 0001 371end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL)TDPDEARKAIAVTGYRLFEEILDAERLsRE
AAAASEKISREAEREDQLREKAKELWQTIYNLEAEKFDLQEKFKQQKYEI NVLRNRINDNQ (SEQ ID NO: 27519)

>345-cTnTf6 (jp636 lfix-nluc312 cTnT345 0001 391end)

MGSHHHHHHGSENLYFQG(SKEAAKKLQDLNIELARKLLEASTKLQRLNI
RLAEALLEAIARLQELNLELVYLAVELTDPKRIRDEIKEVKDKSKEIIRR
AEKEIDDAAKESKKILEEARKAIRDAAEESRKILEEGSGSGSDALDELQK
LNLELAKLLLKAIAETQDLNLRAAKAFLEAAAKLQELNIRAVELLVKLTD
PATIRRALEHAKRRSKEIIDEAERAIRAAKRESERIIEEARRLIEKAKEE
SERIIREGSGSGDPDIKKLQDLNIELARELLRAHAQLQRLNLELLRELLR
ALAQLQELNLDLLRLASEL)TDPDEARKAIAVTGYRLFEEILDAERLsRE
AAAASEKISREAEREDQLREKAKELWQTIYNLEAEKFDLQEKFKQQKYEI NVLRNRINDNQKFKQQKYEINVLRNRINDNQ (SEQ ID NO: 27520)

>lucCageTrop

MGSHHHHHHGSGSENLYFQGSGG (SKEAAKKLQDLNIELARKLLEASTK
LQRLNIRIVYLAVELTDPKRIRDEIKEVKDKSKEIIRRAEKEIDDAAKES
KKILEEARKAIRDAAEESRKILEEGSGSGSDALDELQKLNLELAKLLLKA
IAETQDLNLRAAKAFLEAAAKLQELNIRAVELLVKLTDPATIRRALEHAK
RRSKEIIDEAERAIRAAKRESERIIEEARRLIEKAKEESERIIREGSGSG
DPDIKKLQDLNIELARELLRAHAQLQRLNLELLRELLRALAQLQELNLDL
LRLASEL)TDPDEARKAIAVTGYRLFEEILDAERLsREAAAASEDQLREa AKELWQTIYNLEAEKFDLQEKFKQQKYEINVLRNRINDNQKVSKTKDDSK GKSEEELSDLFRMFDKNADGYIDLEELKIMLQATGETITEDDIEELMKDG DKNNDGRIDYDEFLEFMKGVE (SEQ ID NO: 27521)

### lucCageBot Variants (Botulinum Neurotoxin B Sensors)

- Bot.0671.2 sequence: MFAELKAKFFLEIGDRDAARNALRKA GYSDEEAERIIRKYELE (SEQID NO: 27381)

>BoNTB_338_1S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL) TDPDEARKAIAVTGYRLFEEILDAERLS REAAAASEKMFAELKAKFFLEIGDRDAARNALRKAGYSDEEAERIIRKYE LE* (SEQ ID NO: 27522)

> BoNTB_341_1S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL) TDPDEARKAIAVTGYRLFEEILDAERLS
REAAAASEKISRMFAELKAKFFLEIGDRDAARNALRKAGYSDEEAERIIR KYELE* (SEQ ID NO: 27523)

>BoNTB_342_1S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREMFAELKAKFFLEIGDRDAARNALRKAGYSDEEAERIIR KYELE* (SEQ ID NO: 27524)

>BoNTB_345_1S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERMFAELKAKFFLEIGDRDAARNALRKAGYSDEEAER IIRKYELE* (SEQ ID NO: 27525)

>BoNTB_348_2S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIRMFAELKAKFFLEIGDRDAARNALRKAGYSDEE AERIIRKYELE* (SEQ ID NO: 27526)

>BoNTB_349_2S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEAR [symbol] KAIRDAAEESRKILEEG
SGSGSDALDELQKLNLELAKLLLKAIAETQDLNLRAAKARAAKLQELNIR
AVELLVKLTDPATIRRALEHAKRRSKEIIDEAERAIRAAKRESERIIRRL
IEKAKEESERIIREGSGSGDPDIKKLQDLNIELARELLRAHAQLQRLNLE
LLRELLRALAQLQELNLDLLRLASEL)TDPDEARKAIAVTGYRLFEEILD
AERLSREAAAASEKISREAERSIREMFAELKAKFFLEIGDRDAARNALRK AGYSDEEAERIIRKYELE* (SEQ ID NO: 27527) [symbol]
indicates text missing or illegible when filed

>BoNTB_352_2S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAAAMFAELKAKFFLEIGDRDAARNALRKAGY SDEEAERIIRKYELE* (SEQ ID NO: 27528)

>BoNTB_355_2S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAAAASEMFAELKAKFFLEIGDRDAARNALRK AGYSDEEAERIIRKYELE* (SEQ ID NO:27529)

>BoNTB_GGG _2S

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAAAASEKISREGGGMFAELKAKFFLEIGDRD AARNALRKAGYSDEEAERIIRKYELE* (SEQ ID NO: 27530)

>BoNTB_GGG_2S_fullBotBinder

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAAAASEKISREGGGSHMQPMFAELKAKFFLE IGDRDAARNALRKAGYSDEEAERIIRKYELE* (SEQ ID NO: 27531
)

### lucCageProA Variants (Fc Domain Biosensors)

- Staphylococcus aureus Protein A domain C (SpaC) sequence:

EQQNAFYEILHLPNLTEEQRNGFIQSLKDDPSVSKEILAEAKKLNDAQAP K (SEQ ID NO:27382)

>SpaC_360GGG

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEKISREGGGFNKEQQNAFYEILHLP NLTEEQRNGFIQSLKDDPSVSKEILAEAKKLNDAQAPK* (SEQ ID NO
: 27532)

>SpaC_354-2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKIARKAIRDAAEESRKILEEGSGSGSDALDELQKL
NLELAKLLLKAIAETQDLNLRAAFEAAAKLQELNIRAVELLVKLTDPATI
RRALEHAKRRSKEIIDEAERAIRAAKRESERIIEEARRLIEKAKEESERI
IREGSGSGDPDIKKLQDLNIELARELLRAHAQLQRLNLELLRELLRALAQ
LQELNLDLLRLASEL)TDPDEARKAIAVTGYRLFEEILDAERLSREAAAA
SEKISREAERSIREAAAASEQQNAFYEILHLPNLTEEQRNGFIQSLKDDP SVSKEILAEAKKLNDAQAPK*(SEQ ID NO: 27533)

>SpaC_351_2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAEQQNAFYEILHLPNLTEEQRNGFIQSLK DDPSVSKEILAEAKKLNDAQAPK*(SEQ ID NO: 27534)

>SpaC_350_2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAEQQNAFYEILHLPNLTEEQRNGFIQSLKD DPSVSKEILAEAKKLNDAQAPK*(SEQ ID NO: 27535)

>SpaC_347_2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIEQQNAFYEILHLPNLTEEQRNGFIQSLKDDPS VSKEILAEAKKLNDAQAPK*(SEQ ID NO: 27536)

>SpaC_347_1S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERLIEQQNAFYEILHLPNLTEEQRNGFIQSLKDDPS VSKEILAEAKKLNDAQAPK*(SEQ ID NO: 27537)

### lucCageHer2 Variants (Fc Domain Biosensors)

- Her2 affibody sequence:

EMRNAYWEIALLPNLNNQQKRAFIRSLYDDPSQSANLLAEAKKLNDAQAP K (SEQ ID NO:27383)

>AffiHer2_347_1S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERLIEMRNAYWEIALLPNLNNQQKRAFIRSLYDDPS QSANLLAEAKKLNDAQAPK*(SEQ ID NO: 27538)

>AffiHer2 347 2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIEMRNAYWEIALLPNLNNQQKRAFIRSLYDDPS QSANLLAEAKKLNDAQAPK*(SEQ ID NO: 27539)

E

>AffiHer2 350 2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAEMRNAYWEIALLPNLNNQQKRAFIRSLYD DPSQSANLLAEAKKLNDAQAPK*(SEQ ID NO: 27540)

>AffiHer2 351 2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAEMRNAYWEIALLPNLNNQQKRAFIRSLY DDPSQSANLLAEAKKLNDAQAPK*(SEQ ID NO: 27541)

>AffiHer2 354-2S

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEMRNAYWEIALLPNLNNQQKRAFIR SLYDDPSQSANLLAEAKKLNDAQAPK*(SEQ ID NO: 27542)

>AffiHer2_360GGG

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEKISREGGGVDNKFNKEMRNAYWEI ALLPNLNNQQKRAFIRSLYDDPSQSANLLAEAKKLNDAQAPK* (SEQ I
D NO: 27543)

>AffiHer2 354-2S 2x1

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEMRNAYWEIALLPNLNNQQKRAFIR SLYDDPSQSANLLAEAKKLNDAQAPKGGGNKEMRNAYWEIALLPNLNNQQ KRAFIRSLYDDPSQSANLLAEAKKLNDAQAPK* (SEQ ID NO: 2754
4)

[symbol]

>AffiHer2 354-2S 2x2

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEMRNAYWEIALLPNLNNQQKRAFIR SLYDDPSQSANLLAEAKKLNDAQAPKGGGNKEMRNAYWEIALLPNLNNQQ KRAFIRSLYDDPSQSANLLAEAKKLNDAQAPK* (SEQ ID NO: 2754
5)

>AffiHer2 354-2S 3x

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEMRNAYWEIALLPNLNNQQKRAFIR SLYDDPSQSANLLAEAKKLNDAQAPKGGGNKEMRNAYWEIALLPNLNNQQ KRAFIRSLYDDPSQSANLLAEAKKLNDAQAPKGGGNKEMRNAYWEIALLP NLNNQQKRAFIRSLYDDPSQSANLLAEAKKLNDAQAPK * (SEQ ID N
O: 27546)

### lucCageSARS2N Variants (Anti-SARS-CoV-2 Nucleocapsid Protein Antibodies Sensors)

- SARS-Cov-2 Nucleocapsid protein epitope peptides used:

- N6: PKKDKKKKADETQALPQRQKKGGSGGPKKDKKKKADETQALPQR QKK (SEQ ID NO:27547)

- N62: KKDKKKKADETQALGGSGGKKDKKKKADETQAL (SEQ ID N
O:27548)

>lucCageSARS2-N6_368-388_339

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKPKKDKKKKADETQALPQRQKKGGSGGPKKDKKKKADETQALP QRQKK* (SEQ ID NO: 27549)

>lucCageSARS2-N6_368-388_346

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSPKKDKKKKADETQALPQRQKKGGSGGPKKDKKKK ADETQALPQRQKK* (SEQ ID NO: 27550)

>lucCageSARS2-N6_368-388_353

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAAAAPKKDKKKKADETQALPQRQKKGDRADL RKTKRRKPTKPKHCRNVKKS (SEQ ID NO: 27551)

>lucCageSARS2-N62_369-382_336

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEA [symbol] AAKLQEL
NIRAVELLVKLTDPATIRRALEHAKRRSKEIIDEAERAIRAAKRESERII
RRLIEKAKEESERIIREGSGSGDPDIKKLQDLNIELARELLRAHAQLQRL
NLELLREALAQLQELNLDLLRLASEL)TDPDEARKAIAVTGYRLFEEILD
AERLSREAAAASKKKKADETQALGGSGGKKDKKKKADETQAL* (SEQ I
D NO: 27552) [symbol]
indicates text missing or illegible when filed

>lucCageSARS2-N62_369-382_340

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKIKKDKKKKADETQALGGSGGKKDKKKKADETQAL* (SEQ I
D NO: 27553)

>lucCageSARS2-N62_369-382_343

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAKKDKKKKADETQALGGSGGKKDKKKKADETQAL* (S
EQ ID NO: 27554)

>lucCageSARS2-N62_369-382_347

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIKKDKKKKADETQALGGSGGKKDKKKKADETQAL * (SEQ ID NO: 27555)

>lucCageSARS2-N62_369-382_350

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAKKDKKKKADETQALGGSGGKKDKKKKADET QAL* (SEQ ID NO: 27556)

>lucCageSARS2-N62_369-382_354

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAAAASKKDKKKKADETQALGGSGGKKDKKKK ADETQAL* (SEQ ID NO: 27557)

### lucCageSARS2M Variants (anti-SARS-Cov-2 Membrane Protein Antibodies Sensors)

- SARS-Cov-2 Membrane protein epitope peptides used:

- M1_1-31:MADSNGTITVEELKKLLEQWNLVIGFLFLTWIGGSGGMAD SNGTITVEELKKLLEQWNLVIGFLFLTWI(SEQ ID NO:27393)

- M3_1-17:MADSNGTITVEELKKLLEGGSGGMADSNGTITVEELKKLL E (SEQ ID NO:27392) - M4_8-24:ITVEELKKLLEQWNLVIGGS GGITVEELKKLLEQWNLVI (SEQ ID NO:27394)

>lucCageSARS2-M3_1-17_3414

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAEAAKLQELNIRAVELLVKLTDPA
TIRRALEHAKRRSKEIIDEAERAIRAAKRESERIIRRLIEKAKEESERII
REGSGSGDPDIKKLQDLNIELARELLRAHAQLQRLNLELLRELLRALAQL
QELNLDLLRLASEL)TDPDEARKAIAVTGYRLFEEILDAERLSREAAAAS
EKISRMADSNGTITVEELKKLLEGGSGGMADSNGTITVEELKKLLE* (S
EQ ID NO: 27558)

>lucCageSARS2-M3_1-17_343

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL) TDPDEARKAIAVTGYRLFEEILDAERLS
REAAAASEKISREAMADSNGTITVEELKKLLEGGSGGMADSNGTITVEEL KKLLE* (SEQ ID NO: 27559)

>lucCageSARS2-M3_1-17_348

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIRMADSNGTITVEELKKLLEGGSGGMADSNGTIT VEELKKLLE* (SEQ ID NO: 27560)

>lucCageSARS2-M3_1-17_350

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIREAMADSNGTITVEELKKLLEGGSGGMADSNGT ITVEELKKLLE* (SEQ ID NO: 27561)

>lucCageSARS2-M4_8-24_334

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAITVEELKKLLEQWNLVIGGSGGITVEELKKLLEQWNLVI* (SEQ
ID NO: 27562)

>lucCageSARS2-M4_8-24_340

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISITVEELKKLLEQWNLVIGGSGGITVEELKKLLEQWNLVI*
(SEQ ID NO: 27563)

>lucCageSARS2-M4_8-24_341

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISRITVEELKKLLEQWNLVIGGSGGITVEELKKLLEQWNLVI * (SEQ ID NO: 27564)

>lucCageSARS2-M4_8-24_348

[symbol]

GSHHHHHHGSGSENLYFQG (SKEAAKKLQDLNIELARKLLEASTKLQRL
NIRLAEAIARLQELNLELVYLAVELTDPKRIRDEIKEVKDKSKEIIRRAE
KEIDDAAKESKKILEKAIRDAAEESRKILEEGSGSGSDALDELQKLNLEL
AKLLLKAIAETQDLNLRAAKAFLEAAAKLQELNIRAVELLVKLTDPATIR
RALEHAKRRSKEIIDEAERAIRAAKRESERIIEEARRLIEKAKEESERII
REGSGSGDPDIKKLQDLNIELARELLRAHAQLQRLNLELLRELLRALAQL
QELNLDLLRLASEL)TDPDEARKAIAVTGYRLFEEILDAERLSREAAAAS
EKISREAERSIRITVEELKKLLEQWNLVIGGSGGITVEELKKLLEQWNLV I* (SEQ ID NO: 27565)

>lucCageM3 334 SmBit position301

GSHHHHHHGSGSENLYFQGSGG(SKEAAKKLQDLNIELARKLLEASTKLQ
RLNIRLAEALLEAIARLQELNLELVYLAVELTDPKRIRDEIKEVKDKSKE
IIRRAEKEIDDAAKESKKILEEARKAIRDAAEESRKILEEGSGSGSDALD
ELQKLNLELAKLLLKAIAETQDLNLRAAKAFLEAAAKLQELNIRAVELLV
KLTDPATIRRALEHAKRRSKEIIDEAERAIRAAKRESERIIEEARRLIEK
AKEESERIIREGSGSGDPDIKKLQDLNIELARELLRAHAQLQRLNLELLR
ELLRALAQLQELNLDLLRLASEL)ggsVTGYRLFEEILRVKRESKRIVED
AERLSREAAAMADSNGTITVEELKKLLEGGSGGMADSNGTITVEELKKLL
E (SEQ ID NO: 27566)

>lucCageM3 334 SmBit position308

GSHHHHHHGSGSENLYFQGSGG(SKEAAKKLQDLNIELARKLLEASTKLQ
RLNIRLAEALLEAIARLQELNLELVYLAVELTDPKRIRDEIKEVKDKSKE
IIRRAEKEIDDAAKESKKILEEARKAIRDAAEESRKILEEGSGSGSDALD
ELQKLNLELAKLLLKAIAETQDLNLRAAKAFLEAAAKLQELNIRAVELLV
KLTDPATIRRALEHAKRRSKEIIDEAERAIRAAKRESERIIEEARRLIEK
AKEESERIIREGSGSGDPDIKKLQDLNIELARELLRAHAQLQRLNLELLR
ELLRALAQLQELNLDLLRLASEL)TDPDEARVTGYRLFEEILRIVEDAER
LSREAAAMADSNGTITVEELKKLLEGGSGGMADSNGTITVEELKKLLE (
SEQ ID NO: 27567)

>lucCageM3_334_7loop

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDGGGSGGGPDEARKAIAVTGYRLFEEIL
DAERLSREAAAMADSNGTITVEELKKLLEGGSGGMADSNGTITVEELKKL
LE (SEQ ID NO: 27568)

>lucCageM3_334_3loop

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDGGGPDEARKAIAVTGYRLFEEILDAER
LSREAAAMADSNGTITVEELKKLLEGGSGGMADSNGTITVEELKKLLE (
SEQ ID NO: 27569)

>lucCageM3 341 SmBit position301

GSHHHHHHGSGSENLYFQGSGG(SKEAAKKLQDLNIELARKLLEASTKLQ
RLNIRLAEALLEAIARLQELNLELVYLAVELTDPKRIRDEIKEVKDKSKE
IIRRAEKEIDDAAKESKKILEEARKAIRDAAEESRKILEEGSGSGSDALD
ELQKLNLELAKLLLKAIAETQDLNLRAAKAFLEAAAKLQELNIRAVELLV
KLTDPATIRRALEHAKRRSKEIIDEAERAIRAAKRESERIIEEARRLIEK
AKEESERIIREGSGSGDPDIKKLQDLNIELARELLRAHAQLQRLNLELLR
ELLRALAQLQELNLDLLRLASEL)ggsVTGYRLFEEILRVKRESKRIVED
AERLSREAAAASEKISRMADSNGTITVEELKKLLEGGSGGMADSNGTITV
EELKKLLE(SEQ ID NO: 27570)

>lucCageM3 341 SmBit position308

GSHHHHHHGSGSENLYFQGSGG(SKEAAKKLQDLNIELARKLLEASTKLQ
RLNIRLAEALLEAIARLQELNLELVYLAVELTDPKRIRDEIKEVKDKSKE
IIRRAEKEIDDAAKESKKILEEARKAIRDAAEESRKILEEGSGSGSDALD
ELQKLNLELAKLLLKAIAETQDLNLRAAKAFLEAAAKLQELNIRAVELLV
KLTDPATIRRALEHAKRRSKEIIDEAERAIRAAKRESERIIEEARRLIEK
AKEESERIIREGSGSGDPDIKKLQDLNIELARELLRAHAQLQRLNLELLR
ELLRALAQLQELNLDLLRLASEL)TDPDEARVTGYRLFEEILRIVEDAER
LSREAAAASEKISRMADSNGTITVEELKKLLEGGSGGMADSNGTITVEEL
KKLLE (SEQ ID NO: 27571)

>lucCageM3_341_7loop

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELIRALAQLQELNLDLLRLAS
EL)TDGGGSGGGPDEARKAIAVTGYRLFEEILDAERLSFITVEELKKLLE
GGSGGMADSNGTITVEELKKLLE (SEQ ID NO: 27572)

>lucCageM3_341_3loop

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDGGGPDEARKAIAVTGYRLFEEILDAER
LSREAAAASEKISRMADSNGTITVEELKKLLEGGSGGMADSNGTITVEEL
KKLLE (SEQ ID NO: 27573)

>LUCCAGEM3_334_4copies

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAMADSNGTITVEELKKLLEGGSGGMADSNGTITVEELKKLLEG
GSGGMADSNGTITVEELKKLLEGGSGGMADSNGTITVEELKKLLE*(SEQ
ID NO: 27574)

>LUCCAGEM3_337_4copies

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEMADSNGTITVEELKKLLEGGSGGMADSNGTITVEELKKL
LEGGSGGMADSNGTITVEELKKLLEGGSGGMADSNGTITVEELKKLLE(S
EQ ID NO: 27575)

>LUCCAGEM3_341_4copies

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISRMADSNGTITVEELKKLLEGGSGGMADSNGTITVEE
LKKLLEGGSGGMADSNGTITVEELKKLLEGGSGGMADSNGTITVEELKKL
LE (SEQ ID NO: 27576)

>LUCCAGEM3_348_4copies

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISREAERSIRMADSNGTITVEELKKLLEGGSGGMADSN
GTITVEELKKLLEGGSGGMADSNGTITVEELKKLLEGGSGGMADSNGTIT
VEELKKLLE (SEQ ID NO: 27577)

>LUCCAGEM3 334 2copiesnolinker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAMADSNGTITVEELKKLLEMADSNGTITVEELKKLLE (SEQ
ID NO: 27578)

>LUCCAGEM3 337 2copiesnolinker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAKLLEMADSNGTITVEELKKLLE (SEQ ID NO: 27579)

>LUCCAGEM3 341 2copiesnolinker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISRMADSNGTITVEELKKLLEMADSNGTITVEELKKLL
E (SEQ ID NO: 27580)

>LUCCAGEM3 348 2copiesnolinker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISREAERSIRMADSNGTITVEELKKLLEMADSNGTITV
EELKKLLE (SEQ ID NO: 27581)

>LUCCAGEM3 334 4copies linker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAMADSNGTITVEELKKLLEGGSGGMADSNGTITVEELKKLLEG
GSGGGSGGSGGSGGMADSNGTITVEELKKLLEGGSGGMADSNGTITVEEL
KKLLE (SEQ ID NO: 27582)

>LUCCAGEM3 337 4copies linker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEMADSNGTITVEELKKLLEGGSGGMADSNGTITVEELKKL
LEGGSGGGSGGSGGSGGMADSNGTITVEELKKLLEGGSGGMADSNGTITV
EELKKLLE (SEQ ID NO: 27583)

>LUCCAGEM3 341 4copies linker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISRMADSNGTITVEELKKLLEGGSGGMADSNGTITVEE
LKKLLEGGSGGGSGGSGGSGGMADSNGTITVEELKKLLEGGSGGMADSNG
TITVEELKKLLE(SEQ ID NO: 27584)

>LUCCAGEM3 348 4copies linker

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISREAERSIRMADSNGTITVEELKKLLEGGSGGGSGGS
GGSGGMADSNGTITVEELKKLLEGGSGGMADSNGTITVEELKKLLEGGSG
GMADSNGTITVEELKKLLE (SEQ ID NO: 27585)

>LUCCAGEM3_334_2copies_linker_SpaC_Z

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAEGGSGGMADSNGTITVEELKKLLEGGGGSGGGSGGSGGSGGSG
GNKFNKEQQNAFYELKDDPSVSKEILAEAKKLNDAQAPKGGVDNKFNKEQ
QNAFYEILHLPNLNEEQRNAFIQSLKDDPSQSANLLAEAKKLNDAQAPK
(SEQ ID NO: 27586)

>LUCCAGEM3_337_2copies_linker_SpaC_Z

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEMADSNGTITVEELKKLLEGGSGGMADSNGTITVEELKKL
LEGGGGSGGGSGGSGGSGGSGGNKFNKEQQNAFYEILHLPNLTEEQRNGF
IQSLKDDPSVSKEILAEAKKLNDAQAPKGGVDNKFNKEQQNAFYEILHLP
NLNEEQRNAFIQSLKDDPSQSANLLAEAKKLNDAQAPK (SEQ ID NO:
27587)

>LUCCAGEM3_341_2copies_linker_SpaC_Z

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISRMADSNGTITVEELKKLLEGGSGGMADSNGTITVEE
LKKLLEGGGGSGGGSGGSGGSGGSGGNKFNKEQQNAFYEILHLPNLTEEQ
RNGFIQSLKDDPSVSKEILAEAKKLNDAQAPKGGVDNKFNKEQQNAFYEI
LHLPNLNEEQRNAFIQSLKDDPSQSANLLAEAKKLNDAQAPK (SEQ ID
NO: 27588)

>LUCCAGEM3_348_2copies_linker_SpaC_Z

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERDAE
RLSREAAAASEKISREAERSIRMADSNGTITVEELKKLLEGGSGGMADSN
GTITVEELKKLLEGGGGSGGGSGGSGGSGGSGGNKFNKEQQNAFYEILHL
PNLTEEQRNGFIQSLKDDPSVSKEILAEAKKLNDAQAPKGGVDNKFNKEQ
QNAFYEILHLPNLNEEQRNAFIQSLKDDPSQSANLLAEAKKLNDAQAPK
(SEQ ID NO: 27589)

### lucCageRBD Variants (SARS-CoV2 Spike Protein Receptor Binding Domain (RBD) Biosensors)

- LCB1: DKEWILQKIYEIMRLLDELGHAEASMRVSDLIYEFMKKGDE RLLEEAERLLEEVER (SEQID NO: 27397)

- LCB1_delta4: ILQKIYEIMRLLDELGHAEASMRVSDLIYEFMKK GDERLLEEAERLLEEVER(SEQ ID NO: 27590)

>lucCageRBD 336

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL) TDPDEARKAIAVTGYRLFEEILDAERL
SREAAAASDKEWILQKIYEIMRLLDELGHAEASMRVSDLIYEFMKKGDER LLEEAERLLEEVER* (SEQ ID NO: 27591)

>lucCageRBD 340

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISDKEWILQKIYEIMRLLDELGHAEASMRVSDLIYEFMKKG DERLLEEAERLLEEVER* (SEQ ID NO: 27592)

>lucCageRBD_344

[symbol]

MGSHHHHHHGSGSENLYFQG (SKEAAKKLQDLNIELARKLLEASTKLQR
LNIRLAEAAVELTDPKRIRDEIKEVKDKSKEIIRRAEKEIDDAAKESKKI
LEEARKAIRDAAEESQKLNLELAKLLLKAIAETQDLNLRAAKAFLEAAAK
LQELNIRAVELLVKLTDPATIRRALEHAKRRSKEIIDEAERAIRAAKRES
ERIIEEARRLIEKAKEESERIIREGSGSGDPDIKKLQDLNIELARELLRA
HAQLQRLNLELLRELLRALAQLQELNLDLLRLASEL)TDPDEARKAIAVT GYRLFEEILDAERLSREAAAASEKISREAEDKEWILQKIYEIMRLLDELG HAEASMRVSDLIYEFMKKGDERLLEEAERLLEEVER* (SEQ ID NO:
27593)

>lucCageRBD 347

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIDKEWILQKIYEIMRLLDELGHAEASMRVSDLI YEFMKKGDERLLEEAERLLEEVER* (SEQ ID NO: 27594)

>lucCageRBD 351

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAADKEWILQKIYEIMRLLDELGHAEASMRV SDLIYEFMKKGDERLLEEAERLLEEVER* (SEQ ID NO: 27595)

>lucCageRBD 354

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASDKEWILQKIYEIMRLLDELGHAEAS MRVSDLIYEFMKKGDERLLEEAERLLEEVER* (SEQ ID NO:27596)

>lucCageRBD_GGG_360

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEKISREGGGDKEWILQKIYEIMRLL DELGHAEASMRVSDLIYEFMKKGDERLLEEAERLLEEVER* (SEQ IDN
O: 27597)

>lucCageRBDdelta4 336

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASILQKIYEIMRLLDELGHAEASMRVSDLIYEFMKKGDERLLEEA ERLLEEVER* (SEQ ID NO: 27598)

>lucCageRBDdelta4 340

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISILQKIYEIMRLLDELGHAEASMRVSDLIYEFMKKGDERL LEEAERLLEEVER* (SEQ ID NO: 27599)

>lucCageRBDdelta4 344

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAE [symbol] RAIRAAKRESERI
IEEARRLIEKAKEESERIIREGSGSGDPDIKKLQDLNIELARELLRALAQ
LQELNLDLLRLASEL)TDPDEARKAIAVTGYRLFEEILDAERLSREAAAA
SLLDELGHAEASMRVSDLIYEFMKKGDERLLEEAERLLEEVER* (SEQ
ID NO: 27600) [symbol]
indicates text missing or illegible when filed

>lucCageRBDdelta4 347

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL) TDPDEARKAIAVTGYRLFEEILDAERL
SREAAAASEKISREAERSIILQKIYEIMRLLDELGHAEASMRVSDLIYEF MKKGDERLLEEAERLLEEVER* (SEQ ID NO: 27601)

>lucCageRBDdelta4 348

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL) TDPDEARKAIAVTGYRLFEEILDAERL
SREAAAASEKISREAERSIRILQKIYEIMRLLDELGHAEASMRVSDLIYE FMKKGDERLLEEAERLLEEVER* (SEQ ID NO: 27602)

>lucCageRBDdelta4 351

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL) TDPDEARKAIAVTGYRLFEEILDAERL
SREAAAASEKISREAERSIREAAILQKIYEIMRLLDELGHAEASMRVSDL IYEFMKKGDERLLEEAERLLEEVER* (SEQ ID NO: 27603)

>lucCageRBDdelta4 354

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASILQKIYEIMRLLDELGHAEASMRVS DLIYEFMKKGDERLLEEAERLLEEVER* (SEQ ID NO: 27604)

>lucCageRBDdelta4 357

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEKIILQKIYEIMRLLDELGHAEASM RVSDLIYEFMKKGDERLLEEAERLLEEVER* (SEQ ID NO: 27605)

>lucCageRBDdelta4_GGG_360

MGSHHHHHHGSGSENLYFQG(SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIREAAAASEKISREGGGILQKIYEIMRLLDELG HAEASMRVSDLIYEFMKKGDERLLEEAERLLEEVER* (SEQ ID NO:2
7606)

>lucCageRBD_348_d4LCB1v1.3

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEEILDAERLSR
EAAAASEKISREAERSIRILQKIYEIMKTLEQLGHAEASMQVSDLIYEFM
KQGDERLLEEAERLLEEVER* (SEQ ID NO: 27607)

> lucCageRBD_delta4_348

GSHHHHHHGSGSENLYFQG (SKEAAKKLQDLNIELARKLLEASTKLQRL
NIRLAEALLEAIARLQELNLELVYLAVELTDPKRIRDEIKEVKDKSKEII
RRAEKEIDDAAKESKKILEEARKAIRDAAEESRKILEEGSGSGSDALDEL
QKLNLELAKLLLKAIAETQDLNLRAAKAFLEAAAKLQELNIRAVELLVKL
TDPATIRRALEHAKRRSKEIIDEAERAIRAAKRESERIIEEARRLIEKAK
EESERIIREGSGSGDPDIKKLQDLNIELARELLRAHAQLQRLNLELLREL
LRALAQLQELNLDLLRLASEL)TDPDEARKAIAVTGYRLFEEILDAERLS
REAAAASEKISREAERSIRILQKIYEIMRLLDELGHAEASMRVSDLIYEF
MKKGDERLLEEAERLLEEVER (SEQ ID NO: 27608)

>lucCageRBD smbit128

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEkILDAERLSR
EAAAASEKISREAERSIRILQKIYEIMRLLDELGHAEASMRVSDLIYEFM
KKGDERLLEEAERLLEEVER (SEQ ID NO: 27609)

>lucCageRBD smbit99

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEkIsDAERLSR
EAAAASEKISREAERSIRILQKIYEIMRLLDELGHAEASMRVSDLIYEFM
KKGDERLLEEAERLLEEVER (SEQ ID NO: 27610)

>lucCageRBD smbit86

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVsGwRLFkkIsDAERLSR
EAAAASEKISREAERSIRILQKIYEIMRLLDELGHAEASMRVSDLIYEFM
KKGDERLLEEAERLLEEVER (SEQ ID NO: 27611)

>lucCageRBD_smbit104

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVeGYRLFEkIsDAERLSR
EAAAASEKISREAERSIRILQKIYEIMRLLDELGHAEASMRVSDLIYEFM
KKGDERLLEEAERLLEEVER (SEQ ID NO: 27612)

>lucCageRBD smbit101

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFEkesDAERLSR
EAAAASEKISREAERSIRILQKIYEIMRLLDELGHAEASMRVSDLIYEFM
KKGDERLLEEAERLLEEVER (SEQ ID NO: 27613)

>lucCageRBD_smbit_Y315W_E320K

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGwRLFEkILDAERLSR
EAAAASEKISREAERSIRILQKIYEIMRLLDELGHAEASMRVSDLIYEFM
KKGDERLLEEAERLLEEVER (SEQ ID NO: 27614)

>lucCageRBD_smbit_Y315W_E319K

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAER [symbol] AIRAAKRESERII
EEARRLIEKAKEESERIIREGSGSGDPDIKKLQDLNIELARELIRALAQL
QELNLDLLRLASEL)TDPDEARKAIAVTGwRLFkEILDAERLSREAAAAS
EIMRLLDELGHAEASMRVSDLIYEFMKKGDERLLEEAERLLEEVER (SE
Q ID NO: 27615)

>lucCageRBD smbit E319K

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDPDEARKAIAVTGYRLFkEILDAERLSR
EAAAASEKISREAERSIRILQKIYEIMRLLDELGHAEASMRVSDLIYEFM
KKGDERLLEEAERLLEEVER (SEQ ID NO: 27616)

>lucCageRBD SmBit position301

GSHHHHHHGSGSENLYFQGSGG(SKEAAKKLQDLNIELARKLLEASTKLQ
RLNIRLAEALLEAIARLQELNLELVYLAVELTDPKRIRDEIKEVKDKSKE
IIRRAEKEIDDAAKESKKILEEARKAIRDAAEESRKILEEGSGSGSDALD
ELQKLNLELAKLLLKAIAETQDLNLRAAKAFLEAAAKLQELNIRAVELLV
KLTDPATIRRALEHAKRRSKEIIDEAERAIRAAKRESERIIEEARRLIEK
AKEESERIIREGSGSGDPDIKKLQDLNIELARELLRAHAQLQRLNLELLR
ELLRALAQLQELNLDLLRLASEL)ggsVTGYRLFEEILRVKRESKRIVED
AERLsREAAAASEKISREAERSIRILQKIYEIMRLLDELGHAEASMRVSD
LIYEFMKKGDERLLEEAERLLEEVER (SEQ ID NO: 27617)

>lucCageRBD SmBit position308

GSHHHHHHGSGSENLYFQGSGG(SKEAAKKLQDLNIELARKLLEASTKLQ
RLNIRLAEALLEAIARLQELNLELVYLAVELTDPKRIRDEIKEVKDKSKE
IIRRAEKEIDDAAKESKKILEEARKAIRDAAEESRKILEEGSGSGSDALD
ELQKLNLELAKLLLKAIAETQDLNLRAAKAFLEAAAKLQELNIRAVELLV
KLTDPATIRRALEHAKRRSKEIIDEAERAIRAAKRESERIIEEARRLIEK
AKEESERIIREGSGSGDPDIKKLQDLNIELARELLRAHAQLQRLNLELLR
ELLRALAQLQELNLDLLRLASEL)TDPDEARVTGYRLFEEILRIVEDAER
LsREAAAASEKISREAERSIRILQKIYEIMRLLDELGHAEASMRVSDLIY
EFMKKGDERLLEEAERLLEEVER (SEQ ID NO: 27618)

>lucCageRBD loop

GSHHHHHHGSGSENLYFQG(SKEAAKKLQDLNIELARKLLEASTKLQRLN
IRLAEALLEAIARLQELNLELVYLAVELTDPKRIRDEIKEVKDKSKEIIR
RAEKEIDDAAKESKKILEEARKAIRDAAEESRKILEEGSGSGSDALDELQ
KLNLELAKLLLKAIAETQDLNLRAAKAFLEAAAKLQELNIRAVELLVKLT
DPATIRRALEHAKRRSKEIIDEAERAIRAAKRESERIIEEARRLIEKAKE
ESERIIREGSGSGDPDIKKLQDLNIELARELLRAHAQLQRLNLELLRELL
RALAQLQELNLDLLRLASEL)TDGGSGGPDEARKAIAVTGYRLFEEILDA
ERLSREAAAASEKISREAERSIRILQKIYEIMRLLDELGHAEASMRVSDL
IYEFMKKGDERLLEEAERLLEEVER (SEQ ID NO: 27619)

LacATrop (split β-lactamase A in bold; underline cTnT and cTnC) :

MGSHHHHHHGSGSENLYFQG (SGGSVFAHPETLVK VKDAEDQLGA RVGYIELDLN
SGKILESFRP EERFPMMSTF KVLLCGAVLS RVDAGQEQLG RRIHYSQNDL
VEYSPVTEKH LTDGMTVREL CSAAITMSDN TAANLLLTTI GGPKELTAFL
HNMGDHVTRL DRWEPELNEA IPNDERDTTT PAAMATTLRK LLTGENGR
SGGGGSGGGGSGGGG (SKEAAKKLQDLNIELARKLLEASTKLQRLNIRLAEALLEAIARLQELNLELVYLAVELT
DPKRIRDEIKEVKDKSKEIIRRAEKEIDDAAKESKKILEEARKAIRDAAEESRKILEEGSGSGSDALDELQKLNL
ELAKLLLKAIAETQDLNLRAAKAFLEAAAKLQELNIRAVELLVKLTDPATIRRALEHAKRRSKEIIDEAERAIRA
AKRESERIIEEARRLIEKAKEESERIIREGSGSGDPDIKKLQDLNIELARELLRAHAQLQRLNLELLRELLRALA
QLQELNLDLLRLASEL) TDPDEARKAIAVTGYRLFEEILDAERLIREAAAASEDQLREAAKELWQTIYNLEAEKF
DLQEKFKQQKYEINVLRNRINDNQKVSKTKDDSKGKSEEELSDLFRMFDKNADGYIDLEELKIMLQATGETITED
DIEELMKDGDKNNDGRIDYDEFLEFMKGVE (SEQ ID NO: 27620)

In another aspect, the disclosure provides key proteins capable of binding to the structural region of a cage protein of any embodiment or combination of embodiments disclosed herein that does not include the second reporter protein domain, wherein binding of the key protein to the cage protein only occurs in the presence of a target to which the cage protein one or more target binding polypeptide can bind, wherein the k [symbol] second reporter protein domain, wherein interaction of the key protein second reporter protein domain and the cage protein first reporter protein domain causes a detectable change in reporting activity from the first reporter protein domain.

As disclosed herein, the key proteins of this aspect can be used, for example, in conjunction with the cage polypeptides to displace the latch through competitive intermolecular binding that induces conformational change, leading to interaction of the key protein second reporter protein domain and the cage protein first reporter protein domain causes a detectable change in reporting activity from the first reporter protein domain.

In one embodiment, wherein the second reporter protein domain is at the N-terminus or the C-terminus of the key protein, or is within 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acid of the N-terminus or the C-terminus of the key protein.

In another embodiment, the second reporter protein domain comprises a reporter protein domain selected from the group consisting of luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease). In various non-liming embodiments, the second reporter protein domain comprises an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27360-23379, wherein underlined residues are optional residues that may be present or absent, and when present may be any amino acid sequence, and wherein any N-terminal methionine residue may be present or absent.

In another embodiment, the key protein, not including the second reporter protein domain, comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues, to the amino acid sequence of a key polypeptide disclosed in US20200239524 (or WO2020/018935), or a key polypeptide selected from the group consisting of SEQ ID NOS:14318-26601, 26602-27015, 27016-27050, 27,322 to 27,358, and key polypeptides with an odd-numbered SEQ ID NOS: 27127 and 27277), Table 3 (table 8 herein), and/or Table 4 (table 9 herein) of WO2020/018935.

In a further embodiment, the key protein comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues in parentheses, to the amino acid sequence of a key protein selected from the group consisting of SEQ ID NOS: 27621-27623, wherein residues in parentheses are optional and may be present or absent.

> lucKey: MGS-(His)6-TEV site-linker-LgBit-linker-latch sequence

(MGSHHHHHHGSGSENLYFQG)SGMVFTLEDFVGDWEQTAAYNLDQVLEQ GGVSSLLQNLAVSVTPIQRIVRSGENALKIDIHIIPYEGLSADQMAQIEE VFKVVYPVDDHHFKVILPYGTLVIDGVTPNMLNYFGRPYEGIAVFDGKKI TVTGTLWNGNKIIDERLITPDGSMLFRVTINSGGSGGGGSGGGSGGSDEA
RKAIARVKRESKRIVEDAERLIREAAAASEKISREAERLIREAAAASEKI
SRE (SEQ ID NO:27621)

Key-2GGSGG-CyOFP (CyOFP sequence in bold/underline) :

(M) DPDEARKAIARVKRESKRIVEDAERLIREAAAASEKISREAERLIREAAAASEKISREGGSGG GGVSKGEELIK ENMRSKLYLE GSVNGHQFKC THEGEGKPYE GKQTNRIKW EGGPLPFAFD ILATHFMYGSKVFIKYPADL PDYFKQSFPE GFTWERVMVF EDGGVLTATQ DTSLQDGELI YNVKVRGVNFPANGPVMQKK TLGWEPSTET MYPADGGLEG RCDKALKLVG GGHLHVNFKT TYKSKKPVKMPGVHYVDRRL ERIKEADNET YVEQYEHAVA RYSNLGGGMD ELYK (SEQ ID NO: 27622)

Key-LacB (split β-lactamase B in bold/underline) :

SGSGDPDEARKAIARVKRESKRIVEDAERLIREAAAASEKISREAERLIREAAAASEKISRESGGGGSGGGGSGG
GG LLTLASRQQLIDWME ADKVAGPLLR SALPAGWFIA DKSGAGERGS RGIIAALGPD GKPSRIVVIYTTGSQATMDE RNRQIAEIGA SLIKHW (SEQ ID NO: 27623)

In another aspect, the disclosure provides a biosensor, comprising (a) a cage protein of any embodiment or combination of embodiments herein, wherein the cage does not include the second reporter protein domain; and (b) the key protein of embodiment or combination of embodiments herein; wherein the key protein can only bind to the cage protein in the presence of a target to which the cage protein one or more target binding polypeptide can bind; and wherein binding of the first reporter protein domain of the cage protein to the second reporter protein domain of the key protein causes a detectable change in reporting activity from the first reporter protein domain.

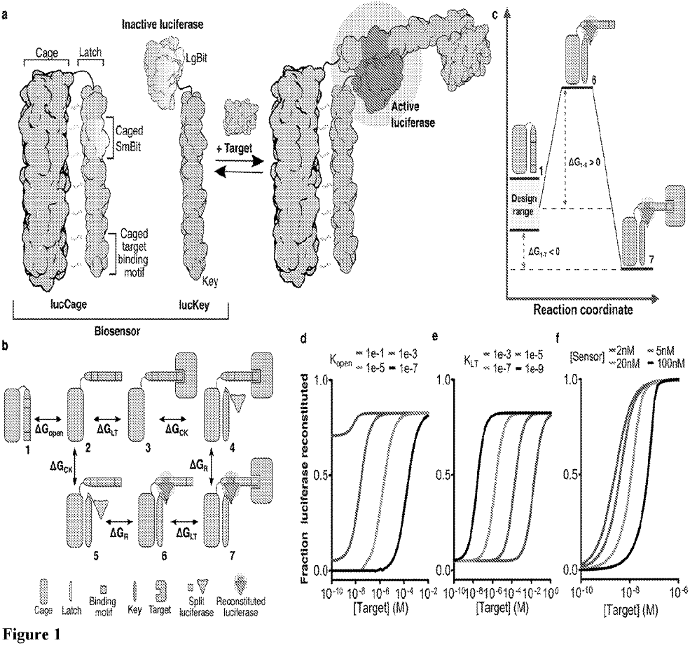

As described herein the inventors have developed an inverted LOCKR system exemplified by a cage protein comprising a structural region and a latch region containing a first reporter protein domain and one or more target binding polypeptide (sometimes referred to as an analyte binding motif/target epitope in the examples), and a key protein which contains the second reporter protein domain linked to a key peptide. This system has at least three important states (FIG. 1C). State 1 is a closed OFF state in whi [symbol] region interacts with the latch region, sterically occluding the one or more target binding polypeptide from binding its target and the first reporter protein domain from combining with the second reporter protein domain to reconstitute reporter protein activity. States 2 or 3 are open states in which these binding interactions are not blocked, and the key protein can bind the cage protein structural domain. State 7 is a stable ON state established when tri-molecular association of key protein with cage protein structural domain and the one or more target polypeptide with its target results in reconstitution of reporter protein activity. Mixing the cage protein with either a key protein or target alone is not sufficient to activate reporter activity. Both key protein and target together in the same solution with the cage protein results in reconstitution of reporter protein activity. Strong latch region-target interaction provides the driving force to populate the ON State 7 (signal) over State 6 (background). Further details are provided in the examples that follow.

As discussed above, the detectable change may be any increase or a decrease in the relevant reporting activity, as deemed suitable for an intended purpose. In various nonlimiting embodiments, the detectable change in reporting activity may include, but is not limited to:
The first reporter protein domain is a split fluorescent or luminescent protein domain that emits no fluorescence/luminescence, or detectably less fluorescence/luminescence then when bound to the second split reporter protein domain.
The first and second reporter protein domains are BRET or FRET pairs that emit detectable signal at different wavelengths when bound to each other versus when not bound to each other.
Cell survival selection by dihydrofolate reductase (DHFR) complementation in the presence of chosen target, when the first and second reporter protein domains reconstitute DHFR activity.
Next generation sequencing as the readout to profile chemical or genetic perturbations on target-selective pathway when the first and second reporter protein domains reconstitute TEV protease activity for use as a molecular barcode.
Positron emission tomography (PET) when the first and second reporter protein domains reconstitute thymidine kinase.
Electrochemical readout when the first and second reporter pro reconstitute APEX2 activity.
Colorimetry readout when the first and second reporter protein domains reconstitute beta-lactamase or horseradish peroxidase activity.

In various embodiments of the biosensor of the disclosure:
(a) the first reporter protein domain comprises an amino acid sequence at least 70%, 75%, 80%, 85%, 90%, 91%, 92%, 93%, 94%, 95%, 96%, 97%, 98%, or 100% identical to the amino acid sequence SEQ ID NO: 27359, and 27664-27672 and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO 27379, wherein the N-terminal methionine residue may be present or absent
(b) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27360,and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27361;
(c) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27362,and the other comprises an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27363-27365;
(d) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27366,and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO 27368:
(e) one of the first reporter protein domain and the second m [symbol] comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27367, wherein the N-terminal methionine residue may be present or absent,and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO 27368, wherein the N-terminal methionine residue may be present or absent;
(f) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27369, wherein underlined residues are optional residues that may be present or absent, and when present may be any amino acid sequence; and the other comprises an amino acid sequence at least 70%, 75%, 80%, 85%, 90%, 91%, 92%, 93%, 94%, 95%, 96%, 97%, 98%, or 100% identical to the amino acid of SEQ ID NO:27370, wherein underlined residues are optional residues that may be present or absent, and when present may be any amino acid sequence;
(g) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27371, wherein underlined residues are optional residues that may be present or absent, and when present may be any amino acid sequence, and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27372, wherein underlined residues are optional residues that may be present or absent, and when present may be any amino acid sequence;
(h) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27373, wherein underlined residues are optional residues that may be present or absent, and when present may be any amino acid sequence, and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27374, wherein underlined residues are optional residues that may be present or absent, and when present may be any amino acid sequence;
(i) one of the first reporter protein domain and the second [symbol] comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27375, wherein underlined residues are optional residues that may be present or absent, and when present may be any amino acid sequence, and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27376, wherein underlined residues are optional residues that may be present or absent, and when present may be any amino acid sequence;
(j) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27377, wherein the N-terminal methionine residue may be present or absent, and wherein underlined residues are optional residues that may be present or absent, and when present may be any amino acid sequence, and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27378, wherein underlined residues are optional residues that may be present or absent, and when present may be any amino acid sequence.

In one specific embodiment of the biosensor, the cage protein comprises a cage protein comprising an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues, to the amino acid sequence of a cage protein listed in Table 10, wherein the N-terminal protein purification tag (MGSHHHHHHGSGSENLYFQGSGG (SEQ ID NO:27624); or MGSHHHHHHGSENLYFQG (SEQ ID NO:27625); or GSHHHHHHGSGSENLYFQG (SEQ ID NO:27626)) is optional, and can be present or absent, and the key protein comprises an amino acid sequence at least 70%, 75%, 80%, 85%, 90%, 91%, 92%, 93%, 94%, 95%, 96%, 97%, 98%, or 100% identical, not including optional amino acid residues in parentheses, to the amino acid sequence of SEQ ID NO:27621.

> lucKey: MGS-(His)6-TEV site-linker-LgBit-linker-latch sequence

(MGSHHHHHHGSGSENLYFQG)SGMVFTLEDFVGDWEQTAAYNLDQVLEQ GGVSSLLQNLAVSVTPIQRIVRSGENALKIDIHIIPYEGLSADQMAQIEE VFKWYPVDDHHFKVILPYGTLVIDGVTPNMLNYFGRPYEGIAVFDGKKI [symbol] TVTGTLWNGNKIIDERLITPDGSMLFRVTINSGGSGGGG
SGGGSGGSDEARKAIARVAAASEKISREAERLIREAAAASEKISRE (SE
Q ID NO: 27621) [symbol]
indicates text missing or illegible when filed

In another specific embodiment of the biosensor, the cage protein and the key protein comprise a protein pair comprising:

(i) a cage protein comprising an amino acid sequence at least 70%, 75%, 80%, 85%, 90%, 91%, 92%, 93%, 94%, 95%, 96%, 97%, 98%, or 100% identical to the amino acid sequence of SEQ ID NO: 27620, wherein the residues in parentheses are optional and may be present or absent: LacATrop (split β-lactamase A in bold; underline cTnT and cTnC) :

(MGSHHHHHHGSGSENLYFQG SGGS)VFAHPETLVK VKDAEDQLGA RVGYIELDLN
SGKILESFRP EERFPMMSTF KVLLCGAVLS RVDAGQEQLG RRIHYSQNDL
VEYSPVTEKH LTDGMTVREL CSAAITMSDN TAANLLLTTI GGPKELTAFL
HNMGDHVTRL DRWEPELNEA IPNDERDTTT PAAMATTLRK LLTGENGR
SGGGGSGGGGSGGGGSKEAAKKLQDLNIELARKLLEASTKLQRLNIRLAEALLEAIARLQELNLELVYLAVELTD
PKRIRDEIKEVKDKSKEIIRRAEKEIDDAAKESKKILEEARKAIRDAAEESRKILEEGSGSGSDALDELQKLNLE
LAKLLLKAIAETQDLNLRAAKAFLEAAAKLQELNIRAVELLVKLTDPATIRRALEHAKRRSKEIIDEAERAIRAA
KRESERIIEEARRLIEKAKEESERIIREGSGSGDPDIKKLQDLNIELARELLRAHAQLQRLNLELLRELLRALAQ
LQELNLDLLRLASELTDPDEARKAIAVTGYRLFEEILDAERLIREAAAASEDQLREAAKELWQTIYNLEAEKFDL
QEKFKQQKYEINVLRNRINDNQKVSKTKDDSKGKSEEELSDLFRMFDKNADGYIDLEELKIMLQATGETITEDDI
EELMKDGDKNNDGRIDYDEFLEFMKGVE (SEQ ID NO:27620); and

(ii) a key protein comprising an amino acid sequence at least 70%, 75%, 80%, 85%, 90%, 91%, 92%, 93%, 94%, 95%, 96%, 97%, 98%, or 100% identical to the amino acid sequence of SEQ ID NO:27361:

LLTLASRQQLIDWME ADKVAGPLLR SALPAGWFIA DKSGAGERGS RGIIAALGPD GKPSRIVVIY
TTGSQATMDE RNRQIAEIGA SLIKHW (SEQ ID NO:27361)

In another aspect, the disclosure provides methods for detecting a target, comprising
(a) contacting the cage protein of any embodiment disclosed herein where the cage protein comprises the second reporter protein domain, or the biosensor of any embodiment herein with a biological sample under conditions to promote binding of the cage protein one or more target binding polypeptide to a target present in the biological sample, causing a detectable change in reporting activity from the first reporter protein domain; and
(b) detecting the change in reporting activity from the reporter protein domain, wherein the change in reporting activity identifies the sample as containing the target.

As described above, the inventors have developed an inverted LOCKR system exemplified by a cage protein comprising a structural region and a latch region containing a first reporter protein domain and one or more target binding polypeptic to as an analyte binding motif/target epitope in the examples), and a key protein wmcn contains the second reporter protein domain linked to a key peptide. As also discussed above, the detectable change may be any increase or a decrease in the relevant reporting activity, as deemed suitable for an intended purpose. Various non-limiting embodiments of the detectable change in reporting activity are described above, and methods for detecting such detectable changes are exemplified in detail in the examples that follow. Based on the teachings herein, those of skill in the art can determine the appropriate technique for measuring a detectable change of interest.

As exemplified in FIG. 19 and discussed in example 3, the methods can accommodate an “indirect detection” approach, in which the reporter protein (intermolecular (second reporting domain in cage protein) or intramolecular (second reporter protein on key) embodiments; is reconstituted by pre-incubation of the biosensor with the target for the target binding polypeptide, resulting in restoration of reporter activity. The activated biosensor is then incubated with a sample to detect the presence of an target to which the one or more target binding polypeptide binds, resulting in binding of the target to the one or more target binding polypeptide, loss of interaction between the reporter protein components, and reduction/elimination of reporting activity.

Any suitable biological sample may be used, including but not limited to blood, serum, saliva, urine, semen, vaginal fluid, lymph, tissue fluid, digestive fluid, sweat, tears, nasal discharge, amniotic fluid, and breast milk.

Any target may be detected as deemed appropriate for an intended use and for which one or more target binding polypeptide is available for inclusion in the cage protein. In non-limiting embodiments, the target is selected from the group including but not limited to an antibody, a toxin, a diagnostic biomarker, a viral particle, or a disease biomarker. In one specific embodiment, the target is an antibody. In a further embodiment, the target comprises antibodies selective for a virus. In various such embodiments, the one or more target binding polypeptide may comprises the amino acid sequence selected from the group consisting of SEQ ID NOS: 27292-27394 and 27547-27548, and a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27397-27494. In these embodiments, the methods may be used to detect the presence of antibodies against a SARS coronavirus, or SARS-CoV-2.

In various further embodiments, the cage polypeptide comprises the amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues, to the amino acid sequence of a cage protein listed in Table 10.

In another embodiment, the target is a disease marker or toxin. In one such embodiment, the disease marker or toxin comprises Bcl-2, Her2 receptor, Botulinum neurotoxin B, albumin, epithelial growth factor receptor, prostate-specific membrane antigen (PSMA), citrullinated peptides, brain natriuretic peptides, and/or cardiac Troponin I. In another embodiment, the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 27380-27390, wherein any N-terminal amino acid is optional and may be present or absent.

In various further embodiments, the cage polypeptide comprises the amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues, to the amino acid sequence of a cage protein listed in Table 10.

The disclosure also provides methods for designing/making a biosensor, cage protein, or key protein comprising the steps of any method described herein, such as in the examples that follow.

In another aspect, the disclosure provides nucleic acids encoding a cage protein, key protein, or epitope of the disclosure. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In another aspect, the disclosure provides expression vectors comprising the nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In one aspect, the present disclosure provides cells comprising the cage protein, key protein, epitope, biosensor, nucleic acid, and/or expression vector of any embodiment or combination of embodiments of the disclosure, wherein the cells can be either prokaryotic or eukaryotic, such as mammalian cells. In one embodiment the cells may be transiently or stably transfected with the nucleic acids or expression vectors of the disclosure. Such transfection of expression vectors into prokaryotic and eukaryotic cells can be accomplished via any technique known in the art. A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

In another aspect, the disclosure provides pharmaceutical compositions comprising
(a) the cage protein, key protein, biosensor, epitope, recombinant nucleic acid, expression vector, and/or the cell of any embodiment or combination of embodiments herein; and
(b) a pharmaceutically acceptable carrier.

The compositions may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, be chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate- 60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

In a further aspect, the disclosure provide an epitope, comprising or consisting of the amino acid sequence of SEQ ID NO:27384

| | | |
|---|---|---|
| lucCageTrop | cTnI + cTnC | EDQLREKAKELWQTIYNLEAEKFDLQEKFKQQKYEINVL RNRINDNQKVSKTKDDSKGKSEEELSDLFRMFDKNADGY IDLEELKIMLQATGETITEDDIEELMKDGDKNNDGRIDY DEFLEFMKGVE (SEQ ID NO:27384) |

The epitope can be used, for example, in the biosensors of the disclosure. In one aspect, the disclosure provides methods for detecting Troponin I in a sample, comprising contacting a biological sample with the epitope under conditions suitable to promote binding of Troponin I in the sample to the epitope to form a binding complex, and detecting binding complexes that demonstrate presence of Troponin I in the sample. All embodiments of biological samples and detection as disclosed herein case be used in these methods as well.

### EXAMPLES

Here, we show that a very general class of allosteric protein-based biosensors can be created by inverting the flow of information through de novo designed protein switches in which binding of a peptide key triggers biological outputs of interest. Using broadly applicable design principles, we allosterically couple binding of protein the reconstitution of luciferase activity and a bioluminescent readout through the association of designed lock and key proteins. Because the sensor is based purely on thermodynamic coupling of analyte binding to switch activation, only one target binding domain is required, which simplifies sensor design and allows direct readout in solution. We demonstrate the modularity of this platform by creating biosensors that, with little optimization, sensitively detect the anti-apoptosis protein Bcl-2, the hIgG1 Fc domain, the Her2 receptor, and Botulinum neurotoxin B, as well as biosensors for cardiac Troponin I and an anti-Hepatitis B virus (HBV) antibody that achieve the sub-nanomolar sensitivity necessary to detect clinically relevant concentrations of these molecules. We also use the approach to design sensors of antibodies against SARS-CoV-2 protein epitopes and of the receptor-binding domain (RBD) of the SARS-CoV-2 Spike protein. The latter, which incorporates a de novo designed RBD binder, has a limit of detection of 15pM with an up to seventeen fold increase in luminescence upon addition of RBD. The modularity and sensitivity of the platform enable the rapid construction of sensors for a wide range of analytes and highlights the power of de novo protein design to create multi-state protein systems with new and useful functions.

A protein biosensor can be constructed from a system with two nearly isoenergetic states - the equilibrium between which is modulated by the analyte being sensed. Desirable properties in such a sensor are (i) the analyte triggered conformational change should be independent of the details of the analyte (so the same overall system can be used to sense many different compounds) (ii) the system should be tunable so that analytes with different binding energies and relevant concentrations can be detected over a large dynamic range, and (iii) the conformational change should be coupled to a sensitive output. We hypothesized that these attributes could be attained by inverting the information flow in de novo designed protein switches in which binding to a target protein of interest is controlled by the presence of a peptide actuator. These switches consist of a constant “cage” region that sequesters a “latch” that binds the target of interest; addition of a peptide “key” displaces the latch from the cage leading to target binding and associated downstream events. However, from a thermodynamic viewpoint, the key and the target are equivalent: the binding of the two to the cage is thermodynamically coupled since the latch has to open, with free energy cost ΔGₒₚₑₙ (FIG. 1b), in order for either to bind. Hence, the free energy associated with binding both target and key is more favorable than the sum of the free energies of binding the two individually (FIG. 1c). The difference between key and target is in their variability; the key is constant while the target can be any desired interaction. For an actuator, it is desirable to have a constant input drive a wide range of customizable responses, and hen work, the input was the (constant) key and the output was binding to a variety of targets associated with protein degradation, nuclear export, etc. We reasoned that the input to the system could be inverted to create biosensors with a constant readout -- addition of a (variable) target could induce binding of the (constant) key to the (constant) cage, and that this association could be coupled to an enzymatic readout. Such a system would satisfy properties (i) and (ii) above, as a wide range of binding activities can be caged, and since the switch is thermodynamically controlled, it is straightforward to adjust the relative energies of key and target binding to achieve activation at the relevant target concentrations. Because the key and the cage are always the same, the system is modular: the same molecular association can be coupled to the binding of many different targets.

To achieve property (iii), we reasoned that bioluminescence could provide a rapid and sensitive readout of analyte driven cage-key association, and explored the use of a reversible split luciferase complementation system. We developed a system consisting of two protein components: a ‘lucCage’ comprising a cage domain and a latch domain containing the short split luciferase fragment (SmBiT) and an analyte binding motif of choice; and a “lucKey”, which comprises the larger split luciferase fragment (LgBit) and a key peptide (FIG. 1a). lucCage has two states: a closed state in which the cage domain binds the latch and sterically occludes the analyte binding motif from binding its target and SmBiT from combining with LgBit to reconstitute luciferase activity; and an open state in which these binding interactions are not blocked, and lucKey can bind the cage domain. Association of lucKey with lucCage results in the reconstitution of luciferase activity (FIG. 1a, right). The target may be viewed as allosterically regulating luciferase activity, since binding to the sensor is at a site distant from the enzyme active site.

The states of such a system are in thermodynamic equilibrium, with the tunable parameters ΔGₒₚₑₙ and ΔG_{CK} governing the populations of the possible species, along with the free energy of association of the analyte to the binding domain ΔG_{LT} (FIG. 1b). To achieve high sensitivity, the closed state (species 1) must be substantially lower in free energy than the open state in the absence of target (species 6) to avoid background signal ΔG₁₋₆>0), but higher in free energy than the open state in the presence of target (species 7, ΔG₁₋₇<0), so that target detection is energetically favorable (FIG. 1c). To guide the optimization of biosensor sensitivity, we simulated the dependence of the sensor system on ΔGₒₚₑₙ(FIG. 1d), ΔG_{LT} (FIG. 1e), and the concentration of analyte and the sensor components (FIG. 1f) (See Supplementary Methods for details). As expected, the sensitivity of analyte detection is a function of ΔG_{LT}, with a lower limit of roughly one-tenth binding (FIG. 1e; below this concentration, the free energy of binding is too small to open the switch). Hence sensing domains with high affinity to their target will yield more sensitive biosensors. The sensitivity of the system can be further tuned above this lower limit by varying the concentration of lucCage and lucKey, resulting in sensing systems responding to different target concentration ranges (FIG. 1f). Tuning the strength of the intramolecular cage-latch interaction (ΔGₒₚₑₙ) affects the equilibrium population of the catalytically active species (species 6 and 7, FIG. 1d), which in turn affects the sensitivity: too tight interaction results in low signal in the presence of target, and too weak an interaction results in high background in the absence of target. Our design strategy aims to find this balance by designing sensors in the closed state (species 1) with a range of ΔGₒₚₑₙ values: ΔGₒₚₑₙ can be increased (decreased) by increasing (decreasing) the length of the latch helix and by introducing either favorable hydrophobic interactions or unfavorable steric clashes and buried polar atoms at the cage-latch interface; we employ both strategies to tune the sensors described below (ΔG_{CK} can also be tuned, but we did not find this necessary for the sensors described here).

To streamline the design of new sensors based on these principles, we developed a Rosetta™-based computational method for the incorporation of diverse sensing domains into the LOCKR switches called GraftSwitchMover. This method identifies the most suitable position for embedding a target binding peptide within the latch such that the resulting protein is stable in the closed state and the interactions with the target are blocked. This is done by maximizing favorable hydrophobic packing interactions between the peptide and the cage and minimizing the number of unfavorable buried hydrophilic residues. This method takes as input the 3-dimensional model of the switch, the sequence of a peptide that binds the target of interest, and a list of the residues in this peptide that interact with the target (interface residues), and returns a set of designs in which the binding of the peptide to the target is predicted to be blocked by association with the cage (See supplementary methods). The final set of designs covers a range of ΔGₒₚₑₙvalues (FIG. 1c), which can be further tuned through introducing destabilizing mutations in the latch: I328S (“1S”) or I328S/L345S (“2S”). These designs are then experimentally characterized to find the most sensitive biosensors.

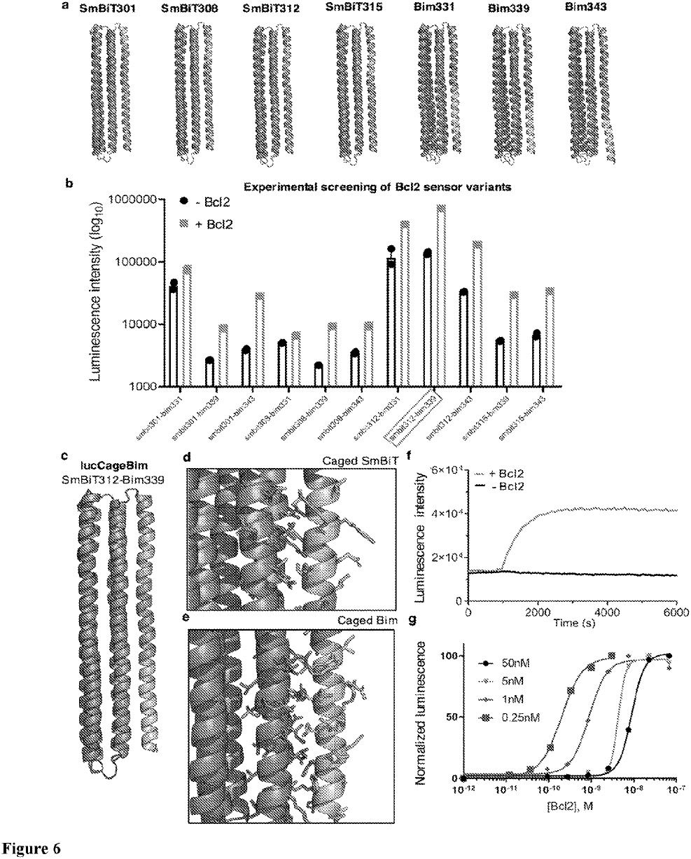

We first set out to test our hypothesis by grafting the SmBiT peptide and the Bim peptide in the closed state of the optimized asymmetric LOCKR switch described in Langan et al, 2020² (FIG. 6). SmBiT naturally adopts a β-strand conformation within the luciferase holoenzyme, but we assumed that it will adopt a helical secondary structure in the context of the helical bundle scaffold, consistent with the observation that some p adopt diverse secondary structures in a context-dependent manner. We sampled different threadings for the two peptide sequences across the latch, built three-dimensional models, selected the lowest energy solutions (3 positions for SmBiT, and 4 positions for the Bim peptide) (FIG. 6a) and expressed twelve designs in E. coli. We mixed the designs with lucKey in a 1:1 ratio, then added Bcl-2, which binds with nanomolar affinity to Bim, and monitored luciferase activity (FIG. 6b). We found that upon the addition of Bcl-2 to a solution containing the new Cage designs, lucKey, and furimazine substrate, there was a rapid increase in luminescence (FIG. 6f), suggesting that the inverse LOCKR system can indeed function as a biosensor. Further characterization of the best Bcl-2 sensor candidate, lucCageBim, demonstrated that the analyte detection range could be tuned by varying the concentration of the sensor (lucCage + lucKey) (FIG. 6g) as anticipated in our model simulations (FIG. 1f). Experimental characterization of the different designs showed that inserting SmBiT into position 312 of the LOCKR cage (SmBiT312) yielded the highest stability and brightness (FIG. 6b), therefore we used this design, henceforward referred to as “lucCage”, as the base scaffold for the biosensors described below.

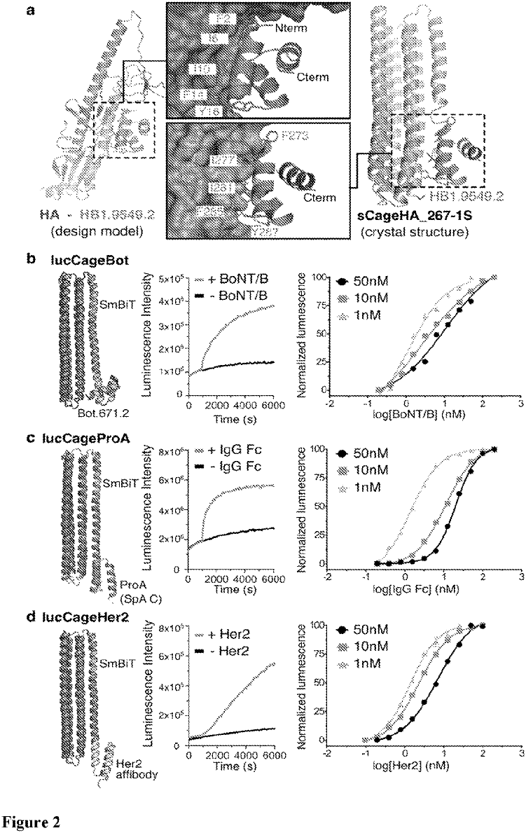

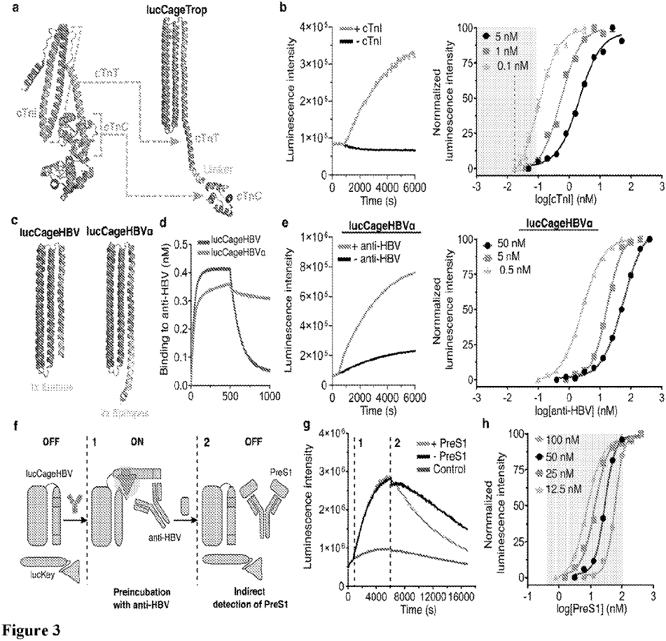

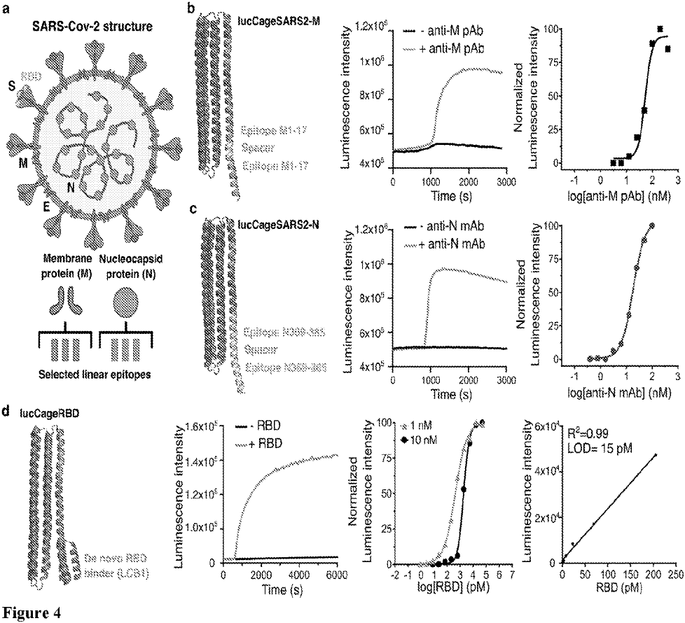

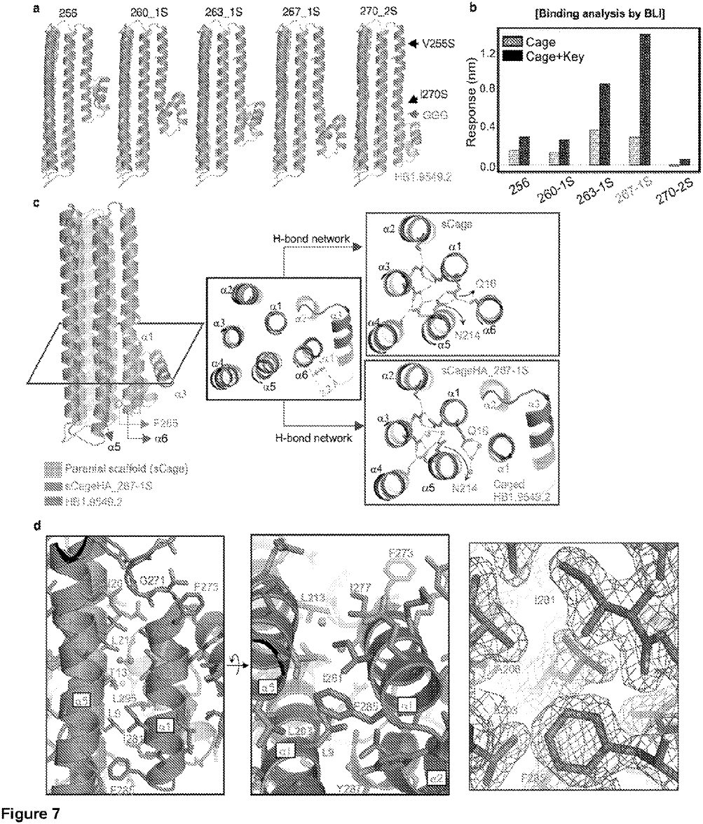

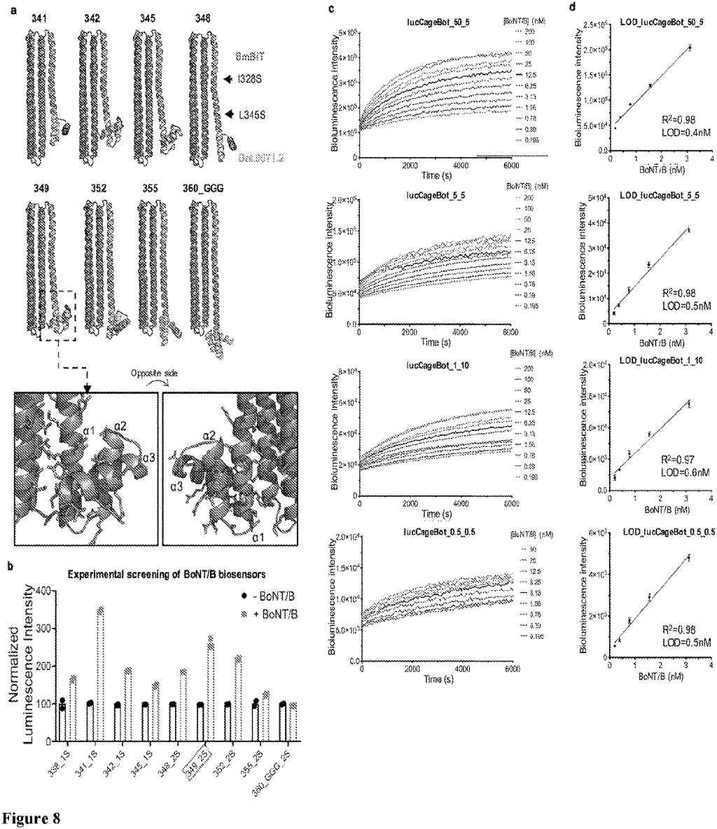

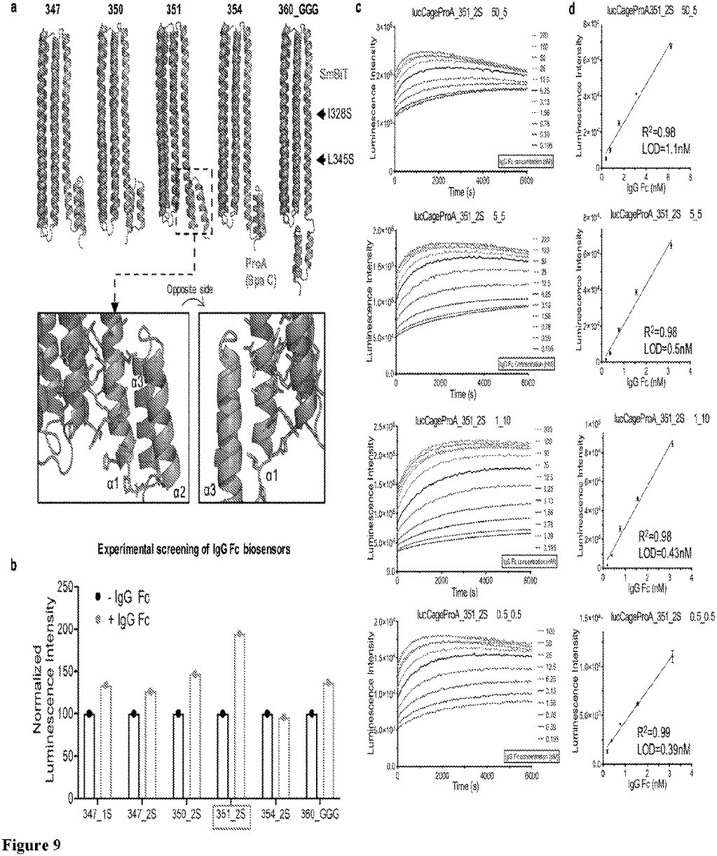

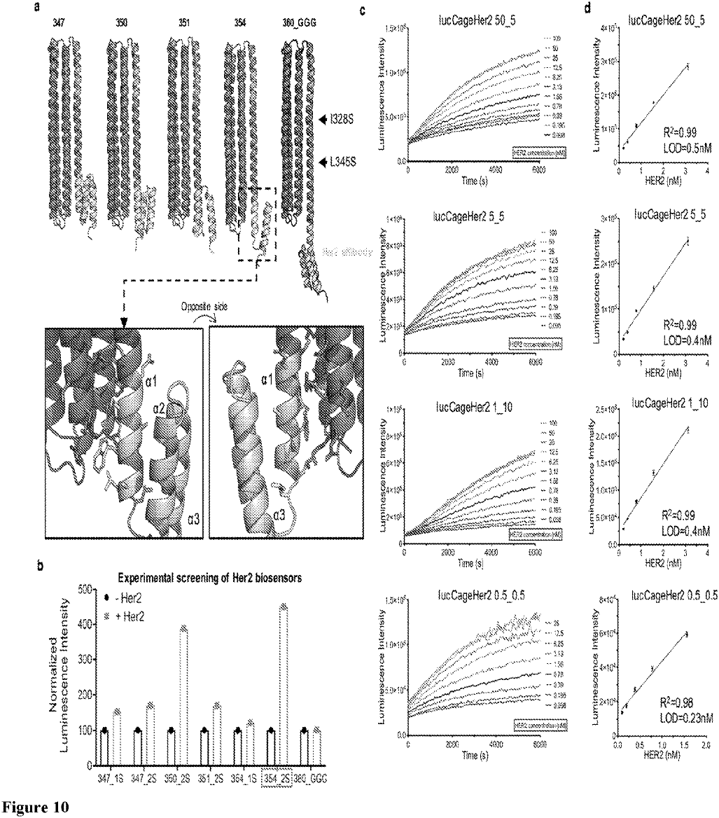

To explore the versatility of our new biosensor platform, we next investigated the incorporation of a range of binding modalities for analytes of interest within lucCage. First, we set out to explore how to computationally cage target-binding proteins, rather than peptides, in the closed state. We identified the primary interaction surface of the binding protein to its target, extracted the main secondary structure elements involved in it to use them in the computational protocol described above, and selected the best designs from the many threadings generated. Then, we used Rosetta™ Remodel to model the full-length binding domain in the context of the switch and selected designs in which this interface was buried against the cage with minimal steric clashes (See supplementary methods). As a test case, we caged the de novo designed protein, HB1.9549.2, which binds to Influenza A H1 hemagglutinin (HA)¹⁵ into a shortened version of the LOCKR switch (sCage), optimized to improve stability and facilitate crystallization efforts (FIG. 2a). Two of five designs were functional, and bound HA in the presence but not the absence of key (FIG. 7b). The crystal structure of the best design, sCageHA _267-1S, determined to 2.0 Å resolution (Table 11), showed that all HA-binding residues except one (F273) interact with the cage domain (blocking binding of the latch to the switch) as intended by design (FIG. 2a, FIGS. 7a-c). With this structural validation of the design concept in hand, we next sought to develop new sensors using small proteins as sensing domains for the detection of botulinum neurotoxin, the immunoglobulin Fc domain, and the Her2 receptor. To do so, we g designed binder for Botulinum neurotoxin B (BoNT/B)¹⁵, the C domain of the generic antibody binding protein Protein A¹⁶, and a Her2-binding affibody¹⁷, into lucCage. After screening a few designs for each target (FIGS. 8-10), we obtained highly sensitive lucCages (lucCageBot, lucCageProA, and lucCageHer2) that can detect BoNT/B (FIG. 2b, FIG. 8), hIgG Fc domain (FIG. 2c, FIG. 9), and Her2 receptor (FIG. 2d; FIG. 10) respectively, demonstrating the modularity of the platform. The designed sensors responded within minutes upon adding the target, and their sensitivity could be tuned by changing the concentration of lucCage and lucKey (FIG. 2), as predicted by our model simulations (FIG. 1f). These sensors may be used in multiple applications, such as rapid and low-cost detection of highly toxic botulinum neurotoxins in the food industry, which currently relies heavily on live-animal bioassays, or detection of high serological levels of soluble Her2 (>15 ng/mL) associated with metastatic breast cancer, levels that could be detected with the current sensitivity of lucCageHer2.

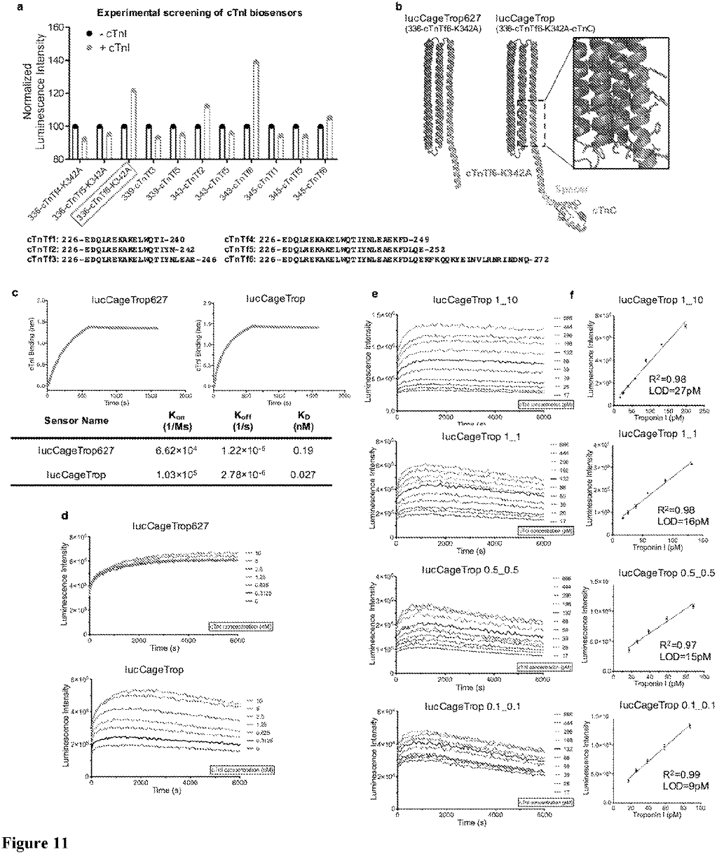

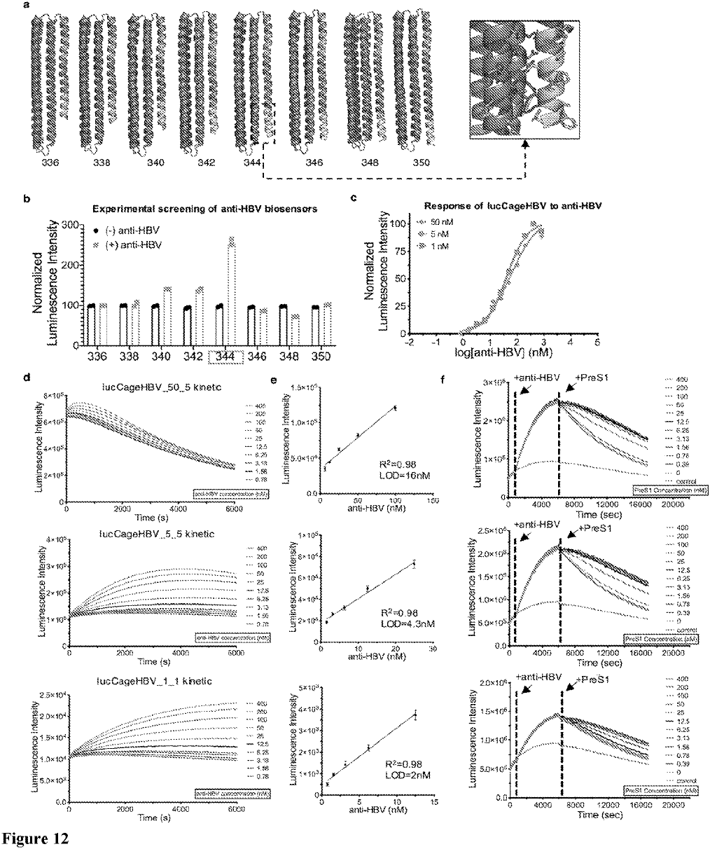

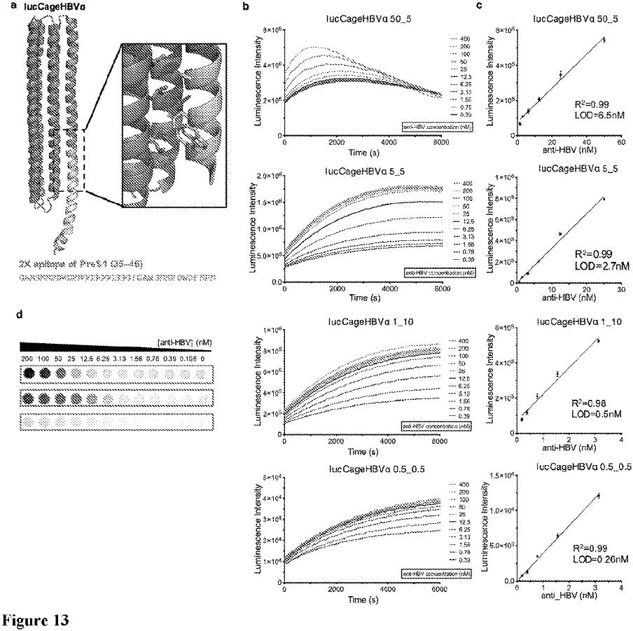

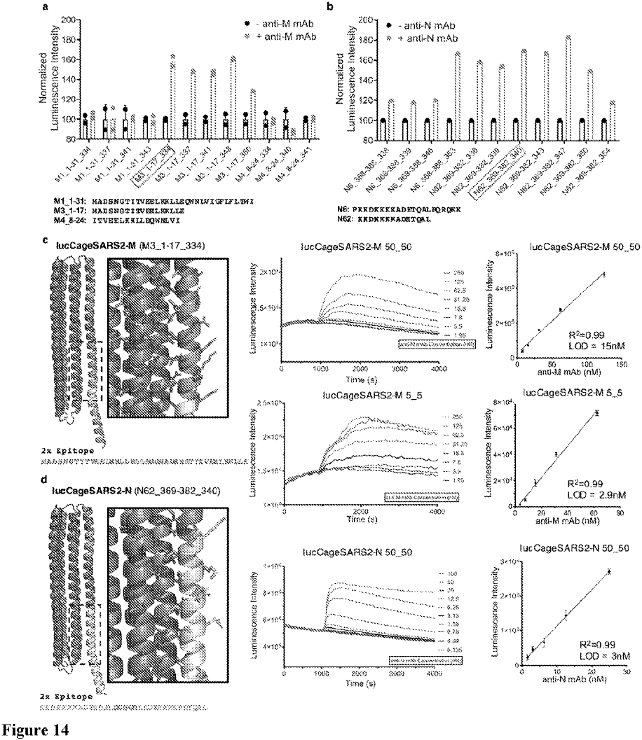

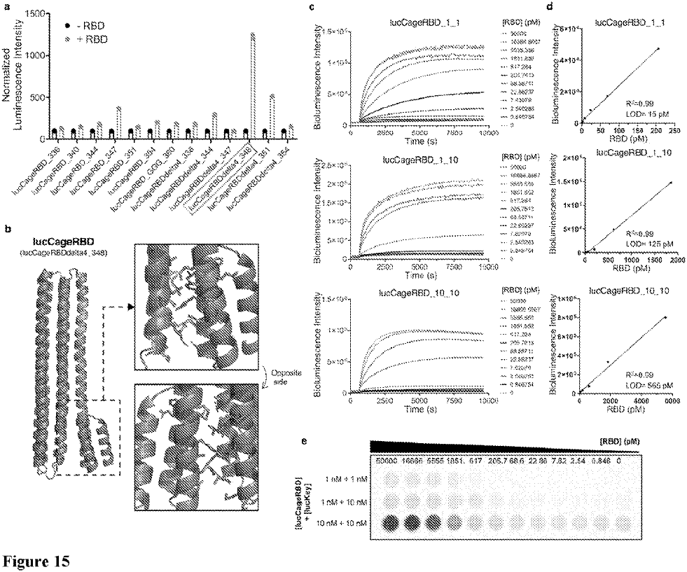

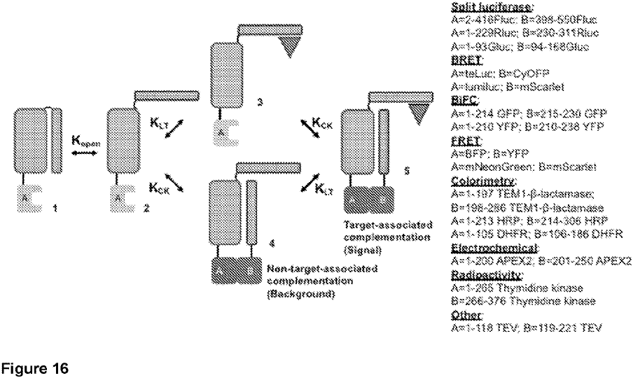

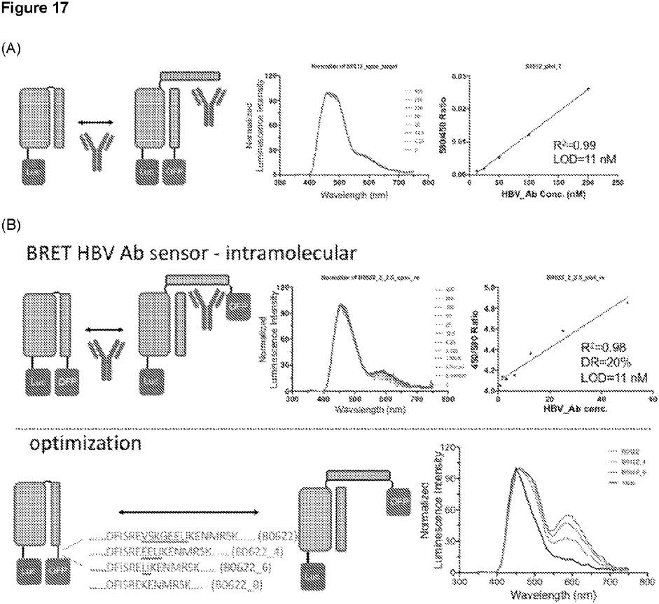

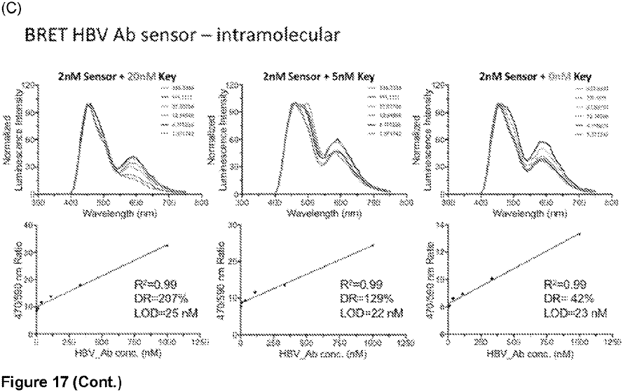

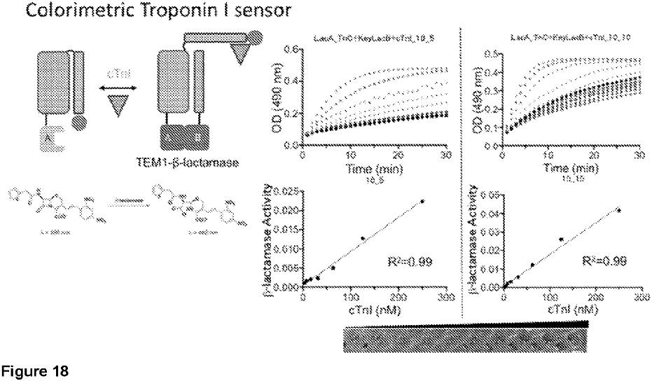

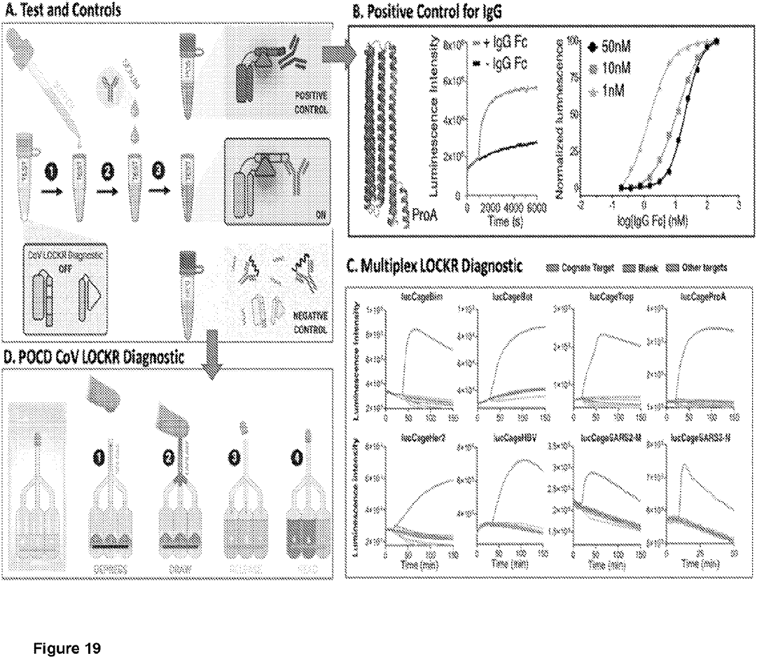

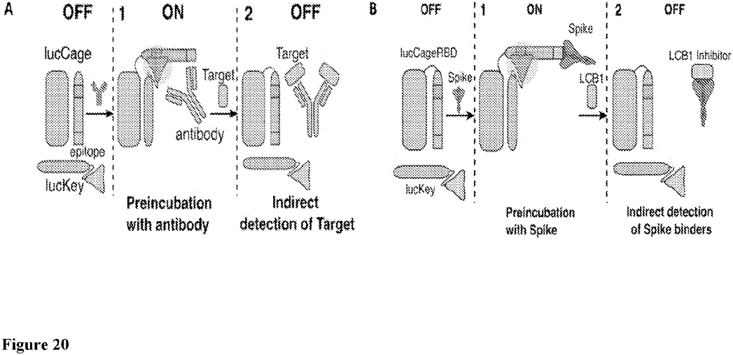

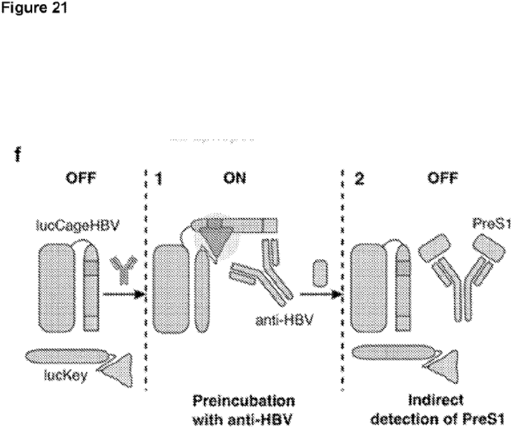

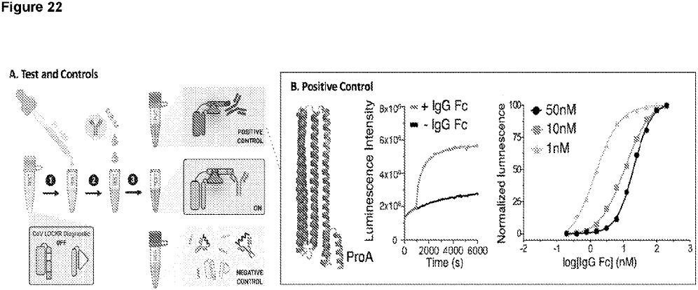

We next designed sensors for additional targets relevant in clinical settings. Since bioluminescent sensors do not require light for excitation, highly sensitive and low background readout is more suited than fluorescence to directly measure analytes in biological media such as blood and serum for point-of-care applications . We first targeted cardiac troponin I (cTnI), which is the standard early diagnostic biomarker for acute myocardial infarction (AMI). We took advantage of the high-affinity interaction between cTnT, cTnC, and cTnI (FIG. 3a) and designed eleven biosensor candidates by inserting 6 truncated cTnT sequences at different latch positions (FIG. 11a). The best candidate, lucCageTrop627, was able to detect cTnI but not at sufficiently low levels for clinical use (FIG. 11d). Because the rule-in and rule-out levels of cTnI assay for diagnosis of AMI in patients are in the low pM range, and because as noted above the limit of detection (LOD) of our sensor platform is about 0.1 x Kd of the latch-target affinity (K_{LT}), we further increased the affinity of our sensor to cTnI by fusing cTnC to its terminus (FIG. 3a, FIGS. 11b,c). The resulting sensor, lucCageTrop, has a single-digit pM LOD suitable for quantification of clinical samples (FIG. 3b, FIGS. 11e,f ).

Detection of specific antibodies is important for monitoring the spread of a pathogen in a population (antibodies remain long after the pathogen has been eliminated), the success of vaccination, and levels of therapeutic antibodies. To adapt our system to be used in such antibody serological analyses, we sought to incorporate linear epitopes recognized by the antibodies of interest into lucCage, so that binding of an antibody would open the switch allowing lucKey binding and reconstitution of luciferase activity. We first developed a sensor for anti-Hepatitis B virus (HBV) antibodies based on the crystal structu antibody (HzKR127) bound to a peptide from the PreS1 domain of the viral surface protein L ²⁵. The best of 8 designs tested, lucCageHBV (HBV344), had a ~150% increase in luciferase activity upon addition of HzKR127-3.2, an improved version of HzKR127 ²⁶ (FIGS. 12a,b). To further improve the dynamic range and LOD of lucCageHBV (~2 nM, FIGS. 12c-e), we increased the latch-target affinity (K_{LT}) by introducing an additional copy of the peptide at the end of the latch to take advantage of the antibody bivalent interaction with its epitope (FIGS. 3c,d). The resulting design, named lucCageHBVα, had a LOD of 260 pM and a dynamic range of 225% (FIG. 3e; FIGS. 13a-c), with a luminescence intensity easily detectable with a camera (FIG. 13d). Hence the platform to detect specific antibodies with a LOD in the range for monitoring therapeutic antibodies. We next demonstrated the use of the lucCageHBV sensor to detect hepatitis B surface antigen (HBsAg). Since our sensors are under thermodynamic control, we hypothesized that the pre-assembly of sensor-antibody complex would re-equilibrate in the presence of the target HBsAg protein, PreS1, with antibody redistributing to bind free PreS1 instead of the epitope on lucCageHBV (FIG. 3f). Indeed, the luminescence of lucCageHBV plus HzKR127-3.2 mixture decreased shortly upon addition of the PreS1 domain (FIG. 3g); the sensitivity of this readout enabled quantification of PreS 1 concentration in a clinically relevant range²⁸ (FIG. 3h, FIG. 12f). HBsAg seroclearance is one of the major biomarkers to monitor therapeutic progress following hepatitis diagnosis and vaccination efficacy, but current commercial HBsAg assays are unable to differentiate between the three HBsAg protein subtypes. Our PreS1 sensor (detecting HBsAg L antigen) shows that the system can achieve subtype-specific recognition.

The COVID-19 pandemic has showcased the urgent need for developing new diagnostic tools for tracking active infections by detecting the SARS-CoV-2 virus itself, and for detection of antiviral antibodies to evaluate the extent of the spread of the virus in the population and to identify individuals at lower risk of future infection. To design sensors for anti-SARS-CoV-2 antibodies, we first identified from the literature highly immunogenic linear epitopes in the SARS-CoV ^{31,32} and SARS-CoV-2 proteomes ^{33,34} that are not present in “common” strains of coronaviridae (i.e., HCoV-OC43, HCoV-HKU1, HCoV-229E, HCoV-NL63; we did not exclude reactivity against SARS-CoV or MERS as they are much less broadly distributed). Among these, we focused on two epitopes in the Membrane and Nucleocapsid proteins found to be recognized by SARS and COVID-19 patient sera for which cross-reactive animal-derived antibodies are commercially available (see FIG. 4 legend and Materials and methods for epitope and antibody description). We designed sensors for each epitope (FIGS. 14a,b) and identified designs that specifically respor pure anti-M and anti-N protein antibodies (FIGS. 4b,c). These sensors were fast (2-5 minutes to reach full signal) and had a ~50-70% dynamic range in response to low nanomolar amounts of antibodies (FIGS. 4b,c, FIGS. 14c,d).

To create sensors capable of detecting SARS-CoV-2 viral particles directly, we integrated into the LucCage format a designed picomolar affinity binder to the receptor-binding domain (RBD) of the SARS-CoV-2 Spike protein named LCB1 (FIG. 4d). Of 13 candidates tested, the best, which we refer to as lucCageRBD, had minimal background, an outstanding dynamic range (1700%) easily detectable with a camera and low LOD (15 pM) (FIG. 4d, FIG. 15). The superior dynamic range and sensitivity of this sensor are consequences of the high affinity of LCB1 to RBD (K_{LT}), consistent with our thermodynamic model, highlighting the synergy of the LucCage sensor platform and de novo binder design.

Because of the modularity and engineerability of the LucCage system, it took only three weeks to design the SARS-CoV-2 antibody and RBD sensors, obtain synthetic genes, express and purify the proteins, and evaluate sensor performance.

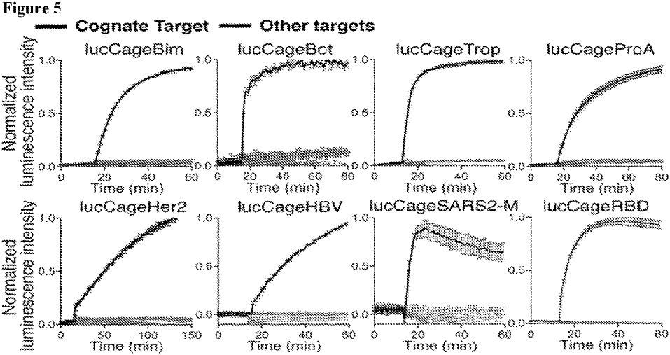

To test the specificity of the biosensors developed in this work (excluding the indirect detection of PreS1 by lucCageHBV and lucCageRBD), we measured the activation kinetics of each in response to all the targets (Bcl-2, botulinum neurotoxin B, IgG Fc, Her2, cardiac Troponin I, the monoclonal anti-HBV antibody (HzKR127-3.2), the anti-SARS-CoV-1-M polyclonal antibody (clone 3527), the anti-SARS-CoV-1-N monoclonal antibody (clone 18F629.1), and PreS1). As shown in FIG. 5, each sensor responded rapidly and sensitively to its cognate target, but not to any of the others. A summary of each lucCage sensor characteristics and sensing domains used can be found in Table12 and Table 13, respectively.

Most previous protein-based biosensor platforms depend on the specific geometry of a target-sensor interaction to trigger a conformational change in the reporter component and hence are specialized for a subset of detection challenges. Because of this target dependence, considerable optimization can be required to achieve high sensitivity detection of a new target. Our sensor platform is based on the thermodynamic coupling between defined closed and open states of the system, thus, its sensitivity depends on the free energy change upon the sensing domain binding to the target but not the specific geometry of the binding interaction. This enables the incorporation of various binding modalities, including small peptides, globular mini proteins, antibody epitopes and de novo designed binders, to generate sensitive sensors for a wide range of protein targets with little or no optimization. For point of care (POC) applications, our system has the advantages of being homogeneous, no-wash, all-in-solution, a nearly instantaneous readout, and its quantification of lumir performed by means of inexpensive and accessible devices such as a cell pnone camera. In hospital settings, the ability to predictably make a wide range of sensors under the same principle could enable quick readout of large numbers of different compounds using an array of hundreds of different sensors on, for example, a 384-well plate.

Up until recently, the focus of de novo protein design was on the design of proteins with new structures corresponding to single deep free energy minima; our results highlight the progress in the field which now enables more complex multistate systems to be readily generated. Our sensors are expressed at high levels in cells and are very stable, which considerably facilitates the further manufacturing process. The general “molecular device” architecture of our platform synergizes particularly well with complementary advances in the de novo design of high-affinity miniprotein binders, which can be designed with three dimensional structures readily compatible with the lucCage platform. LucCageRBD highlights the potential of this fully de novo approach, with a 1700% dynamic range and 15 pM LOD from a sensor coming straight out of the computer, without any experimental optimization.

References
1. Stein, V. & Alexandrov, K. Synthetic protein switches: design principles and applications. Trends Biotechnol. 33, 101-110 (2015).
2. Langan, R. A. et al. De novo design of bioactive protein switches. Nature 572, 205-210 (2019).
3. Adams, E. R. et al. Antibody testing for COVID-19: A report from the National COVID Scientific Advisory Panel. medRxiv 2020.04.15.20066407 (2020).
4. Yeh, H.-W. & Ai, H.-W. Development and Applications of Bioluminescent and Chemiluminescent Reporters and Biosensors. Annu. Rev. Anal. Chem. 12, 129-150 (2019).
5. Greenwald, E. C., Mehta, S. & Zhang, J. Genetically Encoded Fluorescent Biosensors Illuminate the Spatiotemporal Regulation of Signaling Networks. Chem. Rev. 118, 11707-11794 (2018).
6. Schena, A., Griss, R. & Johnsson, K. Modulating protein activity using tethered ligands with mutually exclusive binding sites. Nat. Commun. 6, 7830 (2015).
7. Arts, R. et al. Semisynthetic Bioluminescent Sensor Proteins for Direct Detection of Antibodies and Small Molecules in Solution. ACS Sens 2, 1730-1736 (2017).
8. Xue, L., Prifti, E. & Johnsson, K. A General Strategy for the Semisynthesis of
Ratiometric Fluorescent Sensor Proteins with Increased Dynamic Soc. 138, 5258-5261 (2016).
9. Guo, Z. et al. Generalizable Protein Biosensors Based on Synthetic Switch Modules. J. Am. Chem. Soc. 141, 8128-8135 (2019).
10. Edwardraja, S. et al. Caged activators of artificial allosteric protein biosensors. ACS Synth. Biol. (2020) doi:10.1021/acssynbio.9b00500.
11. Ribeiro, L. F., Warren, T. D. & Ostermeier, M. Construction of Protein Switches by Domain Insertion and Directed Evolution. Methods Mol. Biol. 1596, 43-55 (2017).
12. Dixon, A. S. et al. NanoLuc Complementation Reporter Optimized for Accurate Measurement of Protein Interactions in Cells. ACS Chem. Biol. 11, 400-408 (2016).
13. Minor, D. L., Jr & Kim, P. S. Context-dependent secondary structure formation of a designed protein sequence. Nature 380, 730-734 (1996).
14. Huang, P.-S. et al. RosettaRemodel: a generalized framework for flexible backbone protein design. PLoS One 6, e24109 (2011).
15. Chevalier, A. et al. Massively parallel de novo protein design for targeted therapeutics. Nature 550, 74-79 (2017).
16. Deis, L. N. et al. Suppression of conformational heterogeneity at a protein-protein interface. Proc. Natl. Acad. Sci. U. S. A. 112, 9028-9033 (2015).
17. Eigenbrot, C., Ultsch, M., Dubnovitsky, A., Abrahmsen, L. & Härd, T. Structural basis for high-affinity HER2 receptor binding by an engineered protein. Proc. Natl. Acad. Sci. U. S. A. 107, 15039-15044 (2010).
18. Hobbs, R. J., Thomas, C. A., Halliwell, J. & Gwenin, C. D. Rapid Detection of Botulinum Neurotoxins-A Review. Toxins 11, (2019).
19. Perrier, A., Gligorov, J., Lefevre, G. & Boissan, M. The extracellular domain of Her2 in serum as a biomarker of breast cancer. Lab. Invest. 98, 696-707 (2018).
20. Yu, Q. et al. Semisynthetic sensor proteins enable metabolic assays at the point of care. Science 361, 1122-1126 (2018).
21. Rubini Gimenez, M. et al. One-hour rule-in and rule-out of acute myocardial infarction using high-sensitivity cardiac troponin I. Am. J. Med. 128, 861-870.e4 (2015).
22. Collins, M. H. Serologic Tools and Strategies to Support Intervention Trials to Combat Zika Virus Infection and Disease. Trop Med Infect Dis 4, (2019).
23. Pondé, R. A. de A. Expression and detection of anti-HBs antibodies after hepatitis B virus infection or vaccination in the context of protective immunity. Arch. Virol. 164, 2645-2658 (2019).
24. van Rosmalen, M. et al. Dual-Color Bioluminescent Sensor Prote Drug Monitoring of Antitumor Antibodies. Anal. Chem. 90, 3592-3599 (2018).
25. Chi, S.-W. et al. Broadly neutralizing anti-hepatitis B virus antibody reveals a complementarity determining region H3 lid-opening mechanism. Proc. Natl. Acad. Sci. U.S.A. 104, 9230-9235 (2007).
26. Kim, J. H. et al. Enhanced humanization and affinity maturation of neutralizing anti-hepatitis B virus preS1 antibody based on antigen-antibody complex structure. FEBS Lett. 589, 193-200 (2015).
27. Ovacik, M. & Lin, K. Tutorial on Monoclonal Antibody Pharmacokinetics and Its Considerations in Early Development. Clin. Transl. Sci. 11, 540-552 (2018).
28. Locarnini, S. & Bowden, S. Hepatitis B surface antigen quantification: Not what it seems on the surface. Hepatology vol. 56 411-414 (2012).
29. Cornberg, M. et al. The role of quantitative hepatitis B surface antigen revisited. Journal of Hepatology vol. 66 398-411 (2017).
30. Perera, R. A. et al. Serological assays for severe acute respiratory syndrome coronavirus2 (SARS-CoV-2), March 2020. Euro Surveill. 25, (2020).
31. Chow, S. C. S. et al. Specific epitopes of the structural and hypothetical proteins elicit variable humoral responses in SARS patients. J. Clin. Pathol. 59, 468-476 (2006).
32. He, Y., Zhou, Y., Siddiqui, P., Niu, J. & Jiang, S. Identification of immunodominant epitopes on the membrane protein of the severe acute respiratory syndrome-associated coronavirus. J. Clin. Microbiol. 43, 3718-3726 (2005).
33. Wang, H. et al. SARS-CoV-2 proteome microarray for mapping COVID-19 antibody interactions at amino acid resolution. (2020) doi:10.1101/2020.03.26.994756.
34. Dahlke, C. et al. Distinct early IgA profile may determine severity of COVID-19 symptoms: an immunological case series. medRxiv 2020.04.14.20059733 (2020).
35. Yu, Q. et al. A biosensor for measuring NAD levels at the point of care. Nature Metabolism vol. 1 1219-1225 (2019).
36. Arts, R. et al. Detection of Antibodies in Blood Plasma Using Bioluminescent Sensor Proteins and a Smartphone. Anal. Chem. 88, 4525-4532 (2016).
37. Tenda, K. et al. Paper-Based Antibody Detection Devices Using Bioluminescent BRET-Switching Sensor Proteins. Angewandte Chemie vol. 130 15595-15599 (2018).
38. Adamson, H. et al. Affimer-Enzyme-Inhibitor Switch Sensor for Rapid Wash-free Assays of Multimeric Proteins. ACS Sens. 4, 3014-3022 (2019).
39. Schena, A., Griss, R. & Johnsson, K. Corrigendum: Modulating protein activity using
tethered ligands with mutually exclusive binding sites. Nat. Comn
40. Berger, S. et al. Computationally designed high specificity inhibitors delineate the roles of BCL2 family proteins in cancer. Elife 5, (2016).
41. Jin, R., Rummel, A., Binz, T. & Brunger, A. T. Botulinum neurotoxin B recognizes its protein receptor with high affinity and specificity. Nature 444, 1092-1095 (2006).
42. Shen, A. et al. Mechanistic and structural insights into the proteolytic activation of Vibrio cholerae MARTX toxin. Nat. Chem. Biol. 5, 469-478 (2009).
43. Otwinowski, Z. & Minor, W. [20] Processing of X-ray diffraction data collected in oscillation mode. Methods Enzymol. 276, 307-326 (1997).
44. Liebschner, D. et al. Macromolecular structure determination using X-rays, neutrons and electrons: recent developments in Phenix. Acta Crystallogr D Struct Biol 75, 861-877 (2019).
45. Potterton, L. et al. Developments in the CCP4 molecular-graphics project. Acta Crystallogr. D Biol. Crystallogr. 60, 2288-2294 (2004).

### METHODS

### Design of the Sensor System: lucCage and lucKey

SmBit (VTGYRLFEEIL; SEQ ID NO: 27359) was grafted into the latch of the asymmetric LOCKR switch described in Langan et al, 2019 using GraftSwitchMover, a RosettaScripts™-based protein design algorithm (See Supplementary Methods for details). The grafting sampling range was assigned between residues 300-330. The resulting designs were energy-minimized, visually inspected and selected for subsequent gene synthesis, protein production and biochemical analyses. The best SmBit position on the latch was experimentally determined to be an insertion at residue 312, as described in FIG. 6. lucKey was assembled by genetically fusing the LgBit of NanoLuc ¹² to the key peptide described in Langan et al, 2019. (See Table 10 for the full sequence list)

### Computational Grafting of Sensing Domains Into lucCage

Peptides and epitopes: The amino acid sequence for each sensing domain was grafted using Rosetta™ GraftSwitchMover into all α-helical registers between residues 325-360 of lucCage (See Supplementary Methods for details). The resulting lucCages were energy-minimized, visually inspected and typically less than ten designs were selected for subsequent protein production and biochemical characterization.

Protein domains: First, the main secondary structure elements surface of the binding protein were identified, their amino acid sequence was extracted ana grafted into lucCage using theGraftSwitchMover as described above. Then, we used Rosetta™ Remodel ¹⁴ to model the full-length binding domain in the context of the switch in which this interface was buried against the cage (See Supplementary Methods for details). The designs were energy-minimized and visually inspected for selection. Typically, less than ten designs were selected for biochemical characterization.

### Synthetic Gene Construction

The designed protein sequences were codon optimized for E. coli expression (IDT codon optimization tool) and ordered as synthetic genes in pET21b+ or pET29b+ E. coli expression vectors (IDT). The synthetic gene was inserted at the Ndel and XhoI sites of each vector, including an N-terminal hexahistidine tag followed by a TEV protease cleavage site and a stop codon was added at the C terminus.

### General Procedures for Bacterial Protein Production and Purification

The E. coli LEMO21(DE3) strain (NEB) was transformed with a pET21b+ or pET29b+ plasmid encoding the synthesized gene of interest. Cells were grown for 24 hours in LB media supplemented with carbenicillin or kanamycin. Cells were inoculated at a 1:50 mL ratio in the Studier TBM-5052 autoinduction media supplemented with carbenicillin or kanamycin, grown at 37° C. for 2-4 hours, and then grown at 18° C. for an additional 18 h. Cells were harvested by centrifugation at 4000 g at 4° C. for 15 min and resuspended in 30 ml lysis buffer (20 mM Tris-HCl pH 8.0, 300 mM NaCl, 30 mM imidazole, 1 mM PMSF, 0.02 mg/mL DNAse). Cell resuspensions were lysed by sonication for 2.5 minutes (5 second cycles). Lysates were clarified by centrifugation at 24,000 gat 4° C. for 20 min and passed through 2 ml of Ni-NTA nickel resin (Qiagen, 30250) pre-equilibrated with wash buffer, (20 mM Tris-HCl pH 8.0, 300 mM NaCl, 30 mM imidazole). The resin was washed twice with 10 column volumes (CV) of wash buffer, and then eluted with 3 CV of elution buffer (20 mM Tris-HCl pH 8.0, 300 mM NaCl, 300 mM imidazole). The eluted proteins were concentrated using Ultra-15 Centrifugal Filter Units (Amicon) and further purified by using a Superdex™ 75 Increase 10/300 GL (GE Healthcare) size exclusion column in Tris Buffered Saline (TBS; 25 mM Tris-HCl pH 8.0, 150 mM NaCl). Fractions containing monomeric protein were pooled, concentrated, and snap-frozen in liquid nitrogen and stored at -80° C.

### In Vitro Bioluminescence Characterization

A Synergy™ Neo2 Microplate Reader (BioTek) was used for an in vitro bioluminescence measurements. Assays were performed in 1:1=HBS-EP:Nano-Glo assay buffer for anti-HBV and RBD sensors while 1:1=DPBS:Nano-Glo assay buffer was used for other sensors. 10X lucCage, 10X lucKey, and 10X target proteins of desired concentrations were first prepared from stock solutions. For each well of a white opaque 96-well plate, 10 µL of 10X lucCage, 10 µL of 10X lucKey, and 20 µL of buffer were mixed to reach the indicated concentration and ratio. The plate was centrifuged at 1000 × g for 1 min and incubated at RT for additional 10 min. Then, 50 µL of 50X diluted furimazine (Nano-Glo™ luciferase assay reagent, Promega) was added to each well. Bioluminescence measurements in the absence of target were taken every 1 min post-injection (0.1 s integration and 10 s shaking during intervals). After ~15 min, 10 µL of serially diluted 10X target protein plus a blank was injected and bioluminescence kinetic acquisition continued for a total of 2 h. To derive EC₅₀ values from the bioluminescence-to-analyte plot, the top three peak bioluminescence intensities at individual analyte concentrations were averaged, subtracted from blank, and used to fit the sigmoidal 4PL curve. To calculate the LOD, the linear region of bioluminescence responses of sensors to its analyte was extracted and a linear regression curve was obtained. It was used to derive the standard deviation of the response (SD) and the slope of the calibration curve (S). The LOD was determined as 3×(SD/S). The experimental measurements were taken in triplicate and the mean values are shown where applicable. The results were successfully replicated using different batches of pure proteins on different days.

### Biolayer Interferometry (BLI)

Protein-protein interactions were measured by using an Octet® RED96 System (ForteBio) using streptavidin-coated biosensors (ForteBio). Each well contained 200 µL of solution, and the assay buffer was HBS-EP+ Buffer (10 mM HEPES pH 7.4, 150 mM NaCl, 3 mM EDTA, 0.05% v/v Surfactant P20, 0.5% non-fat dry milk). The biosensor tips were loaded with analyte peptide/protein at 20 µg/mL for 300 s (threshold of 0.5 nm response), incubated in HBS-EP+ Buffer for 60 s to acquire the baseline measurement, dipped into the solution containing Cage and/or Key for 600 s (association step) and dipped into the HBS-EP+ Buffer for 600 s (dissociation steps). The binding data were analyzed with the ForteBio Data Analysis Software version 9.0.0.10.

### Design and Characterization of lucCageBim

The Bim peptide sequence (EIWIAQELRRIGDEFNAYYAAA was threaded into the lucCage scaffold as described in the “Design of sensing domains into lucCage” section. The selected designs were expressed in E. coli, purified and characterized for luminescence activation. The bioluminescence detection signal was measured for each design lucCage at 20 nM mixed with lucKey at 20 nM, in the presence or absence of target Bcl-2 protein at 200 nM. Bcl-2 was expressed as described somewhere else ⁴⁰.

### Design and Characterization of lucCageHer2, lucCageProA, lucCageBot and lucCageRBD

The main binding motifs of the Bot.0671.2 de novo binder, S. aureus Protein A domain C (SpaC), the Her2 affibody and the de novo RBD binder LCB1 were threaded into lucCage as described in the “Design of sensing domains into lucCage” section (See Table 13 for sequences of sensing domains). The selected designs were expressed in E. coli, purified and characterized for luminescence activation. The bioluminescence detection signal was measured for each design lucCage at 20 nM mixed with lucKey at 20 nM, in the presence or absence of 200 nM target protein. The target proteins used were: Botulinum Neurotoxin B HcB expressed as previously described ⁴¹, human IgG1 Fc-HisTag (AcroBiosystems, Cat. No. IG1-H5225) and human Her2-HisTag (AcroBiosystems, Cat. No. HE2-H5225).

### Design and Characterization of lucCageTrop

The cardiac Troponin T (cTnT) binding motif (EDQLREKAKELWQTIYNLEAEKFDLQEKFKQQKYEINVLRNRINDNQ; SEQ ID NO: 27390) was split into fragments of different length (see FIG. 11) and threaded into the lucCage scaffold as described in the “Design of sensing domains into lucCage” section. The selected designs were expressed in E. coli, purified and characterized for luminescence activation. The bioluminescence detection signal was measured for each design lucCage at 20 nM mixed with lucKey at 20 nM in the presence or absence of 100 nM cardiac Troponin I (Genscript, Cat. No. Z03320-50). Subsequently, lucCageTrop, an improved version by fusion to cardiac Troponin C (cTnC), was created by genetically fusing the following sequence to the C terminus of lucCageTrop627

(KVSKTKDDSKGKSEEELSDLFRMFDKNADGYIDLEELKIMLQATGETIT
EDDIEELMKDGDKNNDGRIDYDEFLEFMKGVE; SEQ ID NO: 27627
).

### Design and Characterization of lucCageHBV and lucCageHBVα

The binding motif (GANSNNPDWDFN (SEQ ID NO: 27629)) was threaded into the lucCage scaffold at every position after residues 336 using the Rosetta™ GraftSwitchMover. Following the Rosetta™ FastRelax protocol, eight designs were selected for protein production. Bioluminescence was measured with the designed lucCages (20 nM) and lucKey (20 nM) in the presence or absence of the anti-HVB antibody HzKR127-3.2 (100 nM) to select lucCageHBV. Subsequently, lucCageHBVα was constructed by genetically fusing a sequence containing a second antigenic motif (GGSGGGSSGFGANSNNPDWDFNPN; SEQ ID No:27628) to lucCageHBV.

### Design and Characterization of lucCageSARS2-M and lucCageSARS2-N

Antigenic epitopes of the SARS-CoV-2 membrane protein (a.a. 1-31, 1-17 and 8-24) and the nucleocapsid protein (a.a. 368-388 and 369-382) were computationally grafted into lucCage as described in the “Design of sensing domains into lucCage” section. The selected designs were expressed in E. coli, purified and characterized for luminescence activation. All designs at 50 nM were mixed with 50 nM lucKey and experimentally screened for an increase in luminescence in the presence of rabbit anti-SARS-CoV Membrane polyclonal antibodies (ProSci, Cat. No.: 3527) at 100 nM or mouse anti-SARS-CoV Nucleocapsid monoclonal antibody (clone 18F629.1, NovusBio Cat. No. NBP2-24745) at 100 nM.

### Design and Characterization of sCageHA Variants

HB 1.9549.2 was embedded into the parental six-helix bundle for sCage design at different positions along the latch helix of the scaffold. To promote more favorable intramolecular interactions, three consecutive residues on the latch were intentionally substituted with glycine to allow for conformational freedom. The five designs were produced in E. coli. Biolayer interferometry analysis was performed with purified Cages (1 µM) and biotinylated Influenza A H1 hemagglutinin (HA)¹⁵ loaded onto streptavidin-coated biosensor tips (ForteBio) in the presence or absence of the key (2 µM) using an Octet™ instrument (ForteBio).

### Production and Purification of HzKR127-3.2

The synthetic V_{H} and V_{L} DNA fragments were subcloned into the pdCMV-dhfrC-cA10A3 plasmid containing the human Cγ1 and Cκ DNA sequences. The vector was introduced into HEK 293T cells using Lipofectamine™ (Invitrogen), and the cells were grown in FreeStyle™ 293 (GIBCO) in 5% CO₂ in a 37° C. humidified incubator. The culture supernatant was loaded onto a protein A-Sepharose™ column (Millipc antibody was eluted by the addition of 0.2 M glycine-HCl (pH 2.7), followed by immediate neutralization with 1 M Tris-HCl (pH 8.0). The solution was dialyzed against 10 mM HEPES-NaOH (pH 7.4), and the purity of the protein was analyzed by SDS-PAGE.

### Production and Purification of the PreS1 Domain

The DNA fragment encoding the PreS1 domain (residues 1-56) was cloned into the pGEX-2T (GE Healthcare) plasmid, and the protein was produced in the E. coli BL21(DE3) strain (NEB) at 18° C. as a fusion protein with glutathion-S-transferase (GST) at the N-terminus. The cell lysates were prepared in a buffer solution (25 mM Tris-HCl pH 8.0, 300 mM NaCl), and clarified supernatant was loaded onto GSTBind™ Resin (Novagen). The GST-PreS1 domain was eluted with the same buffer containing additional 10 mM reduced glutathione, further purified using a Superdex™75 Increase 10/300 GL (GE Healthcare) size exclusion column, and concentrated to 34 µM.

### Production of SCageHA 267-1S and Its Variants

sCageHA_267-1S and sCageHA_267-1S(E99Y/T144Y) were expressed at 18° C. in the E. coli LEMO21(DE3) strain (NEB) as a fusion protein containing a (His)₁₀-tagged cysteine protease domain (CPD) derived from Vibrio cholerae ⁴² at the C-terminus. The protein was purified using HisPur™ nickel resin (Thermo), a HiTrap™ Q anion exchange column (GE Healthcare) and a HiLoad 26/60 Superdex™75 gel filtration column (GE Healthcare). For Selenomethionine (SelMet)-labeling, an I30M mutation was introduced additionally to generate a sCageHA_267-1S(E99Y/T144Y/I30M) variant. This protein was expressed in the E. coli B834 (DE3) RIL strain (Novagen) in the minimal media containing SeMet, and purified according to the same procedure for purifying the other variants.

### Crystallization and Structure Determination of sCageHA 267-1S

Two point mutations (Glu99Tyr and Thr144Tyr) were introduced in an attempt to induce favorable crystal packing interactions. Good-quality single crystals of sCageHA_267-1S(E99Y/T144Y/I30M) were obtained in a hanging-drop vapor-diffusion setting by micro-seeding in a solution containing 11% (v/v) ethanol, 0.25 M NaCl, 0.1 M TrisHCl (pH 8.5). The crystals required strict maintenance of the temperature at 25° C. For cryoprotection, the crystals were soaked briefly in the crystallization solution supplemented with 15% 2,3-butanediol and flash-cooled in the liquid nitrogen. A single-wavelength anomalous dispersion (SAD) data set was collected at the Se absorption peak and processed positions and initial electron density map were calculated using the Autosol™ module in PHENIX⁴⁴. The model building and structure refinement were performed by using COOT⁴⁵ and PHENIX.

### SUPPLEMENTARY INFORMATION

### Supplementary Discussion

Our generalized protein sensory system based on a de novo switch relies on the thermodynamic coupling (see FIGS. 1a-c) between a defined close state (Kₒₚₑₙ) and a defined open state (K_{LT} and K_{CK}). With our system), the target specificity to arbitrary targets can be achieved not only by incorporating known binding domains but also de novo binders where we have full control over protein fold and geometry. Because there is no flexible or semi-flexible linker in our system and we are capable of designing different types of interaction to cage binding domains, the conformational change is thus decoupled from the binder-target interaction, which makes this system more structurally predictable at open state. A newly developed GraftSwitchMover in Rosetta™ allows sensor design in one step, bypassing the need with the other formats to empirically re-engineer sensor configuration. The intermolecular association of the LucKey with the open form of the sensor generates the luminescent signal, providing an additional tunable parameter K_{CK} that can be optimized along with Kₒₚₑₙ to maximize sensor dynamic range, analytical range, specificity, and sensitivity.

### Supplementary Methods

### 1. Thermodynamic Model

The equilibrium constants were defined as Kₒₚₑₙ for latch opening (Equation 1), K_{CK} for the dissociation constant of the lucCage and lucKey (Equation 2 and 3), and K_{LT} for the dissociation constant of the latch and target (Equation 4 and 5). K_{R} describes the equilibrium of the reconstituted luciferase, which is determined by the reported dissociation constant of the NanoBit system (190 µM ¹⁹) and the effective local concentration (C_{eff}) of split counterparts (Equation 6 and 7). We set C_{eff} to 1 mM here as the literature suggested high micromolar to low millimolar range for intramolecular interaction partners ²⁰, and our modular switch should span much shorter distance than flexible linkers. The total amount of each component is constant, so Equations 8, 9, and 10 were introduced. Given four equilibrium constants (Kₒₚₑₙ, K_{CK}, K_{LT}, and K_{R}) and three total concentrations ([lucCage]total, [lucKey]ₜₒₜₐₗ, and [target]ₜₒₜₐₗ), python module sympy.nsolve was used to equations numerically and find the concentration of each species at equilibrium. The total concentration of luminescent species 6 and 7 was extracted from the solution, divided by [lucCage]ₜₒₜₐₗ, and plotted for corresponding figures with various Kₒₚₑₙ for FIG. 1d, K_{LT} for FIG. 1e, and [lucCage]ₜₒₜₐₗ, [lucKey]ₜₒₜₐₗ for FIG. 1f. Numbers for FIG. 1f are normalized between 0-1.

$${\text{K}}_{\text{open}} = \frac{\left[2\right]}{\left[1\right]} \qquad \text{Equation 1:}$$

$${\text{K}}_{\text{CK}} = \frac{\left[2\right] \times {\left[\text{luckey}\right]}_{f r e e}}{\left[5\right]} \qquad \text{Equation 2:}$$

$${\text{K}}_{\text{CK}} = \frac{\left[3\right] \times {\left[\text{luckey}\right]}_{f r e e}}{\left[4\right]} \qquad \text{Equation 3:}$$

$${\text{K}}_{\text{LT}} = \frac{\left[6\right] \times {\left[\text{target}\right]}_{f r e e}}{\left[7\right]} \qquad \text{Equation 4:}$$

$${\text{K}}_{\text{LT}} = \frac{\left[2\right] \times {\left[\text{target}\right]}_{f r e e}}{\left[3\right]} \qquad \text{Equation 5:}$$

$${\text{K}}_{\text{R}} = \frac{190 μ \text{} \text{M}}{{\text{C}}_{\text{eff}}} = \frac{\left[5\right]}{\left[6\right]} \qquad \text{Equation 6:}$$

$${\text{K}}_{\text{R}} = \frac{\left[4\right]}{\left[7\right]} \qquad \text{Equation 7:}$$

$${\left[\text{lucCage}\right]}_{\text{total}} = \left[1\right] + \left[2\right] + \left[3\right] + \left[4\right] + \left[5\right] + \left[6\right] + \left[7\right] \qquad \text{Equation 8:}$$

$${\left[\text{lucKey}\right]}_{\text{total}} = {\left[\text{lucKey}\right]}_{\text{free}} + \left[4\right] + \left[5\right] + \left[6\right] + \left[7\right] \qquad \text{Equation 9:}$$

$${\left[\text{target}\right]}_{\text{total}} = {\left[\text{target}\right]}_{\text{free}} + \left[3\right] + \left[4\right] + \left[7\right] \qquad \text{Equation 10:}$$

### 2. Computational Grafting of Sensing Domains Into lucCage

The structural models of the lucCage sensors were created by grafting each sensing domain onto the latch of the lucCage scaffold (See Table 13). The design was performed using a RosettaScripts™ protocol, (GraftSwitch relax.xml, See code availability) to thread a list of sensing domains with annotated interface residues (sensing_domains.fasta, See Code Availability) into the model of lucCage (lucCage.pdb, See Code Availability). A bash script (run_GraftSwitch.sh, See Code Availability) was used to call RosettaScripts™. This protocol uses two successive Rosetta™ movers: (i) GraftSwitchMover to thread the desired sensing domain sequence into a defined region of the lucCage latch (amino acids 325-359) and to select designs with the defined “important resides” buried in the cage/latch interface; (ii) and MultiplePoseMover to relax (FastRelax to find the lowest energy structure given the mutations from the previous mover.), filter and score each output model resulting from the previous mover. The resulting designs were further evaluated by eye ir done by selecting designs showing favorable hydrophobic packing interactions between the newly threaded sequence and the cage and discarding designs with unfavorable buried hydrophilic residues that could destabilize the closed state of the sensor (unless these residues were annotated as “important residues”).

For grafting mini-protein binders with a pre-defined tertiary structure (i.e., Bot.671.2, SpaC, and the Her2 affibody) we first identified the primary interaction surface of the binding protein to its target and identified the main secondary structure elements involved in it. We added the amino acid sequence of these elements in the sensing domains.fasta file to use them in the protocol described above. The outputs were lucCage design models with the grafted interface element. Then, we used Rosetta™ Remodel domain insertion²¹ to model the full-length sensing domain in the context of the switch (remodel domain insertion.sh, See Code Availability), followed by Relax to find the lowest energy structure (relax.sh, See Code Availability). Finally, the best designs were selected by eye in PyMol 2.0.

### Supplementary Information References

1. Bahadir, E. B. & Sezgintürk, M. K. Lateral flow assays: Principles, designs and labels. Trends Analyt. Chem. 82, 286-306 (2016).
2. Yeh, H.-W. & Ai, H.-W. Development and Applications of Bioluminescent and Chemiluminescent Reporters and Biosensors. Annu. Rev. Anal. Chem. 12, 129-150 (2019).
3. Greenwald, E. C., Mehta, S. & Zhang, J. Genetically Encoded Fluorescent Biosensors Illuminate the Spatiotemporal Regulation of Signaling Networks. Chem. Rev. 118, 11707-11794 (2018).
4. Glasgow, A. A. et al. Computational design of a modular protein sense-response system. Science 366, 1024-1028 (2019).
5. Guo, Z. et al. Generalizable Protein Biosensors Based on Synthetic Switch Modules. J. Am. Chem. Soc. 141, 8128-8135 (2019).
6. Yu, Q. et al. Semisynthetic sensor proteins enable metabolic assays at the point of care. Science 361, 1122-1126 (2018).
7. van Rosmalen, M. et al. Dual-Color Bioluminescent Sensor Proteins for Therapeutic Drug Monitoring of Antitumor Antibodies. Anal. Chem. 90, 3592-3599 (2018).
8. Adamson, H. et al. Affimer-Enzyme-Inhibitor Switch Sensor for Rapid Wash-free Assays of Multimeric Proteins. ACS Sens. 4, 3014-3022 (2019).
9. Tenda, K. et al. Paper-Based Antibody Detection Devices Using Bioluminescent BRET-Switching Sensor Proteins. Angewandte Chemie vol. 130 1
10. Griss, R. et al. Bioluminescent sensor proteins for point-ot-care therapeutic drug monitoring. Nat. Chem. Biol. 10, 598-603 (2014).
11. Arts, R. et al. Detection of Antibodies in Blood Plasma Using Bioluminescent Sensor Proteins and a Smartphone. Anal. Chem. 88, 4525-4532 (2016).
12. Lopez-Ruiz, N. et al. Smartphone-based simultaneous pH and nitrite colorimetric determination for paper microfluidic devices. Anal. Chem. 86, 9554-9562 (2014).
13. Leippe, D. M. et al. A bioluminescent assay for the sensitive detection of proteases. Biotechniques 51, 105-110 (2011).
14. Troy, T., Jekic-McMullen, D., Sambucetti, L. & Rice, B. Quantitative comparison of the sensitivity of detection of fluorescent and bioluminescent reporters in animal models. Mol. Imaging 3, 9-23 (2004).
15. Yeh, H.-W., Wu, T., Chen, M. & Ai, H.-W. Identification of Factors Complicating Bioluminescence Imaging. Biochemistry 58, 1689-1697 (2019).
16. Yeh, H.-W. et al. ATP-Independent Bioluminescent Reporter Variants To Improve in Vivo Imaging. ACS Chem. Biol. 14, 959-965 (2019).
17. Edwardraja, S. et al. Caged activators of artificial allosteric protein biosensors. ACS Synth. Biol. (2020) doi:10.1021/acssynbio.9b00500.
18. Langan, R. A. et al. De novo design of bioactive protein switches. Nature 572, 205-210 (2019).
19. Dixon, A. S. et al. NanoLuc Complementation Reporter Optimized for Accurate Measurement of Protein Interactions in Cells. ACS Chem. Biol. 11, 400-408 (2016).
20. Krishnamurthy, V. M., Semetey, V., Bracher, P. J., Shen, N. & Whitesides, G. M. Dependence of Effective Molarity on Linker Length for an Intramolecular Protein-Ligand System. Journal of the American Chemical Society vol. 129 1312-1320 (2007).
21. Huang, P.-S. et al. RosettaRemodel: a generalized framework for flexible backbone protein design. PLoS One 6, e24109 (201

TABLE 11

X-ray data collection and structure refinement statis

| Data Collection | SelMET-sCageHA_267-1S(E99Y/T144Y/I30M) |
|---|---|
| Space group | C2 |
| Unit cell dimensions | |
| a, b, c (Å) | 178.993 60.127 71.799 |
| a, β, γ (°) | 90, 112.463, 90 |
| Wavelength (Å) | 0.9794 |
| Resolution (Å) | 50-2.03 (2.03-2.00)^{a} |
| R_{sym} | 6.6 (16.5)^{a} |
| I/σ(I) | 24.0 (3.5)^{a} |
| Completeness (%), >1σ | 70.6 (33.8)^{a} |
| Redundancy | 2.5 (1.6)^{4a} |
| Refinement | |
| Resolution (Å) | 46.09-1.99 (2.06-1.99)^{a} |
| No. of reflections | 37603 |
| R_{work} / R_{free} | 0.2078/0.2515 |
| R.m.s deviations | |
| bond (Å)/ angle (°) | 0.007/0.910 |
| Average B-values (Å²) | 38.19 |
| Ramachandran plot (%) | |
| Favored / Additional allowed | 97.7/2.3 |
| Generously allowed | 0.0 |

^{a}The numbers in parentheses are the statistics from the highest resolution shell.

TABLE 12

Summary of biosensors in this work

| Biosensor | Analytical target | Dynamic range^{a} | LOD (nM) | Detection range (nM) |
|---|---|---|---|---|
| lucCageBim | Bcl-2 | 200% | 0.2 | 0.2-12.5 |
| lucCageBoT | Botulinum neurotoxin B | 130% | 0.4 | 0.4-50 |
| lucCageProA | Fc domain | 350% | 0.39 | 0.39-12.5 |
| lucCagHer2 | Her2 receptor | 380% | 0.23 | 0.23-25 |
| lucCageTrop | Troponin I | 250% | 0.009 | 0.009-0.3 |
| lucCageHBV | Anti-HBV antibody (HzKR127-3.2) | 98% | 2 | 2-100 |
| lucCageHBVα | Anti-HBV antibody (HzKR127-3.2) | 215% | 0.26 | 0.26-100 |
| lucCageHBV+ HzKR127-3.2 | PreS1 | 80% | 0.6 | 0.6-100 |
| lucCageSARS2-M | anti-SARS-Cov-M | 50% | 2.9 | 2.9-250 |
| lucCageSARS2-N | anti-SARS-Cov-NP | 70% | 3.0 | 3.0-100 |
| lucCageRBD | SARS-CoV-2 RBD | 1700% | 0.015 | 0.015-6 |

^{a}Defined as intensiometric change (ΔE/Eₘᵢₙ) of total bioluminescence intensity. ΔE is the maximal change in total bioluminescence emission at saturated target concentration and Eₘᵢₙ is the emission in the absence of the analytical target.

TABLE 13

List of sensing domains used in this work

| | | |
|---|---|---|
| Biosensors | Sensing domain | Sensing domain sequence |
| lucCageBim | Bim | EIWIAQELRRIGDEFNAYYAAA (SEQ ID NO:27380) |
| lucCageBoT | Bot.0671.2 | MFAELKAKFFLEIGDRDAARNALRKAGYSDEEAER IIRKYELE (SEQ ID NO:27381) |
| lucCageProA | Protein A domain C (SpaC) | EQQNAFYEILHLPNLTEEQRNGFIQSLKDDPSVSK EILAEAKKLNDAQAPK (SEQ ID NO:27382) |
| lucCagHer2 | Her2 affibody | EMRNAYWEIALLPNLNNQQKRAFIRSLYDDPSQSA NLLAEAKKLNDAQAPK (SEQ ID NO:27383) |
| lucCageTrop | cTnI + cTnC | EDQLREKAKELWQTIYNLEAEKFDLQEKFKQQKYE INVLRNRINDNQKVSKTKDDSKGKSEEELSDLFRM FDKNADGYIDLEELKIMLQATGETITEDDIEELMK DGDKNNDGRIDYDEFLEFMKGVE (SEQ ID |
| | | NO:27384) |
| lucCageHBV | preS1 (a.a. 35-46) | GANSNNPDWDFN (SEQ ID NO:27629) |
| lucCageHBVα | preS1 (a.a. 35-46) 2x | GANSNNPDWDFNGGSGGGSSGFGANSNNPDWDFNP N (SEQ ID NO:27630) |
| lucCageSARS2-M | SARS-CoV-2 nucleocapsid protein (a.a. 369-382) 2x | MADSNGTITVEELKKLLEGGSGGMADSNGTITVEE LKKLLE (SEQ ID NO:27392) |
| lucCageSARS2-N | SARS-CoV-2 membrane protein (a.a. 1-17) 2x | KKDKKKKADETQALGGSGGKKDKKKKADETQAL (SEQ ID No:27548) |
| lucCageRBD | LCB1_delta4 | ILQKIYEIMRLLDELGHAEASMRVSDLIYEFMKKG DERLLEEAERLLEEVER (SEQ ID NO:27590) |

### Example 2

### Expanding the Universal Readouts for LOCKR-Based Biosensors

The abovementioned sensor platform can be repurposed to accommodate almost all split reporters where one complementary reporter fragment is genetically fused onto the N-terminal of the cage and the other fragment to the C-terminal of the latch (intramolecular) or key (intermolecular). Various types of split-protein pairs or RET pairs (FIG. 16) can enable a wide range of readouts, such as bioluminescence (firefly¹, Renilla², and Gaussia³ luciferase), bioluminescence resonance energy transfer⁴⁻⁶ (BRET), bimolecular fluorescence complementation^{7,8} (BiFC), fluorescence resonance energy transfer (FRET), colorimetry (β-lactamase⁹, β-galactosidase¹⁰, and horseradish peroxidase¹¹), cell survival (dihydrofolate reductase¹²), electrochemical (APEX2¹³), radioactive (thymidine kinase¹⁴), and molecular barcode reporter (TEV protease¹⁵).

The de novo switch platforms of the disclosure can be generalizable and customized to detect arbitrary targets of interest, but can also be reprogramed with a wide range of readouts for different sensing purposes. For cellular imaging, sensors with BiFC or FRET readout can provide excellent spatiotemporal resolution to monitoring the dynamic of intracellular target. In the broad synthetic biology field, the sensors can, for example, 1) facilitate multiplex cell-based assays that use genetic biosensors for drug discovery; 2) profile chemical or genetic perturbations on target-selective pathway using molecular barcodes (TEV protease) with next-generation sequencing (NGS) as the readout technology; and 3) conduct cell survival selection by dihydrofolate reductase (DHFR) complementation in the presence of chosen target. For in vivo imaging, the biological activities and protein targets can be monitored by split-luminescent proteins or by positron emission tomography (PET) with split-thymidine kinase, which allow for imaging in deep tissue. For poi applications, colorimetry readout provides the most convenient setup since no instrument is required for signal acquisition. Besides, an electrochemical readout is readily compatible with the most successful POC device - glucometer, which can read the electrochemical signal for the detection of low-abundance target. Overall, we anticipate that the combination of our de novo sensor design, binder design, and split-protein reassembly can lead to a veritable explosion of applications with user-defined inputs.

To provide proof of concept, we designed an intermolecular BRET sensor (S0512) to detect HBV antibody where teLuc was genetically fused to the cage and CyOFP was tethered to the C-terminal of the key (FIG. 17A). Two copies of epitope sequences were threaded on the latch. In the presence of HBV antibody, ~5% ratiometric change (580/450 nm) was observed with a limit of detection ~11 nM. Meanwhile, we also design an intramolecular BRET sensor (S0622) containing teLuc (BRET donor) and CyOFP (BRET acceptor) on the N- and C- terminal of cage. The design leads to high initial BRET efficiency. In the presence of HBV antibody, the antibody-latch driving force will break the interaction of cage-latch and then increase the distance of BRET donor and acceptor, leading to a decrease in BRET efficiency (FIG. 17B). The limit of detection of S0622 was determined to be ~11 nM with ~20% 450/580 nm ratiometric change. To improve the ratiometric change, we optimized the linker length between key and CyOFP. B0622_6 showed the highest initial BRET efficiency. Up to ~207% 450/580 nm ratiometric change was observed while the sensor retained low nM sensitivity (FIG. 17C). Again, the dynamic range and sensitivity of sensor can be modulated by the key concentration, which is one of the tunable factors in our modular sensor platform.

To expand the readout for point-of-care application, we utilized the split β-lactamase to report the assembly of cage and key upon the actuation. Reconstituted β-lactamase is able to catalyze the hydrolysis of a colorimetric substrate - Nitrocefin, thereby giving reddish product (OD 490). This colorimetry readout is advantageous over optical readout for point-of-care applications because the color change can be directly distinguished by human eyes. Compare to flash type bioluminescence, which generally shows the bursting emission causing a significant complexity on time-dependent signal acquisition, the resultant colorimetric product accumulates in solution overtime. Therefore, it is an end-point assay (more active β-lactamase reaches to the end-point faster). Notably, β-lactamase can remain active in biological fluid e.g., serum and urine¹⁹. The critical design insight here is to lower the background activity as much as possible to reduce the chance of false positives. We demonstrate the conversion of lucCageTrop to LacATrop by simply m [symbol] fusion and a Key-LacB fusion (FIG. 18). The β-lactamase activities were turned on in the presence of human cardiac Troponin I (cTnI). Good standard curves were obtained with low nM sensitivity and the color change from yellow to red can be easily determined by human eyes.

Design sequence:

S0512 (teLuc sequence in bold font; underline HBV
epitopes):MGSHHHHHHGSGSENLYFQGSGGVFTLEDFVGDWRQTAGY NLSQVLEQGGVSSLFQNLGVSVTPIQRIVLSGENGLKIDIHVIIPYEGLS GDQMGQIEKIFKWYPVDNHHFKVILHYGTLVIDGVTPNMIDYFGRPYEGI AVFDGKKITVTGTLWNGNKIIDERLINPDGSLLFRVTINGVTGWRLHERI LAGS(SELARKLLEASTKLQRLNIRLAEALLEAIARLQELNLELVYLAVE
LTDPKRIRDEIKEVKDKSKEIIRRAEKEIDDAAKESEKILEEAREAISGS
GSELAKLLLKAIAETQDLNLRAAKAFLEAAAKLQELNIRAVELLVKLTDP
ATIREALEHAKRRSKEIIDEAERAIRAAKRESERIIEEARRLIEKGSGSG
SELARELLRAHAQLQRLNLELLRELLRALAQLQELNLDLLRLASEL)TDP
DEARKAANSNNPDWDFIVEDAERLIREAAAAANSNNPDWDFLIR (SEQ
ID NO:27651)

Key-2GGSGG-CyOFP (CyOFP sequence in bold font):

MDPDEARKAIARVKRESKRIVEDAERLIREAAAASEKISREAERLIREAAAASEKISREGGSGG
GGVSK GEELIK ENMRSKLYLE GSVNGHQFKC THEGEGKPYE GKQTNRIKVV EGGPLPFAFD
ILATHFMYGS KVFIKYPADL PDYFKQSFPE GFTWERVMVF EDGGVLTATQ DTSLQDGELI
YNVKVRGVNF PANGPVMQKK TLGWEPSTET MYPADGGLEG RCDKALKLVG GGHLHVNFKT
TYKSKKPVKM PGVHYVDRRL ERIKEADNET YVEQYEHAVA RYSNLGGGMD ELYK (SEQ ID
NO: 27622)

B0622 (teLuc sequence in bold font; CyOFP sequence bold and underlined; underline HBV epitopes):

MGSHHHHHHGSGSENLYFQGSGGVFTLEDFVGDWRQTAGYNLSQVLEQGGVSSLFQNLGVSVTPIQRI
VLSGENGLKIDIHVIIPYEGLSGDQMGQIEKIFKWYPVDNHHFKVILHYGTLVIDGVTPNMIDYFGR
PYEGIAVFDGKKITVTGTLWNGNKIIDERLINPDGSLLFRVTINGVTGWRLHERILAGS(SKEAAKKL
QDLNIELARKLLEASTKLQRLNIRLAEALLEAIARLQELNLELVYLAVELTDPKRIRDEIKEVKDKSK
EIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKR
ESERIIEEARRLIEKAKEESERIIREGSGSGDPDIKKLQDLNIELARELLRAHAQLQRLNLELLRELL
RALAQLQELNLDLLRLASEL)TDPDEARKAANSNNPDWDFIVEDAERLIREAAAASEKISREAERLAN
SNNPDWDFISRE VSKGEELIK ENMRSKLYLE GSVNGHQFKC THEGEGKPYE GKQTNRIKVV
EGGPLPFAFD ILATHFMYGS KVFIKYPADL PDYFKQSFPE GFTWERVMVF EDGGVLTATQ
DTSLQDGELI YNVKVRGVNF PANGPVMQKK TLGWEPSTET MYPADGG]
GGHLHVNFKT TYKSKKPVKM PGVHYVDRRL ERIKEADNET YVEQYEHA
ELYK (SEQ ID NO:27652)

B0622 _4:

MGSHHHHHHGSGSENLYFQGSGGVFTLEDFVGDWRQTAGYNLSQVLEQGGVSSLFQNLGVSVTPIQRI
VLSGENGLKIDIHVIIPYEGLSGDQMGQIEKIFKWYPVDNHHFKVILHYGTLVIDGVTPNMIDYFGR
PYEGIAVFDGKKITVTGTLWNGNKIIDERLINPDGSLLFRVTINGVTGWRLHERILAGS(SKEAAKKL
QDLNIELARKLLEASTKLQRLNIRLAEALLEAIARLQELNLELVYLAVELTDPKRIRDEIKEVKDKSK
EIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKR
ESERIIEEARRLIEKAKEESERIIREGSGSGDPDIKKLQDLNIELARELLRAHAQLQRLNLELLRELL
RALAQLQELNLDLLRLASEL)TDPDEARKAANSNNPDWDFIVEDAERLIREAAAASEKISREAERLAN
SNNPDWDFISRE EELIK ENMRSKLYLE GSVNGHQFKC THEGEGKPYE GKQTNRIKVV
EGGPLPFAFD ILATHFMYGS KVFIKYPADL PDYFKQSFPE GFTWERVMVF EDGGVLTATQ
DTSLQDGELI YNVKVRGVNF PANGPVMQKK TLGWEPSTET MYPADGGLEG RCDKALKLVG
GGHLHVNFKT TYKSKKPVKM PGVHYVDRRL ERIKEADNET YVEQYEHAVA RYSNLGGMD
ELYK (SEQ ID NO:27653)

B0622 _6:

MGSHHHHHHGSGSENLYFQGSGGVFTLEDFVGDWRQTAGYNLSQVLEQGGVSSLFQNLGVSVTPIQRI
VLSGENGLKIDIHVIIPYEGLSGDQMGQIEKIFKWYPVDNHHFKVILHYGTLVIDGVTPNMIDYFGR
PYEGIAVFDGKKITVTGTLWNGNKIIDERLINPDGSLLFRVTINGVTGWRLHERILAGS(SKEAAKKL
QDLNIELARKLLEASTKLQRLNIRLAEALLEAIARLQELNLELVYLAVELTDPKRIRDEIKEVKDKSK
EIIRRAEKEIDDAAKESKKILEEARKAIRDAAEESRKILEEGSGSGSDALDELQKLNLELAKLLLKAI
AETQDLNLRAAKAFLEAAAKLQELNIRAVELLVKLTDPATIRRALEHAKRRSKEIIDEAERAIRAAKR
ESERIIEEARRLIEKAKEESERIIREGSGSGDPDIKKLQDLNIELARELLRAHAQLQRLNLELLRELL
RALAQLQELNLDLLRLASEL)TDPDEARKAANSNNPDWDFIVEDAERLIREAAAASEKISREAERLAN
SNNPDWDFISRE LIK ENMRSKLYLE GSVNGHQFKC THEGEGKPYE GKQTNRIKVV
EGGPLPFAFD ILATHFMYGS KVFIKYPADL PDYFKQSFPE GFTWERVMVF EDGGVLTATQ
DTSLQDGELI YNVKVRGVNF PANGPVMQKK TLGWEPSTET MYPADGGLEG RCDKALKLVG
GGHLHVNFKT TYKSKKPVKM PGVHYVDRRL ERIKEADNET YVEQYEHAVA RYSNLGGMD
ELYK (SEQ ID NO:27654)

Key-LacB (split β-lactamase B in bold):

SGSGDPDEARKAIARVKRESKRIVEDAERLIREAAAASEKISREAERLIREAAAASEKISRESGGGGS
GGGGSGGGG LLTLASRQQLIDWME ADKVAGPLLR SALPAGWFIA DKSGAGERGS
RGIIAALGPD GKPSRIVVIY TTGSQATMDE RNRQIAEIGA SLIKHW
27623)

LacATrop (split β-lactamase A in bold; underline cTnT and cTnC):

MGSHHHHHHGSGSENLYFQGSGGSVFAHPETLVK VKDAEDQLGA RVGYIELDLN
SGKILESFRP EERFPMMSTF KVLLCGAVLS RVDAGQEQLG RRIHYSQNDL
VEYSPVTEKH LTDGMTVREL CSAAITMSDN TAANLLLTTI GGPKELTAFL
HNMGDHVTRL DRWEPELNEA IPNDERDTTT PAAMATTLRK LLTGENGR
SGGGGSGGGGSGGGG(SKEAAKKLQDLNIELARKLLEASTKLQRLNIRLAEALLEAIARLQELNLELV
YLAVELTDPKRIRDEIKEVKDKSKEIIRRAEKEIDDAAKESKKILEEARKAIRDAAEESRKILEEGSG
SGSDALDELQKLNLELAKLLLKAIAETQDLNLRAAKAFLEAAAKLQELNIRAVELLVKLTDPATIRRA
LEHAKRRSKEIIDEAERAIRAAKRESERIIEEARRLIEKAKEESERIIREGSGSGDPDIKKLQDLNIE
LARELLRAHAQLQRLNLELLRELLRALAQLQELNLDLLRLASEL)TDPDEARKAIAVTGYRLFEEILD
AERLIREAAAASEDQLREAAKELWQTIYNLEAEKFDLQEKFKQQKYEINVLRNRINDNQKVSKTKDDS
KGKSEEELSDLFRMFDKNADGYIDLEELKIMLQATGETITEDDIEELMKDGDKNNDGRIDYDEFLEFM
KGVE (SEQ ID NO:27620)

References:
(1) Luker, K. E.; Smith, M. C.; Luker, G. D.; Gammon, S. T.; Piwnica-Worms, H.; Piwnica-Worms, D. Proc Natl Acad Sci USA 2004, 101, 12288-12293.
(2) Kaihara, A.; Kawai, Y.; Sato, M.; Ozawa, T.; Umezawa, Y. Anal Chem 2003, 75, 4176-4181.
(3) Remy, I.; Michnick, S. W. Nat Methods 2006, 3, 977-979.
(4) Chu, J.; Oh, Y.; Sens, A.; Ataie, N.; Dana, H.; Macklin, J. J.; Laviv, T.; Welf, E. S.; Dean, K. M.; Zhang, F.; Kim, B. B.; Tang, C. T.; Hu, M.; Baird, M. A.; Davidson, M. W.; Kay, M. A.; Fiolka, R.; Yasuda, R.; Kim, D. S.; Ng, H. L., et al. Nat Biotechnol 2016, 34, 760-767.
(5) Yeh, H. W.; Karmach, O.; Ji, A.; Carter, D.; Martins-Green, M. M.; Ai, H. W. Nat Methods 2017, 14, 971-974.
(6) Yeh, H. W.; Xiong, Y.; Wu, T.; Chen, M.; Ji, A.; Li, X.; Ai, H. W. Acs Chem Biol 2019, 14, 959-965.
(7) Zhou, J.; Lin, J.; Zhou, C.; Deng, X.; Xia, B. Acta Biochim Biophys Sin (Shanghai) 2011, 43, 239-244.
(8) Ohashi, K.; Kiuchi, T.; Shoji, K.; Sampei, K.; Mizuno, K. Biotechniques 2012, 52, 45-50.
(9) Galarneau, A.; Primeau, M.; Trudeau, L. E.; Michnick, S. W. Nat Biotechnol 2002, 20, 619-622.
(10) Wehrman, T. S.; Casipit, C. L.; Gewertz, N. M.; Blau, H. M. Nat Methods 2005, 2, 521-527.
(11) Martell, J. D.; Yamagata, M.; Deerinck, T. J.; Phan, S.; Kwa, C. G.; Ellisman, M. H.; Sanes, J. R.; Ting, A. Y. Nat Biotechnol 2016, 34, 774-780.
(12) Remy, I.; Michnick, S. W. Proc Natl Acad Sci USA 1999, 96, 5394-5399.
(13) Han, Y.; Branon, T. C.; Martell, J. D.; Boassa, D.; Shechner, D.; Ellisma Chem Biol 2019, 14, 619-635.
(14) Massoud, T. F.; Paulmurugan, R.; Gambhir, S. S. Nat Med 2010, 16, 921-926.
(15) Wehr, M. C.; Holder, M. V.; Gailite, I.; Saunders, R. E.; Maile, T. M.; Ciirdaeva, E.; Instrell, R.; Jiang, M.; Howell, M.; Rossner, M. J.; Tapon, N. Nat Cell Biol 2013, 15, 61-U132.
(16) Landry, C. R.; Levy, E. D.; Abd Rabbo, D.; Tarassov, K.; Michnick, S. W. Cell 2013, 155, 983-989.
(17) Bowes, J.; Brown, A. J.; Hamon, J.; Jarolimek, W.; Sridhar, A.; Waldron, G.; Whitebread, S. Nat Rev Drug Discov 2012, 11, 909-922.
(18) Geyer, P. E.; Holdt, L. M.; Teupser, D.; Mann, M. Mol Syst Biol 2017, 13, 942.
(19) Adamson, H.; Ajayi, M. O.; Campbell, E.; Brachi, E.; Tiede, C.; Tang, A. A.; Adams, T. L.; Ford, R.; Davidson, A.; Johnson, M.; McPherson, M. J.; Tomlinson, D. C.; Jeuken, L. J. C. ACS Sens 2019, 4, 3014-3022.

### Example 3

As exemplified in FIG. 20, (Panel A) the above-mentioned sensor platform can be repurposed to accommodate an “indirect detection” approach, in which the split reporter protein (intermolecular or intramolecular embodiments; an intermolecular embodiment is shown below) is reconstituted by pre-incubation of the biosensor with the target (exemplified by an antibody) for the target binding polypeptide, resulting in luminescence activation in this example. The activated biosensor is then incubated with a sample to detect the presence of an antigen to which the antibody binds, resulting in binding of the antibody to the antigen, loss of interaction between the split reporter protein components, and reduction/elimination of reporting activity (in this case, loss of luminescence activity). As will be clear based on the disclosure herein, this embodiment can be used for indirect detection of any analyte of interest. This approach is not limited to using antibodies and their cognate antigens. In another embodiment (Panel B) the split reporter protein (intermolecular or intramolecular embodiments; an intermolecular embodiment is shown below) is reconstituted by pre-incubation of the biosensor with the target (exemplified by the SARS-CoV-2 Spike protein) for the target binding polypeptide, resulting in luminescence activation in this example. The activated biosensor is then incubated with a sample to detect the presence of an inhibitor (exemplified by LCB1 inhibitor) to which the Spike binds, resulting in binding of the antibody to the antigen, loss of interaction between the split reporter protein components, and reduction/elimination of reporting activity (in this case, loss of luminescence activity). This approach can be used for detection of an inhibitor, but also as a tool to evaluate the inhibitory potency of multiple variants. As will be clear based on the disclosure l [symbol] embodiment can be used for indirect detection of any analyte of interest. Another example is shown in FIG. 21.

### Exemplary Uses

Diagnostic sensors herein (lucCageBim, lucCageBot, lucCageTrop, lucCageProA, lucCageHer2, lucCageHBV, lucCageSARS2-M, lucCageSARS2-N) and measured the activation kinetics of each in response to all of their targets (Bcl-2, botulinum neurotoxin B, cardiac Troponin I, IgG Fc, Her2, anti-HBV (HzKR127-3.2), the anti-SARS-M polyclonal antibody (3527,), the anti-SARS-N monoclonal antibody (18F629.1)). Each sensor responded rapidly and sensitively to its cognate target, but not to any others (FIG. 19C). Through SeroNet, the best CoV LOCKR Diagnostics can be formatted into various POCDs (FIG. 19D).

LOCKR Diagnostic combinations that activate chemiluminescence in the presence of anti-coronavirus “anti-epitope” specific antibodies from drop of blood or serum, and that can be turned off by addition of an antigen that contains the epitope of interest are exemplified in FIG. 22.

### Example 4

SARS -CoV-2 infection is thought to often start in the nose, with virus replicating there for several before spreading to the broader respiratory system. Delivery of a high concentration of a viral inhibitor into the nose and into the respiratory system generally could therefore potentially provide prophylactic protection, and therapeutic efficacy early in infection, and could be particularly useful for health care workers and others coming into frequent contact with infected individuals. A number of monoclonal antibodies are in development as systemic SARS-CoV-2 therapeutics, but these compounds are not ideal for intranasal delivery as antibodies are large and often not extremely stable molecules, and the density of binding sites is low (two per 150Kd antibody); the Fc domain provides little added benefit. More desirable would be protein inhibitory with the very high affinity for the virus of the monoclonals, but with higher stability and very much smaller size to maximize the density of inhibitory domains and enable direct delivery into the respiratory system through nebulization.

We set out to de novo design high affinity binders to the RBD that compete with Ace2 binding. We explored two strategies: first we attempted to scaffold the alpha helix in Ace2 that makes the majority of the interactions with the RBD in a small des [symbol] makes additional interactions with the RBD to attain higher affinity, ana second, we sougnt to design binders completely from scratch that do not incorporate any known binding interaction with the RBD. An advantage of the second approach is that the range of possibilities for design is much larger, and so potentially higher affinity binding modes can be identified. For the first approach, we used the Rosetta™ blue print builder to generate small proteins which incorporate the Ace2 helix and for the second approach, RIF docking and design using large miniprotein libraries. The designs interact with distinct regions of the RBD surface surrounding the Ace2 binding sites. Designs for approach 1, and approach 2, were encoded in long oligonucleotides, and screened for binding to fluorescently tagged RBD on the yeast cell surface. Deep sequencing identified 3 Ace2 helix scaffolded designs (approach 1), and 150 de novo interface designs (approach 2) that were clearly enriched following FACS sorting for RBD binding. Designs were expressed in E. coli and purified, and many were found to be have soluble expression and to bind RBD in biolayer interferometry experiments and could effectively compete with ACE-2 for binding to RBD (example shown in FIG. 2). Based on BLI data the RBD binding affinities of minibinders are: LCB1 <1 nM, LCB3 <1 nM. The affinities of LCB2, LCB4, LCB5, LCB6, LCB7, LCB8 range from 1~20nM, with relative strength of different binders being LCB4 > LCB2 > LCB9 = LCB5 > LCB6 > LCB7.

To determine whether the designs binding the RBD through the designed interfaces, site saturation libraries in which every residue in each design was substituted with each of the 20 amino acids one at a time were constructed, and subjected to FACS sorting for RBD binding. Deep sequencing showed that the binding interface residues and protein core residues were conserved in many of the designs for which such site saturation libraries (SSM’s) were constructed (SSMs were used to define allowable positions for amino acid changes in Table 3 ). For most of the designs, a small number of substitutions were enriched in the FACS sorting, suggesting they increase binding affinity for RBD. For the highest affinity of the approach 1 designs, and 8 of the approach 2 designs, combinatorial libraries incorporating these substitutions were constructed and again screened for binding with FACS; because of the very high binding affinity the concentrations used in the sorting were as low as 20pM. Each library converged on a small number of closely related sequences, and for each design, one of the optimized variants was expressed in E. coli and purified.

The binding of the 8 optimized designs with different binding modes to RBD was investigated by biolayer interferometry. For a number of the designs, the Kd’s ranged from 1-20 nM, and for the remainder, the Kd’s were below 1 nM, too strong t [symbol] with this technique. Circular dichroism spectra of the designs were consistent with the design models, and the designs retained full binding activity after a number of days at room temperature.

We investigated the ability of the designs to block infection of human cells by live virus. 100 FFU of SARS-CoV-2 was added to 2.5-3x10^4 vero cells in the presence of varying amounts of the designed binders. We observed potent inhibition of infection for all of the designs with IC50′s ranging from 1 nM to 0.02 nM.

The designed binders have several advantages over antibodies as potential therapeutics. Together, they span a range of binding modes, and in combination viral escape would be quite unlikely. The retention of activity after extended time at elevated temperatures suggests they would not require a cold chain. The designs are 20 fold smaller than a full antibody molecule, and hence in an equal mass have 20 fold more potential neutralizing sites, increasing the potential efficacy of a locally administered drug. The cost of goods and the ability to scale to very high production should be lower for the much simpler miniproteins, which unlike antibodies, do not require expression in mammalian cells for proper folding. The small size and high stability should make them amenable to direct delivery into the respiratory system by nebulization. Immunogenicity is a potential problem with any foreign molecule, but for previously characterized small de novo designed proteins little or no immune response has been observed, perhaps because the high solubility and stability together with the small size makes presentation on dendritic cells less likely.

References
1. Yuan M, Wu NC, Zhu X, Lee CD, So RTY, Lv H, Mok CKP, Wilson IA: A highly conserved cryptic epitope in the receptor binding domains of SARS-CoV-2 and SARS-CoV. Science 2020, 368(6491):630-633.
2. Case JB, Rothlauf PW, Chen RE, Liu Z, Zhao H, Kim AS, Bloyet L-M, Zeng Q, Tahan S, Droit L et al: Neutralizing antibody and soluble ACE2 inhibition of a replication-competent VSV-SARS-CoV-2 and a clinical isolate of SARS-CoV-2. bioRxiv 2020:2020.2005.2018.102038.

We claim:

1. A cage protein comprising a helical bundle, wherein the cage protein comprises a structural region and a latch region, wherein the latch region comprises one or more target binding polypeptide, wherein the cage protein further comprises a first reporter protein domain, wherein the first reporter protein domain undergoes a detectable change in reporting activity when bound to a second split reporter protein domain, and wherein the structural region interacts with the latch region to prevent solution access to the one or more target binding polypeptide.

2. The cage protein of claim 1, further comprising the second reporter protein domain, wherein one of the first reporter protein domain and the second reporter domain is present in the latch region and the other is present in the structural region, wherein an interaction of the first reporter protein domain and the second reporter protein domain is diminished in the presence of target to which the one or more target binding polypeptide binds.

3. The cage protein of claim 1, wherein the second reporter protein domain is not present in the cage protein.

4. The cage protein of any one of claims 1-3, wherein the helical bundle comprises between 2-9, 2-8, 2-7, 3-9, 3-8, 3-7, 4-9, 4-8, 4-7, 5-9, 5-8, 5-7, 6-9, 6-8, 6-7, 2-6, 3-6, 4-6, 5-6, 2-5, 3-5, 4-5, 2-4, 3-4, 2-3, 2, 3, 4, 5, 6, 7, 8, or 9 alpha helices.

5. The cage protein of any one of claims 1-4, wherein each helix in the structural region is independently between 18-60, 18-55, 18-50, 18-45, 22-60, 22-55, 22-50, 22-45, 25-60, 25-55, 25-50, 25-45, 28-60, 28-55, 28-50, 28-45, 32-60, 32-55, 32-50, 32-45, 35-60, 35-55, 35-50, 35-45, 38-60, 38-55, 38-50, 38-45, 40-60, 40-58, 40-55, 40-50, or 40-45 amino acids in length.

6. The cage protein of any one of claims 1-5, comprising amino acid linkers connecting adjacent alpha helices.

7. The cage protein of claim 6, wherein the amino acid linkers are independently between 2 and 10 amino acids in length, not including any further functional sequences that may be fused to the linker, or independently 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-7, 3-7, 4-7, 5-7, 6-7, 2-6, 3-6, 4-6, 5-6, 2-5, 3-5, 4-5, 2-4, 3-4, 2-3, 2, 3, 4, 5, 6, 7, 8, 9, or 10 amino acids in length.

8. The cage protein of any one of claims 1-7, wherein the latch region is at the C-terminus of the cage protein. In other embodiments, the latch region may be at the N-terminus of the cage protein.

9. The cage protein of any one of claims 1-8, wherein the first reporter protein domain is present in the latch region.

10. The cage protein of claim 9, wherein the second reporter protein, when present, is present in the structural region.

11. The cage protein of claim 10, wherein the second reporter protein is at the N-terminus of the structural region, or is within 10, 9, 8, 7, 6, 5, 4, 3, 2, or 1 amino acid of the N-terminus of the structural region.

12. The cage protein of claim 9, wherein the one or more target binding polypeptide and the first reporter protein domain are separated by at least 10 amino acids in the latch region.

13. The cage protein of any one of claims 1-12, wherein the one or more target binding polypeptide is at or within 10, 9, 8, 7, 6, 5, 4, 3, 2, or 1 amino acid of the C-terminus of the latch region.

14. The cage protein of any one of claims 1-12, wherein the first reporter protein domain is at the C-terminus of the latch region or within 20, 19, 18, 17, 16, 15, 14, 13, 12, 11, 10, 9, 8, 7, 6, 5, 4, 3, 2, or 1 amino acid of the C-terminus of the latch region, or wherein the first reporter protein domain is at the N-terminus of the latch region or within 20, 19, 18, 17, 16, 15, 14, 13, 12, 11, 10, 9, 8, 7, 6, 5, 4, 3, 2, or 1 amino acid of the N-terminus of the latch region.

15. The cage protein of any one of claims 1-14, wherein the first reporter protein domain, and the second reporter domain when present, comprise reporter protein domains selected from the group consisting of luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease).

16. The cage protein of any one of claims 1-15 wherein the latch region is at the C-terminus of the cage protein.

17. The cage protein of any one of claims 1-15, wherein the latch region is at the N-terminus of the cage protein.

18. The cage protein of any one of claims 1-8, wherein the first reporter protein domain is present in the latch region.

19. The cage protein of claim 18, wherein the second reporter protein, when present, is present in the structural region.

20. The cage protein of claim 19, wherein the second reporter protein is at the N-terminus of the structural region, or is within 10, 9, 8, 7, 6, 5, 4, 3, 2, or 1 amino acid of the N-terminus of the structural region.

21. The cage protein of claim 18, wherein the one or more target binding polypeptide and the first reporter protein domain are separated by at least 10 amino acids in the latch region.

22. The cage protein of any one of claims 1-21, wherein the one or more target binding polypeptide is at or within 10, 9, 8, 7, 6, 5, 4, 3, 2, or 1 amino acid of the C-terminus of the latch region.

23. The cage protein of any one of claims 1-21, wherein the first reporter protein domain is at the C-terminus of the latch region.

24. The cage protein of any one of claims 1-23, wherein the first reporter protein domain, and the second reporter domain when present, comprise a split reporter protein domain from a reporter selected from the group consisting of luciferase (including but not limited to firefly, Renilla, and Gaussia luciferase), bimolecular fluorescence complementation (BiFC) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease).

25. The cage protein of any one of claims 1-24, wherein the cage does not include the second split reporter domain, wherein the first split reporter protein domain comprises:
(a) an amino acid sequence at least 70%, 75%, 80%, 85%, 90%, 91%, 92%, 93%, 94%, 95%, 96%, 97%, 98%, or 100% identical to the amino acid sequence of SEQ ID NO: 27359 and 27664-27672;
(b) an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS: 27360-27361:
VFAHPETL VKVKDAEDQLGA RVGYIELDLN SGKILESFRP EERFPMMSTF KVLLCGAVLS RVDAGQEQLG RRIHYSQNDL VEYSPVTEKH LTDGMTVREL CSAAITMSDN TAANLLLTTI GGPKELTAFL HNMGDHVTRL DRWEPELNEA IPNDERDTTT PAAMATTLRK LLTGENGR (split β-lactamase A; SEQ ID NO:27360) and
LLTLASRQQLIDWME ADKVAGPLLR SALPAGWFIA DKSGAGERGS RGIIAALGPD GKPSRIVVIY TTGSQATMDE RNRQIAEIGA SLIKHW (Split beta lactamase B; SEQ ID NO:27361);
(c) an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27362-27378, wherein underlined residues are amino acid linkers or other optional residues that may be present or absent, and when present may be any amino acid sequence, and wherein any N-terminal methionine residues may be present or absent:
VFTLEDFVGDWRQTAGYNLSQVLEQGGVSSLFQNLGVSVTPIQRIVLSGENGLKIDIHVIIPYEGLSG DQMGQIEKIFKVVYPVDNHHFKVILHYGTLVIDGVTPNMIDYFGRPYEGIAVFDGKKITVTGTLWNGN KIIDERLINPDGSLLFRVTINGVTGWRLHERILA (TeLuc; SEQ ID NO: 27362) (full luminescent or fluorescent protein that can be used to create FRET and/or BRET sensors);
LIK ENMRSKLYLE GSVNGHQFKC THEGEGKPYE GKQTNRIKW EGGPLPFAFD ILATHFMYGS KVFIKYPADL PDYFKQSFPE GFTWERVMVF EDGGVLTATQ DTSLQDGELI YNVKVRGVNF PANGPVMQKK TLGWEPSTET MYPADGGLEG RCDKALKLVG GGHLHVNFKT TYKSKKPVKM PGVHYVDRRL ERIKEADNET YVEQYEHAVA RYSNLGGMD ELYK (CyOFP variant; SEQ ID NO: 27363) (full luminescent or fluorescent protein that can be used to create FRET and/or BRET sensors);
VSKGEELIK ENMRSKLYLE GSVNGHQFKC THEGEGKPYE GKQTNRIKW EGGPLPFAFD ILATHFMYGS KVFIKYPADL PDYFKQSFPE GFTWERVMVF EDGGVLTATQ DTSLQDGELI YNVKVRGVNF PANGPVMQKK TLGWEPSTET MYPADGGLEG RCDKALKLVG GGHLHVNFKT TYKSKKPVKM PGVHYVDRRL ERIKEADNET YVEQYEHAVA RYSNLGGMD ELYK (CyOFP variant SEQ ID NO: 27364) (full luminescent or fluorescent protein that can be used to create FRET and/or BRET sensors);
EELIK ENMRSKLYLE GSVNGHQFKC THEGEGKPYE GKQTNRIKW EGGPLPFAFD ILATHFMYGS KVFIKYPADL PDYFKQSFPE GFTWERVMVF EDGGVLTATQ DTSLQDGELI YNVKVRGVNF PANGPVMQKK TLGWEPSTET MYPADGGLEG RCDKALKLVG GGHLHVNFKT TYKSKKPVKM PGVHYVDRRL ERIKEADNET YVEQYEHAVA RYSNLGGMD ELYK (CuOFP variant; SEQ ID NO: 27365) (full luminescent or fluorescent protein that can be used to create FRET and/or BRET sensors);
KVFTLGDFVGDWRQTAGYNQAQVLEQGGLTSLFQNLGVSVTPIQRIVLSGENGLKIDIHV IIPYEGLSCDQMAQIEKIFKVVYPVDDHHFKAILHYGTLVIDGVTPNMIDYFGQPYEGIA KFDGKKITVTGTLWNGNTIIDERLINPDGSLLFRVTINGVTGWRLHERILA (LumiLuc; SEQ ID NO: 27366) (full luminescent or fluorescent protein that can be used to create FRET and/or BRET sensors);
MVSKGEEDNM ASLPATHELH IFGSINGVDF DMVGQGTGNP NDGYEELNLK STKGDLQFSP W ILVPHIGYG FHQYLPYPDG MSPFQAAMVD GSGYQVHRTM QFEDGASLTV NYRYTYEGSH IKG EAQVKGT GFPADGPVMT NSLTAADWCR SKKTYPNDKT IISTFKWSYT TGNGKRYRST ARTTY TFAKP MAANYLKNQP MYVFRKTELK HSKTELNFKE WQKAFTDVMG MDELYK (mNeonGreen; SEQ ID NO:27367) (full luminescent or fluorescent protein that can be used to create FRET and/or BRET sensors);
MVSKGEAVIK EFMRFKVHME GSMNGHEFEI EGEGEGRPYE GTQTAKLKVT KGGPLPFSWD ILSPQFMYGS RAFIKHPADI PDYYKQSFPE GFKWERVMNF EDGGAVTVTQ DTSLEDGTL I YKVKLRGTNF PPDGPVMQKK TMGWEASTER LYPEDGVLKG DIKMALRLKD GGRYLADF KT TYKAKKPVQM PGAYNVDRKL DITSHNEDYT WEQYERSEG RHSTGGMDEL YK (mScarlet-i; SEQ ID NO: 27368) (full luminescent or fluorescent protein that can be used to create FRET and/or BRET sensors);
SGKSYPTVSADYQKAVEKAKKRLGGFIAEKRCAPLMLRLAWHSAGTFDKRTKTGGPFGTIRYPAELAH SANSGLDIAVRLLEPLKAEFPILSYADFYQLAGVVAVEVTGGPEVPFHPGREDKPELPPEGRLPDATK GSDHLRDVFGKAMGLTDQDIVALSGGHTLGAAHKERSGFEGPWTSNPLVFDNSYFTELLSGEKEGGGG SGGGGS (APEX2-1-200; SEQ ID NO: 27369);
GGGGSGGGGS GLLQLPSDKALLSDPVFRPLVDKYAADEDAFFADYAEAHQKLSELGFADA (APEX2-201-250; SEQ ID NO: 27370);
MGSHHHHHHGSGSENLYFQGSGGS VRPLNCIVA VSQNMGIGKN GDLPWPPLRN ESKYFQRMTT TSSVEGKQNL VIMGRKTWFS IPEKNRPLKD RINIVLSREL KEPPRGAHFL AKSLDDALRL IEQPELGGGGSGGGGS (DHFR A (1-105); SEQ ID NO:27371);
SGSGDPDEARKAIARVKRESKRIVEDAERLIREAAAASEKISREAERLIREAAAASEKISRE GGGGSGGGGS ASKV DMVWIVGGSS VYQEAMNQPG HLRLFVTRIM QEFESDTFFP EIDLGKYKLL PEYPGVLSEV QEEKGIKYKF EVYEKKD (DHFR_B (106-186); SEQ ID NO: 27372);
QLTPTFYDNSCPNVSNIVRDIIVNELRSDPRIAASILRLHFHDCFVNGCDASILLDNTTSFRTEKDAF GNANSARGFSVIDR MKAAVESACPGTVSCADLLTIAAQQSVTLAGGPSWRVPLGRRDSLQAFLDLANANLPAPFFTLPQLKD SFRNVGLNRSSDLVALSGGHTFGKSQCRFIMDRLYNFSNTGLPDPTLNTTYLQTLRGLCPLNGGSGS (sHRPa is the large split HRP fragment. It consists of amino acids 1-213 of horseradish peroxidase (HRP) with the following 4 mutations: T21I, P78S, R93G, N175S) (SEQ ID NO: 27373);
NLSALVDFDLRTPTIFDNKYYVNLEEQKGLIQSDQELFSSPDATDTIPLVRSFANSTQTFFNAFVEAM DRMGNITPLTGTQGQIRRNCRVVNSNGGSGS (sHRPb is the small split HRP fragment. It consists of amino acids 214-308 of horseradish peroxidase (HRP) with the following 2 mutations: N255D, L299R) (SEQ ID NO: 27374);
GESLFKGPRDYNPISSTICHLTNESDGHTTSLYGIGFGPFIITNKHLFRRNNGTLLVQSLHGVFKVKN TTTLQQHLIDGRDMIIIRMPKDFPPFPQKLKFREPQREERICLVTTNFQTGGGGSGGGGS (N Tev (1-118) (SEQ ID NO:27375);
GGGGSGGGGSKSMSSMVSDTSCTFPSSDGIFWKHWIQTKDGQCGSPLVSTRDGFIVGIHSASNFTNTN NYFTSVPKNFMELLTNQEAQQWVSGWRLNADSVLWGGHKVFMDKP C_Tev (119-221) (SEQ ID NO:27376);
MASYPCHQHA SAFDQAARSR GHSNRRTALR PRRQQEATEV RLEQKMPTLL RVYIDGPHGM GKTTTTQLLV ALGSRDDIVY VPEPMTYWQV LGASETIANI YTTQHRLDQG EISAGDAAVV MTSAQITMGM PYAVTDAVLA PHIGGEAGSS HAPPPALTLI FDRHPIAALL CYPAARYLMG SMTPQAVLAF VALIPPTLPG TNIVLGALPE DRHIDRLAKR QRPGERLDLA MLAAIRRVYG LLANTVRYLQ GGGSWREDWG QLSGT GGGGSGGGGS (thymidine kinase_TK_A (1-265) (SEQ ID NO: 27377) ; and/or
GGGGSGGGGS AVPPQ GAEPQSNAGP RPHIGDTLFT LFRAPELLAP NGDLYNVFAW ALDVLAKRLR PMHVFILDYD QSPAGCRDAL LQLTSGMVQT HVTTPGSIPT ICDLARTFAR EMGEAN (thymidine kinase_TK_B (266-376) (SEQ ID NO: 27378).

26. The cage protein of any one of claims 1-24, wherein the cage comprises the second split reporter protein domain, wherein
(a) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NOS: 27359, and 27664-27672;
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27379, wherein the N-terminal methionine residue may be present or absent: MVFTLEDFVGDWEQTAAYNLDQVLEQGGVSSLLQNLAVSVTPIQRIVRSGENALKIDIHIIPYEGLSADQMAQIE EVFKWYPVDDHHFKVILPYGTLVIDGVTPNMLNYFGRPYEGIAVFDGKKITVTGTLWNGNKIIDERLITPDGSM LFRVTINS (LgBiT) (SEQ ID NO: 27379) ;
(b) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27360
VFAHPETL VKVKDAEDQLGA RVGYIELDLN SGKILESFRP EERFPMMSTF KVLLCGAVLS RVDAGQEQLG RRIHYSQNDL VEYSPVTEKH LTDGMTVREL CSAAITMSDN TAANLLLTTI GGPKELTAFL HNMGDHVTRL DRWEPELNEA IPNDERDTTT PAAMATTLRK LLTGENGR (split β-lactamase A) (SEQ ID NO: 27360),
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27361:
LLTLASRQQLIDWME ADKVAGPLLR SALPAGWFIA DKSGAGERGS RGIIAALGPD GKPSRIVVIY TTGSQATMDE RNRQIAEIGA SLIKHW (Split beta lactamase B) (SEQ ID NO: 27361) ;
(c) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27362:
VFTLEDFVGDWRQTAGYNLSQVLEQGGVSSLFQNLGVSVTPIQRIVLSGENGLKIDIHVIIPYEGLSG DQMGQIEKIFKVVYPVDNHHFKVILHYGTLVIDGVTPNMIDYFGRPYEGIAVFDGKKITVTGTLWNGN KIIDERLINPDGSLLFRVTINGVTGWRLHERILA (TeLuc) (SEQ ID NO: 27362), (full luminescent or fluorescent protein that can be used to create FRET and/or BRET sensors)
and the other comprises an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27363-27365:
LIK ENMRSKLYLE GSVNGHQFKC THEGEGKPYE GKQTNRIKW EGGPLPFAFD ILATHFMYGS KVFIKYPADL PDYFKQSFPE GFTWERVMVF EDGGVLTATQ DTSLQDGELI YNVKVRGVNF PANGPVMQKK TLGWEPSTET MYPADGGLEG RCDKALKLVG GGHLHVNFKT TYKSKKPVKM PGVHYVDRRL ERIKEADNET YVEQYEHAVA RYSNLGGMD ELYK (CyOFP variant) (SEQ ID NO: 27363) (full luminescent or fluorescent protein that can be used to create FRET and/or BRET sensors);
VSKGEELIK ENMRSKLYLE GSVNGHQFKC THEGEGKPYE GKQTNRIKW EGGPLPFAFD ILATHFMYGS KVFIKYPADL PDYFKQSFPE GFTWERVMVF EDGGVLTATQ DTSLQDGELI YNVKVRGVNF PANGPVMQKK TLGWEPSTET MYPADGGLEG RCDKALKLVG GGHLHVNFKT TYKSKKPVKM PGVHYVDRRL ERIKEADNET YVEQYEHAVA RYSNLGGMD ELYK(CyOFP variant) (SEQ ID NO: 27364) (full luminescent or fluorescent protein that can be used to create FRET and/or BRET sensors); and
EELIK ENMRSKLYLE GSVNGHQFKC THEGEGKPYE GKQTNRIKW EGGPLPFAFD ILATHFMYGS KVFIKYPADL PDYFKQSFPE GFTWERVMVF EDGGVLTATQ DTSLQDGELI YNVKVRGVNF PANGPVMQKK TLGWEPSTET MYPADGGLEG RCDKALKLVG GGHLHVNFKT TYKSKKPVKM PGVHYVDRRL ERIKEADNET YVEQYEHAVA RYSNLGGMD ELYK(CyOFP variant) (SEQ ID NO: 27365) (full luminescent or fluorescent protein that can be used to create FRET and/or BRET sensors);
(d) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27366:
KVFTLGDFVGDWRQTAGYNQAQVLEQGGLTSLFQNLGVSVTPIQRIVLSGENGLKIDIHV IIPYEGLSCDQMAQIEKIFKVVYPVDDHHFKAILHYGTLVIDGVTPNMIDYFGQPYEGIA KFDGKKITVTGTLWNGNTIIDERLINPDGSLLFRVTINGVTGWRLHERILA (LemiLuc) (SEQ ID NO: 27366) (full luminescent or fluorescent protein that can be used to create FRET and/or BRET sensors),
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27368, wherein the N-terminal methionine residue may be present or absent:
MVSKGEAVIK EFMRFKVHME GSMNGHEFEI EGEGEGRPYE GTQTAKLKVT KGGPLPFSWD ILSPQFMYG S RAFIKHPADI PDYYKQSFPE GFKWERVMNF EDGGAVTVTQ DTSLEDGTLI YKVKLRGTNF PPDGPVM QKK TMGWEASTER LYPEDGVLKG DIKMALRLKD GGRYLADFKT TYKAKKPVQM PGAYNVDRKL DITSH NEDYT WEQYERSEG RHSTGGMDEL YK (mScarlet-i) _(SEQ ID NO:27368) (full luminescent or fluorescent protein that can be used to create FRET and/or BRET sensors);
(e) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27367, wherein the N-terminal methionine residue may be present or absent:
MVSKGEEDNM ASLPATHELH IFGSINGVDF DMVGQGTGNP NDGYEELNLK STKGDLQFSP WILVPHIGY G FHQYLPYPDG MSPFQAAMVD GSGYQVHRTM QFEDGASLTV NYRYTYEGSH IKGEAQVKGT GFPADGP VMT NSLTAADWCR SKKTYPNDKT IISTFKWSYT TGNGKRYRST ARTTYTFAKP MAANYLKNQP MYVFR KTELK HSKTELNFKE WQKAFTDVMG MDELYK (mNeonGreen) (SEQ ID NO:27367), (full luminescent or fluorescent protein that can be used to create FRET and/or BRET sensors),
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27368, wherein the N-terminal methionine residue may be present or absent:
MVSKGEAVIK EFMRFKVHME GSMNGHEFEI EGEGEGRPYE GTQTAKLKVT KGGPLPFSWD ILSPQFMYG S RAFIKHPADI PDYYKQSFPE GFKWERVMNF EDGGAVTVTQ DTSLEDGTLI YKVKLRGTNF PPDGPVM QKK TMGWEASTER LYPEDGVLKG DIKMALRLKD GGRYLADFKT TYKAKKPVQM PGAYNVDRKL DITSH NEDYT WEQYERSEG RHSTGGMDEL YK (mScarlet-i) (SEQ ID NO: 27368) (full luminescent or fluorescent protein that can be used to create FRET and/or BRET sensors);
(f) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence SEQ ID NO: 27369, wherein underlined residues are optional residues that may be present or absent, and when present may be any amino acid sequence
SGKSYPTVSADYQKAVEKAKKRLGGFIAEKRCAPLMLRLAWHSAGTFDKRTKTGGPFGTIRYPAELAHSANSGLD IAVRLLEPLKAEFPILSYADFYQLAGWAVEVTGGPEVPFHPGREDKPELPPEGRLPDATKGSDHLRDVFGKAMG LTDQDIVALSGGHTLGAAHKERSGFEGPWTSNPLVFDNSYFTELLSGEKEGGGGSGGGGS (APEX2-1-200) (SEQ ID NO: 27369) (split engineered variant of soybean ascorbate peroxidase protein for chemiluminescent and colorimetric detection system);
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27370, wherein underlined residues are optional residues that may be present or absent, and when present may be any amino acid sequence
GGGGSGGGGS GLLQLPSDKALLSDPVFRPLVDKYAADEDAFFADYAEAHQKLSELGFADA (APEX2-201-250) (SEQ ID NO: 27370) (split engineered variant of soybean ascorbate peroxidase protein for chemiluminescent and colorimetric detection system);
(g) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27371, wherein underlined residues are optional residues that may be present or absent, and when present may be any amino acid sequence
MGSHHHHHHGSGSENLYFQGSGGS VRPLNCIVA VSQNMGIGKN GDLPWPPLRN ESKYFQRMTT TSSVEGKQNL VIMGRKTWFS IPEKNRPLKD RINIVLSREL KEPPRGAHFL AKSLDDALRL IEQPELGGGGSGGGGS (DHFR_A (1-105)); (SEQ ID NO: 27371) (split dihydrofolate reductase protein reporter for cell survival or fluorescence)
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27372, wherein underlined residues are optional residues that may be present or absent, and when present may be any amino acid sequence
SGSG DPDEARKAIARVKRESKRIVEDAERLIREAAAASEKISREAERLIREAAAASEKISRE GGGGSGGGGS ASKV DMVWIVGGSS VYQEAMNQPG HLRLFVTRIM QEFESDTFFP EIDLGKYKLL PEYPGVLSEV QEEKGIKYKF EVYEKKD (DHFR_B (106-186)) ; (SEQ ID NO: 27372) (split dihydrofolate reductase protein reporter for cell survival or fluorescence);
(h) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27373, wherein underlined residues are optional residues that may be present or absent, and when present may be any amino acid sequence
QLTPTFYDNSCPNVSNIVRDIIVNELRSDPRIAASILRLHFHDCFVNGCDASILLDNTTSFRTEKDAFGNANSA RGFSVIDRMKAAVESACPGTVSCADLLTIAAQQSVTLAGGPSWRVPLGRRDSLQAFLDLANANLPAPFFTLPQLK DSFRNVGLNRSSDLVALSGGHTFGKSQCRFIMDRLYNFSNTGLPDPTLNTTYLQTLRGLCPLNGGSGS (sHRPa is the large split HRP fragment. It consists of amino acids 1-213 of horseradish peroxidase (HRP) with the following 4 mutations: T21I, P78S, R93G, N175S: plasmid 73147 (SEQ ID NO: 27373);
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27374, wherein underlined residues are optional residues that may be present or absent, and when present may be any amino acid sequence
NLSALVDFDLRTPTIFDNKYYVNLEEQKGLIQSDQELFSSPDATDTIPLVRSFANSTQTFFNAFVEAMDRMGNIT PLTGTQGQIRRNCRVVNSNGGSGS (sHRPb is the small split HRP fragment. It consists of amino acids 214-308 of horseradish peroxidase (HRP) with the following 2 mutations: N255D, L299R: plasmid 73148) (SEQ ID NO: 27374);
(i) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27375, wherein underlined residues are optional residues that may be present or absent, and when present may be any amino acid sequence
GESLFKGPRDYNPISSTICHLTNESDGHTTSLYGIGFGPFIITNKHLFRRNNGTLLVQSLHGVFKVKNTTTLQQH LIDGRDMIIIRMPKDFPPFPQKLKFREPQREERICLVTTNFQTGGGGSGGGGSNTev (1-118) (SEQ ID NO: 27375) (Split TEV protease);
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27376, wherein underlined residues are optional residues that may be present or absent, and when present may be any amino acid sequence
GGGGSGGGGSKSMSSMVSDTSCTFPSSDGIFWKHWIQTKDGQCGSPLVSTRDGFIVGIHSASNFTNTNNYFTSV PKNFMELLTNQEAQQWVSGWRLNADSVLWGGHKVFMDKP (c_Tev (119-221)) ; (SEQ ID NO: 27376) ( Split TEV protease);
(j) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27377, wherein underlined residues are optional residues that may be present or absent, and when present may be any amino acid sequence, and wherein the N-terminal methionine residue may be present or absent:
MASYPCHQHA SAFDQAARSR GHSNRRTALR PRRQQEATEV RLEQKMPTLL RVYIDGPHGM GKTTTTQLLV ALGSRDDIVY VPEPMTYWQV LGASETIANI YTTQHRLDQG EISAGDAAVV MTSAQITMGM PYAVTDAVLA PHIGGEAGSS HAPPPALTLI FDRHPIAALL CYPAARYLMG SMTPQAVLAF VALIPPTLPG TNIVLGALPE DRHIDRLAKR QRPGERLDLA MLAAIRRVYG LLANTVRYLQ GGGSWREDWG QLSGT GGGGSGGGGS (thymidine kinase_TK_A (1-265)) (SEQ ID NO: 27377) ;
and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27378, wherein underlined residues are optional residues that may be present or absent, and when present may be any amino acid sequence
GGGGSGGGGS AVPPQ GAEPQSNAGP RPHIGDTLFT LFRAPELLAP NGDLYNVFAW ALDVLAKRLR PMHVFILDYD QSPAGCRDAL LQLTSGMVQT HVTTPGSIPT ICDLARTFAR EMGEAN (thymidine kinase_TK_B (266-376) (SEQ ID NO: 27378) .

27. The cage protein of any one of claims 1-26, wherein the one or more target binding polypeptide is capable of binding to a target including but not limited to an antibody, a toxin, a diagnostic biomarker, a viral particle, a disease biomarker, a metabolite or a biochemical analyte.

28. The cage protein of any one of claims 1-26, wherein the one or more target binding polypeptide is capable of binding to an antibody target.

29. The cage protein of claim 28, wherein the one or more target binding polypeptide comprises one or more epitope recognized by antibodies against a viral target.

30. The cage protein of claim 28 or 29, wherein the one or more target binding polypeptide comprises one or more epitope recognized by antibodies against SARS-Cov-2.

31. The cage protein of any one of claims 126, wherein the one or more target binding polypeptide is capable of binding to a disease marker or toxin.

32. The cage protein of any one of claims 1-26, wherein the one or more target binding polypeptide is capable of binding to Bcl-2, Her2 receptor, Botulinum neurotoxin B, albumin, epithelial growth factor receptor, prostate-specific membrane antigen (PSMA), citrullinated peptides, brain natriuretic peptides, and/or cardiac Troponin I.

33. The cage protein of any one of claims 1-32, wherein the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27380-27430.

34. The cage protein of claim 33, wherein the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:27397-27430, or selected from SEQ ID NOS:27397-27406, 27409-27416, and 27427-27430.

35. The cage protein of claim 34, wherein amino acid substitutions relative to the reference target binding polypeptide amino acid are selected from the allowable amino acid substitutions provided in Table 3.

36. The cage protein of claim 34 or 35, wherein interface residues are identical to those in the reference target binding polypeptide or are conservatively substituted relative to interface residues in the reference target binding polypeptide as detailed in Table 2.

37. The cage protein of any one of claims 34-36, wherein the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27397-27406 and 27431-27466.

38. The cage protein of claim 37, wherein the one or more target binding polypeptide comprises an amino acid substitution relative to the amino acid sequence of SEQ ID NO:27397 at 1, 2, 3, 4, 5, 6, 7, 8, 9, 10, 11, 12, 13, 14, 15, 16, 17, or all 18 residues selected from the group consisting of 2, 4, 5, 14, 15, 17, 18, 27, 28, 32, 37, 38, 39, 41, 42, 49, 52, and 55.

39. The cage protein of claim 38, wherein the substitutions in the one or more target binding polypeptide are selected from the substitutions listed in Table 5, either individually or in combinations in a given row.

40. The cage protein of any one of claims 34-36, wherein the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27409-27416 and 27467-27493.

41. The cage protein of claim 40, wherein the target binding comprises an amino acid substitution relative to the amino acid sequence of SEQ ID NO:27409 at 1, 2, 3, 4, 5, 6, 7, 8, 9, 10, 11, 12, 13, 14, 15, 16, 17, 18, 19, or all 20 residues selected from the group consisting 2, 6, 8, 9, 13, 14, 19, 22, 25, 26, 28, 29, 34, 35, 37, 40, 43, 45, 49, and 62.

42. The cage protein of claim 41, wherein the substitutions are selected from the substitutions listed in Table 7, either individually or in combinations in a given row.

43. The cage protein of any one of claims 34-36, wherein the target binding comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 27427-27430 and 27494.

44. The cage protein of claim 44, wherein the one or more target binding polypeptide comprises an amino acid substitution relative to the amino acid sequence of SEQ ID NO:27429 at or both residues selected from the group consisting 63 and 75.

45. The cage protein of claim 44, wherein the substitutions comprise R63A and/or K75T.

46. The cage protein of any one of claims 1-45, comprising the amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical to the amino acid sequence of a cage protein selected from the group consisting of SEQ ID NOS:1-49, 51-52, 54-59, 61, 65, 67-91, 92 -2033, 2034-14317, 27094-27117, 27120-27125, 27,278 to 27,321, even-numbered SEQ ID NOS between SEQ ID NOS: 27126 and 27276, and cage proteins listed in Tables 8 and 9, not including optional amino acid residues (, and not including amino acid residues in the latch region, and wherein the N-terminal and/or C-terminal 60 amino acids of each cage protein may be optional.

47. The cage protein of any one of claims 1-47, comprising the amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues, to the amino acid sequence of a cage protein selected from the consisting of SEQ ID NOS: 27497-27620, wherein the N-terminal protein purification tag (MGSHHHHHHGSGSENLYFQGSGG (SEQ ID NO:27624); or MGSHHHHHHGSENLYFQG (SEQ ID NO:27625); or GSHHHHHHGSGSENLYFQG (SEQ ID NO:27626) ) is optional, are not considered in the percent identity comparison, and can be present or absent and preferably are absent.

48. A key protein capable of binding to the structural region of a cage protein of any one of claims 1-47 that does not include the second reporter protein domain, wherein binding of the key protein to the cage protein only occurs in the presence of a target to which the cage protein one or more target binding polypeptide can bind, wherein the key protein comprises a second reporter protein domain, wherein interaction of the key protein second reporter protein domain and the cage protein first reporter protein domain causes a detectable change in reporting activity from the first reporter protein domain .

49. The key protein of claim 48, wherein the second reporter protein domain is at the N-terminus or the C-terminus of the key protein, or is within 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acid of the N-terminus or the C-terminus of the key protein.

50. The key protein of any one of claims 48-49, wherein the second reporter protein domain comprises a reporter protein domain selected from the group consisting of luciferase (including but not limited to firefly, Renilla, and Gaussia luciferase), bioluminescence resonance energy transfer (BRET) reporters, bimolecular fluorescence complementation (BiFC) reporters, fluorescence resonance energy transfer (FRET) reporters, colorimetry reporters (including but not limited to β-lactamase, β-galactosidase, and horseradish peroxidase), cell survival reporters (including but not limited to dihydrofolate reductase), electrochemical reporters (including but not limited to APEX2), radioactive reporters (including but not limited to thymidine kinase), and molecular barcode reporters (including but not limited to TEV protease).

51. The key protein of any one of claims 48-50, wherein the second reporter protein domain comprises an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27360-23379, wherein underlined residues are optional residues that may be present or absent, and when present may be any amino acid sequence, and wherein any N-terminal methionine residue may be present or absent.

52. The key protein of any one of claims 48-51, wherein the key protein, not including the second split reporter protein domain, comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues, to the amino acid sequence selected from the group consisting of SEQ ID NOS:14318-26601, 26602-27015, 27016-27050, 27,322 to 27,358, and key polypeptides with an odd-numbered SEQ ID NO between SEQ ID NOS: 27127 and 27277), key proteins in Table 8, and key proteins in Table 9.

53. The key protein of any one of claims 48-52, wherein the key protein comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99%, or 100% identical, not including optional amino acid residues in parentheses, to the amino acid sequence of a key protein selected from the group consisting of SEQ ID NOS: 27621-27623, wherein residues in parentheses are optional and may be present or absent.
> lucKey: MGS-(His)6-TEV site-linker-LgBit-linker-latch sequence
(MGSHHHHHHGSGSENLYFQG)SGMVFTLEDFVGDWEQTAAYNLDQVLEQGGVSSLLQNLAVSVTPIQRIVRSGE NALKIDIHIIPYEGLSADQMAQIEEVFKVVYPVDDHHFKVILPYGTLVIDGVTPNMLNYFGRPYEGIAVFDGKKI TVTGTLWNGNKIIDERLITPDGSMLFRVTINSGGSGGGGSGGGSGGSDEARKAIARVKRESKRIVEDAERLIREA
AAASEKISREAERLIREAAAASEKISRE (SEQ ID NO:27621)
Key-2GGSGG-CyOFP (CyOFP sequence in bold/underline):
(M)DPDEARKAIARVKRESKRIVEDAERLIREAAAASEKISREAERLIREAAAASEKISREGGSGG GGVSK GEELIK ENMRSKLYLE GSVNGHQFKC THEGEGKPYE GKQTNRIKW EGGPLPFAFD ILATHFMYGS KVFIKYPADL PDYFKQSFPE GFTWERVMVF EDGGVLTATQ DTSLQDGELI YNVKVRGVNF PANGPVMQKK TLGWEPSTET MYPADGGLEG RCDKALKLVG GGHLHVNFKT TYKSKKPVKM PGVHYVDRRL ERIKEADNET YVEQYEHAVA RYSNLGGGMD ELYK (SEQ ID NO: 27622)
Key-LacB (split β-lactamase B in bold/underline):
SGSGDPDEARKAIARVKRESKRIVEDAERLIREAAAASEKISREAERLIREAAAASEKISRESGGGGSGGGGSGG
GG LLTLASRQQLIDWME ADKVAGPLLR SALPAGWFIA DKSGAGERGS RGIIAALGPD GKPSRIVVIY TTGSQATMDE RNRQIAEIGA SLIKHW (SEQ ID NO: 27623).

54. A biosensor, comprising
(a) the cage protein of any one of claims 1-47 wherein the cage does not include the second reporter protein domain; and
(b) the key protein of any one of claims 48-53;
wherein the key protein can only bind to the cage protein in the presence of a target to which the cage protein one or more target binding polypeptide can bind; and wherein binding of the first reporter protein domain of the cage protein to the second reporter protein domain of the key protein causes a detectable change in reporting activity from the first reporter protein domain.

55. The biosensor of claim 54, wherein
(a) the first reporter protein domain comprises an amino acid sequence at least 70%, 75%, 80%, 85%, 90%, 91%, 92%, 93%, 94%, 95%, 96%, 97%, 98%, or
100% identical to the amino acid sequence SEQ ID NO: 27359, and 27664-27672; and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO 27379, wherein the N-terminal methionine residue may be present or absent
(b) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27360,and the other comprises_an amino acid sequence at least 70%, 75%, 80%, 85%, 90%, 91%, 92%, 93%, 94%, 95%, 96%, 97%, 98%, or 100% identical to the amino acid sequence of SEQ ID NO: 27361;
(c) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27362,and the other comprises_an amino acid sequence at least 70%, 75%, 80%, 85%, 90%, 91%, 92%, 93%, 94%, 95%, 96%, 97%, 98%, or 100% identical to the amino acid sequence selected from the group consisting of SEQ ID NOS:27363-27365;
(d) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27366,and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO 27368:
(e) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27367, wherein the N-terminal methionine residue may be present or absent,and the other comprises_an amino acid sequence at least 70%, 75%, 80%, 85%, 90%, 91%, 92%, 93%, 94%, 95%, 96%, 97%, 98%, or 100% identical to the amino acid sequence of SEQ ID NO 27368, wherein the N-terminal methionine residue may be present or absent;
(f) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27369, wherein underlined residues are optional residues that may be present or absent, and when present may be any amino acid sequence; and the other comprises an amino acid sequence at least 70%, 75%, 80%, 85%, 90%, 91%, 92%, 93%, 94%, 95%, 96%, 97%, 98%, or 100% identical to the amino acid of SEQ ID NO:27370, wherein underlined residues are optional residues that may be present or absent, and when present may be any amino acid sequence;
(g) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27371, wherein underlined residues are optional residues that may be present or absent, and when present may be any amino acid sequence, and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO: 27372, wherein underlined residues are optional residues that may be present or absent, and when present may be any amino acid sequence;
(h) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27373, wherein underlined residues are optional residues that may be present or absent, and when present may be any amino acid sequence, and the other comprises_an amino acid sequence at least 70%, 75%, 80%, 85%, 90%, 91%, 92%, 93%, 94%, 95%, 96%, 97%, 98%, or 100% identical to the amino acid sequence of SEQ ID NO:27374, wherein underlined residues are optional residues that may be present or absent, and when present may be any amino acid sequence;
(i) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27375, wherein underlined residues are optional residues that may be present or absent, and when present may be any amino acid sequence, and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27376, wherein underlined residues are optional residues that may be present or absent, and when present may be any amino acid sequence;
(j) one of the first reporter protein domain and the second reporter protein domain comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27377, wherein the N-terminal methionine residue may be present or absent, and wherein underlined residues are optional residues that may be present or absent, and when present may be any amino acid sequence, and the other comprises an amino acid sequence at least 70%, 75%, 80%, 85%, 90%, 91%, 92%, 93%, 94%, 95%, 96%, 97%, 98%, or 100% identical to the amino acid sequence of SEQ ID NO:27378, wherein underlined residues are optional residues that may be present or absent, and when present may be any amino acid sequence.

56. The biosensor of claim 54, wherein the cage protein comprises a cage protein of claim 47 and the key protein comprises an amino acid sequence at least 70%, 75%, 80%, 85%, 90%, 91%, 92%, 93%, 94%, 95%, 96%, 97%, 98%, or 100% identical, not including optional amino acid residues in parentheses, to the amino acid sequence of SEQ ID NO: 27621.
> lucKey: MGS-(His)6-TEV site-linker-LgBit-linker-latch sequence (MGSHHHHHHGSGSENLYFQG)SGMVFTLEDFVGDWEQTAAYNLDQVLEQGGVSSLLQNLAVSVTPIQRIVRSGE NALKIDIHIIPYEGLSADQMAQIEEVFKVVYPVDDHHFKVILPYGTLVIDGVTPNMLNYFGRPYEGIAVFDGKKI TVTGTLWNGNKIIDERLITPDGSMLFRVTINSGGSGGGGSGGGSGGSDEARKAIARVKRESKRIVEDAERLIREA AAASEKISREAERLIREAAAASEKISRE.

57. The biosensor of claim 54, wherein the cage protein and the key protein comprise a protein pair comprising
(i) a cage protein comprising an amino acid sequence at least 70%, 75%, 80%, 85%, 90%, 91%, 92%, 93%, 94%, 95%, 96%, 97%, 98%, or 100% identical to the amino acid sequence of SEQ ID NO:27620, wherein the residues in parentheses are optional and may be present or absent:
LacATrop (split β-lactamase A in bold; underline cTnT and cTnC) : (MGSHHHHHHGSGSENLYFQG SGGS)VFAHPETLVK VKDAEDQLGA RVGYIELDLN SGKILESFRP EERFPMMSTF KVLLCGAVLS RVDAGQEQLG RRIHYSQNDL VEYSPVTEKH LTDGMTVREL CSAAITMSDN TAANLLLTTI GGPKELTAFL HNMGDHVTRL DRWEPELNEA IPNDERDTTT PAAMATTLRK LLTGENGR SGGGGSGGGGSGGGG(SKEAAKKLQDLNIELARKLLEASTKLQRLNIRLAEALLEAIARLQELNLELVYLAVELT DPKRIRDEIKEVKDKSKEIIRRAEKEIDDAAKESKKILEEARKAIRDAAEESRKILEEGSGSGSDALDELQKLNL ELAKLLLKAIAETQDLNLRAAKAFLEAAAKLQELNIRAVELLVKLTDPATIRRALEHAKRRSKEIIDEAERAIRA AKRESERIIEEARRLIEKAKEESERIIREGSGSGDPDIKKLQDLNIELARELLRAHAQLQRLNLELLRELLRALA QLQELNLDLLRLASEL)TDPDEARKAIAVTGYRLFEEILDAERLIREAAAASEDQLREAAKELWQTIYNLEAEKF DLQEKFKQQKYEINVLRNRINDNQKVSKTKDDSKGKSEEELSDLFRMFDKNADGYIDLEELKIMLQATGETITED DIEELMKDGDKNNDGRIDYDEFLEFMKGVE; and
(ii) a key protein comprising an amino acid sequence at least 70%, 75%, 80%, 85%, 90%, 91%, 92%, 93%, 94%, 95%, 96%, 97%, 98%, or 100% identical to the amino acid sequence of SEQ ID NO:27631.
LLTLASRQQLIDWME ADKVAGPLLR SALPAGWFIA DKSGAGERGS RGIIAALGPD GKPSRIVVIY TTGSQATMDE RNRQIAEIGA SLIKHW (SEQ ID NO:27361).

58. A method for detecting a target, comprising
(a) contacting the cage protein of any one of claims 47 where the cage protein comprises the second reporter protein domain, or the biosensor of any one of claims 40-43 with a biological sample under conditions to promote binding of the cage protein one or more target binding polypeptide to a target present in the biological sample, causing a detectable change in reporting activity from the first reporter protein domain; and
(b) detecting the change in reporting activity from the first reporter protein domain, wherein the change in reporting activity identifies the sample as containing the target.

59. The method of claim 58, wherein the target is selected from the group including but not limited to an antibody, a toxin, a diagnostic biomarker, a viral particle, a disease biomarker, a metabolite or a biochemical analyte.

60. The method of any one of claims 58-59, wherein the target is an antibody.

61. The method of claim 60, wherein the target comprises antibodies selective for a virus.

62. The method of claim 58, wherein the one or more target binding polypeptide comprises the amino acid sequence selected from the group consisting of SEQ ID NO: 27292-27394 and 27547-27548, and a polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 27397-27494.

63. The method of any one of claims 58-62, wherein the cage polypeptide comprises the cage protein of claim 47.

64. The method of any one of claims 58-59, wherein the target is a disease marker or toxin.

65. The method of claim 64, wherein the disease marker or toxin comprises Bcl-2, Her2 receptor, Botulinum neurotoxin B, albumin, epithelial growth factor receptor, prostate-specific membrane antigen (PSMA), citrullinated peptides, brain natriuretic peptides, and/or cardiac Troponin I.

66. The method of claim 64 or 65, wherein the one or more target binding polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 27380-27390, wherein any N-terminal amino acid is optional and may be present or absent.

67. The method of any one of claims 64-66, wherein the cage protein comprises the cage protein of claim 47.

68. A method for designing a biosensor, cage protein, or key protein comprising the steps of any method described herein.

69. A nucleic acid encoding the cage protein or key protein of any of the preceding claims .

70. An expression vector comprising the nucleic acid of claim 69 operatively linked to a suitable control element, such as a promoter.

71. A cell comprising the cage protein, key protein, composition, nucleic acid, or expression vector of any preceding claim.

72. A pharmaceutical composition comprising the cage protein, key protein, composition, nucleic acid, expression vector, or cell of any preceding claim, and a pharmaceutically acceptable carrier.

73. An epitope, comprising or consisting of the amino acid sequence of SEQ ID NO: 27384.

74. A method for detecting Troponin I in a sample, comprising contacting a biological sample with the epitope of claim 73 under conditions suitable to promote binding of Troponin I in the sample to the epitope to form a binding complex, and detecting binding complexes that demonstrate presence of Troponin I in the sample.
